(19) United States
(12) Patent Application Publication
(10) Pub. No.: US 2025/0144099 A1
(43) Pub. Date: May 8, 2025

(54) INK4 TUMOR SUPPRESSOR PROTEINS MEDIATE RESISTANCE TO CDK4/6 KINASE INHIBITORS

(71) Applicants: MEMORIAL SLOAN-KETTERING CANCER CENTER, New York, NY (US); DANA-FARBER CANCER INSTITUTE, Boston, MA (US); STEVENS INSTITUTE OF TECHNOLOGY, Hoboken, NJ (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Sarat Chandarlapaty, New York, NY (US); Qing Li, New York, NY (US); Nathanael Gray, Boston, MA (US); Baishan Jiang, Boston, MA (US); Abhishek Sharma, Hoboken, NJ (US); Aiswarya Mini, Hoboken, NJ (US)

(21) Appl. No.: 18/690,228

(22) PCT Filed: Sep. 8, 2022
(86) PCT No.: PCT/US2022/042928

§ 371 Date: Mar. 7, 2024

Related U.S. Application Data
(60) Provisional application No. 63/241,787, filed on Sep. 8, 2021.

(51) Int. Cl. A61K 31/519; A61P 35/00; C07D 471/04

(52) U.S. Cl. CPC A61K 31/519; A61P 35/00; C07D 471/04

(57) ABSTRACT

The present disclosure relates to compounds according to Formula (I), Formula (I) or a pharmaceutically acceptable salt and/or solvate thereof, compositions including such compounds, and methods useful for treating, preventing, and/or ameliorating a CDK4 and/or CDK6-mediated disorder, disease, or condition (e.g., cancer such as breast cancer) in a subject.

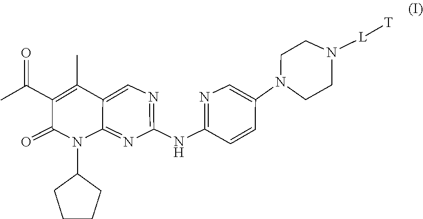

### CROSS-REFERENCE TO RELATED APPLICATIONS

This application claims the benefit of and priority to U.S. Provisional Appl. No. 63/241,787, filed Sep. 8, 2021, the contents of each of which are incorporated herein by reference in their entirety for any and all purposes.

### U.S. GOVERNMENT SUPPORT

This invention was made with government support under P30CA008748, R01GM121505, R01CA218278-03, and R01GM132386 awarded by the National Institutes of Health. The government has certain rights in the invention.

### FIELD

The present technology is directed to bifunctional compounds useful as degraders for CDK4 and/or CDK6, compositions thereof, and methods utilizing such compounds and compositions that are useful for treating, preventing, and/or ameliorating a CDK4 and/or CDK6-mediated disease (e.g., cancer such as breast cancer) in a subject.

### SUMMARY

In an aspect, the present technology provides a compound according to Formula (I)

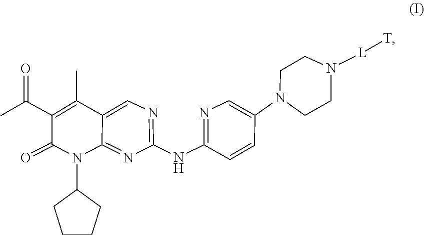

or a pharmaceutically acceptable salt and/or solvate thereof, wherein
   L is selected from the group consisting of

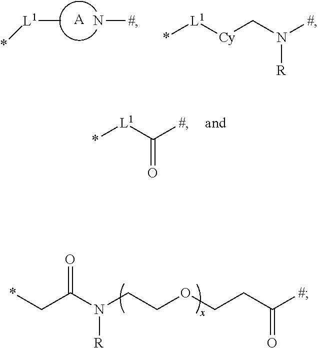

T is selected from the group consisting of

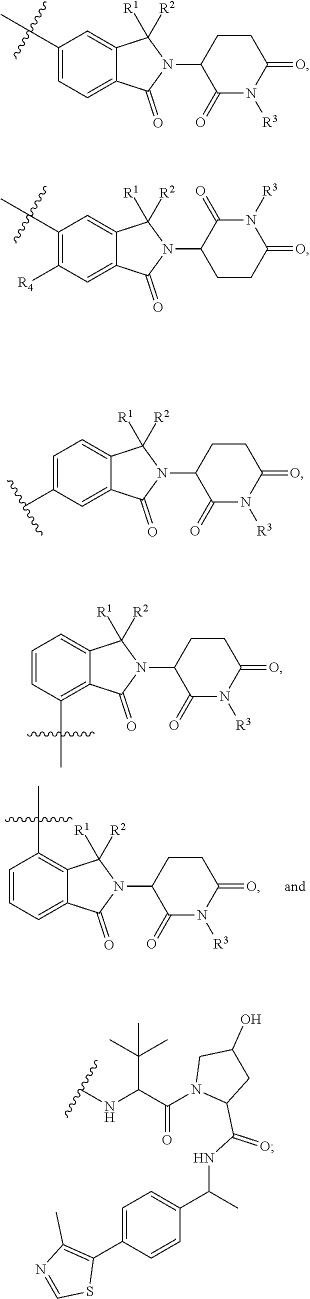

ring A is a 4- to 7-membered N-containing heterocycloalkylene optionally substituted with one or more groups selected from halogen and C₁-C₃ alkyl;
Cy is a C₄-C₆ cycloalkylene optionally substituted with one or more groups selected from halogen and C₁-C₃ alkyl;
R and R³ are each independently H or C₁-C₃ alkyl;
R¹ and R² are both H, or R¹ and R² taken together form an oxo (=O) group;
R⁴ is H, halo, C₁-C₄ alkyl, or C₃-C₆ cycloalkyl;
L¹ is a C₁-C₆ alkylene;
* is a linkage site to the nitrogen atom of the piperazine moiety;
# is a linkage site to the T group; and
x is 1, 2, or 3.

In a related aspect, a composition is provided that includes a compound of any embodiment disclosed herein, a pharmaceutically acceptable carrier or one or more excipients, fillers or agents (collectively referred to hereafter as “pharmaceutically acceptable carrier” unless otherwise indicated and/or specified).

In an aspect, a method for inducing degradation of CDK4 and/or CDK6 in a subject in need thereof is provided that includes administering to the subject a therapeutically effective amount of a compound of any embodiment disclosed herein and optionally a pharmaceutically acceptable carrier.

In a related aspect, a method for treating, preventing, and/or ameliorating a CDK4 and/or CDK6-mediated disorder, disease, or condition in a subject in need thereof is provided that includes administering to the subject a therapeutically effective amount of a compound of any embodiment disclosed herein and optionally a pharmaceutically acceptable carrier.

In further related aspects, a method for treating, preventing, and/or ameliorating breast cancer in a subject in need thereof is provided that includes administering to the subject a therapeutically effective amount of a compound of any embodiment disclosed herein and optionally a pharmaceutically acceptable carrier.

Further aspects and embodiments of the present technology are described herein.

### BRIEF DESCRIPTION OF THE DRAWINGS

FIGS. 1A-IG illustrate INK4-CDK6 complex promotes resistance in cells. FIG. 1A relates to a schematic for analysis of CDK4 and CDK6 interactions and activity via co-immunoprecipitation followed by ADP-Glo kinase assays and mass spectrometry. FIG. 1B shows ADP-Glo kinase assay showing IP-CDK4 and IP-CDK6 kinase activity from MCF-7 parental and CDK6-high cells (cells with FAT1 CRISPR knockout that have high CDK6 expression, previously shown to have resistance to CDK4/6i (8)), with or without 100 nM abemaciclib treatment. Data are shown as means+S.D. of three biologically independent samples. P values were determined by unpaired two-sided Student's t-test. FIG. 1C relates to a Venn diagram showing the number of unique proteins identified by mass spectrometry co-immunoprecipitated from IP-CDK4 and IP-CDK6 in FAT1 loss cells. Percentages were calculated by number of proteins identified in each subgroup divided by total proteins identified by IP of either CDK4 or CDK6. Data are shown as means of three replicates. FIG. 1D shows pathway analysis by Gene Ontology of proteins interacting with CDK6 but not CDK4 in the FAT1 loss cells. The proteins were grouped by their putative biological functions. FIG. 1E shows unique peptide counts of cyclin-dependent kinases and their endogenous inhibitor proteins identified in the Co—IP/mass spectrometry associated with CDK4 or CDK6 in the FAT1 loss cells. N=2. FIG. 1F shows co-immunoprecipitation and immunoblotting illustrating association of p15^{INK4B} and p18^{INK4C} with CDK6, but not CDK4, in CDK6-high cells. FIG. 1G shows cell line screening results illustrating that models with high CDKN2A or low RB1 mRNA expression are correlated with poor response to palbociclib. AUC: area-under-curve of the IC50 curve.

FIGS. 2A-2E illustrate INK4-CDK6 complexes are insensitive to CDK4/6i. FIG. 2A illustrates interface residues in CDK6 in close proximity with INK4 isoforms based on previous INK4-bound CDK6 structures in the Protein Data Bank (PDB) (69) (n.b. no available structure for p15^{INK4B}). CDK6-HA was immunoprecipitated using HA-beads in parental MCF-7 cells, MCF-7 cells expressing HA-WT-CDK6, HA-V16D and R31C mutant CDK6 (disrupted INK4/CDK6 interaction), or HA-K43M-/D163N-mutant CDK6 (kinase dead) and interaction with INK4 proteins was determined by immunoblotting. FIG. 2B illustrates disruption of INK4s and CDK6 binding or impairment of CDK6 kinase activity restores the sensitivity of CDK6-overexpressing cells to CDK4/6 inhibitors. Cells were treated with DMSO or 100 nM abemaciclib for 24 h prior to collection. FIG. 2C illustrates % of cell viability of cells overexpressing wild-type CDK6 or R31C- or D163N-mutant CDK6 treated with increasing concentrations of abemaciclib compared to parental cells. IC₅₀ values were recorded on day 5 following treatment. Data are shown as means±S.D; n=6. FIG. 2D illustrates knockout of p15^{INK4B} and p18^{INK4C} in FAT1 loss cells promotes suppression of Rb phosphorylation in response to abemaciclib to a similar extent as in parental cells. Cells were collected 24 h after 100 nM abemaciclib treatment. Representative blots are shown, which were repeated independently three times. FIG. 2E illustrates the growth rate of p15^{INK4B} and p18^{INK4C} in FAT1 loss cells was inhibited by 100 nM abamaciclib. The cell viability was recorded at day14 and day21. ****p<0.0001. Data are shown as means S.D; n=6.

FIGS. 3A-3F illustrate INK4/CDK6 complexes are insensitive to CDK4/6 inhibitors. FIG. 3A shows in vitro kinase assay utilizing recombinant CDK6/cyclinD3 and Rb substrate demonstrates that preincubation of the complex with p18 (purple) prevents complete inhibition of kinase activity by abemaciclib (LY). Data are shown as means±S.D of two biological replicates. FIG. 3B illustrates effect of preincubation of p18 on CDK6/cyclin D3 in vitro kinase activity. Data are shown as means±S.D of two biological replicates. FIG. 3C shows assay of CDK6/cyclinD3 kinase activity and response to p18 by immunoblotting demonstrating that p18 impairs the ability of abemaciclib to inhibit CDK6 phosphorylation of Rb. FIG. 3D shows computational modeling of the effect of p18 binding to the CDK6 binding pocket expressed as volume change for abemaciclib (top panel) or AMP-PNP (bottom panel). Structures of CDK6: cyclin complex before and after p18 binding are represented by crystallographic structures with PDB ID 2EUF and 1G3N (shown in ribbons). The binding pockets were approximated by spheres (shown in green pointed by red arrows; shown in purple pointed by green arrows). The volume of each binding pocket was quantified using the total volume of the corresponding set of spheres and percentage of changes was calculated. FIG. 3E shows the table summarizing the changes of binding pocket volume for three CDK4/6i (palbociclib, abemaciclib and ribociclib) and AMP-PNP upon binding of INK4s (p16, p18, p19). FIG. 3F shows microscale thermophoresis (MST) assay of CDK6 binding to abemaciclib illustrating the change in Kd as a result of p18 binding (red). Data are shown as means±S.D of two independent measurements.

FIGS. 4A-4J illustrate multiple genetic alterations promote CDK6-mediated resistance in patients. FIG. 4A shows immunohistochemistry of FAT1, CDK6, YAP, p15^{INK4B} and p18^{INK4C} in representative PDX models that are sensitive or resistant to CDK4/6 inhibitors. FIG. 4B shows number of cases that show high or low CDK6 in PDX models that are sensitive or resistant to CDK4/6 inhibitors. Immuno-reactive score (IRS) >2 are recorded as high CDK6 expression. FIG. 4C shows immune-reactive scores of CDK6, nuclear YAP, FAT1, p15 and p18 staining in sensitive and resistant PDX models. FIGS. 4D-4E relate to immunoblotting demonstrating that knockdown of PTEN or ARID1A in MCF-7 cells promotes upregulation of CDK6 and resistance to 100 nM abemaciclib treatment. Cell were treated for 24 h prior to collection. FIG. 4F relates to cell viability (% of control cells) plots showing that both PTEN knockdown cells have decreased sensitivity to abemaciclib compared to parental cells. IC₅₀ values were recorded on day 5. Data are shown as means S.D; n=6. FIG. 4G relates to cell viability (% of control cells) plots showing that ARID1A knockdown cells have decreased sensitivity to abemaciclib compared to parental cells. Knockdown of YAP1 in shARID1A cells restores its sensitivity to abemaciclib. IC₅₀ values were recorded on day 7. Data are shown as means±S.D; n=6. FIG. 4H relates to immunoblotting showing inhibition of AKT (2 M MK-2206) suppresses induction of CDK6 expression in PTEN knockdown cells. I) Immunoblotting showing that knockdown of YAP1 in shARID1A cells decreases CDK6 expression. All blots were repeated at least 3 times and representative blots are shown. FIG. 4J illustrates the pattern, frequency, and type of genomic alterations in CDK6-associated genes in 1366 metastatic tumors from 1115 patients with HR+/HER2− metastatic breast cancer. There is a total of 190 cases showing at least one of the genetic alterations associated with CDK6 upregulation.

FIGS. 5A-5G illustrate compounds targeting the CDK6-INK4 complex inhibit CDK4/6i-resistant tumors. FIG. 5A relates to immunoblotting of MCF-7 parental and cells with high CDK6 expression (CDK6-overexpressing cells and CDK6-high cells with FAT1 loss) treated for 24 h with increasing concentrations of bifunctional degrader compound, BSJ-03-123, demonstrating dose dependent targeting of CDK6 but not CDK4. FIG. 5B relates to assessment of a panel of degrader compounds that target CDK4 and/or CDK6. Immunoblotting after 24 hr drug treatment (500 nM) in FAT1 loss cells shows varying selectivity for CDK4 vs CDK6. Representative blots from three independent experiments are shown. Among them, BSJ-05-017 and BSJ-03-096 show the most significant degradation of both CDK4 and CDK6. FIG. 5C relates to immunoblot depicting dose-responsive effects of BSJ-05-017 in both CDK4/6i sensitive (left) and resistant (right) cells in comparison with palbociclib (500 nM) after 24 h treatment. FIG. 5D shows percentage of growth plot showing BSJ-05-017 inhibits sensitive MCF-7 parental and resistant CDK6-high cells with equal potency, while palbociclib only shows partial inhibition of resistant cells. IC₅₀ values were recorded at day 7. Data are shown as means S.D; n=6. FIG. 5E shows assay for drug-induced senescence (Senescence Green) demonstrating number of senescence-marker positive cells induced by 8 days of treatment with DMSO, BSJ-05-017 (500 nM), abemaciclib (100 nM) and palbociclib (500 nM). BSJ-05-017 induced a significantly higher number of cells into senescence compared with abemaciclib or palbociclib in CDK6-high cells. FIG. 5F relates to immunoblotting showing the degradation of CDK4/6 and decreased phospho-Rb1 and E2F1 levels in CDK6-high (FAT-1 loss) tumor bearing mice administered 25 mg/kg BSJ-05-017 intraperitoneally. Tumors were collected 6 h after 3 consecutive days of vehicle or BSJ-05-017 treatment (N=2). FIG. 5G shows growth curve plots of cell-derived xenografts of MCF7 parental, CDK6-overexpressing and PTEN loss cells. Mice were treated with vehicle, ribociclib (25 mg/kg, p.o.), BSJ-05-017 (50 mg/kg, i.p.) or BSJ-03-096 (50 mg/kg, p.o.) daily for 25-35 days. Tumor volumes were recorded every 3-4 days. Data are shown as means S.D; n=4.

FIGS. 6A-6E illustrate INK4 proteins interact with CDK6 in CDK4/6 inhibitor-resistant cells. FIG. 6A relates to Western blotting showing the phosphorylation of Rb substrate by immunoprecipitated CDK4 and CDK6 from MCF7 parental and FAT1 loss cells. Rb substrate was added to the kinase reaction with or without 100 nM abemaciclib. FIG. 6B relates to Western blotting showing proteins that co-immunoprecipitated with CDK4 and CDK6 in CDK6-overexpressing cells. Phosphorylation of Rb was blotted after kinase assay with Rb substrate. FIGS. 6C-6D illustrate immunoprecipitation of CDK4 and CDK6 in the parental and CDK6-overexpressing T47D and BT474 cells. FIG. 6E illustrates immunoprecipitation of p18 in CDK6-overexpressing cells showing its interaction with cyclin D3. Rb substrate was added and phosphorylation of Rb was shown in the western blot.

FIGS. 7A-7P illustrate interaction of INK4 with CDK6 promotes resistance to CDK4/6 inhibitors. FIG. 7A shows Kinase assay illustrating that immunoprecipitated CDK6 from CDK6-R31C and -V16D mutants phosphorylates Rb to the same level as that from wild-type CDK6. Overexpression of wild-type CDK6, but not CDK6 mutants that block INK4 proteins binding (R31C or V16D) or lack kinase activity (D163N), promotes CDK4/6i resistance in T47D (FIGS. 7B-7C), CAMA-1 (FIGS. 7D-7E), ZR-75-1 (FIGS. 7F-7G) and EFM19 (FIGS. 711-7I) cells. Growth curves showing the suppression of growth by abemaciclib. Western blot showing the overexpression of CDK6 in different cell lines and their phosphorylation of Rb and downstream signaling upon 100 nM abemaciclib treatment. FIG. 7J illustrates growth of cells with overexpression of wild-type CDK6 or CDK6 with R31C or D163N mutation in MCF-7 cells in response to 100 nM palbociclib. FIG. 7K shows Western blot illustrating that the phosphorylation of Rb and downstream cyclin A2 was inhibited by 100 nM palcociclib in cells overexpressing CDK6-WT, but not CDK6-R31C or CDK6-D163N. FIG. 7L shows cell cycle analysis illustrating CDK6-WT cells have increased G1 arrest compared to the parental cells or cells with overexpression of CDK6-R31C and CDK6-D163N mutations. FIG. 7M shows growth curve illustrating knockout of p15 and p18 in T47D CDK6-overexpressing (single clone labeled as T47D CDK6-N1) cells restored its sensitivity to abemaciclib. FIG. 7N relates to Western blot showing the knockout of p15 and p18 in T47D CDK6-overexpressing cells.

FIG. 7O shows inducible overexpression of p16 in T47D cells promotes resistance to abemaciclib and Palbociclib. FIG. 7P shows Western blot illustrating a dose-dependent p16 expression in response to doxycycline. Statistical analysis was performed by two-way ANOVA. *p<0.05, **p<0.01. ****p<0.0001.

FIGS. 8A-8D illustrate in vitro kinase activity of CDK6/cyclin D3. FIG. 8A shows kinase titration using recombinant CDK6/Cyclin D3. Kinase activity using Rb substrate was measured by detection and quantification of luminescence-labeled ADP converted from input ATP. FIG. 8B shows addition of abemaciclib leads to dose dependent inhibition of CDK6/Cyclin D3 kinase activity using same assay as in (A). FIG. 8C shows addition of recombinant p18^{INK4C} suppresses CDK6/cyclin D3 kinase activity. FIG. 8D shows kinase activity of CDK6/cyclin D3 using different amounts of CDK6/Cyclin D3 or p18/CDK6/Cyclin D3. All above experiments were repeated at least three times independently and the representative data were shown. Data are shown as means±S.D., n=2.

FIGS. 9A-9B illustrate mRNA expression of targets in PDXs treated with CDK4/6 inhibitors. FIG. 9A relates to nanostring data showing mRNA levels of CDK6 and CDKN2C are elevated in the PDXs that are resistant to CDK4/6 inhibitors compared to those are sensitive. *p<0.05. Student's t-test was used for statistical analysis. FIG. 9B shows knockdown of ARID1A gene using siRNA in MCF7 cells. Growth assay showing ARID1A loss is resistant to 50 nM and 100 nM abemaciclib treatment. Western blot showing knockdown of ARID1A and upregulation of CDK6.

FIGS. 10A-10D illustrate components of the CDK4/6 degrader library. FIG. 10A relates to cell viability (% of control cells) curve showing BSJ-03-123 does not effectively inhibit the growth of MCF7 parental and CDK6-high cells at day 7. FIG. 10B shows chemical structures of certain CDK4/6 bi-degraders. FIGS. 10C-10D show global proteomics data demonstrating BSJ-05-017 and BSJ-03-096 are highly selective for CDK4 and CDK6. Plots are in the log 2 fold change in abundance of proteins (y axis) as measured using multiplexed quantitative-mass spectrometry-based proteomics of MOLT4 cells treated with BSJ-05-017 or BSJ-03-096 (250 nM) for 5 h versus negative log₁₀ p value (x axis). N=1.

FIGS. 11A-11I illustrate degraders targeting CDK4/6 inhibit cell growth and induce senescence in CDK4/6i sensitive and resistant models. FIG. 11A relates to comparison of dose-dependent effects of three degraders (BSJ-03-123, BSJ-03-189 and BSJ-05-017) in FAT1 loss cells by immunoblotting after 24 hr treatment. FIG. 11B relates to comparison of the CDK6-selective degrader BSJ-03-123, the most potent CDK4/6-dual degrader BSJ-05-017, and the CDK4/6i palbociclib in the inhibition of p-Rb1 in FAT1CR cells. All three drugs were administrated for 24 h at 500 nM. FIG. 11C relates to immunoblotting showing the effect of BSJ-05-017 and its negative control with a reversal of the two chiral centers in the VHL ligand in MCF-7 parental and FAT1CR cells. Cells were treated for 24 h with either drug (500 nM). representative blots are shown from 3 independent experiments. FIG. 11D relates to cell proliferation curve showing BSJ-05-017 inhibits the growth of both T47D parental and CDK6-overexpressing cells. Data were recorded at day3, day5 and day7. Data are shown as means±S.D., n=6. FIG. 11E shows senescence green staining from MCF7 cells treated with DMSO, BSJ-05-017 (500 nM), abemaciclib (100 nM) and Palbociclib (500 nM) for 8 days. Cell cycle distribution was measured after 24 h treatment. BSJ-05-017 induces senescence and cell cycle arrest to a same extent as abemaciclib and palbociclib in the sensitive MCF-7 parental cells. FIG. 11F (Top) shows proposed structural models of the E3 ligase adapter: degrader: CDK ternary complex with binding partners. The E3 ligase adapter (shown in purple) is either CRBN or VHL, in complex with BSJ-03-123 or BSJ-05-017 (shown in green), respectively; the CDK (shown in cyan) is either CDK6 or CDK4, bound with p18 or p27 (shown in gold), respectively; human cyclin D1 in all four structures is shown in pink. The kinases are in a top-down view. FIG. 11F (Bottom) shows backbone RMSD of each component of the ternary structure in molecular dynamics simulations for each of the four models. The variance of the RMSD values (width of the density) indicates the stability of each component during simulation and the orange vertical bar indicates when each model reaches equilibration in simulation. For each model, three parallel simulations with different random seeds were performed; one representative run is shown. FIG. 11G shows representative h-bond interactions (highlighted yellow) in the CDK: E3 ligase adaptor interface in the proposed structural models of the ternary complex for CDK4/6 with various binding partners. The E3 ligase adapter (shown in purple) is CRBN (in complex with BSJ-03-123, green) in the top panel, and VHL (in complex with BSJ-05-017, green) in the bottom panel; the CDK (shown in cyan) is CDK6 in the left column and CDK4 in the right column; p18, p27 and human cyclin D1 in all four structures are not shown for clarity. Each type of E3 ligase adaptor is in the same orientation. FIG. 11H shows representative h-bond interactions (highlighted yellow) the degraders make with the CDKs and the E3 ligase adaptors in the proposed structural models of the ternary complex for CDK4/6 with various binding partners. The E3 ligase adapter (shown in purple) is CRBN (in complex with BSJ-03-123, in green) in the top panel, and VHL (in complex with BSJ-05-017, in green) in the bottom panel; the CDK (shown in cyan) is CDK6 in the left column and CDK4 in the right column. Each type of E3 ligase adaptor is in the same orientation. Residue numbering of CRBN follows PDBID 5FQD; VHL follows PDBID 5NVV; CDK6 follows PDBID 1G3N; CDK4 follows PDBID 3G33. FIG. 11I shows pharmacokinetics data illustrating the plasma concentrations after administration of 25 mg/kg BSJ-05-017 intraperitoneally or 10 mg/kg BSJ-03-096 orally for 0.08, 0.25, 0.5, 1, 2, 4, 6, 8 and 24 hr. The table shows the maximum plasma concentration and their half-life time. N=3.

FIGS. 12A-12I illustrate degraders targeting CDK4/6 inhibit cell cycle and tumor growth in CDK4/6i resistant models. FIG. 12A shows BSJ-03-096 and its derivatives (TM-9, 12 and 13) are effective in degrading both CDK4 and CDK6 and efficiently inhibit the phosphorylation of Rb protein in abemaciclib-resistant cell model (MCF7 cells that lost FAT1 expression and with high CDK6 expression). FIG. 12B shows that BSJ-03-096 and its derivatives, including TM-5 and 7, and AM-01-125 and MS-01-303 effectively degrade CDK4/CDK6 and inhibit phosphorylation of Rb in FAT1 loss cells. FIGS. 12C and D show the quantification of band intensity in FIG. 12B. FIG. 12E shows BSJ-03-096 and its derivates including MS-02-24, AM-01-269, and AM-01-275 are effective in degrading CDK4/6 and inhibiting phosphorylation of Rb compared to abemaciclib in FAT1 loss cells. FIGS. 12F and G show BSJ-03-096 and BSJ-05-017 are effective in inhibiting tumor growth in two patient-derived xenograft models in mice, while clinical equivalent ribociclib dose does not inhibit their growth effectively. FIG. 12H shows a time-course of degradation of CDK6 as treated with a previously patented CDK6-specific degrader BSJ-03-123. Though with effective CDK6 degradation, BSJ-03-123 does not inhibit phosphorylation of Rb protein and other cell cycle signaling proteins, such as E2F1.

FIG. 12I shows both BSJ-03-096 and BSJ-05-017 effectively degrade CDK4 and CDK6 in another CDK6-high cell line, i.e., MCF7 cells with overexpression of CDK6.

FIGS. 13A-13B show cells of other cancer types are also sensitive to BSJ-05-017. A cell line screening was performed to test the effectiveness of BSJ-05-017 in inhibiting cell growth in ˜900 cell lines. FIG. 13A shows the top 20 cell lines that with AUCs of cell viability. This suggests BSJ-05-17 is also effective in other cancer cell lines besides breast cancer cells. FIG. 13B shows an example of cells, prostate cancer cell 22RV1, is more sensitive to BSJ-05-017 than to palbociclib. After 21 days treatment, the colony formation assay shows there are some colonies formed in Palbociclib-treated 22RV1 cells, but not in BSJ-05-017 treated cells.

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof—for example, “A, B, and/or C” would mean “A, B, C, A and B, A and C, B and C, or the combination of A, B, and C.”

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; and nitriles (i.e., CN).

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. Cycloalkylalkyl groups may be substituted or unsubstituted. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)=CH₂, —C(CH₃)═CH(CH₃), —C(CH₂CH₃)=CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups may be substituted or unsubstituted. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, and —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. Heterocyclyl groups may be substituted or unsubstituted. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. The phrase includes heterocyclyl groups that have other groups, such as alkyl, oxo or halo groups, bonded to one of the ring members, referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups may be substituted or unsubstituted. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent cycloalkyl groups are cycloalkylene groups, divalent heterocycloalkyl groups are heterocycloalkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³, R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O—. A “hydroxyalkyl” group is a hydroxyl-substituted alkyl group, such as HO—CH₂—.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹⁰⁰R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “isocyano” refers to —NC.

The term “isothiocyano” refers to —NCS.

The term “pentafluorosulfanyl” refers to —SF₅.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

As understood by one of ordinary skill in the art, “molecular weight” (also known as “relative molar mass”) is a dimensionless quantity but is converted to molar mass by multiplying by 1 gram/mole or by multiplying by 1 Da—for example, a compound with a weight-average molecular weight of 5,000 has a weight-average molar mass of 5,000 g/mol and a weight-average molar mass of 5,000 Da.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism, and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

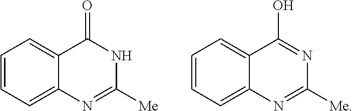 [SMILES: Cc1nc2ccccc2c(=O)[nH]1 ; Cc1nc(O)c2ccccc2n1]

As another example, guanidines may exhibit the following isomeric forms in protic organic solution, also referred to as tautomers of each other:

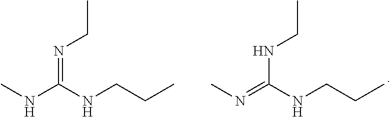

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

As used herein, the terms “cancer,” “neoplasm,” and “tumor,” are used interchangeably and refer to cells that have undergone a malignant transformation that makes them pathological to the host organism. Primary cancer cells (that is, cells obtained from near the site of malignant transformation) may be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

As used herein, the term “metastasis” or “metastatic” refers to the ability of a cancer cell to invade surrounding tissues, to enter the circulatory system and to establish malignant growths at new sites.

“Non-Metastatic” refers to tumors that do not spread beyond their original site of development and specifically do not enter the circulatory system and establish malignant growths at new sites.

As used herein, “prevention,” “prevent,” or “preventing” of a disease or condition refers to one or more compounds that, in a statistical sample, reduces the occurrence of the disease or condition in the treated sample relative to an untreated control sample, or delays the onset of one or more symptoms of the disease or condition relative to the untreated control sample. As used herein, prevention includes preventing or delaying the initiation of symptoms of the disease or condition. As used herein, prevention also includes preventing a recurrence of one or more signs or symptoms of a disease or condition.

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

As used herein, the terms “subject,” “individual,” or “patient” are used interchangeably and refer to an individual organism, a vertebrate, a mammal, or a human. In certain embodiments, the individual, patient or subject is a human.

Throughout this disclosure, various publications, patents and published patent specifications are referenced by an identifying citation. Also within this disclosure are Arabic numerals referring to referenced citations, the full bibliographic details of which are provided subsequent to the Examples section. The disclosures of these publications, patents and published patent specifications are hereby incorporated by reference into the present disclosure to more fully describe the present technology.

### The Present Technology

As Cyclin-dependent kinases 4 and 6 (CDK4/6), represent a major therapeutic vulnerability for breast cancer. The kinases are clinically targeted via ATP competitive inhibitors (CDK4/6i); however, drug resistance commonly emerges over time. To understand CDK4/6i resistance, over 1,300 breast cancers have been surveyed several genetic alterations (e.g. FAT1, PTEN or ARID1A loss) are identified converging on upregulation of CDK6. Mechanistically, CDK6 causes resistance by inducing and binding CDK inhibitor INK4 proteins (e.g. p18^{INK4C}). In vitro binding and kinase assays together with physical modeling reveal that the p18^{INK4C}/D-cyclin/CDK6 complex occludes CDK4/6i binding while only weakly suppressing ATP binding. Suppression of INK4 expression or its binding to CDK6 restores CDK4/6i sensitivity.

Described herein are bifunctional degraders conjugating palbociclib with E3 ligands. The resulting compounds potently degraded CDK4/6, leading to substantial antitumor effects in vivo, demonstrating the promising therapeutic potential for retargeting CDK4/6 despite CDK4/6i resistance.

### Compounds of the Present Technology

For ease of reference, the compounds included in any aspect or embodiment herein may be referred to anywhere in this disclosure as “a compound of the present technology,” “compounds of the present technology,” or the like. Similarly for ease of reference, the compositions, medicaments, and pharmaceutical compositions of the present technology may collectively be referred to herein as “compositions,” “compositions of the present technology,” or the like.

In an aspect, the present technology provides a compound according to Formula (I)

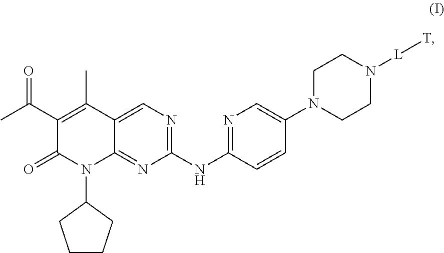

or a pharmaceutically acceptable salt and/or solvate thereof, wherein
   L is selected from the group consisting of

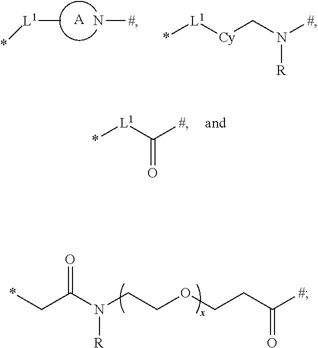

T is selected from the group consisting of

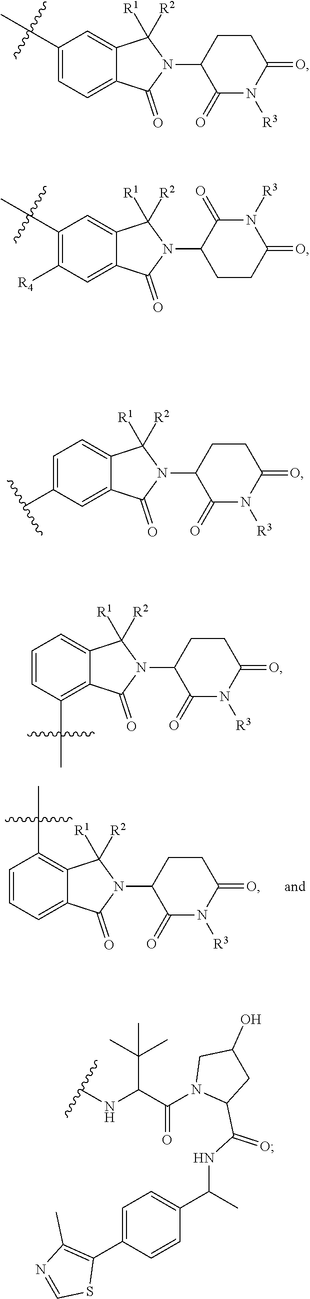

ring A is a 4- to 7-membered N-containing heterocycloalkylene optionally substituted with one or more groups selected from halogen and C₁-C₃ alkyl;
Cy is a C₄-C₆ cycloalkylene optionally substituted with one or more groups selected from halogen and C₁-C₃ alkyl;
R and R³ are each independently H or C₁-C₃ alkyl;
R¹ and R² are both H, or R¹ and R² are taken together to form an oxo (=O) group;
R⁴ is H, halo, C₁-C₄ alkyl, or C₃-C₆ cycloalkyl;
L¹ is a C₁-C₆ alkylene;
* is the linkage site to the nitrogen atom of the piperazine moiety;
# is the linkage site to the T group; and
x is 1, 2, or 3.

In any embodiment herein, it may be that T is selected from the group

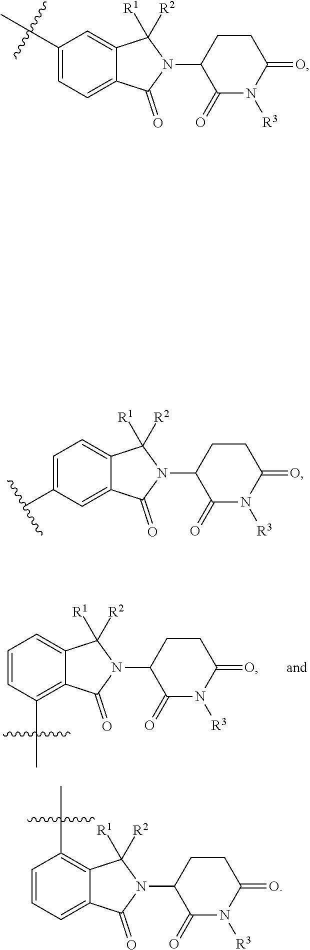

In any embodiment herein, it may be that the compound of Formula (I) is a compound of Formula (II)

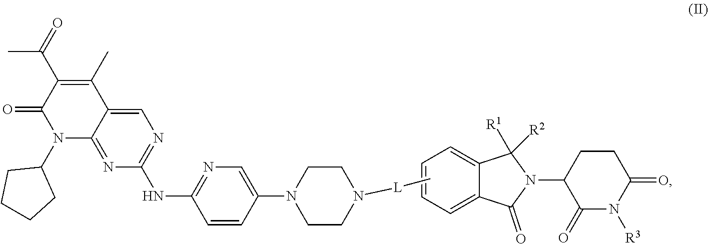

or a pharmaceutically acceptable salt and/or solvate thereof.

In any embodiment herein, it may be that R¹ is H. In any embodiment herein, it may be that R² is H. In any embodiment herein, it may be that R¹ and R² are both H. In any embodiment herein, it may be that R¹ and R² taken together form an oxo (=O) group. In any embodiment herein, it may be that R³ is H or C₁-C₃ alkyl. In any embodiment herein, it may be that R³ is H.

In any embodiment herein, it may be that L is

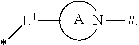

In any embodiment herein, it may be that L is

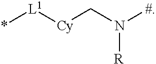

In any embodiment herein, it may be that * is the linkage site to the nitrogen atom of the piperazine moiety. In any embodiment herein, it may be that # is the linkage site to the T group.

In any embodiment herein, it may be that L¹ is a C₁-C₆ alkylene.

In any embodiment herein, it may be that Cy is a C₄-C₆ cycloalkylene. In any embodiment herein, it may be that Cy is a C₄-C₆ unsubstituted cycloalkylene. In any embodiment herein, it may be that Cy is a C₄-C₆ cycloalkylene substituted with one or more groups selected from halogen and C₁-C₃ alkyl.

In any embodiment herein, it may be that R is H or C₁-C₃ alkyl. In any embodiment herein, it may be that R is H.

In any embodiment herein, it may be that L is

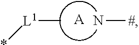

and the compound of Formula (I) or Formula (II) may be a compound of Formula (IIa)

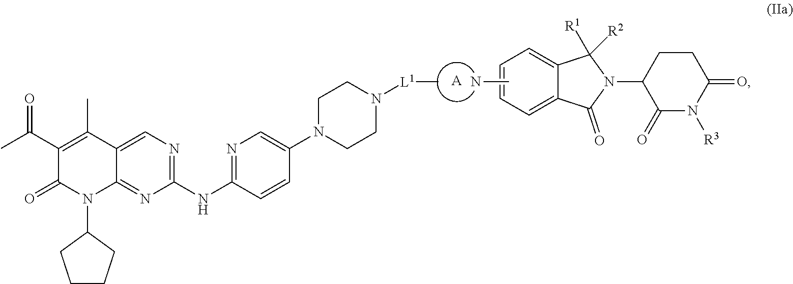

or a pharmaceutically acceptable salt and/or solvate thereof.

In any embodiment herein, it may be that ring A is a 4- to 7-membered N-containing heterocycloalkylene. In any embodiment herein, it may be that ring A is a unsubstituted 4- to 7-membered N-containing heterocycloalkylene. In any embodiment herein, it may be that ring A is a 4- to 7-membered N-containing heterocycloalkylene substituted with one or more groups selected from halogen and C₁-C₃ alkyl. In any embodiment herein, it may be that ring A is a 4- to 7-membered N-containing heterocycloalkylene substituted with one or more Me. In any embodiment herein, it may be that ring A is a 4- to 7-membered N-containing heterocycloalkylene substituted with one or more F.

In any embodiment herein, it may be that ring A is selected from the group consisting of

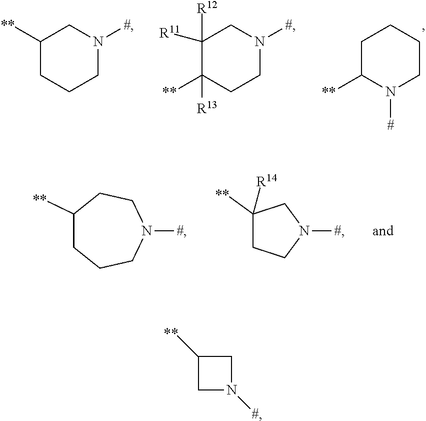

wherein
R¹¹ and R¹² are each independently H or halogen;
R¹³ and R¹⁴ are each independently H, halogen, or C₁-C₃ alkyl;
** is the linkage site to the L¹ group; and
# is the linkage site to the T group.

In any embodiment herein, it may be that R¹¹ is H or halogen. In any embodiment herein, it may be that R¹¹ is H or F. In any embodiment herein, it may be that R¹² is H or halogen. In any embodiment herein, it may be that R¹² is H or F. In any embodiment herein, it may be that R¹³ is H, halogen, or C₁-C₃ alkyl. In any embodiment herein, it may be that R¹³ is H, F, or Me. In any embodiment herein, it may be that R¹⁴ is H, halogen, or C₁-C₃ alkyl. In any embodiment herein, it may be that R¹⁴ is H or F.

In any embodiment herein, it may be that ** is the linkage site to the L¹ group. In any embodiment herein, it may be that # is the linkage site to the T group.

In any embodiment herein, it may be that L¹ is a C₁-C₆ alkylene. In any embodiment herein, it may be that L¹ is a methylene.

In any embodiment herein, it may be that R is H or C₁-C₃ alkyl. In any embodiment herein, it may be that R is H.

In any embodiment herein, it may be that L is selected from the group consisting of

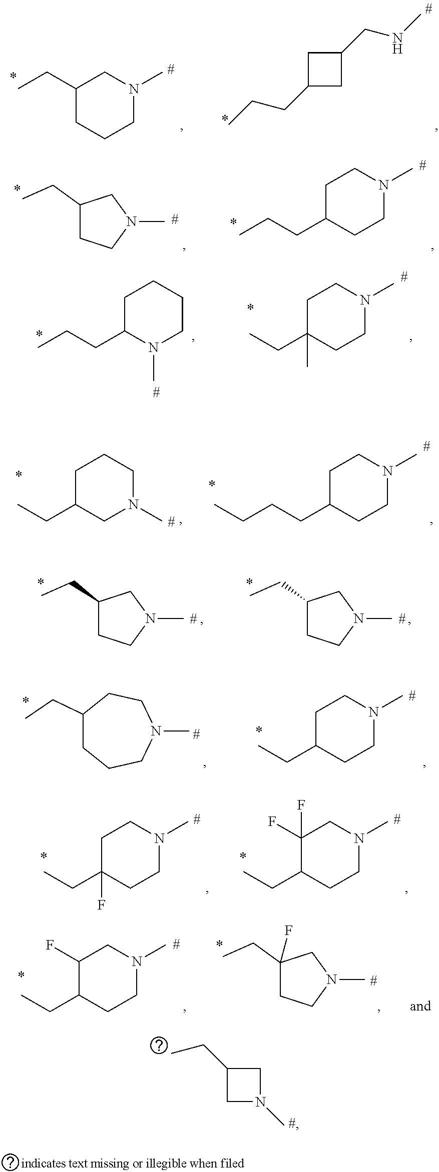

wherein
* is the linkage site to the nitrogen atom of the piperazine moiety; and
# is the linkage site to the T group.

In any embodiment herein, it may be that T is

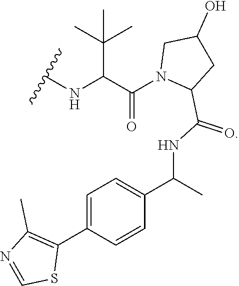 [SMILES: *NC(C(=O)N1CC(O)CC1C(=O)NC(C)c1ccc(-c2scnc2C)cc1)C(C)(C)C]

In any embodiment herein, it may be that T is

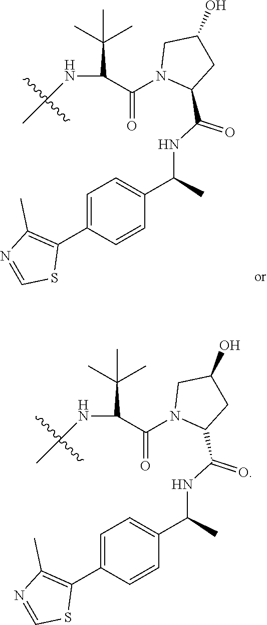

In any embodiment herein, it may be that the compound of Formula (I) is a compound of Formula (III)

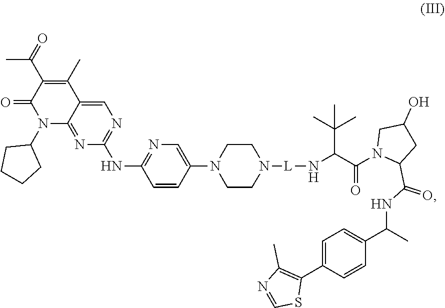

or a pharmaceutically acceptable salt and/or solvate thereof.

In any embodiment herein, it may be that L is

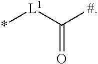

In any embodiment herein, it may be that L is

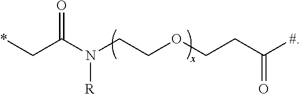

In any embodiment herein, it may be that L is

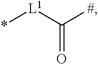

and the compound of Formula (I) or Formula (III) may be a compound of Formula (IIIa)

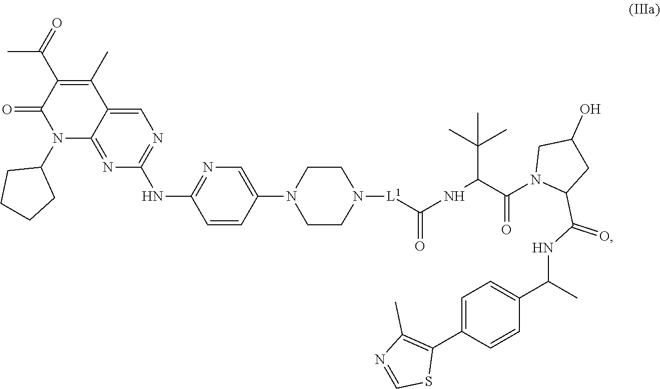

or a pharmaceutically acceptable salt and/or solvate thereof.

In any embodiment herein, it may be that L¹ is a C₁-C₆ alkylene.

In any embodiment herein, it may be that R is H or C₁-C₃ alkyl. In any embodiment herein, it may be that R is H.

In any embodiment herein, it may be that x is 1, 2, or 3.

In any embodiment herein, it may be that L is selected from the group consisting of

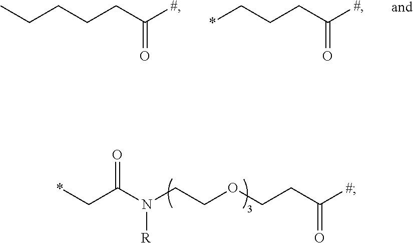

wherein
* is the linkage site to the nitrogen atom of the piperazine moiety; and
# is the linkage site to the T group.

In any embodiment herein, the bifunctional compound may be any one of the compounds in Table 1 or a pharmaceutically acceptable salt and/or solvate thereof (with the exception of the compounds labeled “Comparison Compound”).

TABLE 1

| | Com- |
|---|---|
| | pound |
| Structure | No. |
| 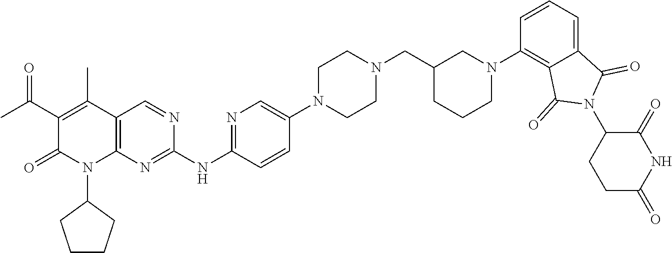 [SMILES: CC(=O)c1c(C)c2cnc(Nc3ccc(N4CCN(CC5CCCN(c6cccc7c6C(=O)N(C6CCC(=O)NC6=O)C7=O)C5)CC4)cn3)nc2n(C2CCCC2)c1=O] | (1) |
| 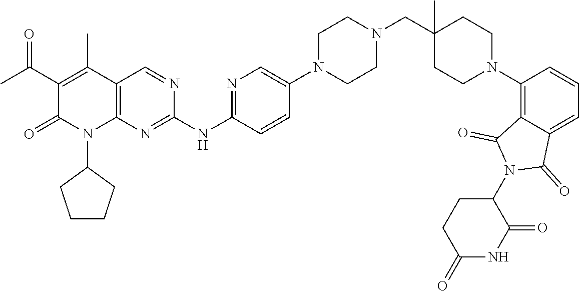 [SMILES: CC(=O)c1c(C)c2cnc(Nc3ccc(N4CCN(CC5(C)CCN(c6cccc7c6C(=O)N(C6CCC(=O)NC6=O)C7=O)CC5)CC4)cn3)nc2n(C2CCCC2)c1=O] | (2) |
| 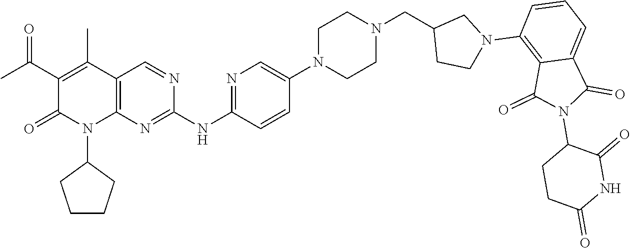 [SMILES: CC(=O)c1c(C)c2cnc(Nc3ccc(N4CCN(CC5CCN(c6cccc7c6C(=O)N(C6CCC(=O)NC6=O)C7=O)C5)CC4)cn3)nc2n(C2CCCC2)c1=O] | (3) |
| 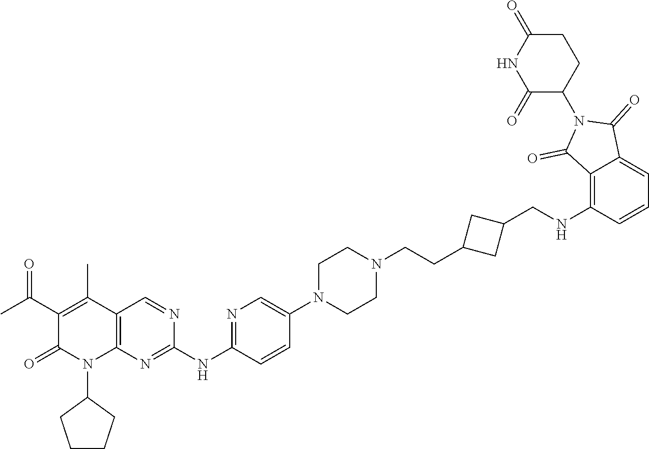 [SMILES: CC(=O)c1c(C)c2cnc(Nc3ccc(N4CCN(CCC5CC(CNc6cccc7c6C(=O)N(C6CCC(=O)NC6=O)C7=O)C5)CC4)cn3)nc2n(C2CCCC2)c1=O] | (4) |
| 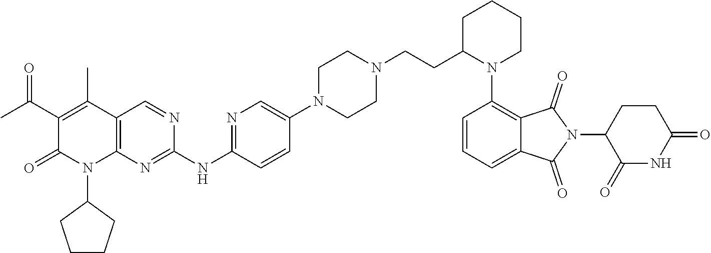 [SMILES: CC(=O)c1c(C)c2cnc(Nc3ccc(N4CCN(CCC5CCCCN5c5cccc6c5C(=O)N(C5CCC(=O)NC5=O)C6=O)CC4)cn3)nc2n(C2CCCC2)c1=O] | (5) |
| 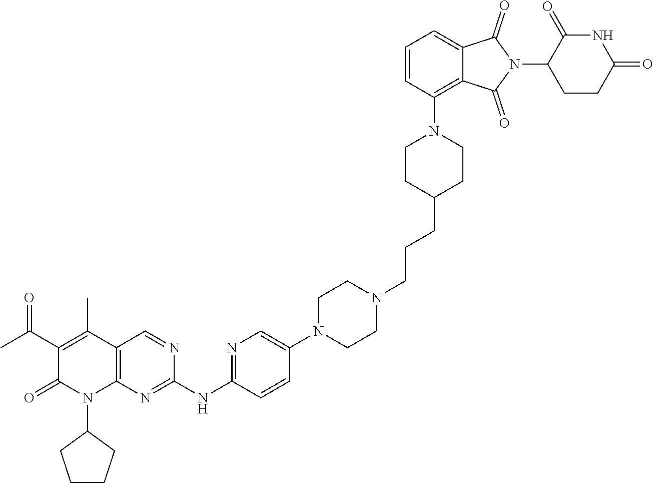 [SMILES: CC(=O)c1c(C)c2cnc(Nc3ccc(N4CCN(CCCC5CCN(c6cccc7c6C(=O)N(C6CCC(=O)NC6=O)C7=O)CC5)CC4)cn3)nc2n(C2CCCC2)c1=O] | (6) |
| 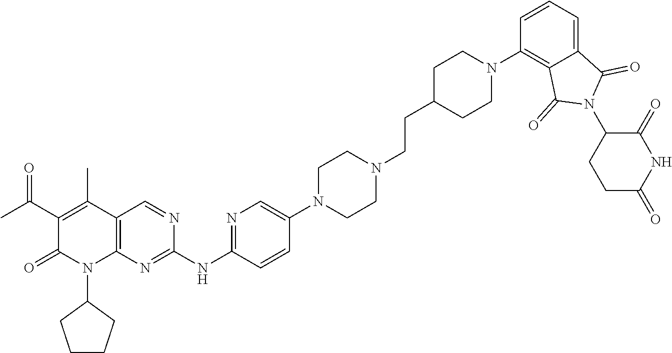 [SMILES: CC(=O)c1c(C)c2cnc(Nc3ccc(N4CCN(CCC5CCN(c6cccc7c6C(=O)N(C6CCC(=O)NC6=O)C7=O)CC5)CC4)cn3)nc2n(C2CCCC2)c1=O] | (7) |
| 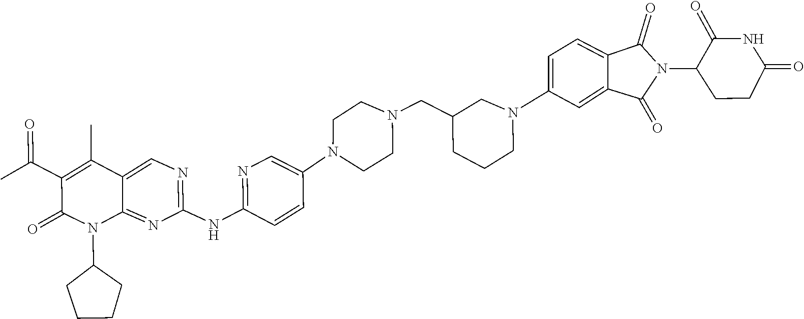 [SMILES: CC(=O)c1c(C)c2cnc(Nc3ccc(N4CCN(CC5CCCN(c6ccc7c(c6)C(=O)N(C6CCC(=O)NC6=O)C7=O)C5)CC4)cn3)nc2n(C2CCCC2)c1=O] | (8) |
| 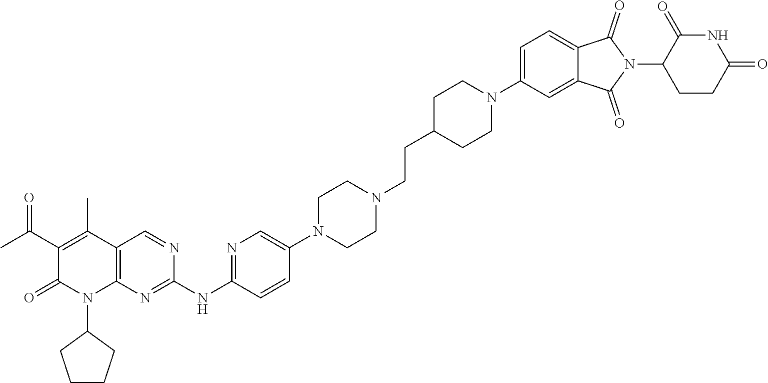 [SMILES: CC(=O)c1c(C)c2cnc(Nc3ccc(N4CCN(CCC5CCN(c6ccc7c(c6)C(=O)N(C6CCC(=O)NC6=O)C7=O)CC5)CC4)cn3)nc2n(C2CCCC2)c1=O] | (9) |
| 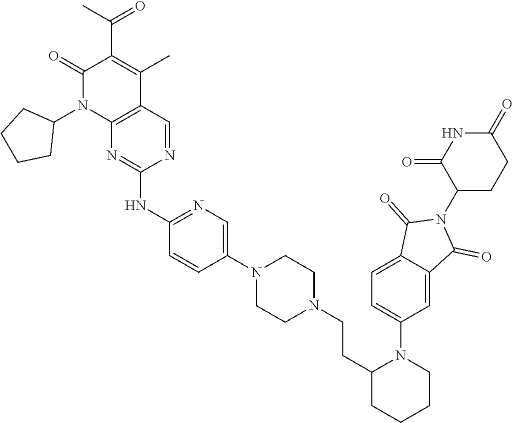 [SMILES: CC(=O)c1c(C)c2cnc(Nc3ccc(N4CCN(CCC5CCCCN5c5ccc6c(c5)C(=O)N(C5CCC(=O)NC5=O)C6=O)CC4)cn3)nc2n(C2CCCC2)c1=O] | (10) |
| 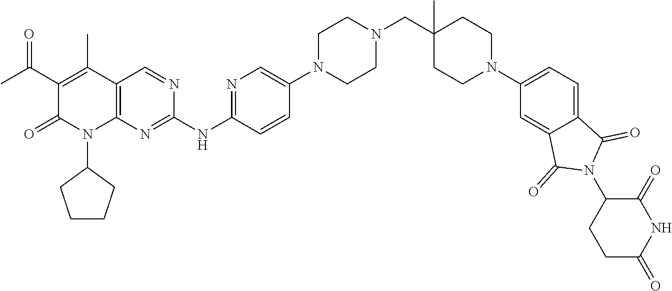 [SMILES: CC(=O)c1c(C)c2cnc(Nc3ccc(N4CCN(CC5(C)CCN(c6ccc7c(c6)C(=O)N(C6CCC(=O)NC6=O)C7=O)CC5)CC4)cn3)nc2n(C2CCCC2)c1=O] | (11) |
| 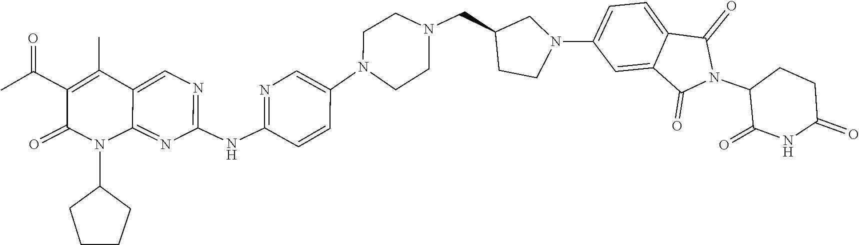 [SMILES: CC(=O)c1c(C)c2cnc(Nc3ccc(N4CCN(C[C@@H]5CCN(c6ccc7c(c6)C(=O)N(C6CCC(=O)NC6=O)C7=O)C5)CC4)cn3)nc2n(C2CCCC2)c1=O] | (12) |
| 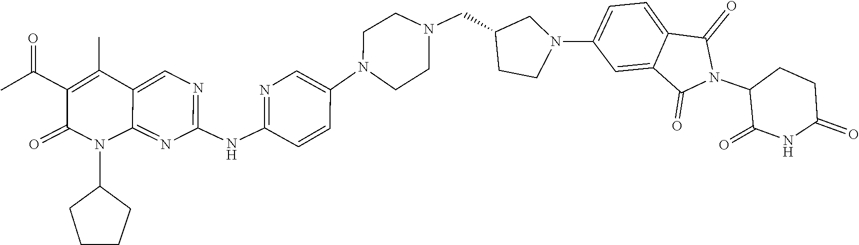 [SMILES: CC(=O)c1c(C)c2cnc(Nc3ccc(N4CCN(C[C@H]5CCN(c6ccc7c(c6)C(=O)N(C6CCC(=O)NC6=O)C7=O)C5)CC4)cn3)nc2n(C2CCCC2)c1=O] | (13) |
| 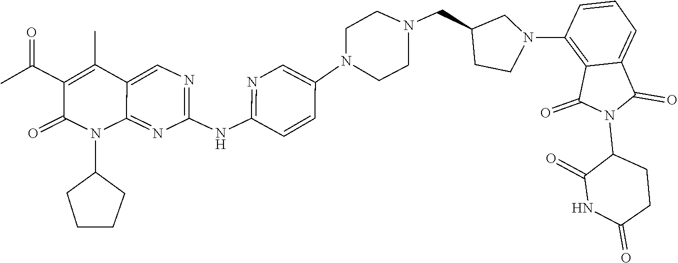 [SMILES: CC(=O)c1c(C)c2cnc(Nc3ccc(N4CCN(C[C@@H]5CCN(c6cccc7c6C(=O)N(C6CCC(=O)NC6=O)C7=O)C5)CC4)cn3)nc2n(C2CCCC2)c1=O] | (14) |
| 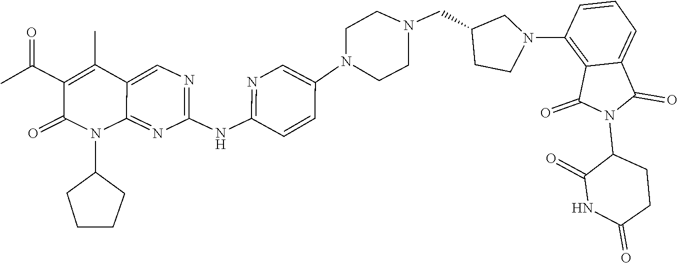 [SMILES: CC(=O)c1c(C)c2cnc(Nc3ccc(N4CCN(C[C@H]5CCN(c6cccc7c6C(=O)N(C6CCC(=O)NC6=O)C7=O)C5)CC4)cn3)nc2n(C2CCCC2)c1=O] | (15) |
| 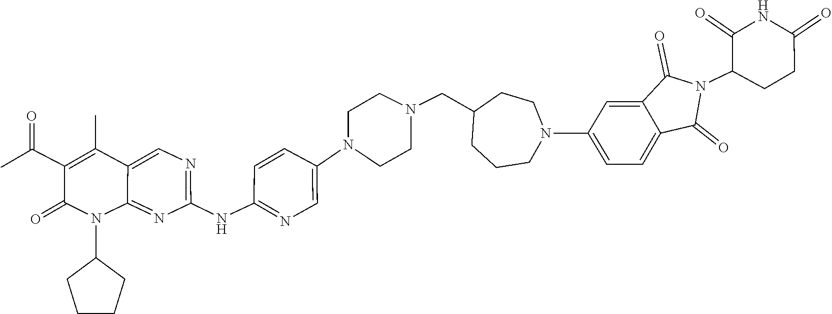 [SMILES: CC(=O)c1c(C)c2cnc(Nc3ccc(N4CCN(CC5CCCN(c6ccc7c(c6)C(=O)N(C6CCC(=O)NC6=O)C7=O)CC5)CC4)cn3)nc2n(C2CCCC2)c1=O] | (16) |
| 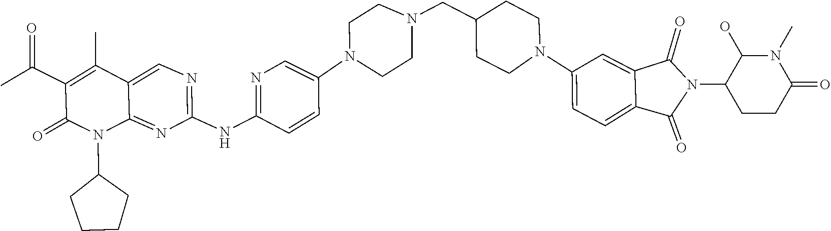 [SMILES: CC(=O)c1c(C)c2cnc(Nc3ccc(N4CCN(CC5CCN(c6ccc7c(c6)C(=O)N(C6CCC(=O)N(C)C6O)C7=O)CC5)CC4)cn3)nc2n(C2CCCC2)c1=O] | (17) |
| 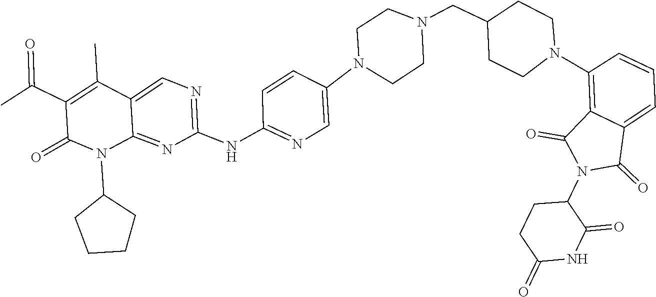 [SMILES: CC(=O)c1c(C)c2cnc(Nc3ccc(N4CCN(CC5CCN(c6cccc7c6C(=O)N(C6CCC(=O)NC6=O)C7=O)CC5)CC4)cn3)nc2n(C2CCCC2)c1=O] | (18) |
| 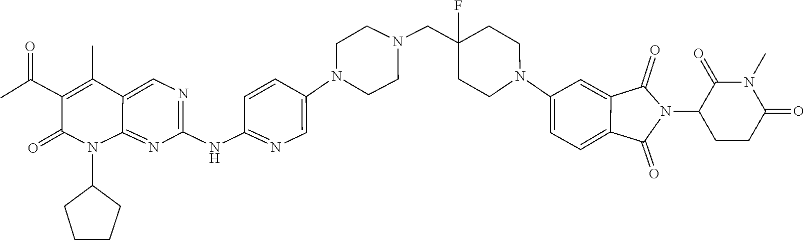 [SMILES: CC(=O)c1c(C)c2cnc(Nc3ccc(N4CCN(CC5(F)CCN(c6ccc7c(c6)C(=O)N(C6CCC(=O)N(C)C6=O)C7=O)CC5)CC4)cn3)nc2n(C2CCCC2)c1=O] | (19) |
| 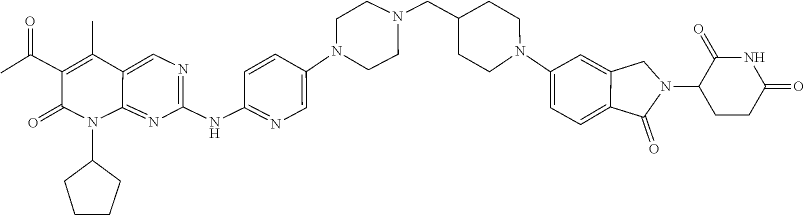 [SMILES: CC(=O)c1c(C)c2cnc(Nc3ccc(N4CCN(CC5CCN(c6ccc7c(c6)CN(C6CCC(=O)NC6=O)C7=O)CC5)CC4)cn3)nc2n(C2CCCC2)c1=O] | (20) |
| 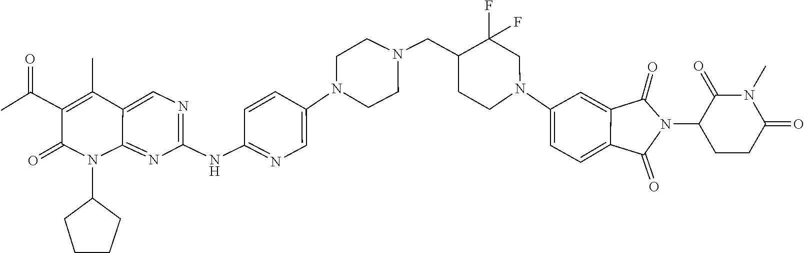 [SMILES: CC(=O)c1c(C)c2cnc(Nc3ccc(N4CCN(CC5CCN(c6ccc7c(c6)C(=O)N(C6CCC(=O)N(C)C6=O)C7=O)CC5(F)F)CC4)cn3)nc2n(C2CCCC2)c1=O] | (21) |
| 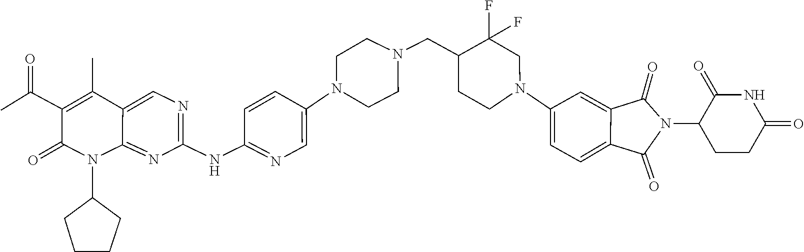 [SMILES: CC(=O)c1c(C)c2cnc(Nc3ccc(N4CCN(CC5CCN(c6ccc7c(c6)C(=O)N(C6CCC(=O)NC6=O)C7=O)CC5(F)F)CC4)cn3)nc2n(C2CCCC2)c1=O] | (22) |
| 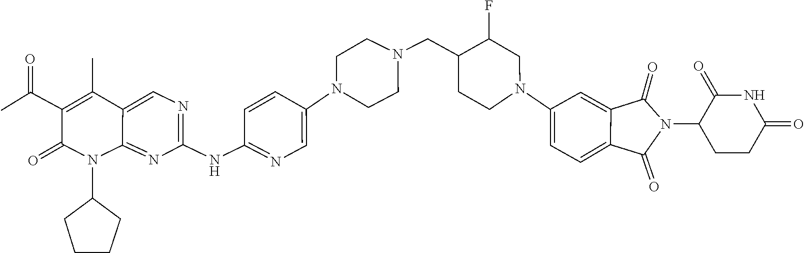 [SMILES: CC(=O)c1c(C)c2cnc(Nc3ccc(N4CCN(CC5CCN(c6ccc7c(c6)C(=O)N(C6CCC(=O)NC6=O)C7=O)CC5F)CC4)cn3)nc2n(C2CCCC2)c1=O] | (23) |
| 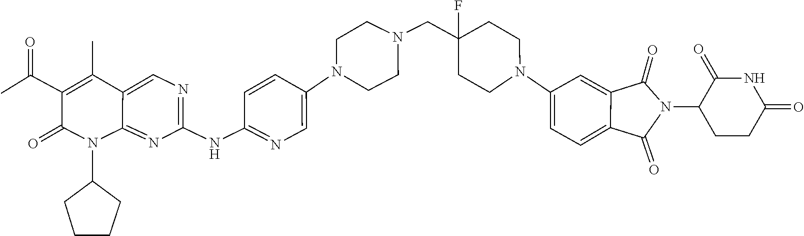 [SMILES: CC(=O)c1c(C)c2cnc(Nc3ccc(N4CCN(CC5(F)CCN(c6ccc7c(c6)C(=O)N(C6CCC(=O)NC6=O)C7=O)CC5)CC4)cn3)nc2n(C2CCCC2)c1=O] | (24) |
| 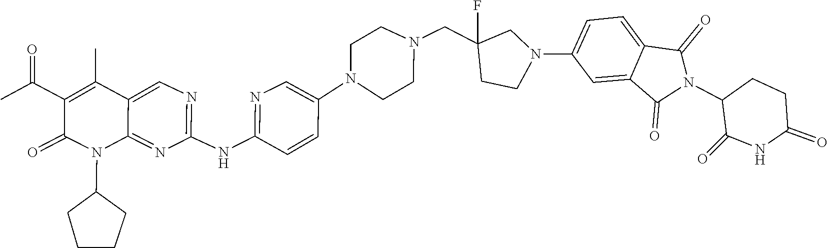 [SMILES: CC(=O)c1c(C)c2cnc(Nc3ccc(N4CCN(CC5(F)CCN(c6ccc7c(c6)C(=O)N(C6CCC(=O)NC6=O)C7=O)C5)CC4)cn3)nc2n(C2CCCC2)c1=O] | (25) |
| 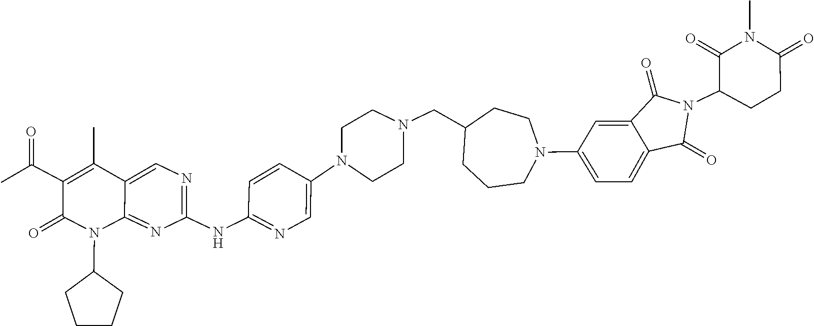 [SMILES: CC(=O)c1c(C)c2cnc(Nc3ccc(N4CCN(CC5CCCN(c6ccc7c(c6)C(=O)N(C6CCC(=O)N(C)C6=O)C7=O)CC5)CC4)cn3)nc2n(C2CCCC2)c1=O] | (26) |
| 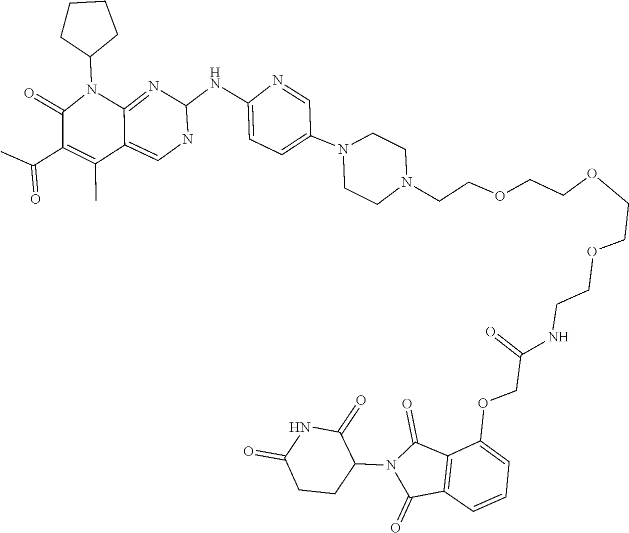 [SMILES: CC(=O)c1c(C)c2c(n(C3CCCC3)c1=O)=NC(Nc1ccc(N3CCN(CCOCCOCCOCCNC(=O)COc4cccc5c4C(=O)N(C4CCC(=O)NC4=O)C5=O)CC3)cn1)NC=2] | (27) Com- parison Com- pound |
| 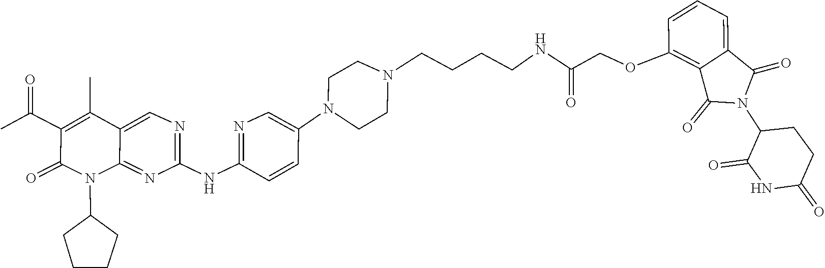 [SMILES: CC(=O)c1c(C)c2cnc(Nc3ccc(N4CCN(CCCCNC(=O)COc5cccc6c5C(=O)N(C5CCC(=O)NC5=O)C6=O)CC4)cn3)nc2n(C2CCCC2)c1=O] | (28) Com- parison Com- pound |
| 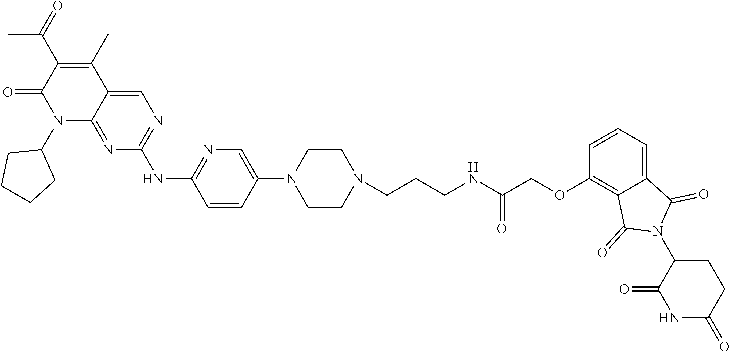 [SMILES: CC(=O)c1c(C)c2cnc(Nc3ccc(N4CCN(CCCNC(=O)COc5cccc6c5C(=O)N(C5CCC(=O)NC5=O)C6=O)CC4)cn3)nc2n(C2CCCC2)c1=O] | (29) Com- parison Com- pound |
| 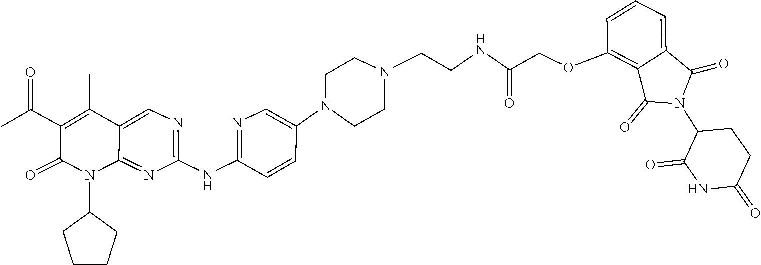 [SMILES: CC(=O)c1c(C)c2cnc(Nc3ccc(N4CCN(CCNC(=O)COc5cccc6c5C(=O)N(C5CCC(=O)NC5=O)C6=O)CC4)cn3)nc2n(C2CCCC2)c1=O] | (30) Com- parison Com- pound |
| 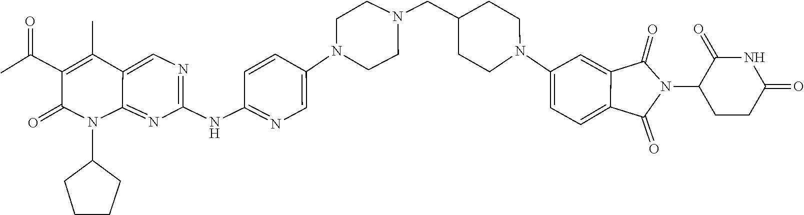 [SMILES: CC(=O)c1c(C)c2cnc(Nc3ccc(N4CCN(CC5CCN(c6ccc7c(c6)C(=O)N(C6CCC(=O)NC6=O)C7=O)CC5)CC4)cn3)nc2n(C2CCCC2)c1=O] | (31) |
| 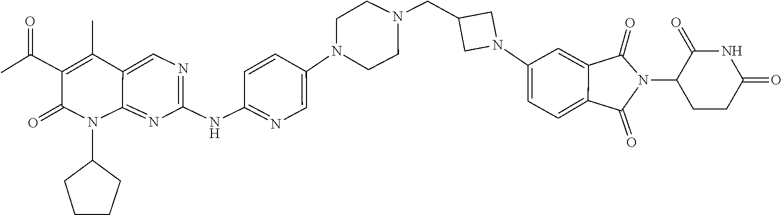 [SMILES: CC(=O)c1c(C)c2cnc(Nc3ccc(N4CCN(CC5CN(c6ccc7c(c6)C(=O)N(C6CCC(=O)NC6=O)C7=O)C5)CC4)cn3)nc2n(C2CCCC2)c1=O] | (32) |
| 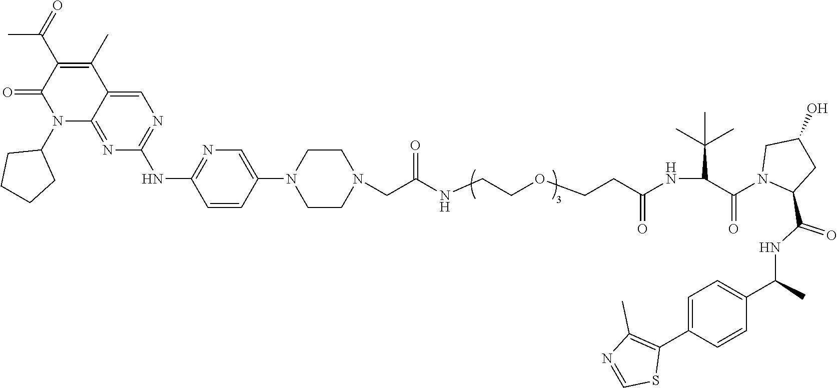 | (33) |
| 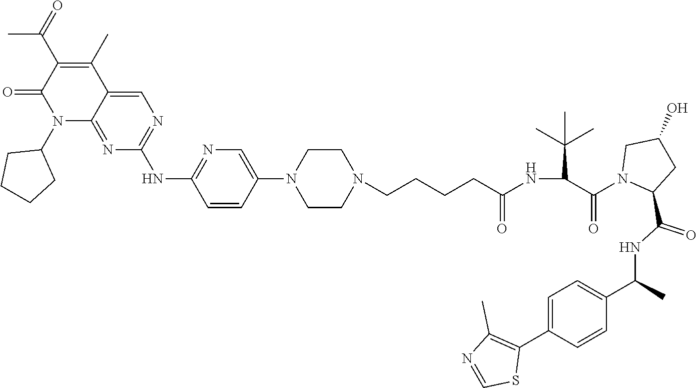 | (34) |
| 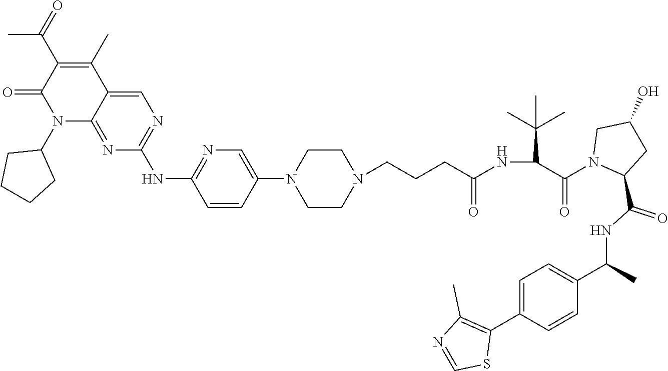 | (35) |
| 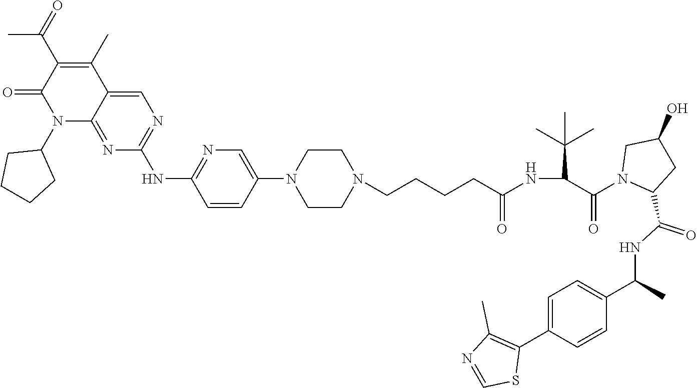 | (36) |

### Compositions and Methods

In another aspect, a composition is provided that includes a compound of any embodiment disclosed herein, a pharmaceutically acceptable carrier or one or more excipients, fillers or agents (collectively referred to hereafter as “pharmaceutically acceptable carrier” unless otherwise indicated and/or specified). In a related aspect, a medicament for treating, preventing, and/or ameliorating a CDK4 and/or CDK6-mediated disorder, disease, or condition (e.g., a disorder, disease, or condition as described herein) in a subject is provided that includes a compound of any embodiment disclosed herein and optionally a pharmaceutically acceptable carrier. The medicament of any embodiment herein may include an effective amount of the compound for treating, preventing, and/or ameliorating the CDK4 and/or CDK6-mediated disorder, disease, or condition. In a related aspect, a pharmaceutical composition is provided that includes (i) an effective amount of a compound of any embodiment disclosed herein, wherein the effective amount of the compound is effective to treat a CDK4 and/or CDK6-mediated disorder, disease, or condition (e.g., a disorder, disease, or condition as described herein); and (ii) a pharmaceutically acceptable carrier. In any embodiment herein, the CDK4 and/or CDK6-mediated disorder, disease, or condition may be a cancer such as breast cancer.

“Effective amount” refers to the amount of a compound or composition required to produce a desired effect. One example of an effective amount includes amounts or dosages that yield acceptable toxicity and bioavailability levels for therapeutic (pharmaceutical) use including, but not limited to, reduction of a tumor mass. In any aspect or embodiment disclosed herein (collectively referred to herein as “any embodiment herein,” “any embodiment disclosed herein,” or the like) of the compositions, pharmaceutical compositions, and methods including compounds of the present technology, the effective amount may be an amount effective in treating, preventing, and/or ameliorating a CDK4 and/or CDK6-mediated disorder, disease, or condition (e.g., a disorder, disease, or condition as described herein such as breast cancer). By way of example, the effective amount of any embodiment herein including a compound of the present technology may be from about 0.01 g to about 1000 mg of the compound (such as from about 0.1 g to about 50 mg of the compound, about 50 mg to about 500 mg, or about 500 mg to 1000 mg of the compound). The methods and uses according to the present technology may include an effective amount of a compound of any embodiment disclosed herein. In any aspect or embodiment disclosed herein, the effective amount may be determined in relation to a subject. As used herein, a “subject” or “patient” is a mammal, such as a cat, dog, rodent or primate. Typically the subject is a human, and, preferably, a human suffering from or suspected of suffering from pain. The term “subject” and “patient” can be used interchangeably.

Thus, the present technology provides pharmaceutical compositions and medicaments including a compound of any embodiment disclosed herein (or a composition of any embodiment disclosed herein such as breast cancer) and a pharmaceutically acceptable carrier. The compositions may be used in the methods and treatments described herein. The pharmaceutical composition may be packaged in unit dosage form. The unit dosage form may be effective in treating, preventing, and/or ameliorating a CDK4 and/or CDK6-mediated disorder, disease, or condition (e.g., a disorder, disease, or condition as described herein). Generally, a unit dosage including a compound of the present technology will vary depending on patient considerations. Such considerations include, for example, age, protocol, condition, sex, extent of disease, contraindications, concomitant therapies and the like. An exemplary unit dosage based on these considerations may also be adjusted or modified by a physician skilled in the art. For example, a unit dosage for a patient comprising a compound of the present technology may vary from 1×10⁻⁴ g/kg to 1 g/kg, preferably, 1×10⁻³ g/kg to 1.0 g/kg. Dosage of a compound of the present technology may also vary from 0.01 mg/kg to 100 mg/kg or, preferably, from 0.1 mg/kg to 10 mg/kg. Suitable unit dosage forms, include, but are not limited to parenteral solutions, oral solutions, powders, tablets, pills, gelcaps, capsules, lozenges, suppositories, patches, nasal sprays, injectables, implantable sustained-release formulations, mucoadherent films, topical varnishes, lipid complexes, liquids, etc.

The pharmaceutical compositions and medicaments may be prepared by mixing one or more compounds and/or compositions of the present technology with pharmaceutically acceptable carriers, excipients, binders, diluents or the like. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars and/or sugar alcohols. Aqueous and nonaqueous (e.g., in a fluorocarbon propellant) aerosols are typically used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs.

Various assays and model systems can be readily employed to determine the therapeutic effectiveness of the treatment according to the present technology. For example, effectiveness of the compositions (as well as determination of effective amounts) and methods of the present technology may be demonstrated by a decrease in the mass of a tumor and/or slowing the growth of a tumor.

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

The compounds of the present technology can also be administered to a patient along with other conventional therapeutic agents that may be useful in the treatment of a disease described herein. The administration may include oral administration, parenteral administration, or nasal administration. In any of these embodiments, the administration may include intratumoral injections, subcutaneous injections, intravenous injections, intraperitoneal injections, or intramuscular injections. In any of these embodiments, the administration may include oral administration. The methods of the present technology can also include administering, either sequentially or in combination with one or more compounds of the present technology, a conventional therapeutic agent in an amount that can potentially or synergistically be effective for the treatment of a CDK4 and/or CDK6-mediated disorder, disease, or condition (e.g., a disorder, disease, or condition as described herein such as breast cancer).

In one aspect, a compound of the present technology is administered to a patient in an amount or dosage suitable for therapeutic use. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

In an aspect a method of inducing degradation of CDK4 and/or CDK6 in a subject in need thereof is provided, where the method includes administering to the subject an effective amount of a compound of any embodiment disclosed herein or administering an effective amount of a composition of any embodiment disclosed herein.

In another aspect a method of treating, preventing, and/or ameliorating a subject suffering from a CDK4 and/or CDK6-mediated disorder, disease, or condition (e.g., a disorder, disease, or condition as described herein such as breast cancer) is provided, where the method includes administering to the subject an effective amount of a compound of any embodiment disclosed herein or administering an effective amount of a composition of any embodiment disclosed herein. In any embodiment herein of the method, the administering may include an administration method as described herein. In any embodiment herein, the CDK4 and/or CDK6-mediated disorder, disease, or condition may be a cancer. In any embodiment herein, the cancer may include breast cancer, prostate cancer, adenocarcinoma, lymphoma, thyroid cancer, lung-NSC (non-small cell lung cancer), rhabdoid tumor, cholangiocarcinoma, small cell lung cancer, bile-duct cancer, acute myeloid leukemia, sarcoma, medulloblastoma, embryonal tumors, and/or urinary-tract cancer. In any embodiment herein, the cancer may be breast cancer.

Accordingly, in another aspect a method of treating, preventing, and/or ameliorating a subject suffering from breast cancer is provided, where the method includes administering to the subject an effective amount of a compound of any embodiment disclosed herein or administering an effective amount of a composition of any embodiment disclosed herein.

In any embodiment herein, the administering may include local administration of the compound to a site in the subject including the disorder, disease, or condition described herein (e.g., cancer such as breast cancer). In any embodiment herein, the administering may include oral, rectal, nasal, vaginal, transdermal, intravenous, intramuscular, or inhalation administration. In any embodiment herein, the administering may include injection of the compound into the site in the subject including the disorder, disease, or condition described herein (e.g., cancer such as breast cancer) or proximal to the site in the subject including the disorder, disease, or condition described herein (e.g., cancer such as breast cancer).

### EXAMPLES

The present technology is further illustrated by the following Examples, which should not be construed as limiting in any way. The examples herein are provided to illustrate advantages of the present technology and to further assist a person of ordinary skill in the art with preparing or using the compounds and compositions of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects, or embodiments of the present technology.

Reagents. Starting materials, reagents and solvents were purchased from commercial suppliers and were used without further purification unless otherwise noted. For example, Abemaciclib (LY2835219) and palbociclib (PD-0332991) were obtained from Selleck Chemicals (Houston, TX, USA) and TargetMol (Wellesley Hills, MA, USA). Ribociclib (LEE011) was obtained from Novartis (Cambridge, MA, USA). These drugs were dissolved in dimethyl sulfoxide. Phospho-Rb1 (Ser780) (#8180), Phospho-Rb1 (Ser807/811) (#8516), Rb1 (#9309), Cyclin D1 (#2978), CDK6 (#3136), CDK4 (#12790), CDK2 (#2546), E2F1 (#3742), Cyclin A2 (#4656), Cyclin E2 (#4132), YAP (#14074), TAZ (#4883), p18 (#2896) and j-actin (#4970) antibodies were purchased from Cell Signaling Technology (Danvers, MA, USA). FAT1 (#ab190242) and p15INK4B (ab53034) antibodies were purchased from Abcam (Cambridge, UK). Recombinant Human CDK6/Cyclin D3 (C35-10H) and CDK4/Cyclin D3 (C31-18G) were purchased from SignalChem (British Columbia, Canada). Rb1 protein (#ab56270) was purchased from Abcam (Cambridge, UK). ADP-Glo™ Kinase Assay Kit (V6930) was purchased from Promega (Madison, WI, USA).

Cell lines. MCF-7, T47D, CAMA-1, ZR-75-1, EFM19 and BT474 cell lines were obtained from the American Type Culture Collection (Manassas, VA, USA). HEK293T was a gift from Dr. Ping Chi's lab. MCF-7 cells were maintained in DMEM/F12 medium. T47D, ZR-75-1, EFM19 and BT474 cells were maintained in RPMI medium. CAMA-1 cells were maintained in DMEM medium. All media were supplemented with 10% FBS, 2 mM L-glutamine, 20 units/ml penicillin and 20 μg/ml streptomycin. All cell lines were tested negative for mycoplasma contamination.

### Example 1. Exemplary Preparation of Compounds

### Preparation of Compounds (1)-(11)

A general scheme for synthesis of Compounds (1)-(11) is shown in Schemes 1-2.

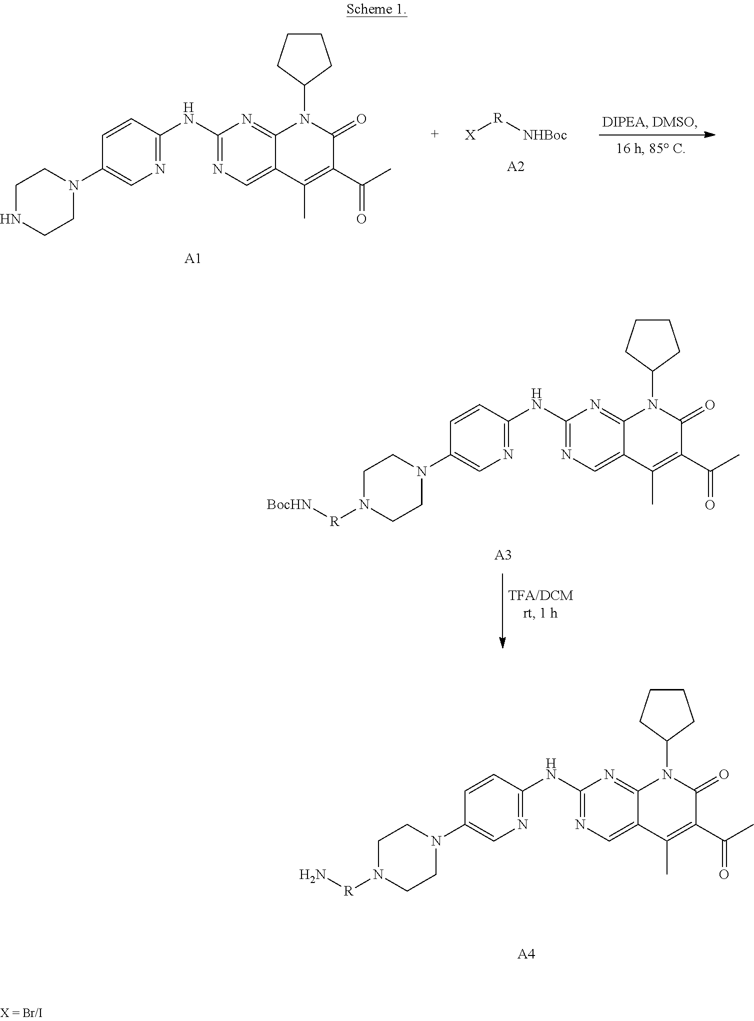

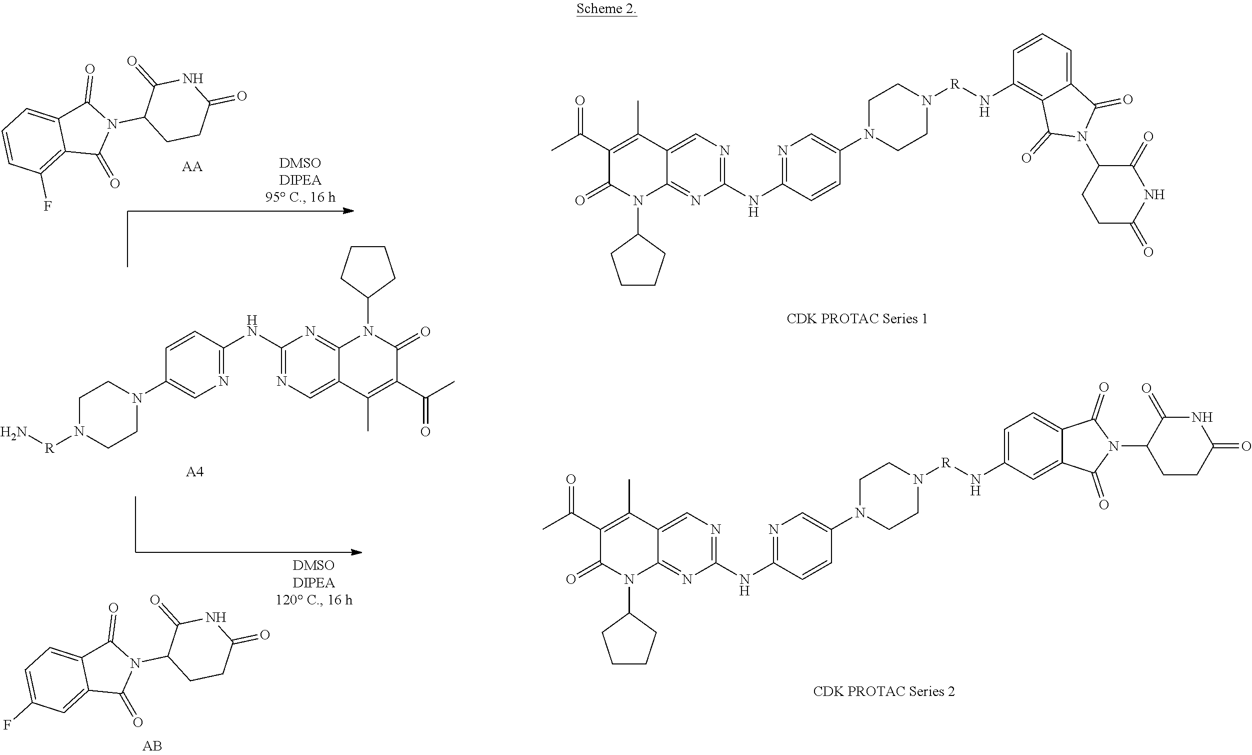

Representative procedure for synthesis of the linkers (A2) is shown in Scheme 3. Structures of the synthesized linkers (A2a-A2e) are shown in Scheme 4.

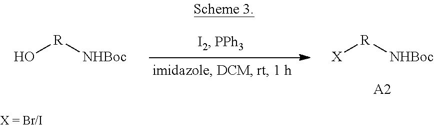

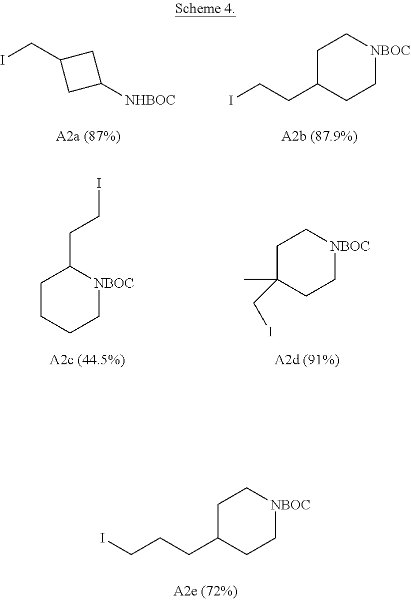

N-(3-(iodomethyl)cyclobutyl)-1-(1-methyl)-1-(1-oxidaneyl)boranamine (A2a). To a solution of triphenyl phosphine (1.431 g, 5.461 mmol) in CH₂Cl₂ (15 mL), iodine (1.388 g, 5.464 mmol), imidazole (0.625 g, 9.177 mmol), hydroxy piperidine (0.5 g, 2.484 mmol) were added with an interval of 10 minutes at room temperature. The flask was wrapped with aluminum foil and the solution was allowed to stir for 1 h. The reaction mixture was quenched by addition of anhydrous Na₂S₂O₃ solution (25 mL) and extracted with CH₂Cl₂ (3×100 mL). The combined organic layers were washed with brine (50 mL), dried over Na₂SO₄, filtered and concentrated to a colorless solid which was purified by column chromatography (silica gel; EtOAc/hexanes, 3:7) to afford A2a as a colorless solid (0.673 g, 87%).

By analogy, 1-(1-methyl) (1-oxidaneyl)boraneyl)-4-(2-iodoethyl) piperidine (A2b), 1-((1-methyl) (1-oxidaneyl)boraneyl)-2-(2-iodoethyl) piperidine (A2c), 1-((1-methyl) (1-oxidaneyl)boraneyl)-4-(iodomethyl)-4-methylpiperidine (A2d), 1-((1-methyl)(1-oxidaneyl)boraneyl)-4-(3-iodopropyl) piperidine (A2e) were formed from a similar set of procedures starting with different hydroxy precursors.

The following two linkers (A2f and A2g) were obtained from commercial sources.

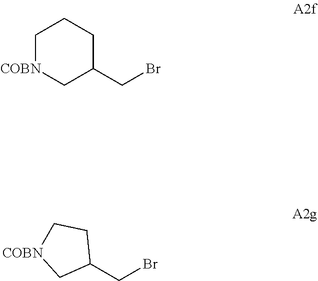

### Preparation of Compound A3

The Structures of compounds A3a-A3g are shown in Scheme 5.

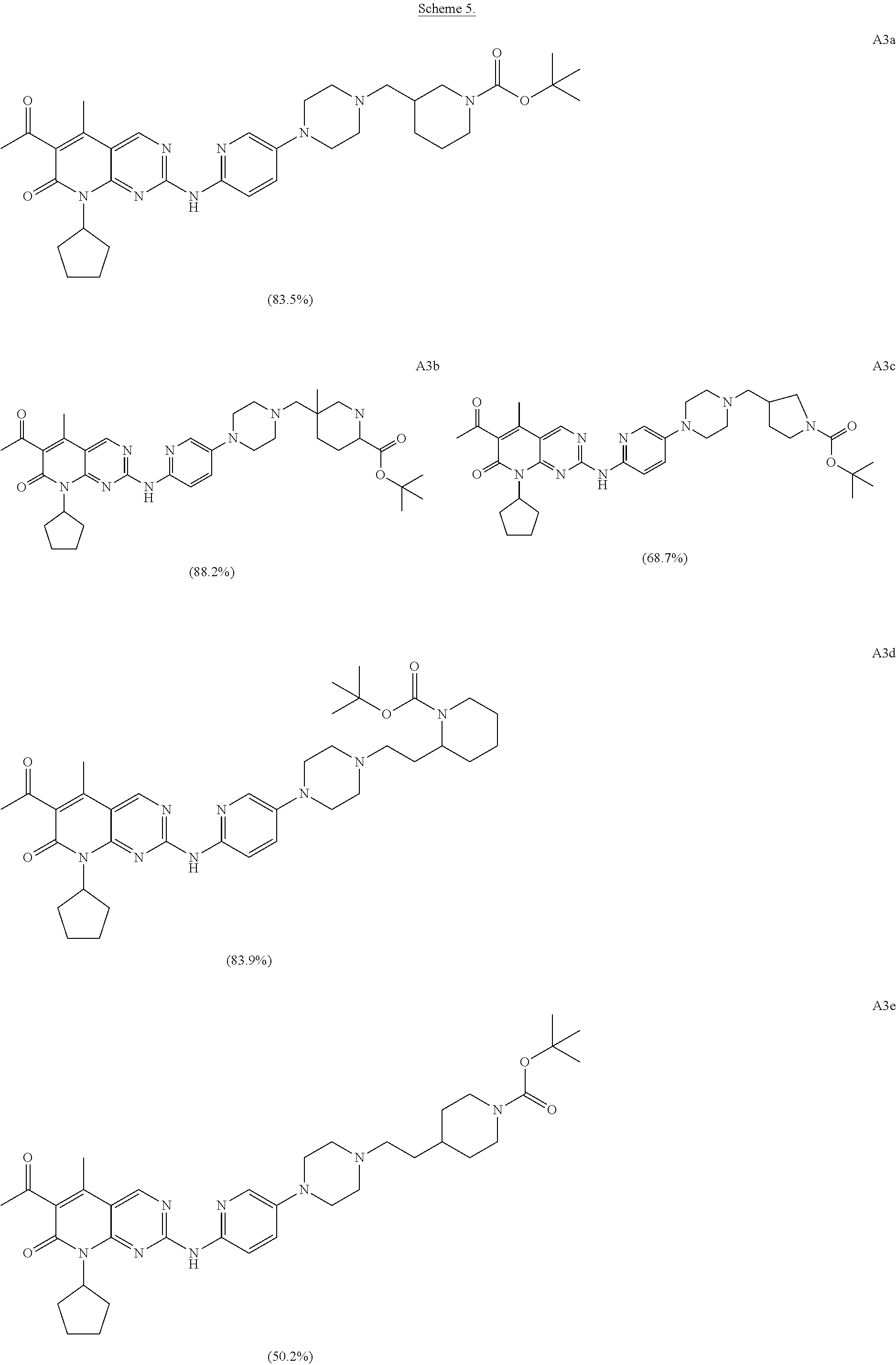

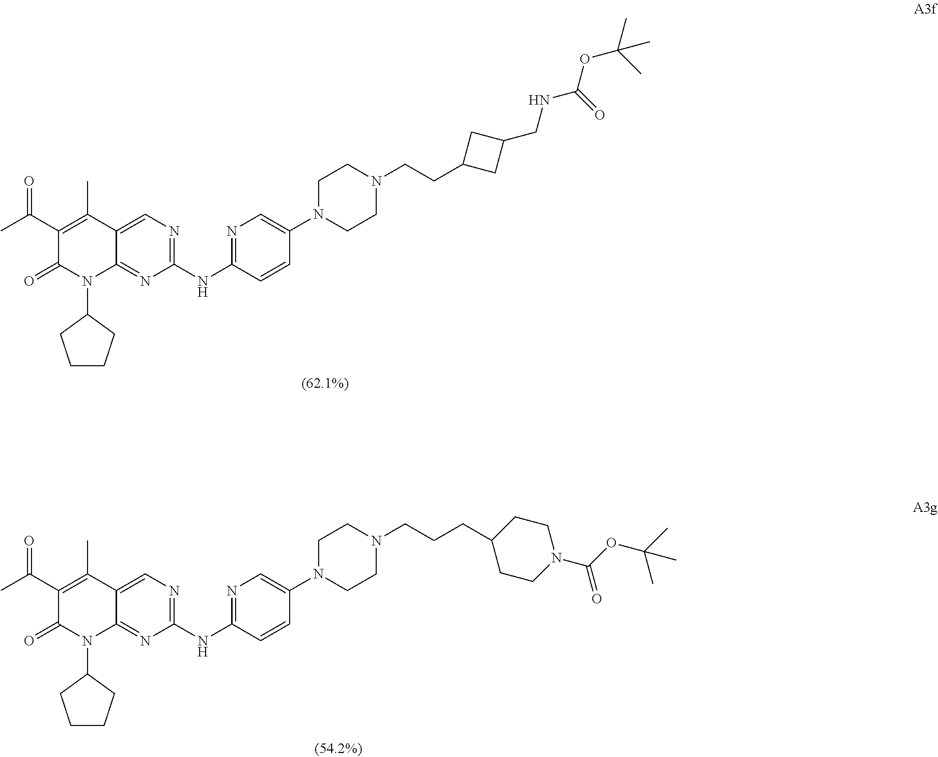

Representative procedure for synthesis of A3—synthesis of compound A3a: Tert-butyl-3-((4-(6-(6-acetyl-8-cyclopentyl-5-methyl-7-oxo-7,8-dihydropyrido[2,3-d]pyrimidin-2-yl) amino) pyridin-3-yl) piperazin-1-yl) methyl) piperidine-1-carboxylate.

To a solution of Palbociclib (0.2 g, 0.447 mmol) in DMSO (15 mL), linker (A2f) (0.124 g, 0.892 mmol), DIPEA (0.229 ml, 1.342 mmol) were added. The mixture was heated to 80° C. and kept stirring for 18 h. The reaction mixture was quenched by water (25 mL) and extracted with EtOAc (3×100 mL). The combined organic layers were washed with brine (50 mL), dried over Na₂SO₄, filtered and concentrated to a yellow solid which was purified by reverse phase column chromatography (C18: 25-80% ACN in H₂O) to afford A3a as a yellow solid (0.184 g, 75%. LC-MS: m/z 545 [M+1]

Compounds A3b-A3g were prepared using a similar set of procedures as compound A3a.

Compounds (1)-(11) were synthesized from compounds A3a-A3g.

### Compound (1): 4-(3-((4-(6-(6-acetyl-8-cyclopentyl-5-methyl-7-oxo-7,8-dihydropyrido[2,3-d]pyrimidin-2-yl) amino) pyridin-3-yl) piperidin-1-yl)-2-(2,6-dioxopiperidin-3-yl) isoindoline-1,3-dione (AM-01-98)

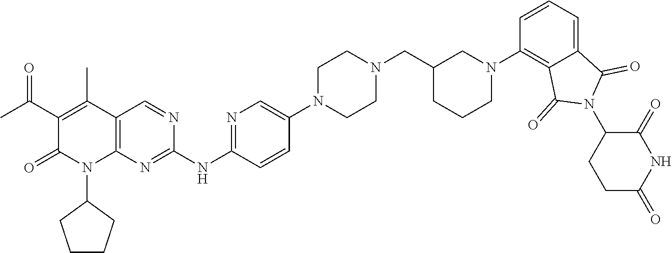 [SMILES: CC(=O)c1c(C)c2cnc(Nc3ccc(N4CCN(CC5CCCN(c6cccc7c6C(=O)N(C6CCC(=O)NC6=O)C7=O)C5)CC4)cn3)nc2n(C2CCCC2)c1=O]

To a solution of deprotected A3a (0.190 g, 0.349 mmol), 2-(2,6-dioxopiperidin-3-yl)-4-fluoroisoindoline-1,3-dione (degron AA 0.0813 g, 0.294 mmol) was added along with DIPEA (0.256 ml, 1.47 mmol) and DMSO (10 ml). The mixture was heated to 95° C. and kept stirring for 18 h. The reaction mixture was quenched by water (25 mL) and extracted with EtOAc (3×100 mL). The combined organic layers were washed with brine (50 mL), dried over Na₂SO₄, filtered and concentrated to a dark yellow solid which was purified by reverse phase column chromatography (C18: 25-80% ACN in H₂O) to afford 5 as a brown solid (0.08g, quantitative yield). LC-MS: m/z 801.38 [M+1], ¹H NMR (500 MHz, DMSO-d₆) δ 1.17 (m, J=12.9, 2.7 Hz, 5H), 1.48-1.65 (m, 17H), 1.66-1.74 (m, 2H), 1.75-1.94 (m, 23H), 1.99-2.08 (m, 3H), 2.32 (s, 16H), 2.43 (s, 17H), 5.05 (s, 1H), 5.83 (s, 1H), 7.29 (s, 2H), 7.41 (s, 1H), 7.68 (s, OH), 7.85 (s, 1H), 8.03 (s, 1H), 8.92 (s, 6H), 9.64 (s, 4H), 10.82 (s, 2H).

### Compound (2): 4-(4-(4-(6-(6-acetyl-8-cyclopentyl-5-methyl-7-oxo-7,8-dihydropyrido[2,3-d]pyrimidin-2-yl) amino) pyridin-3-yl) piperazin-1-yl) methyl)-4-methylpiperidin-1-yl)-2-(2,6-dioxopiperidin-3-yl) isoindoline-1,3-dione (AM-01-277)

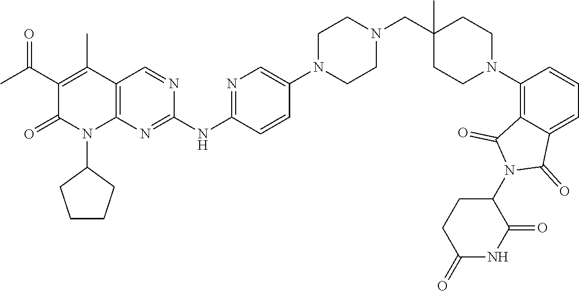 [SMILES: CC(=O)c1c(C)c2cnc(Nc3ccc(N4CCN(CC5(C)CCN(c6cccc7c6C(=O)N(C6CCC(=O)NC6=O)C7=O)CC5)CC4)cn3)nc2n(C2CCCC2)c1=O]

Compound (2) were synthesized using analogous procedures as described in Compound (1) and by employing Palbociclib-linkers shown in Scheme 4. Brown solid (0.016 g, 7%): ¹H NMR (400 MHz, DMSO-d₆) δ 1.42 (t, J=7.1 Hz, 6H), 1.47-1.60 (m, 12H), 1.67-1.79 (m, 8H), 1.84 (s, 9H), 2.12 (t, J=7.5 Hz, 4H), 2.26 (s, 16H), 2.37 (s, 16H), 3.42 (s, 20H), 5.06 (s, 2H), 5.27 (s, 1H), 5.81 (s, 1H), 7.38 (s, 2H), 7.50 (s, 2H), 7.68 (s, 1H), 7.86 (s, 2H), 8.09 (s, 4H), 8.91 (s, 6H), 10.10 (s, 5H), 11.06 (s, 4H). LC-MS: m/z [M+H]⁺ for C₄₄H₅₀N₁₀O₆, calculated 814.95; observed 815.93.

### Compound (3): 4-(3-(4-(6-(6-acetyl-8-cyclopentyl-5-methyl-7-oxo-7,8-dihydropyrido[2,3-d]pyrimidin-2-yl) amino) pyridin-3-yl) piperazin-1-yl) methyl) pyrrolidin-1-yl)-2-(2,6-dioxopiperidin-3-yl) isoindoline-1,3-dione (AM-01-133)

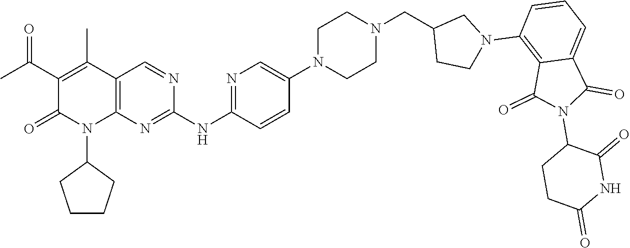 [SMILES: CC(=O)c1c(C)c2cnc(Nc3ccc(N4CCN(CC5CCN(c6cccc7c6C(=O)N(C6CCC(=O)NC6=O)C7=O)C5)CC4)cn3)nc2n(C2CCCC2)c1=O]

Compound (3) were synthesized using analogous procedures as described in Compound (1) and by employing Palbociclib-linkers shown in Scheme 4. Yellow solid (0.04 g, 44.9%): ¹H NMR (500 MHz, DMSO-d₆) δ 1.58 (s, 1H), 1.75 (s, OH), 1.87 (s, 3H), 2.30 (s, 5H), 2.41 (s, 7H), 3.16 (d, J=5.4 Hz, 6H), 5.06 (m, J=12.7, 5.4, 1.3 Hz, 1H), 5.82 (p, J=8.8 Hz, 2H), 7.07-7.13 (m, 3H), 7.47 (dd, J=9.1, 3.0 Hz, 1H), 7.56 (dd, J=8.6, 7.0 Hz, 1H), 7.84 (d, J=9.0 Hz, 2H), 8.05 (d, J=3.0 Hz, 1H), 8.94 (s, 1H), 10.10 (s, 2H), 11.06 (s, 1H). LC-MS: m/z [M+H]⁺ for C₄₃H₄₇N₁₀O₆, calculated 786.89; observed 786.85.

### Compound (4): 4-(((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)cyclobutyl)methyl)amino)-2-(2,6-dioxopiperidin-3-yl)isoindoline-1,3-dione (AM-01-125)

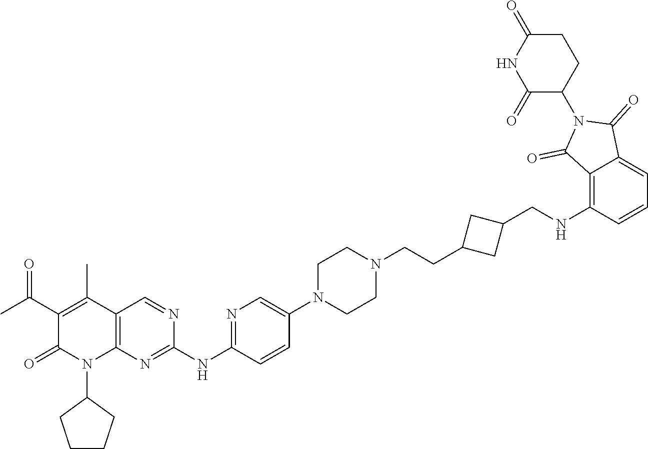 [SMILES: CC(=O)c1c(C)c2cnc(Nc3ccc(N4CCN(CCC5CC(CNc6cccc7c6C(=O)N(C6CCC(=O)NC6=O)C7=O)C5)CC4)cn3)nc2n(C2CCCC2)c1=O]

Compound (4) were synthesized using analogous procedures as described in Compound (1) and by employing Palbociclib-linkers shown in Scheme 4. Yellow solid (0.053 g, 46.6%): ¹H NMR (500 MHz, DMSO-d₆) δ 1.17 (t, J=7.1 Hz, OH), 1.38 (dd, J=8.2, 6.6 Hz, 1H), 1.98-2.06 (m, 1H), 2.18 (t, J=7.4 Hz, 1H), 2.30 (s, 4H), 2.42 (s, 1H), 5.82 (p, J=9.0 Hz, 2H), 6.46 (s, 1H), 7.04 (dd, J=17.0, 7.8 Hz, 3H), 7.46 (dd, J=9.1, 3.1 Hz, 2H), 7.59 (dd, J=8.5, 7.1 Hz, 1H), 7.84 (d, J=9.0 Hz, 2H), 8.04 (s, 1H), 8.95 (s, 1H), 10.10 (s, 1H), 11.11 (s, 1H). LC-MS: m/z [M+H]⁺ for C₄₂H₄₆N₁₀O₆, calculated 786.36; observed 787.38.

### Compound (5): 5-(2-(2-(4-(6-(6-acetyl-8-cyclopentyl-5-methyl-7-oxo-7,8-dihydropyrido[2,3-d]pyrimidin-2-yl) amino) pyridin-1-yl) piperidin-1-yl)-2-(2,6-dioxopiperidin-3-yl) isoindoline-1,3-dione (AM-01-286)

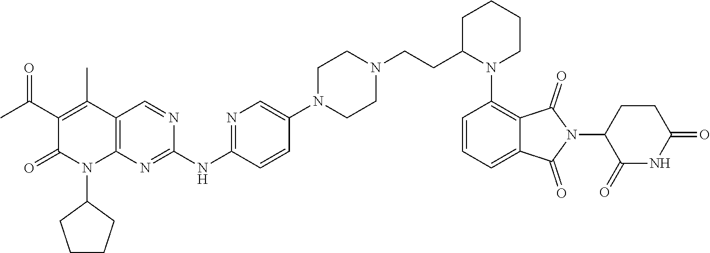 [SMILES: CC(=O)c1c(C)c2cnc(Nc3ccc(N4CCN(CCC5CCCCN5c5cccc6c5C(=O)N(C5CCC(=O)NC5=O)C6=O)CC4)cn3)nc2n(C2CCCC2)c1=O]

Compound (5) was synthesized using analogous procedures as described in Compound (1) and by employing Palbociclib-linkers shown in Scheme 4. Yellow solid (0.022 g, 5.6%): ¹H NMR (400 MHz, DMSO-d₆) δ 1.42 (t, J=7.1 Hz, 6H), 1.47-1.60 (m, 12H), 1.67-1.79 (m, 8H), 1.84 (s, 9H), 2.12 (t, J=7.5 Hz, 4H), 2.26 (s, 16H), 2.37 (s, 16H), 3.42 (s, 20H), 5.06 (s, 2H), 5.27 (s, 1H), 5.81 (s, 1H), 7.38 (s, 2H), 7.50 (s, 2H), 7.68 (s, 1H), 7.86 (s, 2H), 8.09 (s, 4H), 8.91 (s, 6H), 10.10 (s, 5H), 11.06 (s, 4H). LC-MS: m/z [M+H]⁺ for C₄₄H₅₀N₁₀O₆, calculated 814.95; observed 815.09.

### Compound (6): 4-(4-(3-(6-(6-acetyl-8-cyclopentyl-5-methyl-7-oxo-7,8-dihydropyrido[2,3-d]pyrimidin-2-yl) amino) pyridin-3-yl) piperazin-1-yl) propyl) piperidin-1-yl)-2-(2,6-dioxopiperidin-3-yl) isoindoline-1,3-dione (MS-02-24)

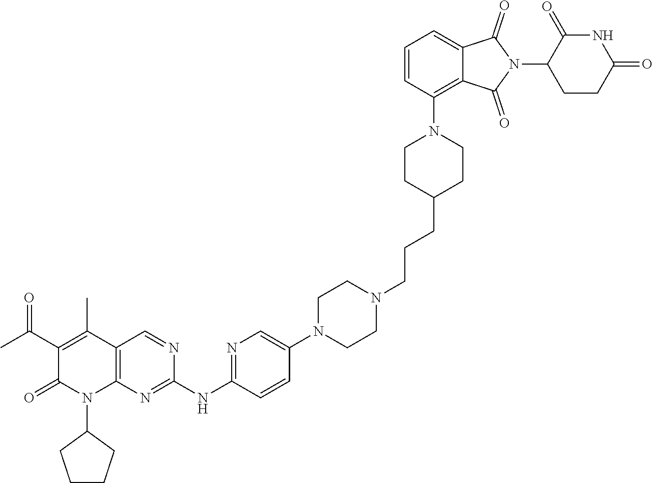 [SMILES: CC(=O)c1c(C)c2cnc(Nc3ccc(N4CCN(CCCC5CCN(c6cccc7c6C(=O)N(C6CCC(=O)NC6=O)C7=O)CC5)CC4)cn3)nc2n(C2CCCC2)c1=O]

Compound (6) were synthesized using analogous procedures as described in Compound (1) and by employing Palbociclib-linkers shown in Scheme 4. Yellow solid (0.050 g, 34.7%): ¹H NMR (400 MHz, DMSO-d₆) δ 1.07 (s, 1H), 1.14 (d, J=14.3 Hz, 6H), 1.40-1.57 (m, 9H), 1.79 (d, J=12.2 Hz, 6H), 1.89 (d, J=12.1 Hz, 3H), 2.19 (d, J=7.4 Hz, 1H), 2.31 (s, 6H), 2.42 (s, 6H), 2.85 (t, J=11.1 Hz, 5H), 3.69 (d, J=11.7 Hz, 4H), 5.09 (dd, J=12.9, 5.5 Hz, 2H), 5.78-5.85 (m, 2H), 7.33 (t, J=7.2 Hz, 4H), 7.47 (d, J=9.2 Hz, 2H), 7.67 (t, J=7.8 Hz, 2H), 7.85 (d, J=9.1 Hz, 2H), 8.05 (s, 1H), 8.95 (s, 2H), 10.11 (s, 2H), 11.10 (s, 2H). LC-MS: m/z [M+H]⁺ for C₄₅H₅₂N₁₀O₆, calculated 828.98; observed 829.15.

### Compound (7): 4-(4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)piperidin-1-yl)-2-(2,6-dioxopiperidin-3-yl)isoindoline-1,3-dione (MS-01-303)

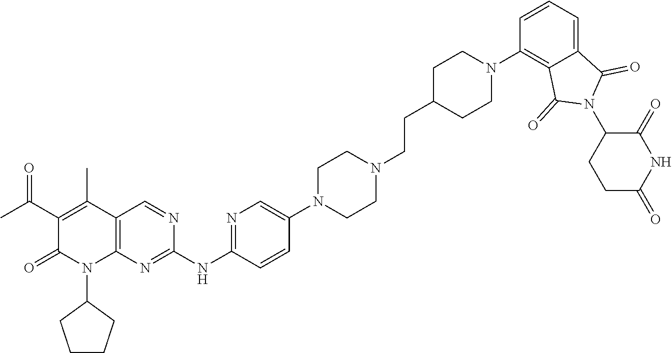 [SMILES: CC(=O)c1c(C)c2cnc(Nc3ccc(N4CCN(CCC5CCN(c6cccc7c6C(=O)N(C6CCC(=O)NC6=O)C7=O)CC5)CC4)cn3)nc2n(C2CCCC2)c1=O]

Compound (7) were synthesized using analogous procedures as described in Compound (1) and by employing Palbociclib-linkers shown in Scheme 4. Yellow solid (0.248 g, 42.5%): ¹H NMR (500 MHz, DMSO-d₆) δ 1.49 (s, 13H), 1.57 (dt, J=6.7, 4.1 Hz, 7H), 1.72-1.83 (m, 18H), 1.88 (s, 12H), 2.23 (m, J=16.0, 7.9, 4.2 Hz, 11H), 2.30 (s, 15H), 2.42 (s, 15H), 3.16 (s, 18H), 5.08 (s, 1H), 5.82 (s, 1H), 7.32 (s, 5H), 7.48 (d, J=2.8 Hz, 2H), 7.67 (s, 3H), 7.84 (s, 3H), 8.05 (d, J=2.9 Hz, 5H), 8.95 (s, 5H), 10.11 (s, 5H), 11.09 (s, 5H). LC-MS: m/z [M+H]⁺ for C₄₄H₅₀N₁₀O₆, calculated 814.39; observed 814.56.

### Compound (8): 5-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidin-1-yl)-2-(2,6-dioxopiperidin-3-yl)isoindoline-1,3-dione (AM-01-251)

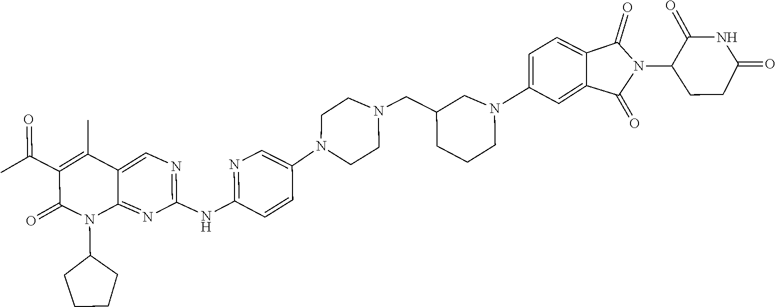 [SMILES: CC(=O)c1c(C)c2cnc(Nc3ccc(N4CCN(CC5CCCN(c6ccc7c(c6)C(=O)N(C6CCC(=O)NC6=O)C7=O)C5)CC4)cn3)nc2n(C2CCCC2)c1=O]

To a solution of deprotected A3e (0.134 g, 0.246 mmol), 2-(2,6-dioxopiperidin-3-yl)-5-fluoroisoindoline-1,3-dione (degron AB 0.0679 g, 0.246 mmol) was added along with DIPEA (0.214 ml, 1231 mmol) and DMSO (10 ml). The mixture was heated to 120° C. and kept stirring for 16 h. The reaction mixture was quenched by water (25 mL) and extracted with EtOAc (3×100 mL). The combined organic layers were washed with brine (50 mL), dried over Na₂SO₄, filtered and concentrated to a dark yellow solid which was purified by reverse phase column chromatography (C18: 25-80% ACN in H₂O) to afford Compound (8) as a yellow solid (0.015 g, 7.6%) ¹H NMR (400 MHz, DMSO-d₆) δ 1.12 (d, J=15.0 Hz, 13H), 1.39-1.42 (m, 2H), 1.71-1.94 (m, 9H), 2.30 (s, 3H), 2.42 (s, 4H), 3.18 (d, J=17.1 Hz, 6H), 4.51-4.71 (m, 4H), 5.66-5.91 (m, 2H), 7.21 (d, J=8.7 Hz, 1H), 7.29 (s, 1H), 7.48 (d, J=8.6 Hz, 2H), 7.66 (d, J=8.6 Hz, 1H), 7.86 (d, J=8.9 Hz, 2H), 8.04-8.09 (m, 2H), 8.95 (s, 4H), 10.13 (s, 2H), 11.08 (s, 1H). LC-MS: m/z 801.23 [M+1].

### Compound (9): 5-(4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)piperidin-1-yl)-2-(2,6-dioxopiperidin-3-yl)isoindoline-1,3-dione (AM-01-269)

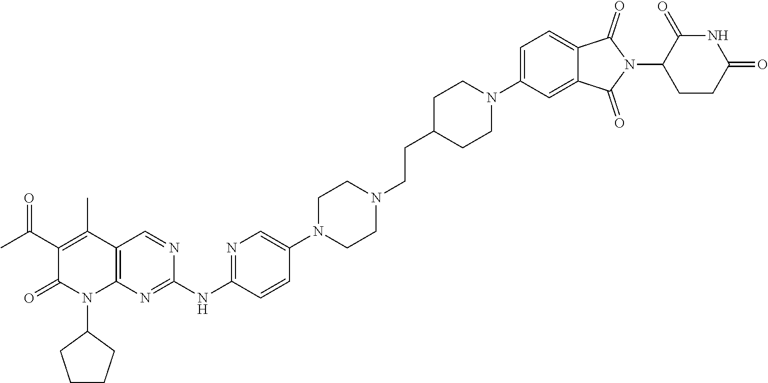 [SMILES: CC(=O)c1c(C)c2cnc(Nc3ccc(N4CCN(CCC5CCN(c6ccc7c(c6)C(=O)N(C6CCC(=O)NC6=O)C7=O)CC5)CC4)cn3)nc2n(C2CCCC2)c1=O]

Compound (9) were synthesized using analogous procedures as described in Compound (8) and by employing Palbociclib-linkers shown in Scheme 4. Pale brown solid (0.030 g, 10.67%): ¹H NMR (400 MHz, DMSO-d₆) δ 1.04-1.19 (m, 16H), 1.70 (s, 4H), 1.82 (s, 3H), 2.25 (s, 5H), 2.37 (s, 6H), 2.86 (dt, J=30.2, 13.9 Hz, 4H), 3.10 (d, J=10.4 Hz, 7H), 3.40-3.48 (m, 1H), 3.99 (d, J=12.9 Hz, 3H), 4.60 (d, J=3.3 Hz, OH), 5.04 (s, OH), 5.71 (s, OH), 7.26 (s, 2H), 7.42 (d, J=9.2 Hz, 2H), 7.81 (s, 1H), 8.00 (s, 2H), 8.90 (s, 2H), 10.07 (s, 2H), 11.04 (s, 2H). LC-MS: m/z [M+H]⁺ for C₄₄H₅₀N₁₀O₆, calculated 814.95; observed 814.97.

### Compound (10): 6-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)piperidin-1-yl)-2-(2,6-dioxopiperidin-3-yl)-3a,4-dihydro-1H-isoindole-1,3(2H)-dione (AM-01-275)

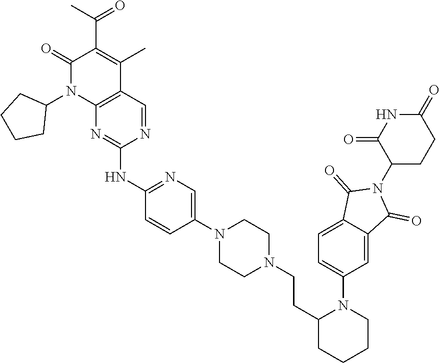 [SMILES: CC(=O)c1c(C)c2cnc(Nc3ccc(N4CCN(CCC5CCCCN5c5ccc6c(c5)C(=O)N(C5CCC(=O)NC5=O)C6=O)CC4)cn3)nc2n(C2CCCC2)c1=O]

Compound (10) were synthesized using analogous procedures as described in Compound (8) and by employing Palbociclib-linkers shown in Scheme 4. Brown solid (0.012 g, 4.33%): ¹H NMR (400 MHz, DMSO-d₆) δ 1.39-1.59 (m, 14H), 1.68-1.91 (m, 17H), 2.27 (s, 10H), 2.38 (s, 15H), 3.23 (s, 7H), 3.46 (s, 3H), 4.96-5.08 (m, 1H), 5.32 (d, J=30.2 Hz, 2H), 5.72 (s, OH), 7.31 (d, J=8.5 Hz, 1H), 7.40 (s, 1H), 7.43-7.63 (m, 8H), 7.67 (s, 1H), 7.85 (s, 3H), 8.05 (s, 4H), 8.92 (s, 4H), 10.12 (s, 4H), 11.04 (s, 1H). LC-MS: m/z [M+H]⁺ for C₄₄H₅₀N₁₀O₆, calculated 814.95; observed 815.67.

### Compound (11): 5-(4-(4-(6-(6-acetyl-8-cyclopentyl-5-methyl-7-oxo-7,8-dihydropyrido[2,3-d]pyrimidin-2-yl) amino) pyridin-3-yl) piperazin-1-yl) methyl)-4-methylpiperidin-1-yl)-2-(2,6-dioxopiperidin-3-yl) isoindoline-1,3-dione (AM-01-284)

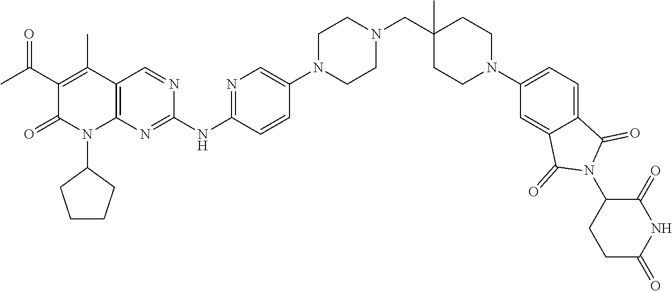 [SMILES: CC(=O)c1c(C)c2cnc(Nc3ccc(N4CCN(CC5(C)CCN(c6ccc7c(c6)C(=O)N(C6CCC(=O)NC6=O)C7=O)CC5)CC4)cn3)nc2n(C2CCCC2)c1=O]

Compound (11) were synthesized using analogous procedures as described in Compound (8) and by employing Palbociclib-linkers shown in Scheme 4. Brown solid (0.023 g, 8.614%): ¹H NMR (400 MHz, DMSO-d₆) δ 1.42 (t, J=7.1 Hz, 6H), 1.47-1.60 (m, 12H), 1.67-1.79 (m, 8H), 1.84 (s, 9H), 2.12 (t, J=7.5 Hz, 4H), 2.26 (s, 16H), 2.37 (s, 16H), 3.42 (s, 20H), 5.06 (s, 2H), 5.27 (s, 1H), 5.81 (s, 1H), 7.38 (s, 2H), 7.50 (s, 2H), 7.68 (s, 1H), 7.86 (s, 2H), 8.09 (s, 4H), 8.91 (s, 6H), 10.10 (s, 5H), 11.06 (s, 4H). LC-MS: m/z [M+H]⁺ for C₄₄H₅₀N₁₀O₆, calculated 814.95; observed 815.78.

### Preparation of Compounds (12)-(15)

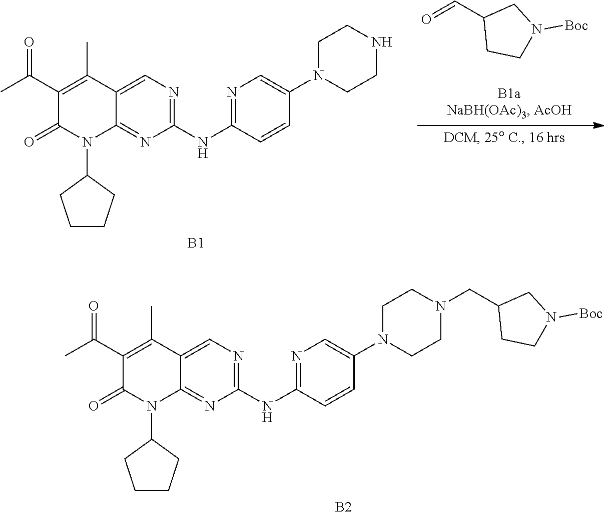 [SMILES: CC(=O)c1c(C)c2cnc(Nc3ccc(N4CCNCC4)cn3)nc2n(C2CCCC2)c1=O ; CC(C)(C)OC(=O)N1CCC(C=O)C1 ; "B1" ; CC(=O)c1c(C)c2cnc(Nc3ccc(N4CCN(CC5CCN(C(=O)OC(C)(C)C)C5)CC4)cn3)nc2n(C2CCCC2)c1=O ; "B2"]

To a solution of compound B1 (400 mg, 894 umol, 0.80 eq) and compound B1a (222 mg, 1.12 mmol, 1.00 eq) in DMF (8.00 mL) was added AcOH (201 mg, 3.35 mmol, 192 μL, 3.00 eq) at 25° C. After stirring at 25° C. for 0.5 hr, NaBH(OAc)₃ (710 mg, 3.35 mmol, 3.00 eq) and NaOAc (137 mg, 1.68 mmol, 1.50 eq) was added to the mixture. The mixture was stirred at 25° C. for 16 hrs. LCMS showed compound B1 was consumed completely and one main peak with desired mass was detected. The reaction mixture was poured into H₂O 4.00 mL and stirred at 25° C. for 0.5 hr. Then the mixture was filtered and the filter cake was dried under reduced pressure to give a residue. Compound B2 (460 mg, 605 umol, 54.2% yield, 83.0% purity) was obtained as a yellow solid checked by LCMS, SFC, HPLC. The crude product was used into next step directly without further purification.

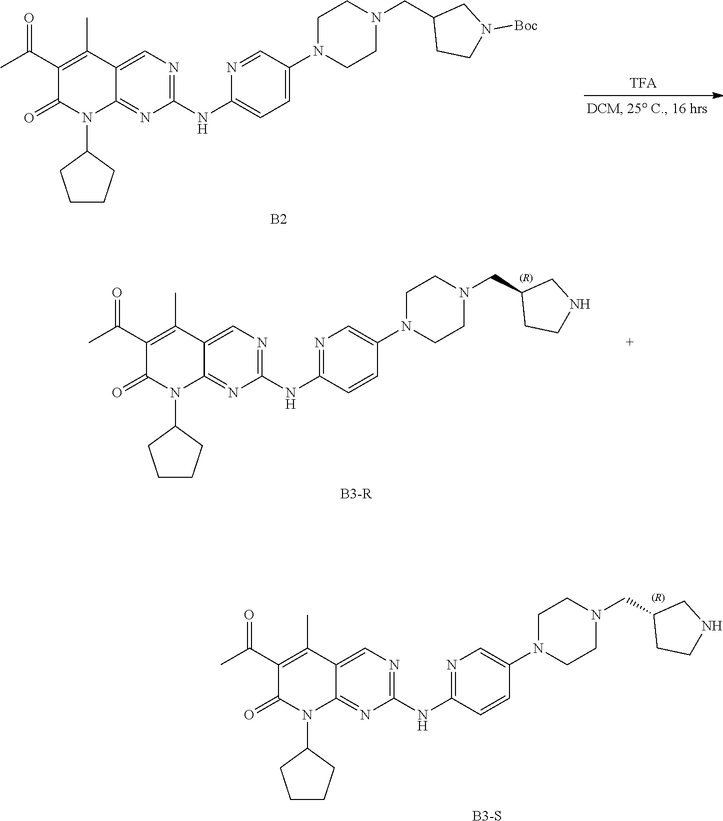

To a solution of compound B2 (460 mg, 729 umol, 1.00 eq) in DCM (9.00 mL) was added TFA (5.67 g, 49.7 mmol, 3.68 mL, 68.2 eq) at 25° C. The mixture was stirred at 25° C. for 16 hrs. LCMS showed compound B2 was consumed completely and one main peak with desired mass was detected. The reaction mixture was concentrated under reduced pressure to give a residue. The pH of the solution was adjusted to around 11-12 by progressively adding 2 N NaOH and extracted with DCM 10.0 mL (5.00 mL*2). The combined organic layers were dried over Na₂SO₄, filtered and concentrated under reduced pressure to give a residue. The residue was further separated by SFC (column: DAICEL CHIRALPAK AD (250 mm*30 mm, 10 um); mobile phase: [0.1% NH₃H₂O IPA]; B %: 50%-50%, 8 min). Compound B3-R or B3-S (Peak 1, 130 mg, 229 umol, 62.7% yield, 93.3% purity) was obtained as a yellow solid checked by SFC.

Compound B3-S or B3-R (Peak 2, 130 mg, 229 umol, 62.7% yield, 93.3% purity) was obtained as a yellow solid checked by SFC.

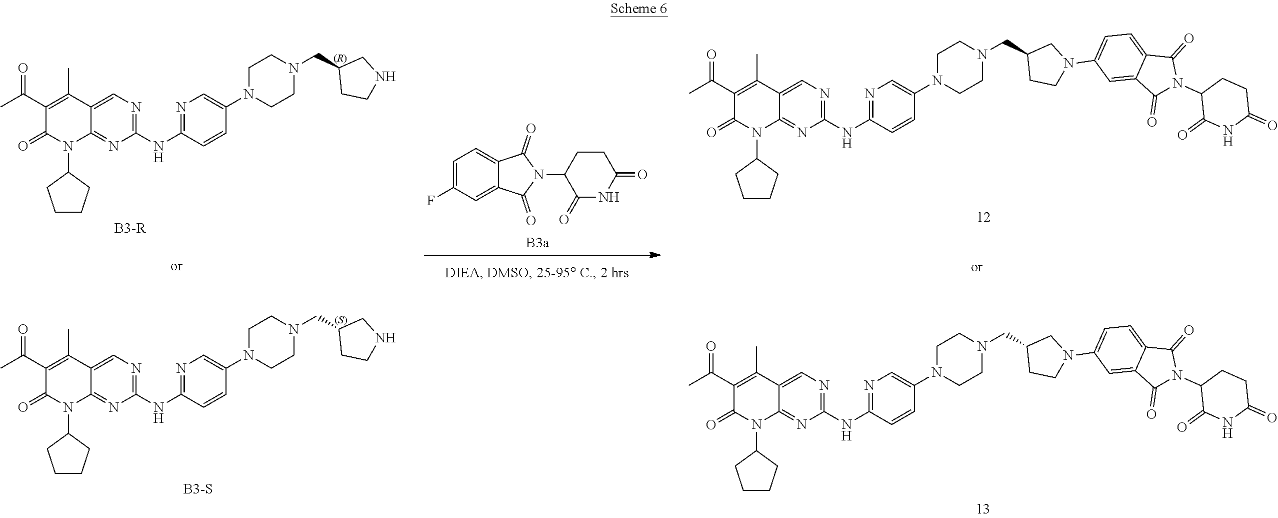

To a solution of compound B3-R or B3-S (Peak 1, 50.0 mg, 94.2 umol, 1.00 eq) in DMSO (1 mL) was added DIEA (48.7 mg, 377 umol, 65.7 uL, 4.00 eq) and compound B3a (52.1 mg, 188 umol, 2.00 eq) at 25° C. The mixture was stirred at 95° C. for 2 hrs. LCMS showed compound B3-R or B3-S was consumed completely and one main peak with desired mass was detected. The reaction mixture was diluted with DMSO (1.00 mL) and purified directly by prep-HPLC (column: Waters Xbridge BEH C18 100*30 mm*10 um; mobile phase: [water (NH₄HCO₃)-ACN]; B %: 45%-75%, 8 min). Compound 12 or 13 (16.7 mg, 20.7 umol, 22.0% yield, 97.6% purity) was obtained as a yellow solid checked by LCMS, HPLC and HNMR. ¹H NMR: (CHLOROFORM-d) 6=8.74 (s, 1H), 8.45 (br s, 1H), 8.11 (br d, J=9.0 Hz, 2H), 7.99 (d, J=2.6 Hz, 1H), 7.59 (d, J=8.4 Hz, 1H), 7.31-7.25 (m, 1H), 6.90 (d, J=1.8 Hz, 1H), 6.63 (dd, J=1.9, 8.6 Hz, 1H), 5.81 (quin, J=8.8 Hz, 1H), 4.87 (dd, J=5.2, 12.2 Hz, 1H), 3.58-3.42 (m, 2H), 3.42-3.35 (m, 1H), 3.21-3.14 (m, 4H), 2.87-2.74 (m, 2H), 2.74-2.56 (m, 6H), 2.48 (s, 4H), 2.30 (s, 4H), 2.27-2.15 (m, 2H), 2.14-2.02 (m, 2H), 2.02-1.91 (m, 2H), 1.87-1.71 (m, 4H), 1.62 (br d, J=5.0 Hz, 2H)

To a solution of compound 3-R or 3-S (Peak 2, 60.0 mg, 113 umol, 1.00 eq) in DMSO (1.00 mL) was added DIEA (58.5 mg, 452 umol, 78.8 uL, 4.00 eq) and compound B3a (62.5 mg, 226 umol, 2.00 eq) at 25° C. The mixture was stirred at 95° C. for 2 hrs. LCMS showed compound B3-R or B3-S was consumed completely and one main peak with desired mass was detected. The reaction mixture was diluted with DMSO (1.00 mL) and then purified directly by prep-HPLC (column: Waters Xbridge BEH C18 100*30 mm*10 um; mobile phase: [water (NH₄HCO₃)-ACN]; B %: 35%-68%, 8 min). Compound 12 or 13 (20.4 mg, 25.5 umol, 22.5% yield, 98.1% purity) was obtained as a yellow solid checked by LCMS, HPLC and HNMR. ¹H NMR: (400 MHz, CHLOROFORM-d) 6=8.73 (s, 1H), 8.30 (br s, 1H), 8.12 (br d, J=9.0 Hz, 1H), 8.02 (br s, 1H), 7.99 (br d, J=2.3 Hz, 1H), 7.60 (d, J=8.4 Hz, 1H), 7.30-7.25 (m, 1H), 6.90 (s, 1H), 6.64 (br d, J=7.1 Hz, 1H), 5.81 (br t, J=8.8 Hz, 1H), 4.87 (br dd, J=5.1, 12.3 Hz, 1H), 3.59-3.44 (m, 2H), 3.41-3.32 (m, 1H), 3.18 (br s, 4H), 2.88-2.58 (m, 8H), 2.48 (s, 3H), 2.30 (s, 3H), 2.27-2.14 (m, 2H), 2.06 (br d, J=8.5 Hz, 2H), 1.99 (br s, 2H), 1.81 (br s, 4H), 1.65-1.61 (m, 2H).

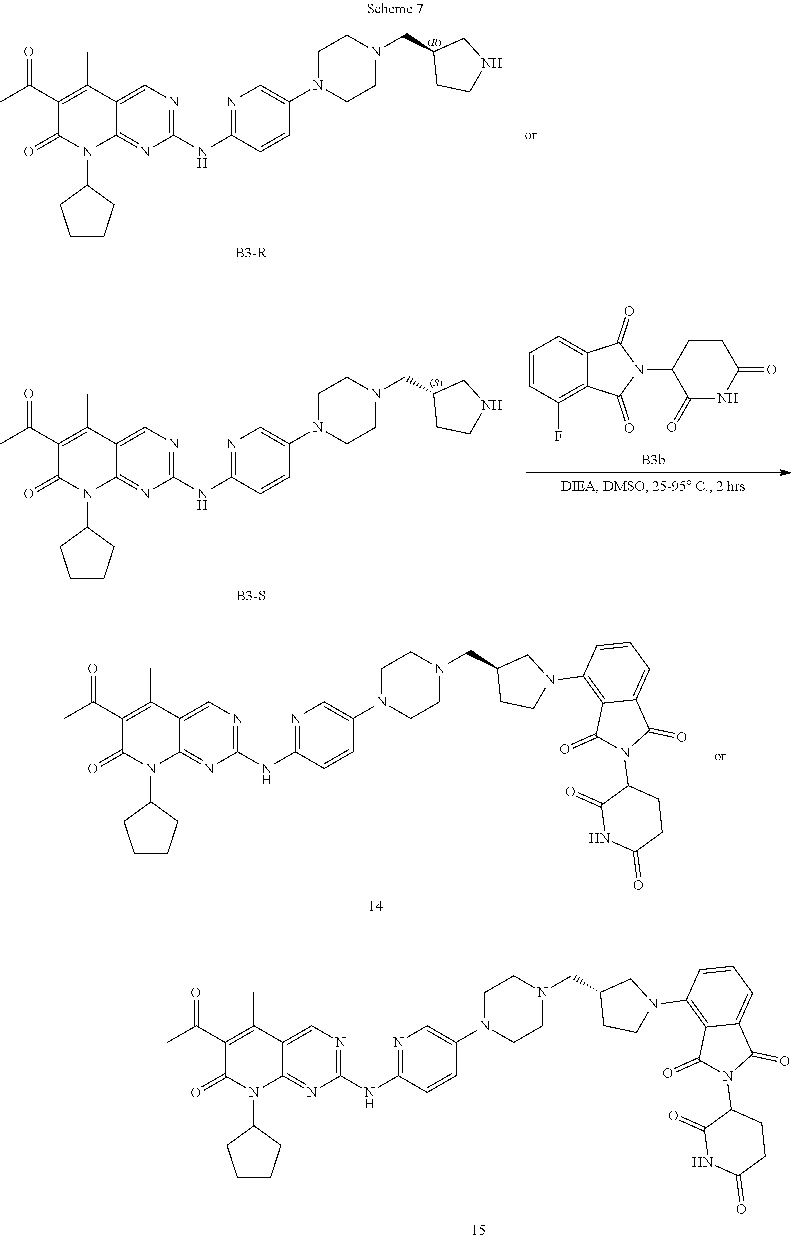

To a solution of compound B3-R or B3-S (Peak 1, 50.0 mg, 94.2 umol, 1.00 eq) in DMSO (1.00 mL) was added DIEA (48.7 mg, 377 umol, 65.7 uL, 4.00 eq) and compound B3b (52.1 mg, 188 umol, 2.00 eq) at 25° C. The mixture was stirred at 95° C. for 2 hrs. LCMS showed compound B3-R or B3-S was consumed completely and one main peak with desired mass was detected. The reaction mixture was diluted with DMSO (1.00 mL) and then purified directly by prep-HPLC (column: Waters Xbridge BEH C18 100*30 mm*10 um; mobile phase: [water (NH₄HCO₃)-ACN]; B %: 45%-75%, 8 min). Compound 14 or 15 (9.89 mg, 12.5 umol, 13.2% yield, 99.1% purity) was obtained as a yellow solid checked by LCMS, HPLC and HNMR. ¹H NMR: (CHLOROFORM-d) 6=8.67 (d, J=3.9 Hz, 1H), 8.16 (br s, 1H), 8.04 (br d, J=8.8 Hz, 1H), 7.95-7.90 (m, 1H), 7.38-7.31 (m, 1H), 7.24-7.19 (m, 1H), 7.12-7.04 (m, 2H), 6.84 (br d, J=8.0 Hz, 1H), 5.80-5.70 (m, 1H), 5.17 (s, 1H), 4.83 (br dd, J=5.4, 11.9 Hz, 1H), 3.64-3.50 (m, 3H), 3.48-3.24 (m, 2H), 3.12 (br s, 3H), 2.81-2.70 (m, 2H), 2.67-2.51 (m, 6H), 2.42 (s, 3H), 2.24 (s, 3H), 2.21-2.06 (m, 2H), 2.05-1.89 (m, 4H), 1.81-1.65 (m, 4H), 1.59-1.55 (m, 2H)

To a solution of compound B3-R or B3-S (60.0 mg, 113 umol, 1 eq) in DMSO (1.00 mL) was added DIEA (58.5 mg, 452 umol, 78.8 uL, 4.00 eq) and compound B3b (62.5 mg, 226 umol, 2 eq) at 25° C. The mixture was stirred at 95° C. for 2 hrs. LCMS (ET62422-15-P1A1) showed compound B3-R or B3-S was consumed completely and one main peak with desired mass was detected. The reaction mixture was diluted with DMSO (1.00 mL) and then purified directly by prep-HPLC (column: Waters Xbridge BEH C18 100*30 mm*10 um; mobile phase: [water (NH₄HCO₃)-ACN]; B %: 45%-75%, 8 min). Compound 14 or 15 (20.0 mg, 24.8 umol, 22.0% yield, 97.7% purity) was obtained as a yellow solid checked by LCMS, HPLC and HNMR. ¹H NMR: (400 MHz, CHLOROFORM-d) 6=8.74 (s, 2H), 8.33-8.18 (m, 1H), 8.09 (d, J=9.1 Hz, 1H), 7.99 (t, J=2.9 Hz, 1H), 7.39 (dd, J=7.2, 8.3 Hz, 1H), 7.30-7.23 (m, 1H), 7.14 (d, J=6.8 Hz, 1H), 6.88 (d, J=8.6 Hz, 1H), 5.81 (quin, J=8.9 Hz, 1H), 4.89 (dd, J=5.4, 12.0 Hz, 1H), 3.71-3.54 (m, 3H), 3.48-3.32 (m, 1H), 3.15 (q, J=5.0 Hz, 4H), 2.88-2.71 (m, 2H), 2.70-2.51 (m, 6H), 2.48 (s, 3H), 2.46-2.36 (m, 2H), 2.30 (s, 3H), 2.29-2.25 (m, 1H), 2.16-1.95 (m, 4H), 1.87-1.59 (m, 6H)

### Preparation of Compound (16)

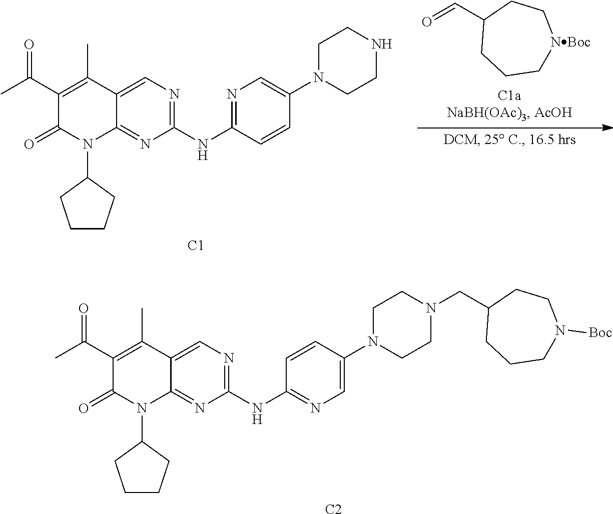

To a solution of compound C1 (100 mg, 223 umol, 0.80 eq) in DCM (2.00 mL) was added compound C1a (63.4 mg, 279 umol, 1.00 eq) and AcOH (50.3 mg, 837 umol, 47.9 uL, 3.00 eq) at 25° C. under N₂. The mixture was stirred at 25° C. for 0.5 hr. Then to the mixture was added NaBH(OAc)₃ (177 mg, 837 umol, 3.00 eq) at 25° C. The mixture was stirred at 25° C. for 16 hrs. LCMS indicated compound C1 was consumed completely and one main peak with desired mass was detected. The mixture was concentrated under reduced pressure to give a residue. The residue was suspended in H₂O (10.0 mL) and after stirring at 25° C. for 0.5 hr, the mixture was filtered. The filter cake was dried under reduced pressure to give the crude product compound C2 (150 mg, crude) as a yellow solid.

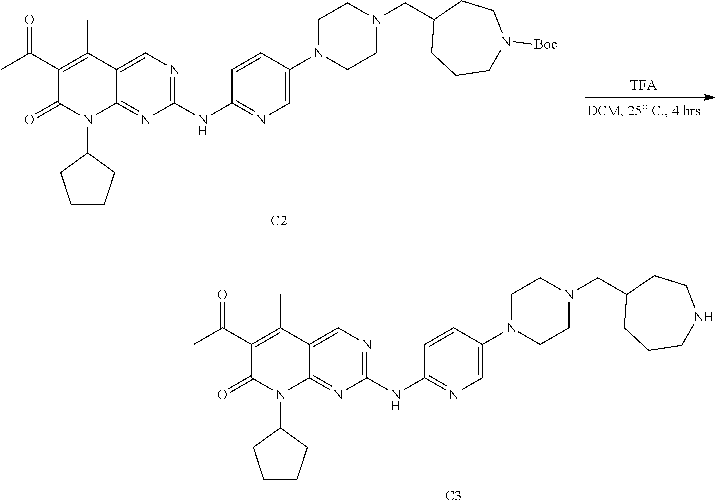 [SMILES: CC(=O)c1c(C)c2cnc(Nc3ccc(N4CCN(CC5CCCN(C(=O)OC(C)(C)C)CC5)CC4)cn3)nc2n(C2CCCC2)c1=O ; "C2" ; CC(=O)c1c(C)c2cnc(Nc3ccc(N4CCN(CC5CCCNCC5)CC4)cn3)nc2n(C2CCCC2)c1=O ; "C3"]

To a solution of compound C2 (150 mg, 227 umol, 1.00 eq) in DCM (2.00 mL) was added TFA (1.54 g, 13.5 mmol, 1.00 mL, 59.3 eq) at 25° C. Then the mixture was stirred at 25° C. for 4 hrs. LC-MS showed compound 2 was consumed completely and one main peak with desired mass was detected. The mixture was concentrated under reduced pressure to give a residue. The residue was diluted with DCM (20 mL) and the pH of the mixture was adjusted to ˜10 by addition of aq. NaOH (2 N, 5 mL). Then the mixture was extracted with DCM 10 mL (5 mL*2). The combined organic layers were concentrated under reduced pressure to give the crude product compound C3 (110 mg, 196 umol, 86.4% yield) as a yellow solid.

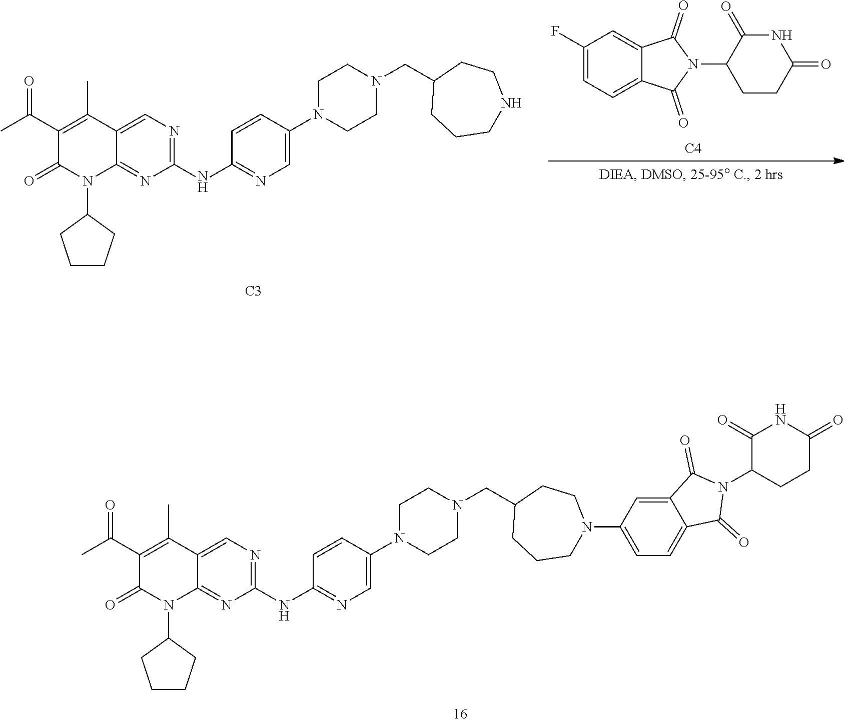 [SMILES: CC(=O)c1c(C)c2cnc(Nc3ccc(N4CCN(CC5CCCNCC5)CC4)cn3)nc2n(C2CCCC2)c1=O ; O=C1CCC(N2C(=O)c3ccc(F)cc3C2=O)C(=O)N1 ; "C4" ; "C3" ; CC(=O)c1c(C)c2cnc(Nc3ccc(N4CCN(CC5CCCN(c6ccc7c(c6)C(=O)N(C6CCC(=O)NC6=O)C7=O)CC5)CC4)cn3)nc2n(C2CCCC2)c1=O]

To a solution of compound C4 (80.0 mg, 289 umol, 2.02 eq) and compound C₃ (80.0 mg, 143 umol, 1.00 eq) in DMSO (2.00 mL) was added DIEA (74.2 mg, 574 umol, 0.10 mL, 4.01 eq) at 25° C. Then the mixture was stirred at 95° C. for 2 hrs. LC-MS showed compound C3 was consumed completely and one main peak with desired m/z was detected. The mixture was diluted with DMSO (2.00 mL) and purified directly by prep-HPLC (column: Waters Xbridge BEH C18 100*30 mm*10 um; mobile phase: [water (NH₄HCO₃)-ACN]; B %: 50%-80%, 8 min). Compound 16 (30 mg, 36.1 umol, 25.7% yield, 98.1% purity) was obtained as a yellow solid checked by HNMR, LCMS and HPLC. ¹H NMR: ET62148-24-P1F (400 MHz, CDCl₃) δ=9.46-9.13 (m, 1H), 8.85 (s, 1H), 8.63 (br s, 1H), 8.16 (br d, J=9.0 Hz, 1H), 8.09 (d, J=2.5 Hz, 1H), 7.67 (d, J=8.6 Hz, 1H), 7.34 (dd, J=2.6, 9.1 Hz, 1H), 7.12 (d, J=1.6 Hz, 1H), 6.90-6.83 (m, 1H), 5.89 (quin, J=8.8 Hz, 1H), 4.97 (br dd, J=5.3, 12.1 Hz, 1H), 3.77-3.64 (m, 2H), 3.59-3.45 (m, 2H), 3.19 (br s, 4H), 2.98-2.70 (m, 3H), 2.56 (s, 7H), 2.44-2.33 (m, 5H), 2.24-2.11 (m, 4H), 2.04 (br d, J=16.0 Hz, 3H), 1.98-1.82 (m, 4H), 1.82-1.72 (m, 3H), 1.50-1.39 (m, 1H), 1.20-1.06 (m, 1H).

### Preparation of Compounds (17)-(18)

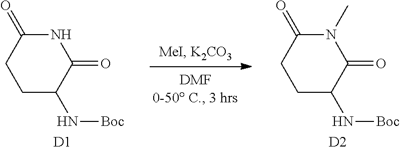

To a mixture of compound D1 (2.50 g, 10.9 mmol, 1.00 eq) in DMF (37.5 mL) was added K₂CO₃ (4.54 g, 32.9 mmol, 3.00 eq) and Mel (4.66 g, 32.9 mmol, 2.05 mL, 3.00 eq) in portions at 0° C. And then the mixture stirred at 50° C. for 3 hrs. LCMS indicated compound D1 was consumed completely. The reaction mixture was quenched by addition H₂O (40.0 mL) and then extracted with EtOAc (50.0 mL*3). The combined organic layers were dried over Na₂SO₄, filtered and concentrated under reduced pressure to give a residue. The residue was purified by column chromatography (SiO₂, Petroleum ether/Ethyl acetate=20/1 to 1/1). Compound D2 (2.40 g, 9.91 mmol, 90.4% yield) was obtained as a white solid checked by HNMR. ¹H NMR: (400 MHz, DMSO-d₆) δ 7.19 (br d, J=8.6 Hz, 1H), 4.36-4.24 (m, 1H), 2.98 (s, 3H), 2.86-2.75 (m, 1H), 2.70-2.60 (m, 1H), 1.98-1.86 (m, 2H), 1.41 (s, 9H).

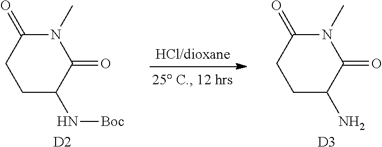

The mixture of compound D2 (200 mg, 825 umol, 1.00 eq) in HCl/dioxane (4.00 M, 1.03 mL, 5.00 eq) was stirred at 25° C. for 12 hrs. LCMS indicated compound D2 was consumed completely. The mixture was concentrated under reduced to give the crude product compound D3 (110 mg, 795 umol, 96.3% yield) as a white solid.

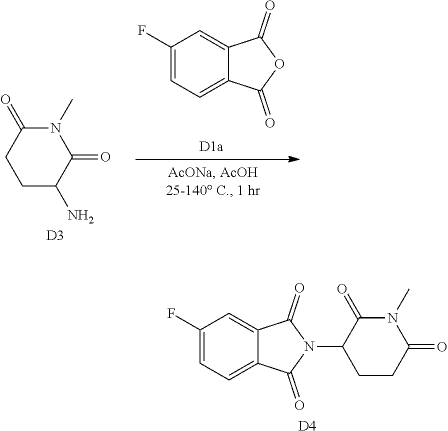

To a mixture of compound D3 (110 mg, 795 umol, 1.00 eq), compound D1a (106 mg, 636 umol, 0.800 eq) in AcOH (1.20 mL) was added AcONa (130 mg, 1.59 mmol, 2.00 eq) at 25° C. The mixture was stirred at 140° C. for 1 hr under N₂. LCMS indicated compound D3 was consumed completely. The reaction mixture was diluted with H₂O 2.00 mL and filtered, the filter cake was dried to give a residue. Compound D4 (140 mg, 482 umol, 60.7% yield) was obtained as a white solid checked by HNMR. ¹H NMR: (400 MHz, CDCl₃-d) δ=7.83 (dd, J=4.5, 8.3 Hz, 1H), 7.49 (dd, J=2.3, 7.0 Hz, 1H), 7.37 (dt, J=2.3, 8.5 Hz, 1H), 4.98-4.82 (m, 1H), 3.15 (s, 3H), 3.00-2.85 (m, 1H), 2.80-2.64 (m, 2H), 2.14-1.97 (m, 1H).

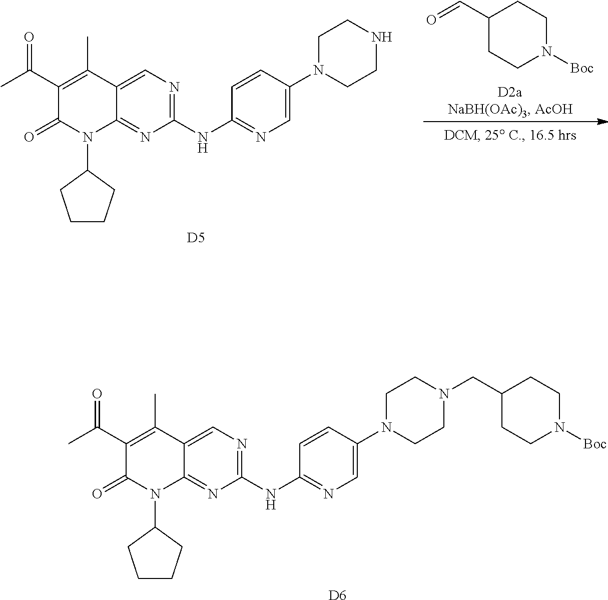 [SMILES: CC(=O)c1c(C)c2cnc(Nc3ccc(N4CCNCC4)cn3)nc2n(C2CCCC2)c1=O ; CC(C)(C)OC(=O)N1CCC(C=O)CC1 ; "D5" ; CC(=O)c1c(C)c2cnc(Nc3ccc(N4CCN(CC5CCN(C(=O)OC(C)(C)C)CC5)CC4)cn3)nc2n(C2CCCC2)c1=O ; "D6"]

To a solution of compound D2a (59.6 mg, 279 umol, 1.00 eq) and compound 5 (100 mg, 223 umol, 0.800 eq) in DMF (2.00 mL) was added AcONa (34.4 mg, 419 umol, 1.50 eq) at 25° C. After stirring at 25° C. for 0.5 hr, NaBH(OAc)₃ (178 mg, 838 umol, 3.00 eq) and AcOH (50.3 mg, 838 umol, 47.9 uL, 3.00 eq) was added to the mixture at 25° C. The mixture was stirred at 25° C. for 16 hrs. LCMS indicated compound D5 was consumed completely. The reaction mixture was suspended in H₂O (2.00 mL) and then filtered. The filter cake was dried in vacuo to give compound D6 (148 mg, 230 umol, 82.2% yield) as a yellow solid. The crude product was used into the next step without further purification.

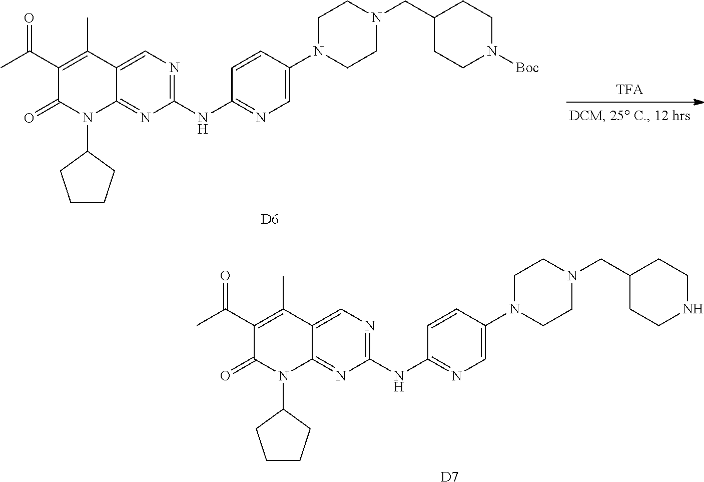 [SMILES: CC(=O)c1c(C)c2cnc(Nc3ccc(N4CCN(CC5CCN(C(=O)OC(C)(C)C)CC5)CC4)cn3)nc2n(C2CCCC2)c1=O ; CC(=O)c1c(C)c2cnc(Nc3ccc(N4CCN(CC5CCNCC5)CC4)cn3)nc2n(C2CCCC2)c1=O ; "D6" ; "D7"]

To a solution of compound D6 (148 mg, 230 umol, 1.00 eq) in DCM (2.00 mL) was added TFA (1.54 g, 13.5 mmol, 1.00 mL, 58.8 eq). The mixture was stirred at 25° C. for 12 hrs. LCMS indicated compound D6 was consumed completely. The reaction mixture was diluted with DCM 3.00 mL and alkalified with 1N NaOH (6.00 mL) under stirring on ice bath. Then the mixture was extracted with DCM (2.00 mL*2). The organic layer was dried over anhydrous Na₂SO₄, filtered and concentrated under reduced pressure to give a residue. Compound D7 (105 mg, 193 umol, 84.0% yield) was obtained as a yellow solid checked by HNMR. The crude product was used into the next step without further purification. ¹H NMR: (400 MHz, CHLOROFORM-d) 6=8.74 (s, 1H), 8.08 (br d, J=9.1 Hz, 1H), 7.97 (d, J=2.8 Hz, 1H), 7.26 (dd, J=2.9, 9.1 Hz, 1H), 5.81 (quin, J=8.8 Hz, 1H), 3.19-3.09 (m, 5H), 2.71-2.58 (m, 2H), 2.55-2.50 (m, 4H), 2.48 (s, 3H), 2.30 (s, 3H), 2.28-2.23 (m, 2H), 2.20 (br d, J=7.1 Hz, 2H), 2.03-1.95 (m, 2H), 1.87-1.73 (m, 4H), 1.72-1.53 (m, 4H), 1.24-1.17 (m, 2H).

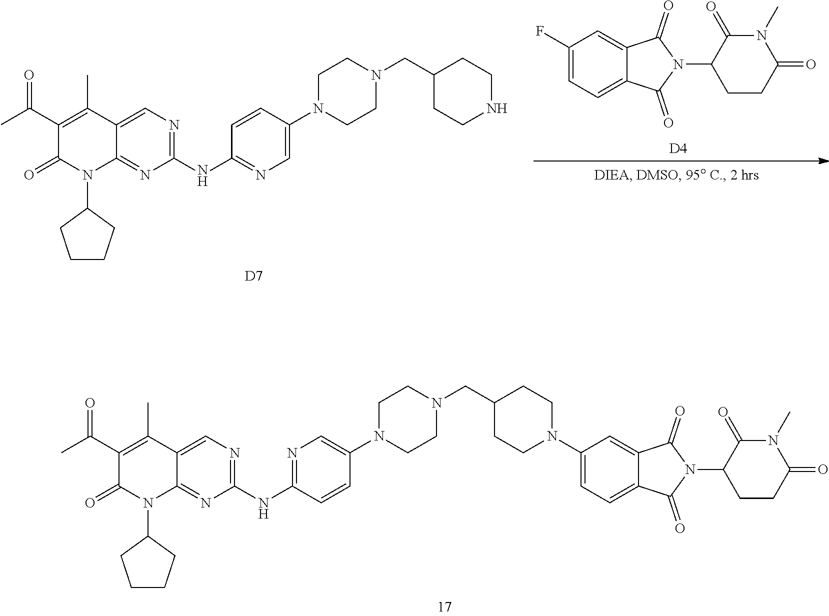

A mixture of compound D7 (50.0 mg, 91.8 umol, 1.00 eq), compound D4 (53.3 mg, 184 umol, 2.00 eq), DIEA (47.5 mg, 367 umol, 63.9 uL, 4.00 eq) in DMSO (8.00 mL) was degassed and purged with N₂ for 3 times, and then the mixture was stirred at 95° C. for 2 hrs under N₂ atmosphere. LCMS indicated compound 7 was consumed completely. The residue was diluted with DMSO (1.00 mL) and then purified directly by prep-HPLC (column: Waters Xbridge BEH C18 100*30 mm*10 um; mobile phase: [water (NH₄HCO₃)-ACN]; B %: 45%-75%, 8 min). Compound 17 (20.4 mg, 24.8 umol, 27.0% yield, 99.1% purity) was obtained as a yellow solid checked by HNMR, LCMS and HPLC. ¹HNMR: (400 MHz, CHLOROFORM-d) 6=8.75 (s, 1H), 8.20-8.05 (m, 2H), 7.98 (d, J=2.9 Hz, 1H), 7.60 (d, J=8.5 Hz, 1H), 7.27 (dd, J=2.9, 9.1 Hz, 1H), 7.21 (d, J=2.3 Hz, 1H), 6.98 (dd, J=2.3, 8.6 Hz, 1H), 5.81 (quin, J=8.9 Hz, 1H), 4.94-4.79 (m, 1H), 3.89 (br d, J=13.0 Hz, 2H), 3.16-3.12 (m, 7H), 2.97-2.84 (m, 3H), 2.78-2.63 (m, 2H), 2.60-2.52 (m, 4H), 2.48 (s, 3H), 2.30 (s, 4H), 2.25-2.18 (m, 2H), 2.06-1.93 (m, 3H), 1.89-1.76 (m, 5H), 1.68-1.60 (m, 3H), 1.30-1.19 (m, 2H).

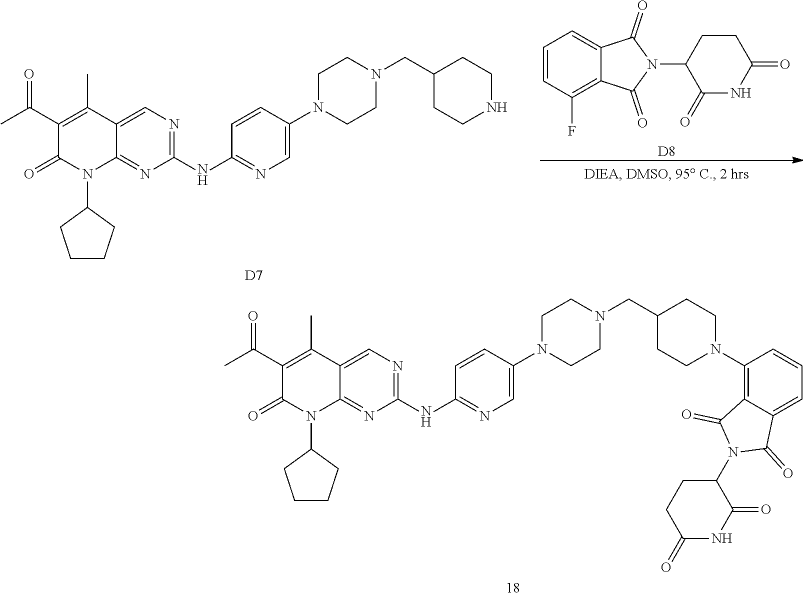

A mixture of compound D7 (50.0 mg, 91.8 umol, 1.00 eq), compound D8 (50.7 mg, 183 umol, 2.00 eq), DIEA (47.5 mg, 367 umol, 63.9 uL, 4.00 eq) in DMSO (2.00 mL) was degassed and purged with N₂ for 3 times, and then the mixture was stirred at 95° C. for 2 hrs under N₂ atmosphere. LCMS showed ˜6% compound D7 was remained and one main peak with desired MS was detected. The mixture was diluted with DMSO (5 mL) and purified directly by prep-HPLC (column: Waters Xbridge Prep OBD C18 150*40 mm*10 um; mobile phase: [water (NH₄HCO₃)-ACN]; B %: 45%-75%, 8 min). Compound 18 (30 mg, 36.9 umol, 26.8% yield, 98.4% purity) was obtained as a yellow solid checked by HNMR, HPLC and LCMS. ¹HNMR: (400 MHz, CDCl₃-d) δ=8.73 (s, 1H), 8.52 (br s, 1H), 8.18-8.05 (m, 2H), 7.98 (d, J=2.4 Hz, 1H), 7.50 (t, J=7.8 Hz, 1H), 7.33-7.24 (m, 2H), 7.14-7.09 (m, 1H), 5.81 (quin, J=8.9 Hz, 1H), 4.90 (br dd, J=5.2, 12.3 Hz, 1H), 3.69 (br t, J=11.2 Hz, 2H), 3.14 (br s, 3H), 2.90-2.62 (m, 5H), 2.56 (br s, 3H), 2.48 (s, 3H), 2.35-2.24 (m, 6H), 2.09-1.93 (m, 3H), 1.93-1.76 (m, 4H), 1.74-1.59 (m, 5H), 1.49-1.34 (m, 3H).

### Preparation of Compound (19)

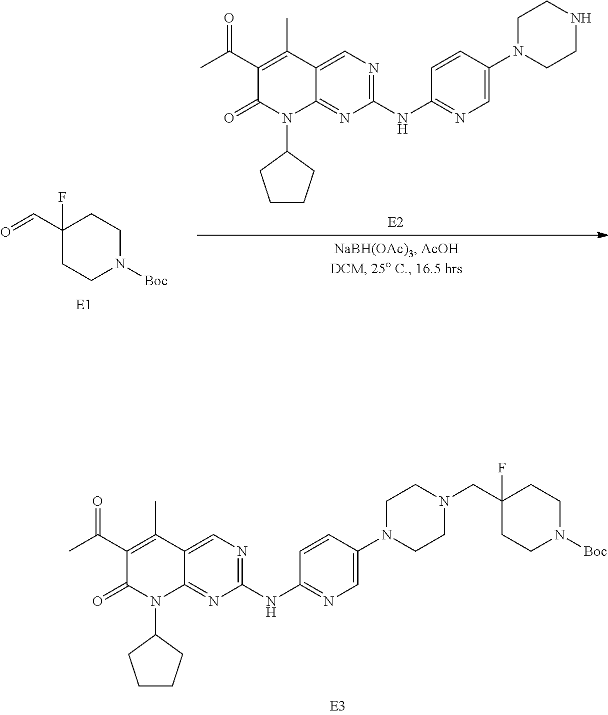 [SMILES: CC(C)(C)OC(=O)N1CCC(F)(C=O)CC1 ; CC(=O)c1c(C)c2cnc(Nc3ccc(N4CCNCC4)cn3)nc2n(C2CCCC2)c1=O ; CC(=O)c1c(C)c2cnc(Nc3ccc(N4CCN(CC5(F)CCN(C(=O)OC(C)(C)C)CC5)CC4)cn3)nc2n(C2CCCC2)c1=O]

To a solution of compound E1 (50.0 mg, 216 umol, 1.55 eq) in DCM (2.00 mL) was added compound E2 (50.0 mg, 111 umol, 0.80 eq) and AcOH (25.1 mg, 418 umol, 23.9 uL, 3.00 eq) at 25° C., the mixture was stirred at 25° C. for 0.5 hr. Then NaBH(OAc)₃ (88.8 mg, 418 umol, 3.00 eq) was added to the mixture at 25° C. The mixture was stirred at 25° C. for 16 hrs. LCMS indicated the compound E2 was consumed completely and desired mass was detected. The mixture was concentrated under reduced pressure to give a residue. The residue was suspended in H₂O (5 mL) and filtered. The filter cake was dried under reduced pressure to give crude product compound E3 (74.0 mg, crude) as a yellow solid.

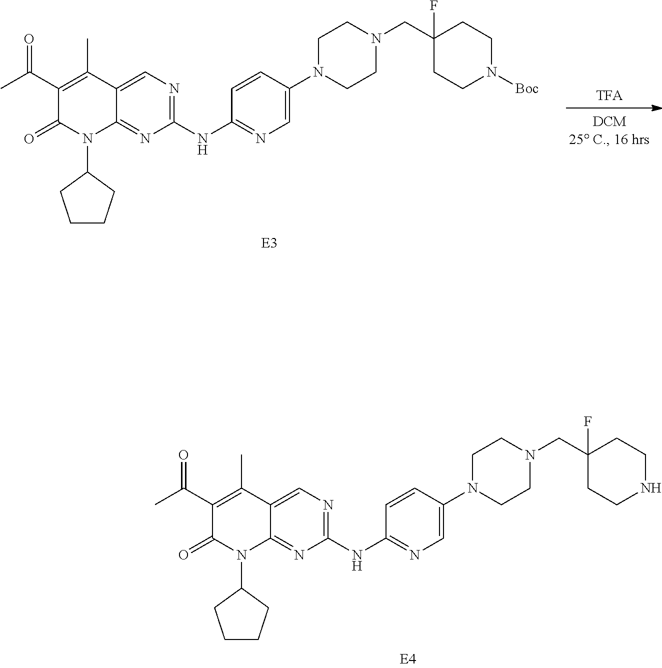 [SMILES: CC(=O)c1c(C)c2cnc(Nc3ccc(N4CCN(CC5(F)CCN(C(=O)OC(C)(C)C)CC5)CC4)cn3)nc2n(C2CCCC2)c1=O ; CC(=O)c1c(C)c2cnc(Nc3ccc(N4CCN(CC5(F)CCNCC5)CC4)cn3)nc2n(C2CCCC2)c1=O]

To a solution of compound E3 (74.0 mg, 111 umol, 1.00 eq) in DCM (2.00 mL) was added TFA (1.54 g, 13.5 mmol, 1.00 mL, 120 eq) at 25° C. The mixture was stirred at 25° C. for 16 hrs. LC-MS showed compound E3 was consumed completely and desired mass was detected. The mixture was concentrated under reduced pressure to give a residue. The residue was diluted with DCM (20.0 mL) and the pH of the mixture was adjusted to ˜10 by addition of NaOH (2 N, 5.00 mL). Then the aqueous layer was extracted with DCM 10.0 mL (5.00 mL*2). The combined organic layers were concentrated under reduced pressure to give compound E4 (40.0 mg, crude) as a yellow solid.

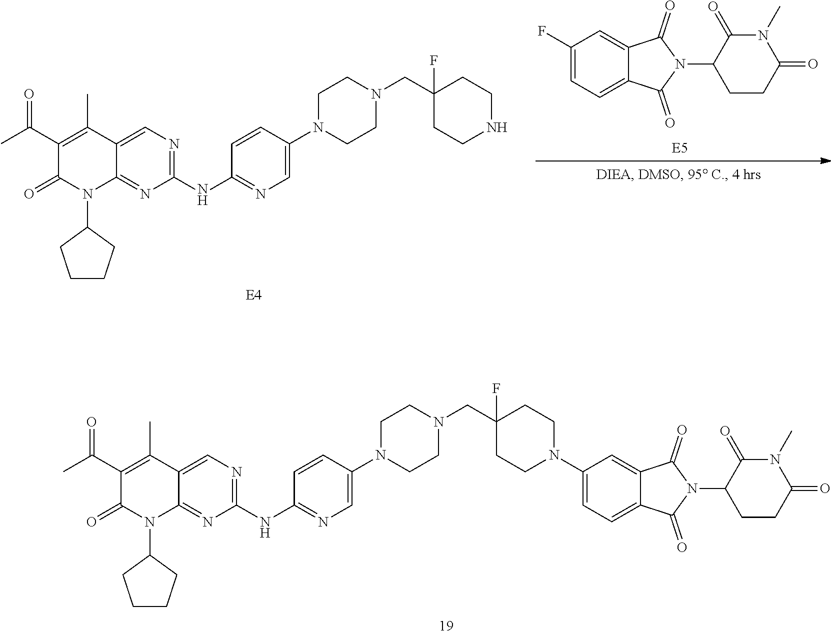

To a solution of compound E5 (37.1 mg, 127 umol, 2.00 eq) and compound E4 (36.0 mg, 63.9 umol, 1.00 eq) in DMSO (0.50 mL) was added DIEA (33.0 mg, 255 umol, 44.5 uL, 4.00 eq) at 25° C., the mixture was stirred at 95° C. for 4 hrs. LC-MS showed compound E4 was consumed completely and desired mass was detected. The mixture was diluted with DMSO (2.00 mL) and then purified directly by prep-HPLC (column: Waters Xbridge BEH C18 100*30 mm*10 um; mobile phase: [water (NH₄HCO₃)-ACN]; B %: 50%-80%, 8 min). Compound 19 (10.09 mg, 12.0 umol, 18.8% yield, 99.5% purity) was obtained as a yellow solid checked by HNMR, FNMR, LCMS and HPLC. ¹H NMR: (400 MHz, CDCl₃) δ=8.73 (s, 1H), 8.10 (br d, J=9.0 Hz, 1H), 8.02 (br s, 1H), 7.95 (br d, J=2.3 Hz, 1H), 7.62 (d, J=8.4 Hz, 1H), 7.30-7.22 (m, 2H), 7.01 (dd, J=1.8, 8.6 Hz, 1H), 5.80 (quin, J=8.9 Hz, 1H), 4.87 (br dd, J=5.3, 12.2 Hz, 1H), 3.72 (br d, J=12.5 Hz, 2H), 3.31 (br t, J=11.8 Hz, 2H), 3.14 (s, 7H), 2.95-2.87 (m, 1H), 2.81-2.63 (m, 6H), 2.61-2.45 (m, 5H), 2.35-2.23 (m, 5H), 2.10-1.96 (m, 5H), 1.85-1.73 (m, 3H), 1.65-1.60 (m, 3H).

### Preparation of Compound (20)

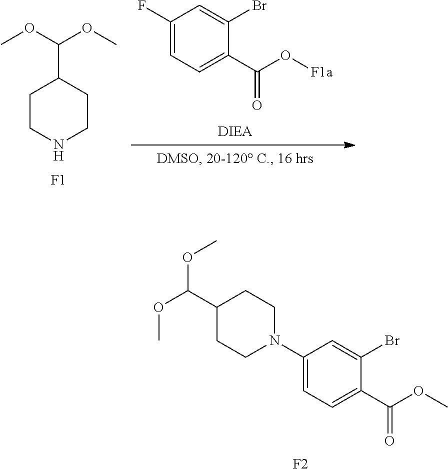

To a solution of compound Fla (484 mg, 2.08 mmol, 1.00 eq) and compound F1 (430 mg, 2.70 mmol, 1.30 eq) in DMSO (6 mL) was added DIEA (805 mg, 6.23 mmol, 1.09 mL, 3.00 eq) at 25° C. The mixture was stirred at 120° C. for 16 hrs. TLC indicated compound Fla was consumed completely and one major new spot with larger polarity was detected. The reaction was clean according to TLC. LC-MS showed compound Fla was consumed completely and one main peak with desired mass was detected. The reaction mixture was quenched by addition H₂O (15 mL) at 25° C., and extracted with ethyl acetate 30 mL (10 mL*3). The combined organic layers were washed with NH₄Cl 15 mL (5 mL*3), dried over Na₂SO₄, filtered and concentrated under reduced pressure to give a residue. The crude was used into next step without further purification. Compound F2 (550 mg, crude) was obtained as a yellow solid. ¹H NMR: (400 MHz, DMSO-d₆) δ=7.79-7.64 (m, 1H), 7.13 (d, J=2.5 Hz, 1H), 6.94 (dd, J=2.5, 9.1 Hz, 1H), 4.11-4.03 (m, 1H), 3.90 (br d, J=13.0 Hz, 2H), 3.76 (s, 3H), 3.26 (s, 6H), 2.80 (dt, J=2.1, 12.6 Hz, 2H), 1.82 (ddt, J=3.6, 7.4, 11.3 Hz, 1H), 1.69 (br d, J=12.9 Hz, 2H), 1.31-1.16 (m, 2H).

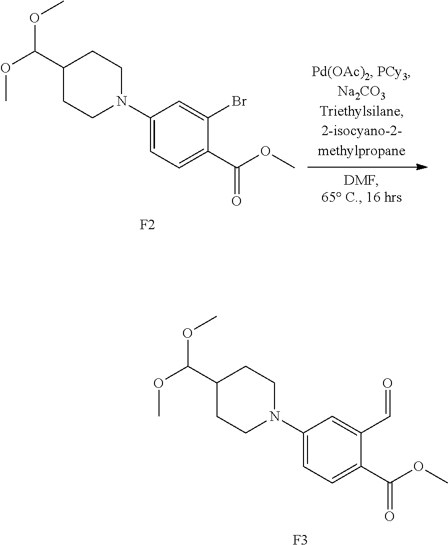 [SMILES: COC(=O)c1ccc(N2CCC(C(OC)OC)CC2)cc1Br ; "F2" ; COC(=O)c1ccc(N2CCC(C(OC)OC)CC2)cc1C=O ; "F3"]

To a solution of compound F2 (300 mg, 806 umol, 1.00 eq) in DMF (3 mL) was added 2-isocyano-2-methyl-propane (134 mg, 1.61 mmol, 182. uL, 2.00 eq), diacetoxypalladium (18.1 mg, 80.6 umol, 0.10 eq), tricyclohexylphosphane (22.6 mg, 80.6 umol, 26.1 uL, 0.10 eq), sodium carbonate (85.4 mg, 806 umol, 1.00 eq) and triethylsilane (281 mg, 2.42 mmol, 386 μL, 3.00 eq) at 25° C. The mixture was stirred at 65° C. for 16 hrs. TLC indicated ˜30% of compound F2 was remained and one new spot formed. The reaction mixture was diluted with H₂O (15 mL) and extracted with ethyl acetate 30 mL (10 mL*3). The combined organic layers were washed with brine (10 mL), dried over Na₂SO₄, filtered and concentrated under reduced pressure to give a residue. Compound F3 (250 mg, crude) was obtained as a red oil. The crude was used into next step without further purification. ¹H NMR: (400 MHz, CHLOROFORM-d) δ=7.91-7.85 (m, 1H), 7.11-6.98 (m, 1H), 6.85 (dd, J=2.8, 8.9 Hz, 1H), 4.07 (dd, J=2.9, 6.7 Hz, 2H), 3.96 (br d, J=12.8 Hz, 4H), 3.86 (s, 3H), 3.40-3.38 (m, 2H), 3.38-3.38 (m, 1H), 3.38 (s, 6H), 0.98 (s, 3H).

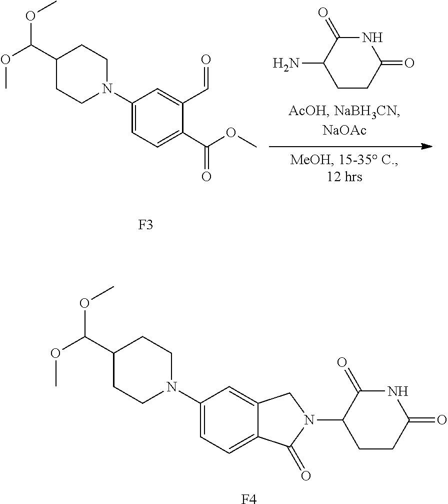 [SMILES: COC(=O)c1ccc(N2CCC(C(OC)OC)CC2)cc1C=O ; NC1CCC(=O)NC1=O ; "F3" ; COC(OC)C1CCN(c2ccc3c(c2)CN(C2CCC(=O)NC2=O)C3=O)CC1 ; "F4"]

To a solution of 3-aminopiperidine-2,6-dione;hydrochloride (101 mg, 616 umol, 1.10 eq) in MeOH (2 mL) was added NaOAc (91.9 mg, 1.12 mmol, 2.00 eq). The mixture was stirred at 25° C. for 10 min. Then AcOH (17.5 M, 320 μL, 10.00 eq) and compound F3 (180 mg, 560 umol, 1.00 eq) was added. The mixture was stirred at 35° C. for 20 min. Then the mixture was cooled to 15° C. and to the mixture was added NaBH₃CN (70.4 mg, 1.12 mmol, 2.00 eq). The reaction was stirred at 35° C. for 11.5 hrs. LC-MS showed compound F3 was consumed completely and one main peak with desired mass was detected. The reaction mixture was diluted with H₂O (15 mL) and extracted with ethyl acetate 30 mL (10 mL*3). The combined organic layers were washed with brine 10 mL (10 mL), dried over Na₂SO₄, filtered and concentrated under reduced pressure to give a residue. The residue was purified by prep-HPLC (column: Waters Xbridge BEH C18 100*30 mm*10 um; mobile phase: [water (NH₄HCO₃)-ACN]; B %: 15%-45%, 8 min). Compound F4 (60 mg, 149 umol, 26.68% yield) was obtained as a white solid. ¹H NMR: (400 MHz, DMSO-d₆) δ=7.49 (d, J=8.6 Hz, 1H), 7.09-6.99 (m, 2H), 5.04 (dd, J=5.2, 13.3 Hz, 1H), 4.36-4.16 (m, 2H), 4.07 (d, J=6.9 Hz, 1H), 3.93-3.83 (m, 2H), 3.27 (s, 6H), 2.97-2.84 (m, 1H), 2.83-2.72 (m, 2H), 2.69-2.54 (m, 2H), 2.04-1.88 (m, 1H), 1.87-1.75 (m, 1H), 1.74-1.66 (m, 2H), 1.36-1.20 (m, 3H).

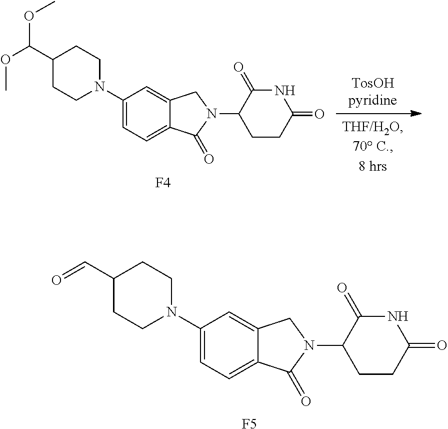

To a solution of compound F4 (50 mg, 125 umol, 1.00 eq) in THF (1.0 mL) and H₂O (0.2 mL) was added 4-methylbenzenesulfonic acid; pyridine (62.6 mg, 249 umol, 2.00 eq). The mixture was stirred at 70° C. for 8 hrs. LC-MS showed compound F4 was consumed completely and one main peak with desired mass was detected. The reaction mixture was concentrated under reduced pressure to give a residue. Compound F5 (60 mg, crude, 50% purity) was obtained as a yellow solid. The crude was used into next step without further purification. ¹H NMR: (400 MHz, CHLOROFORM-d) δ=7.79 (d, J=8.4 Hz, 1H), 7.17 (br s, 2H), 5.18 (dd, J=5.1, 13.1 Hz, 1H), 4.48-4.28 (m, 2H), 3.75-3.67 (m, 2H), 3.37-3.21 (m, 2H), 2.91-2.80 (m, 2H), 2.67-2.54 (m, 1H), 2.25-2.18 (m, 3H), 2.11-1.93 (m, 4H).

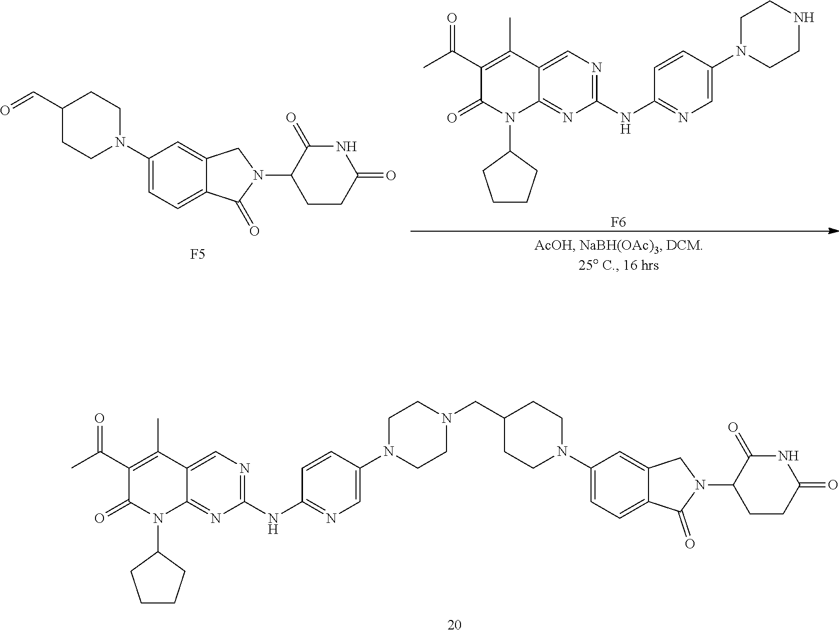

To a solution of compound F6 (30 mg, 67.0 umol, 0.80 eq) and compound F5 (59.6 mg, 83.8 umol, 50% purity, 1.00 eq) in DCM (1 mL) was added AcOH (15.1 mg, 251 umol, 14.4 uL, 3.00 eq) and NaBH(OAc)₃ (53.3 mg, 251 umol, 3.00 eq) at 25° C. The mixture was stirred at 25° C. for 16 hrs. LC-MS showed compound F6 was consumed completely and one peak with desired mass was detected. The reaction mixture was concentrated under reduced pressure to give a residue. The residue was purified by prep-HPLC (column: Waters Xbridge BEH C18 100*30 mm*10 um; mobile phase: [water (NH₄HCO₃)-ACN]; B %: 40%-70%, 8 min). Compound 20 (7 mg, 8.90 umol, 10.6% yield) was obtained as a yellow solid. 1H NMR: (400 MHz, DMSO-d₆) δ=10.94 (br s, 1H), 10.08 (s, 1H), 8.95 (s, 1H), 8.14-7.99 (m, 1H), 7.84 (d, J=9.1 Hz, 1H), 7.58-7.40 (m, 2H), 7.10-6.96 (m, 2H), 5.92-5.73 (m, 1H), 5.15-4.97 (m, 1H), 4.40-4.12 (m, 2H), 3.88 (br d, J=11.4 Hz, 2H), 3.21-3.11 (m, 5H), 2.93-2.77 (m, 3H), 2.69-2.59 (m, 1H), 2.58 (br s, 1H), 2.42 (s, 3H), 2.31 (s, 3H), 2.27-2.16 (m, 4H), 2.12-2.06 (m, 1H), 2.01-1.93 (m, 1H), 1.91-1.71 (m, 8H), 1.68-1.52 (m, 2H), 1.28-1.11 (m, 3H).

### Preparation of Compounds (21)-(22)

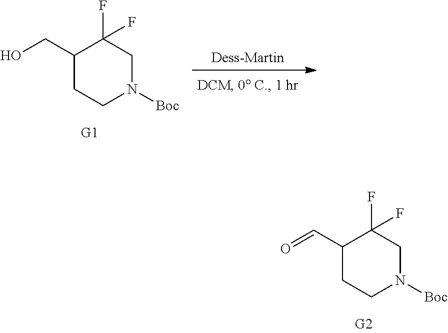 [SMILES: CC(C)(C)OC(=O)N1CCC(CO)C(F)(F)C1 ; CC(C)(C)OC(=O)N1CCC(C=O)C(F)(F)C1]

The solution of compound G1 (300 mg, 1.19 mmol, 1.00 eq) in DCM (4.00 mL) was added Dess-Martin (1.01 g, 2.39 mmol, 739 μL, 2.00 eq) at 0° C., the mixture was stirred at 0° C. for 1 hr. TLC (Petroleum ether/Ethyl acetate=3/1, R_{f} (P)=0.68, KMnO₄) indicated the compound G1 was consumed completely. The mixture was diluted with petroleum ether (10 mL) and then filtered. The filtrate was concentrated under reduced pressure to give compound G2 (300 mg, crude) as a colorless liquid which was used into the next step without further purification.

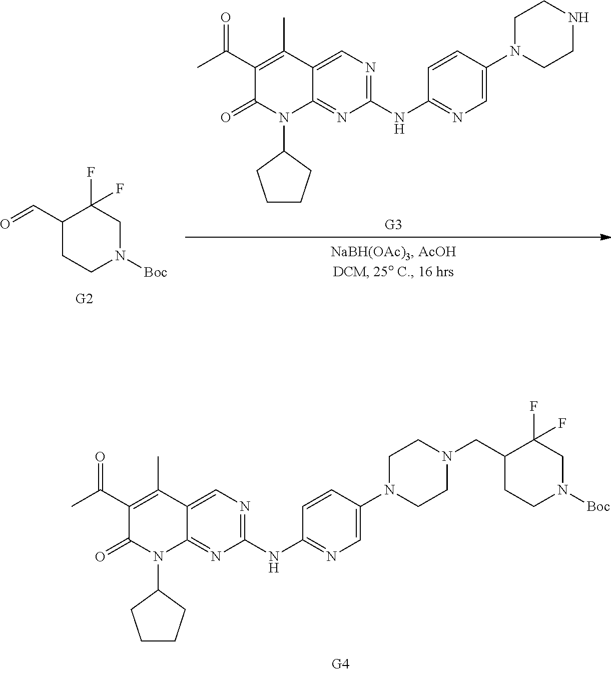 [SMILES: CC(C)(C)OC(=O)N1CCC(C=O)C(F)(F)C1 ; CC(=O)c1c(C)c2cnc(Nc3ccc(N4CCNCC4)cn3)nc2n(C2CCCC2)c1=O ; CC(=O)c1c(C)c2cnc(Nc3ccc(N4CCN(CC5CCN(C(=O)OC(C)(C)C)CC5(F)F)CC4)cn3)nc2n(C2CCCC2)c1=O]

To a solution of compound G2 (150 mg, 601 umol, 3.56 eq) and compound G3 (75.7 mg, 169 umol, 1.00 eq) in DCM (2.00 mL) was added AcOH (30.5 mg, 507 umol, 29.0 uL, 3.00 eq) and NaBH(OAc)₃ (107 mg, 507 umol, 3.00 eq) successively at 25° C. under N₂. The mixture was stirred at 25° C. for 16 hrs. LC-MS showed ˜2% of compound G3 remained and one main peak with desired m/z was detected. The mixture was concentrated under reduced pressure to give a residue. The residue was diluted with DMF (1.00 mL) and H₂O (10 mL). After stirring at 25° C. for 2 hrs, the mixture was filtered and the filter cake was dried under reduced pressure to give a residue. Compound G4 (230 mg, crude, 2 batches) was obtained as a yellow solid.

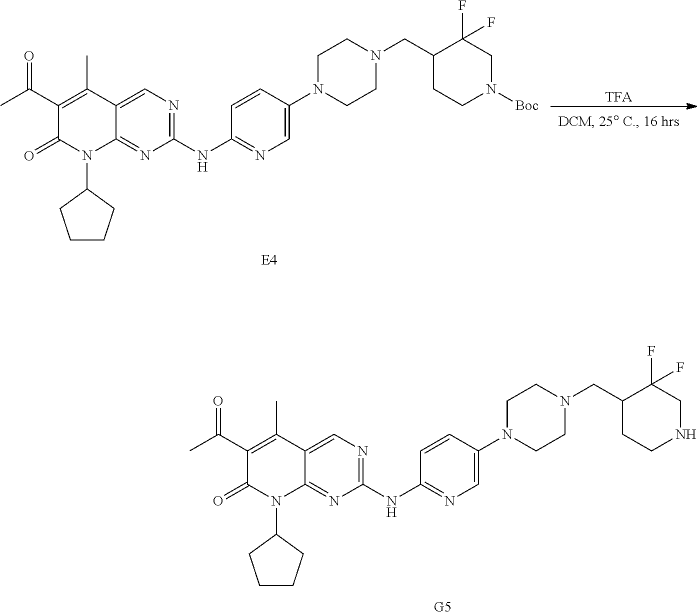 [SMILES: CC(=O)c1c(C)c2cnc(Nc3ccc(N4CCN(CC5CCN(C(=O)OC(C)(C)C)CC5(F)F)CC4)cn3)nc2n(C2CCCC2)c1=O ; "E4" ; CC(=O)c1c(C)c2cnc(Nc3ccc(N4CCN(CC5CCNCC5(F)F)CC4)cn3)nc2n(C2CCCC2)c1=O ; "TFA"]

To a solution of compound G4 (110 mg, 161 umol, 1.00 eq) in DCM (2.00 mL) was added TFA (214 mg, 1.88 mmol, 139 μL, 11.6 eq) at 25° C., the mixture was stirred at 25° C. for 16 hrs. LC-MS showed compound G4 was consumed completely and one main peak with desired m/z was detected. The mixture was concentrated under reduced pressure to give a residue. The residue was diluted with DCM (20 mL) and the pH of the mixture was adjusted to ˜10 by addition of NaOH (2 N, 5 mL). Then the mixture was extracted with DCM 10 mL (5 mL*2). The combined organic layers were concentrated under reduced pressure to give compound G5 (200 mg, crude) as a yellow solid.

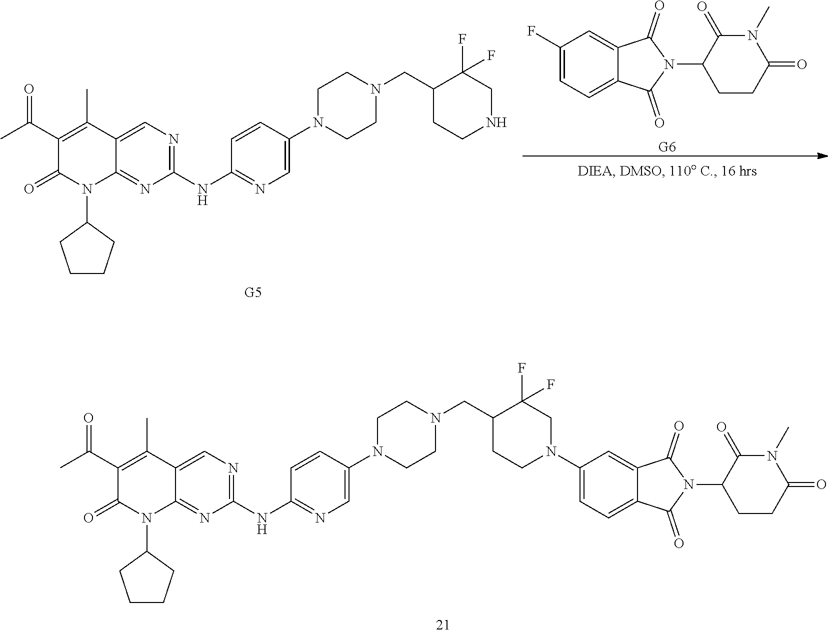

To a solution of compound G5 (20.0 mg, 34.4 umol, 1.00 eq) and compound G6 (20.0 mg, 68.9 umol, 2.00 eq) in DMSO (0.50 mL) was added DIEA (17.8 mg, 137.7 umol, 24.0 uL, 4.00 eq) at 25° C., the mixture was stirred at 110° C. for 16 hrs. LC-MS showed ˜₂₁% of compound G5 was remained. Several new peaks were shown on LC-MS and ˜₂₀% of desired compound was detected. The mixture was diluted with DMSO (2.00 mL) and purified directly by prep-HPLC (column: Waters Xbridge Prep OBD C18 150*40 mm*10 um; mobile phase: [water (NH₄HCO₃)-ACN]; B %: 40%-70%, 8 min). Compound 21 (6.16 mg, 6.90 umol, 5.01% yield, 95.3% purity, 4 batches) was obtained as a yellow solid checked by HNMR, FNMR, HPLC and MS. ¹H NMR: (400 MHz, CDCl₃) δ=8.72 (s, 1H), 8.11-7.80 (m, 2H), 7.71-7.63 (m, 1H), 7.59-7.53 (m, 1H), 7.37-7.33 (m, 1H), 7.28-7.24 (m, 1H), 7.06-6.99 (m, 1H), 5.80 (quin, J=8.8 Hz, 1H), 4.90-4.84 (m, 1H), 4.10-3.98 (m, 1H), 3.90 (br d, J=13.3 Hz, 1H), 3.24-3.07 (m, 7H), 3.05-2.85 (m, 2H), 2.80-2.58 (m, 5H), 2.57-2.46 (m, 5H), 2.41 (br dd, J=9.5, 12.6 Hz, 1H), 2.36-2.20 (m, 5H), 2.18-1.93 (m, 5H), 1.88-1.75 (m, 2H), 1.62 (br dd, J=5.3, 10.8 Hz, 4H).

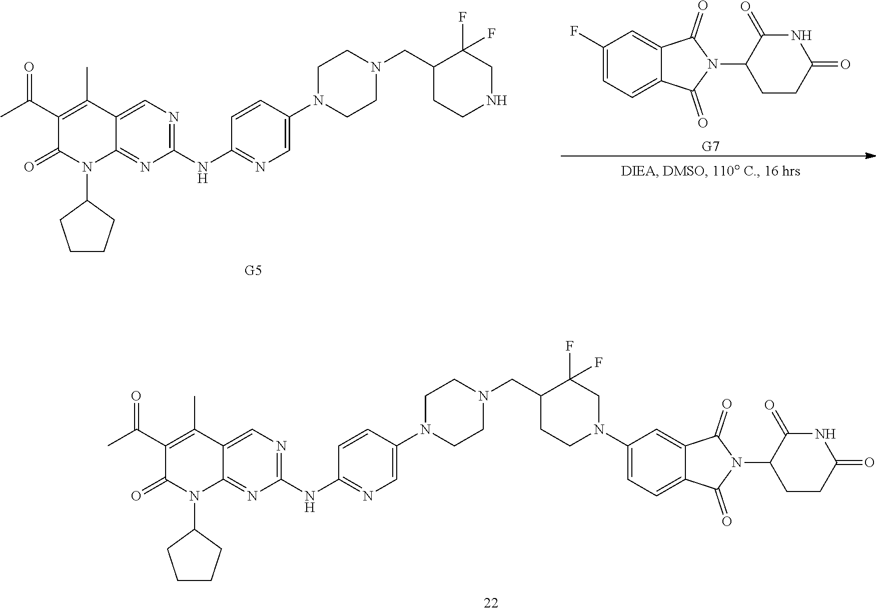

To a solution of compound G5 (20.0 mg, 34.4 umol, 1.00 eq) and compound G7 (20 mg, 72.4 umol, 2.10 eq) in DMSO (0.50 mL) was added DIEA (17.8 mg, 137 umol, 0.024 mL, 4.00 eq) at 25° C., the mixture was stirred at 110° C. for 16 hrs. LC-MS (ET62148-38-P1D1) showed ˜20% of compound G5 was remained. Several new peaks were shown on LC-MS and ˜20% of desired compound was detected. The mixture was diluted with DMSO (2.00 mL) and purified directly by prep-HPLC (column: Waters Xbridge Prep OBD C18 150 * 40 mm*10 um; mobile phase: [water (NH₄HCO₃)-ACN]; B %: 40%-70%, 8 min). Compound 22 (13.86 mg, 16.5 umol, 11.97% yield, 99.6% purity, 4 batches) was obtained as a yellow solid checked by HNMR, FNMR, HPLC and LCMS. ¹H NMR: (400 MHz, CDCl₃) δ=8.73 (s, 1H), 8.29 (br s, 1H), 8.10 (d, J=9.0 Hz, 1H), 7.97 (br d, J=2.8 Hz, 2H), 7.65 (d, J=8.5 Hz, 1H), 7.29-7.24 (m, 2H), 7.04 (dd, J=2.1, 8.6 Hz, 1H), 5.88-5.74 (m, 1H), 4.88 (dd, J=5.3, 12.3 Hz, 1H), 4.10-3.97 (m, 1H), 3.90 (br d, J=14.3 Hz, 1H), 3.24-3.09 (m, 4H), 3.06-2.96 (m, 1H), 2.88-2.75 (m, 2H), 2.73-2.66 (m, 3H), 2.57-2.46 (m, 5H), 2.44-2.37 (m, 1H), 2.34-2.23 (m, 5H), 2.21-2.04 (m, 3H), 2.02-1.92 (m, 3H), 1.86-1.77 (m, 2H), 1.68-1.59 (m, 4H).

### Preparation of Compound (23)

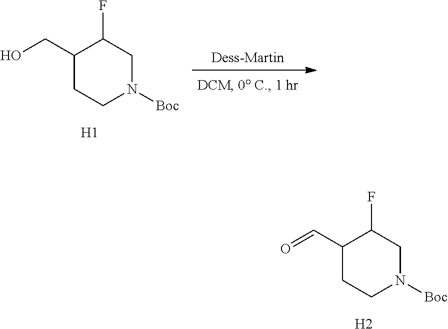

To a solution of compound H1 (150 mg, 643 umol, 1.00 eq) in DCM (2.00 mL) was added Dess-Martin (545 mg, 1.29 mmol, 398 μL, 2.00 eq) at 0° C. The mixture was stirred at 0° C. for 1 hr. TLC (Petroleum ether/Ethyl acetate=3/1, R_{f}(P)=0.41, KMnO₄) indicated compound H1 was consumed completely. The mixture was diluted with petroleum ether (5.00 mL) and filtered. The filtrate was concentrated under reduced pressure to give the crude product compound H2 (150 mg, crude) as a colorless oil which was used into the next step without further purification.

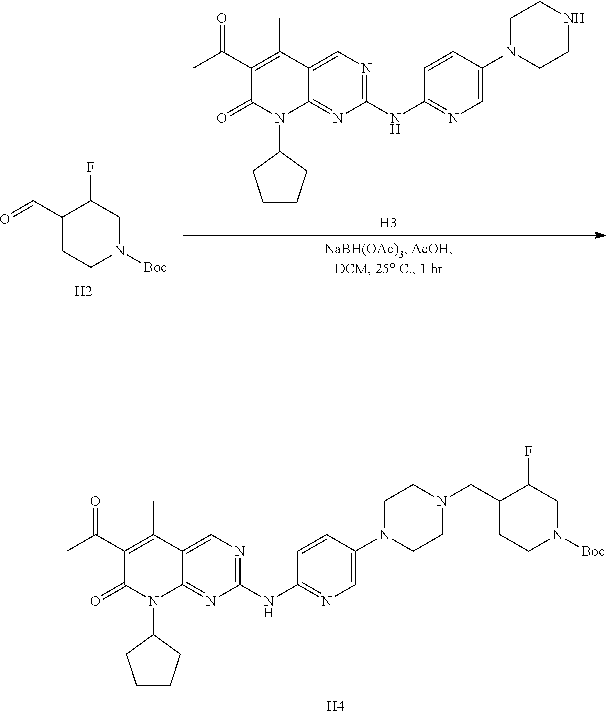

To a solution of compound H3 (100 mg, 223 umol, 1.00 eq) in DCM (4.00 mL) was added compound H2 (142 mg, 617 umol, 2.76 eq) at 25° C. Then to the mixture was added AcOH (40.2 mg, 670 umol, 38.3 uL, 3.00 eq) and NaBH(OAc)₃ (142 mg, 670 umol, 3.00 eq) at 25° C. The mixture was stirred at 25° C. for 1 hr. LC-MS showed compound H3 was consumed completely and one main peak with desired m/z was detected. The mixture was concentrated under reduced pressure to give a residue. The residue was suspended in H₂O (10 mL) and then filtered. The filter cake was dried under reduced pressure to give the crude product compound H4 (140 mg, crude) as a yellow solid.

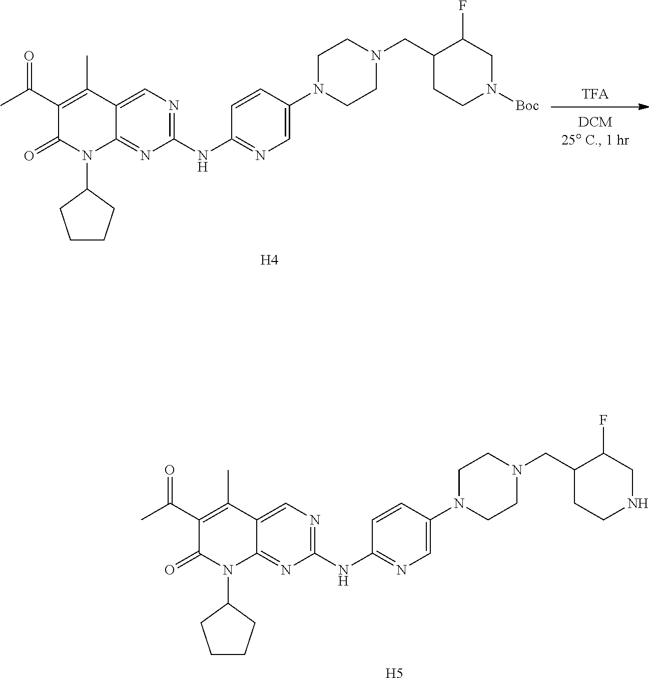 [SMILES: CC(=O)c1c(C)c2cnc(Nc3ccc(N4CCN(CC5CCN(C(=O)OC(C)(C)C)CC5F)CC4)cn3)nc2n(C2CCCC2)c1=O ; "H4" ; CC(=O)c1c(C)c2cnc(Nc3ccc(N4CCN(CC5CCNCC5F)CC4)cn3)nc2n(C2CCCC2)c1=O ; "H5"]

To a solution of compound H4 (140 mg, 211 umol, 1.00 eq) in DCM (2.00 mL) was added TFA (1.54 g, 13.5 mmol, 1.00 mL, 63.9 eq) at 25° C., the mixture was stirred at 25° C. for 1 hr. LC-MS showed compound H4 was consumed completely and one main peak with desired m/z was detected. The mixture was concentrated under reduced pressure to give a residue. The residue was diluted with DCM (20 mL). The pH of the mixture was adjusted to ˜10 by NaOH (2 N, 5 mL). And then the aqueous layer was extracted with DCM 10 mL (5 mL*2). The combined organic layers were concentrated under reduced pressure to give compound H5 (100 mg, crude) as a yellow solid which was used into next step directly.

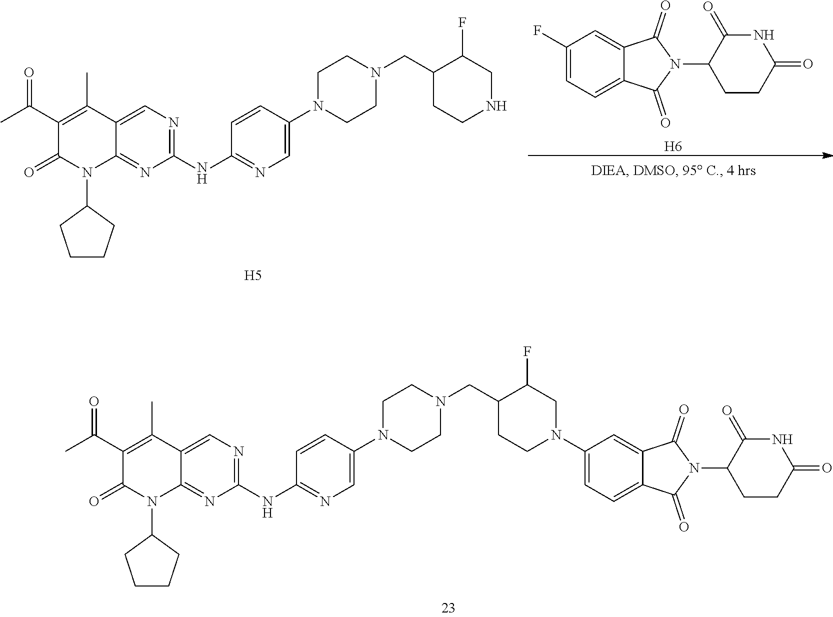

To a solution of compound H6 (80.0 mg, 289 umol, 2.04 eq) and compound H5 (80.0 mg, 142 umol, 1.00 eq) in DMSO (0.50 mL) was added DIEA (74.2 mg, 574 umol, 100 μL, 4.04 eq) at 25° C., the mixture was stirred at 95° C. for 4 hrs. LC-MS showed ˜10% of compound H5 was remained and desired compound was detected. The mixture was diluted with DMSO (2.00 mL) and then purified directly by prep-HPLC (column: Waters Xbridge BEH C18 100*30 mm*10 um; mobile phase: [water (NH₄HCO₃)-ACN]; B %: 40%-75%, 8 min). Compound 23 (42.0 mg, 51.2 umol, 36.0% yield, 99.9% purity) was obtained as a yellow solid checked by HNMR, FNMR, LCMS and HPLC. ¹H NMR: (400 MHz, CDCl₃) δ 8.73 (s, 1H), 8.33-8.25 (m, 1H), 8.10 (d, J=9.0 Hz, 1H), 7.97 (br d, J=2.5 Hz, 2H), 7.64 (d, J=8.4 Hz, 1H), 7.29-7.22 (m, 2H), 7.02 (dd, J=2.1, 8.5 Hz, 1H), 5.80 (quin, J=8.9 Hz, 1H), 4.88 (br dd, J=5.3, 12.3 Hz, 1H), 4.53-4.30 (m, 1H), 4.03-3.92 (m, 1H), 3.71 (br d, J=12.9 Hz, 1H), 3.21-3.07 (m, 4H), 3.06-2.96 (m, 1H), 2.90-2.75 (m, 2H), 2.73-2.54 (m, 5H), 2.48 (s, 3H), 2.39-2.23 (m, 6H), 2.20-1.93 (m, 5H), 1.87-1.76 (m, 2H), 1.62 (br dd, J=5.4, 10.6 Hz, 3H), 1.44-1.32 (m, 2H).

### Preparation of Compound (24)

Compound (24) was prepared from E4, the synthesis of which is described in the preparation of Compound (19)

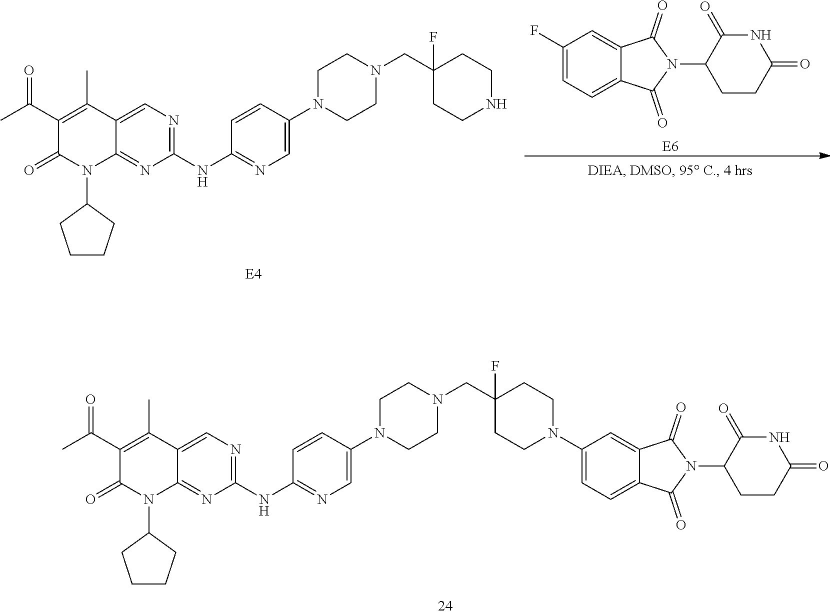

To a solution of compound E4 (40.0 mg, 71.0 umol, 1.00 eq) and compound E6 (40.0 mg, 144 umol, 2.04 eq) in DMSO (0.50 mL) was added DIEA (38.5 mg, 298 umol, 52.0 uL, 4.20 eq) at 25° C., the mixture was stirred at 95° C. for 4 hrs. LC-MS (ET62148-36-P1B) showed compound E4 was consumed completely and desired mass was detected. The mixture was diluted with DMSO (2.00 mL) and then purified directly by prep-HPLC (column: Waters Xbridge BEH C18 100*30 mm*10 um; mobile phase: [water (NH₄HCO₃)-ACN]; B %: 50%-80%, 8 min). Compound 24 (14.7 mg, 17.8 umol, 25.1% yield, 99.6% purity) was obtained as a yellow solid checked by HNMR, FNMR, HPLC and LCMS. ¹H NMR: (400 MHz, CDCl₃) δ=8.84 (br s, 1H), 8.74 (s, 1H), 8.29 (br s, 1H), 8.09 (d, J=9.0 Hz, 1H), 7.98 (d, J=2.5 Hz, 1H), 7.63 (d, J=8.6 Hz, 1H), 7.29-7.21 (m, 2H), 7.01 (dd, J=1.9, 8.5 Hz, 1H), 5.80 (quin, J=8.9 Hz, 1H), 4.88 (dd, J=5.2, 12.2 Hz, 1H), 3.72 (br d, J=13.1 Hz, 2H), 3.31 (br t, J=11.9 Hz, 2H), 3.12 (br s, 4H), 2.88-2.75 (m, 2H), 2.74-2.62 (m, 5H), 2.54 (s, 1H), 2.48 (s, 4H), 2.35-2.23 (m, 5H), 2.11-1.93 (m, 5H), 1.86-1.74 (m, 3H), 1.73-1.62 (m, 3H).

### Preparation of Compound (25)

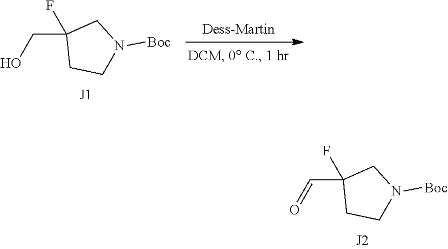 [SMILES: CC(C)(C)OC(=O)N1CCC(F)(CO)C1 ; CC(C)(C)OC(=O)N1CCC(F)(C=O)C1]

The solution of compound J1 (150 mg, 684 umol, 1.00 eq) in DCM (2.00 mL) was added Dess-Martin (580 mg, 1.37 mmol, 423 μL, 2.00 eq) at 0° C. The mixture was stirred at 0° C. for 1 hr. TLC (Petroleum ether/Ethyl acetate=3/1, R_{f}(P)=0.48, KMnO₄) indicated compound J1 was consumed completely. The mixture was diluted with Petroleum ether (5.00 mL) and filtered. The filtrate was concentrated under reduced pressure to give compound J2 (150 mg, crude) as a colorless oil which was used into the next step without further purification.

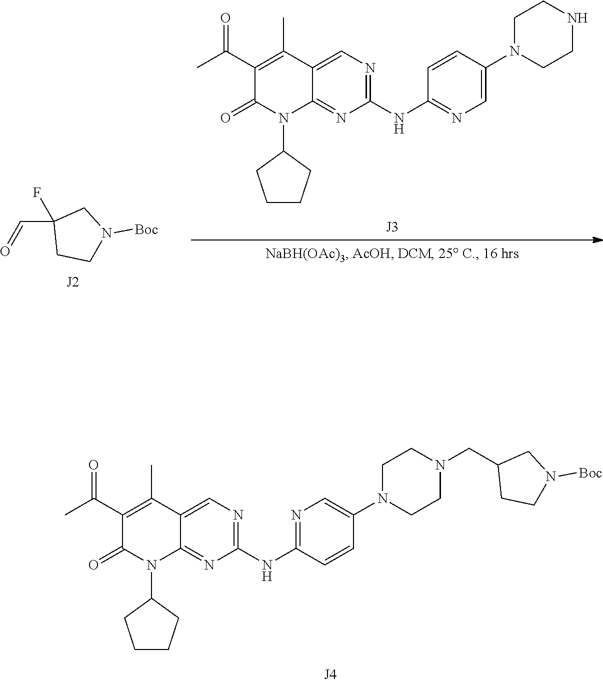 [SMILES: CC(C)(C)OC(=O)N1CCC(F)(C=O)C1 ; CC(=O)c1c(C)c2cnc(Nc3ccc(N4CCNCC4)cn3)nc2n(C2CCCC2)c1=O ; CC(=O)c1c(C)c2cnc(Nc3ccc(N4CCN(CC5CCN(C(=O)OC(C)(C)C)C5)CC4)cn3)nc2n(C2CCCC2)c1=O]

To a solution of compound J3 (247 mg, 552 umol, 0.80 eq) and compound J2 (150 mg, 690 umol, 1.00 eq) in DCM (2.00 mL) was added AcOH (124 mg, 2.07 mmol, 118 uL, 3.00 eq) and NaBH(OAc)₃ (439 mg, 2.07 mmol, 3.00 eq) at 25° C. The mixture was stirred at 25° C. for 16 hrs. LC-MS showed ˜40% of compound J3 was remained. Several new peaks were shown on LC-MS and ˜₃₇% of desired compound was detected. The mixture was diluted with DMSO (5.00 mL) and purified directly by prep-HPLC (column: Phenomenex C18 80*40 mm*3 um; mobile phase: [water (NH₄HCO₃)-ACN]; B %: 40%-75%, 8 min). Compound J4 (25.0 mg, 38.5 umol, 5.58% yield) was obtained as a yellow solid.

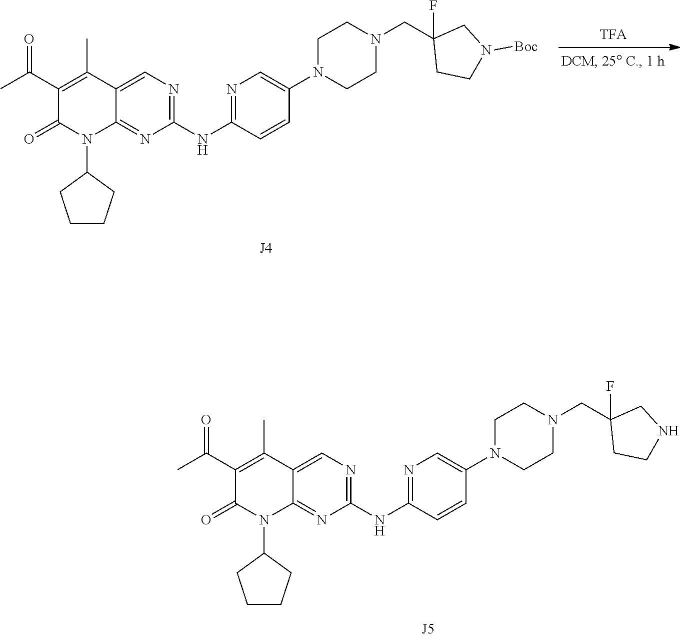 [SMILES: CC(=O)c1c(C)c2cnc(Nc3ccc(N4CCN(CC5(F)CCN(C(=O)OC(C)(C)C)C5)CC4)cn3)nc2n(C2CCCC2)c1=O ; CC(=O)c1c(C)c2cnc(Nc3ccc(N4CCN(CC5(F)CCNC5)CC4)cn3)nc2n(C2CCCC2)c1=O]

To a solution of compound J4 (25.0 mg, 38.5 umol, 1.00 eq) in DCM (0.50 mL) was added TFA (308 mg, 2.70 mmol, 0.20 mL, 70.1 eq) at 25° C., the mixture was stirred at 25° C. for 1 hr. LC-MS showed compound J4 was consumed completely and desired compound was detected. The mixture was concentrated under reduced pressure to give a residue. The residue was diluted with DCM (5.00 mL) and the pH of the mixture was adjusted to ˜10 by NaOH (2 N, 5.00 mL). Then aqueous layer was extracted with DCM 10 mL (5.00 mL*2). The combined organic layers were concentrated under reduced pressure to give compound J5 (21.0 mg, crude) as a yellow solid.

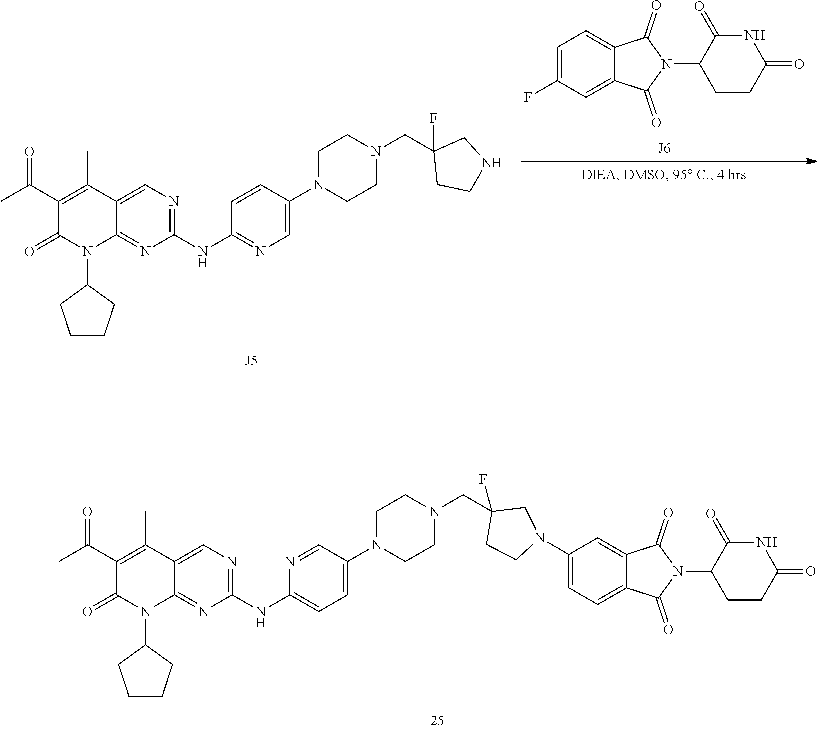 [SMILES: CC(=O)c1c(C)c2cnc(Nc3ccc(N4CCN(CC5(F)CCNC5)CC4)cn3)nc2n(C2CCCC2)c1=O ; O=C1CCC(N2C(=O)c3ccc(F)cc3C2=O)C(=O)N1 ; CC(=O)c1c(C)c2cnc(Nc3ccc(N4CCN(CC5(F)CCN(c6ccc7c(c6)C(=O)N(C6CCC(=O)NC6=O)C7=O)C5)CC4)cn3)nc2n(C2CCCC2)c1=O]

To a solution of compound J5 (20.0 mg, 36.4 umol, 1.00 eq) and compound J6 (20.0 mg, 72.4 umol, 1.99 eq) in DMSO (0.50 mL) was added DIEA (18.8 mg, 145 umol, 25.4 uL, 4.00 eq) at 25° C. The mixture was stirred at 95° C. for 4 hrs. LC-MS showed compound J5 was consumed completely and desired compound was detected. The mixture was diluted with DMSO (2.00 mL) and then purified directly by prep-HPLC (column: Waters Xbridge BEH C18 100*30 mm*10 um; mobile phase: [water (NH₄HCO₃)-ACN]; B %: 45%-75%, 8 min). Compound 25 (10.0 mg, 12.4 umol, 34.0% yield, 100% purity) was obtained as a yellow solid checked by HNMR, FNMR, LCMS and HPLC. ¹H NMR: (400 MHz, CDCl₃) δ=8.74 (s, 1H), 8.34 (br d, J=9.9 Hz, 1H), 8.18 (br d, J=9.3 Hz, 1H), 7.96 (br s, 1H), 7.62 (d, J=8.4 Hz, 1H), 7.33 (br d, J=8.3 Hz, 1H), 6.91 (s, 1H), 6.66 (dd, J=1.6, 8.4 Hz, 1H), 5.87-5.74 (m, 1H), 4.88 (dd, J=5.2, 12.2 Hz, 1H), 3.74-3.53 (m, 4H), 3.20 (br s, 3H), 2.98-2.61 (m, 9H), 2.48 (s, 3H), 2.44-2.36 (m, 1H), 2.33-2.23 (m, 5H), 2.13-1.95 (m, 4H), 1.88-1.75 (m, 2H), 1.62 (br dd, J=5.2, 10.3 Hz, 3H).

### Preparation of Compound (26)

Compound (26) was prepared from C₃, the synthesis of which is described in the preparation of Compound (16)

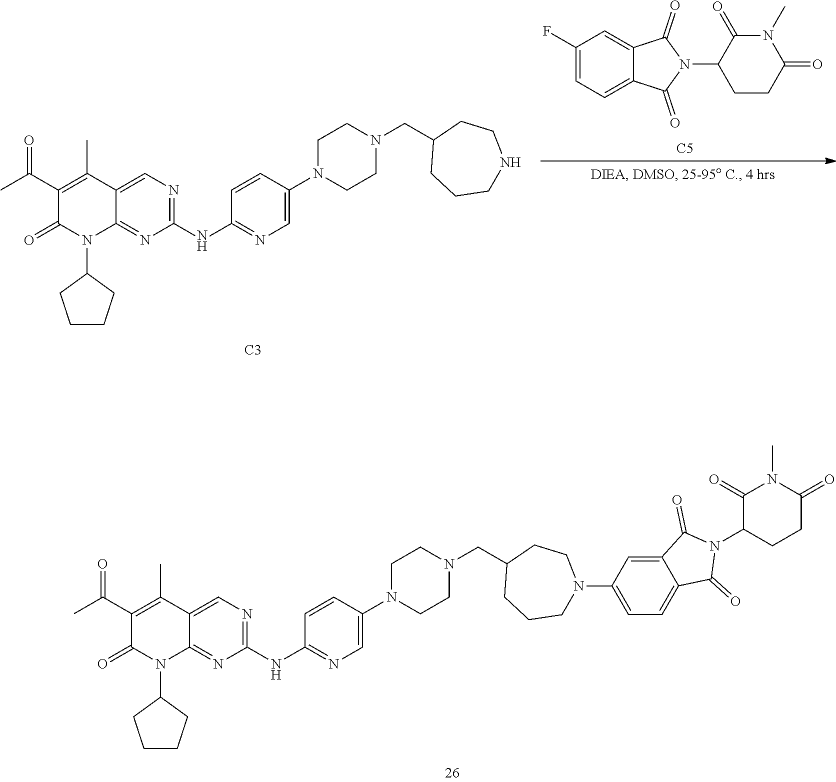 [SMILES: CC(=O)c1c(C)c2cnc(Nc3ccc(N4CCN(CC5CCCNCC5)CC4)cn3)nc2n(C2CCCC2)c1=O ; CN1C(=O)CCC(N2C(=O)c3ccc(F)cc3C2=O)C1=O ; "C5" ; "C3" ; CC(=O)c1c(C)c2cnc(Nc3ccc(N4CCN(CC5CCCN(c6ccc7c(c6)C(=O)N(C6CCC(=O)N(C)C6=O)C7=O)CC5)CC4)cn3)nc2n(C2CCCC2)c1=O]

To a solution of compound C5 (60.0 mg, 206 umol, 1.92 eq) and compound C3 (60.0 mg, 107 umol, 1.00 eq) in DMSO (1.00 mL) was added DIEA (55.5 mg, 429 umol, 74.8 uL, 4.00 eq) at 25° C., the mixture was stirred at 95° C. for 4 hrs. LC-MS showed compound C3 was consumed completely and one peak with desired mass was detected. The mixture was diluted with DMSO (2.00 mL) and purified directly by prep-HPLC (column: Waters Xbridge Prep OBD C18 150*40 mm*10 um; mobile phase: [water (NH₄HCO₃)-ACN]; B %: 50%-80%, 8 min). Compound 26 (30.0 mg, 36.0 umol, 33.5% yield, 99.6% purity) was obtained as a yellow solid checked by HNMR, LCMS and HPLC. ¹H NMR: ET62148-54-P1F (400 MHz, CDCl₃) δ=8.73 (s, 1H), 8.09 (br d, J=8.5 Hz, 1H), 7.95 (d, J=2.6 Hz, 1H), 7.88 (br s, 1H), 7.59 (d, J=8.6 Hz, 1H), 7.26 (dd, J=2.7, 9.1 Hz, 1H), 7.03 (d, J=2.1 Hz, 1H), 6.78 (dd, J=1.9, 8.6 Hz, 1H), 5.80 (quin, J=8.9 Hz, 1H), 4.92-4.82 (m, 1H), 3.63 (td, J=5.0, 9.6 Hz, 2H), 3.51-3.37 (m, 2H), 3.18-3.06 (m, 6H), 2.94-2.87 (m, 1H), 2.80-2.62 (m, 3H), 2.55 (s, 1H), 2.48 (s, 6H), 2.34-2.21 (m, 6H), 2.14 (br s, 2H), 2.06-1.94 (m, 5H), 1.89-1.76 (m, 3H), 1.62 (br dd, J=5.4, 10.6 Hz, 3H), 1.43-1.31 (m, 1H), 1.16-0.98 (m, 1H).

### Preparation of Compounds (27)-(30)

Compounds (27)-(30) were prepared according to Scheme 6.

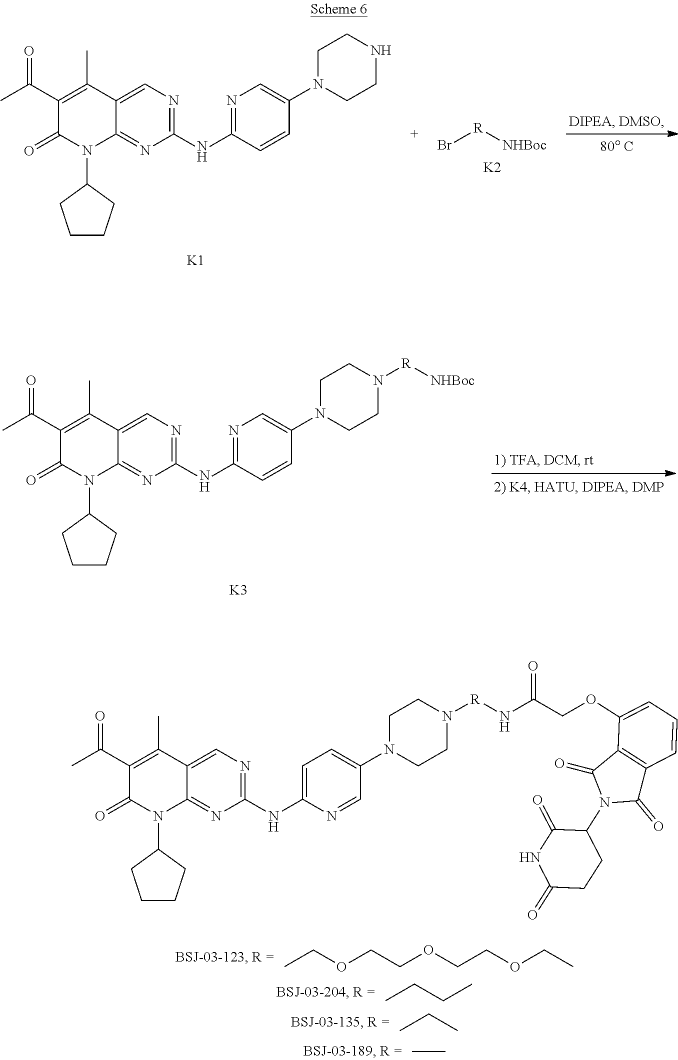

### Compound (27): N-(2-(2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethoxy)ethoxy)ethoxy)ethyl)-2-((2-(2,6-dioxopiperidin-3-yl)-1,3-dioxoisoindolin-4-yl)oxy)acetamide (BSJ-03-123)

Compound (27) was synthesized with the same procedure reported in the reference (29).

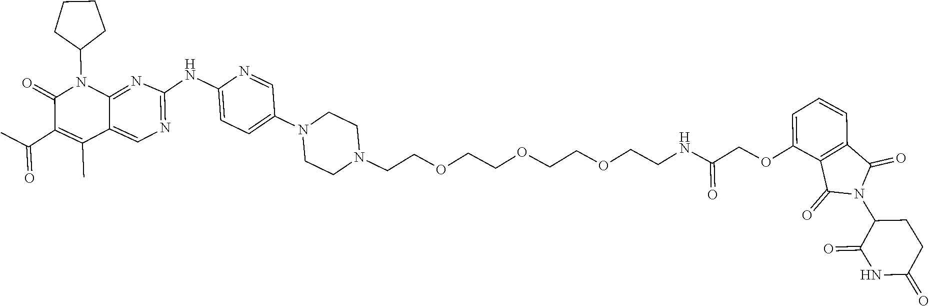 [SMILES: CC(=O)c1c(C)c2cnc(Nc3ccc(N4CCN(CCOCCOCCOCCNC(=O)COc5cccc6c5C(=O)N(C5CCC(=O)NC5=O)C6=O)CC4)cn3)nc2n(C2CCCC2)c1=O]

To a solution of compound K1 (100 mg, 0.22 mmol) in DMSO (5 mL) was PGP-added tert-butyl (2-(2-(2-(2-bromoethoxy)ethoxy)ethoxy)ethyl)carbamate K2 (156 mg, 0.44 mmol) and DIPEA (0.115 mL, 0.66 mmol). The mixture was heated to 80° C. and kept stirring for 24 h. The mixture was then cooled down to room temperature, extracted, dried, filtered and concentrated. The residue was purified by reverse phase HPLC (5-95% MeOH in H₂O) to give K3 (TFA salt) as a yellow solid (103 mg, 65%). LC-MS: m/z 723 [M+1].

To a solution of intermediate K3 (30.5 mg, 0.0422 mmol) in DCM (1 mL) was added TFA (1 mL) and the resulting solution was stirred at room temperature for 0.5h. The mixture was concentrated, and the residue was then dissolved in DMF (1 mL) followed by addition of 2-((2-(2,6-dioxopiperidin-3-yl)-1,3-dioxoisoindolin-4-yl)oxy)acetic acid (K4, 14 mg, 0.0422 mmol), HATU (33 mg, 0.0844 mmol) and DIPEA (37 L, 0.211 mmol). The resulting mixture was stirred for 0.5h at room temperature, then evaporated the solvent and purified by reverse phase HPLC (5-95% MeOH in H₂O) to give Compound (27) (TFA salt) as a yellow solid (34.4 mg, 87%). LC-MS: m/z 937 [M+1]. ¹H NMR (500 MHz, DMSO-d₆) δ 11.12 (s, 1H), 10.39 (s, 1H), 8.96 (s, 1H), 8.10 (d, J=3.0 Hz, 1H), 7.99 (t, J=5.7 Hz, 1H), 7.87 (d, J=9.1 Hz, 1H), 7.83-7.73 (m, 1H), 7.63 (dd, J=9.2, 3.1 Hz, 1H), 7.49 (d, J=7.3 Hz, 1H), 7.40 (d, J=8.5 Hz, 1H), 5.87-5.76 (m, 1H), 5.15-5.04 (m, 1H), 4.78 (s, 2H), 3.91-3.76 (m, 4H), 3.65-3.54 (br, 10H), 3.49-3.41 (m, 6H), 3.36-3.30 (m, 4H), 3.17-3.02 (m, 2H), 2.95-2.83 (m, 1H), 2.62-2.50 (m, 1H), 2.43 (s, 3H), 2.32 (s, 3H), 2.29-2.18 (m, 2H), 2.07-1.99 (m, 1H), 1.95-1.84 (m, 2H), 1.82-1.74 (m, 2H), 1.64-1.53 (m, 2H).

### Compound (28): N-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butyl)-2-((2-(2,6-dioxopiperidin-3-yl)-1,3-dioxoisoindolin-4-yl)oxy)acetamide (BSJ-03-204)

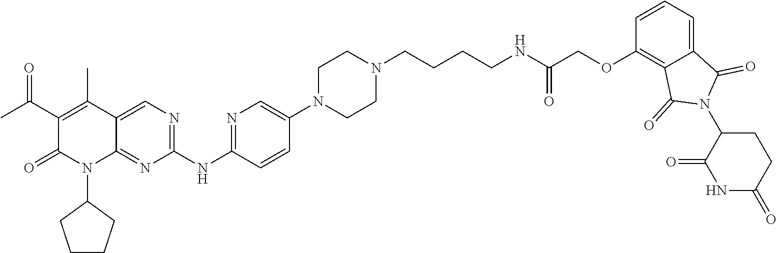 [SMILES: CC(=O)c1c(C)c2cnc(Nc3ccc(N4CCN(CCCCNC(=O)COc5cccc6c5C(=O)N(C5CCC(=O)NC5=O)C6=O)CC4)cn3)nc2n(C2CCCC2)c1=O]

Compound (28) was synthesized with similar procedures as Compound (27) from K1 (47.4 mg, 0.0844 mmol), tert-butyl (4-bromobutyl) carbamate (21.2 mg, 0.0844 mmol) and K4 (26.6 mg, 0.08 mmol). Compound (28) was obtained as a yellow solid (37.3 mg, 51% in 3 steps). LC-MS: m/z 833 [M+1]. ¹H NMR (500 MHz, DMSO-d₆) δ 8.97 (s, 1H), 8.12 (d, J=3.0 Hz, 1H), 8.05 (t, J=6.0 Hz, 1H), 7.91 (d, J=9.0 Hz, 1H), 7.83 (dd, J=8.5, 7.3 Hz, 1H), 7.64-7.57 (m, 1H), 7.52 (d, J=7.3 Hz, 1H), 7.42 (d, J=8.5 Hz, 1H), 5.83 (p, J=8.9 Hz, 1H), 5.17-5.05 (m, 1H), 4.80 (s, 2H), 3.86 (d, J=12.7 Hz, 2H), 3.58 (d, J=11.8 Hz, 2H), 3.26-3.09 (m, 6H), 3.02 (t, J=12.3 Hz, 2H), 2.96-2.84 (m, 1H), 2.63-2.54 (m, 1H), 2.43 (s, 3H), 2.32 (s, 3H), 2.30-2.18 (m, 2H), 2.04 (dtd, J=13.0, 5.3, 2.2 Hz, 1H), 1.94-1.84 (m, 2H), 1.83-1.72 (m, 2H), 1.72-1.64 (m, 2H), 1.63-1.55 (m, 2H), 1.54-1.45 (m, 2H).

### Compound (29): N-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)-2-((2-(2,6-dioxopiperidin-3-yl)-1,3-dioxoisoindolin-4-yl)oxy)acetamide (BSJ-03-135)

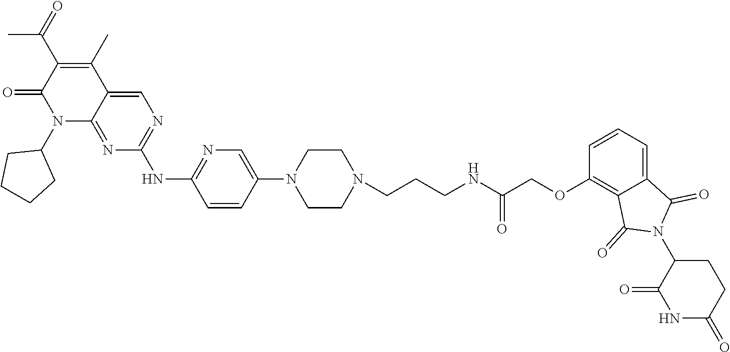 [SMILES: CC(=O)c1c(C)c2cnc(Nc3ccc(N4CCN(CCCNC(=O)COc5cccc6c5C(=O)N(C5CCC(=O)NC5=O)C6=O)CC4)cn3)nc2n(C2CCCC2)c1=O]

Compound (29) was synthesized with similar procedures as Compound (27) from K1 (47.4 mg, 0.0844 mmol), tert-butyl (3-bromopropyl) carbamate (20 mg, 0.0844 mmol) and K4 (26.6 mg, 0.08 mmol). Compound (29) was obtained as a yellow solid (38 mg, 55% in 3 steps). LC-MS: m/z 819 [M+1]. ¹H NMR (500 MHz, DMSO-d₆) δ 11.13 (s, 1H), 10.40 (s, 1H), 9.68 (s, 1H), 8.98 (s, 1H), 8.17 (t, J=5.9 Hz, 1H), 8.12 (d, J=3.0 Hz, 1H), 7.90 (d, J=9.1 Hz, 1H), 7.88-7.82 (m, 1H), 7.64 (dd, J=9.2, 3.1 Hz, 1H), 7.53 (d, J=7.3 Hz, 1H), 7.43 (d, J=8.6 Hz, 1H), 5.91-5.77 (pm, 1H), 5.19-5.05 (m, 1H), 4.81 (s, 2H), 3.65-3.49 (m, 2H), 3.32-3.24 (m, 2H), 3.23-3.09 (m, 4H), 3.08-2.97 (m, 2H), 2.94-2.84 (m, 1H), 2.66-2.53 (m, 2H), 2.43 (s, 3H), 2.32 (s, 3H), 2.30-2.18 (m, 2H), 2.14-1.98 (m, 1H), 1.95-1.84 (m, 4H), 1.83-1.72 (m, 2H), 1.65-1.52 (m, 2H), 1.25 (q, J=7.3 Hz, 2H).

### Compound (30): N-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)-2-((2-(2,6-dioxopiperidin-3-yl)-1,3-dioxoisoindolin-4-yl)oxy)acetamide (BSJ-03-189)

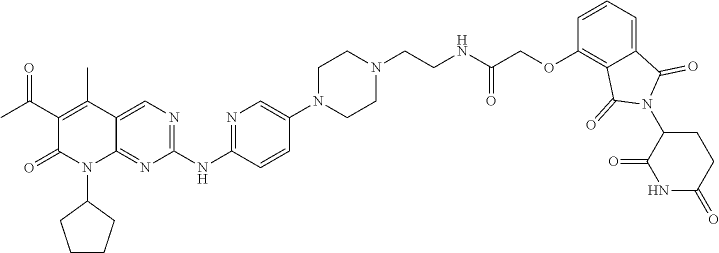 [SMILES: CC(=O)c1c(C)c2cnc(Nc3ccc(N4CCN(CCNC(=O)COc5cccc6c5C(=O)N(C5CCC(=O)NC5=O)C6=O)CC4)cn3)nc2n(C2CCCC2)c1=O]

Compound (30) was synthesized with similar procedures as Compound (27) from K1 (47.4 mg, 0.0844 mmol), tert-butyl (2-bromoethyl) carbamate (18.9 mg, 0.0844 mmol) and K4 (26.6 mg, 0.08 mmol). Compound (30) was obtained as a yellow solid (30.6 mg, 45% in 3 steps). LC-MS: m/z 805 [M+1]. ¹H NMR (500 MHz, DMSO-d₆) δ 8.97 (s, 1H), 8.31 (t, J=6.0 Hz, 1H), 8.12 (d, J=3.0 Hz, 1H), 7.90 (d, J=9.1 Hz, 1H), 7.83 (dd, J=8.5, 7.3 Hz, 1H), 7.62 (dd, J=9.2, 3.1 Hz, 1H), 7.53 (d, J=7.2 Hz, 1H), 7.44 (d, J=8.5 Hz, 1H), 5.89-5.78 (m, 1H), 5.17-5.08 (m, 1H), 4.85 (s, 2H), 3.89 (s, OH), 3.76-3.53 (m, 4H), 3.38-2.96 (m, 8H), 2.90 (ddd, J=16.8, 13.6, 5.3 Hz, 1H), 2.64-2.56 (m, 1H), 2.54 (s, 1H), 2.43 (s, 3H), 2.32 (s, 3H), 2.29-2.20 (m, 2H), 2.09-1.98 (m, 1H), 1.95-1.84 (m, 2H), 1.94-1.84 (m, 2H), 1.83-1.72 (t, J=6.7 Hz, 2H).

### Preparation of Compounds (31)-(36)

### Compound (31): 5-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidin-1-yl)-2-(2,6-dioxopiperidin-3-yl)isoindoline-1,3-dione (BSJ-03-096)

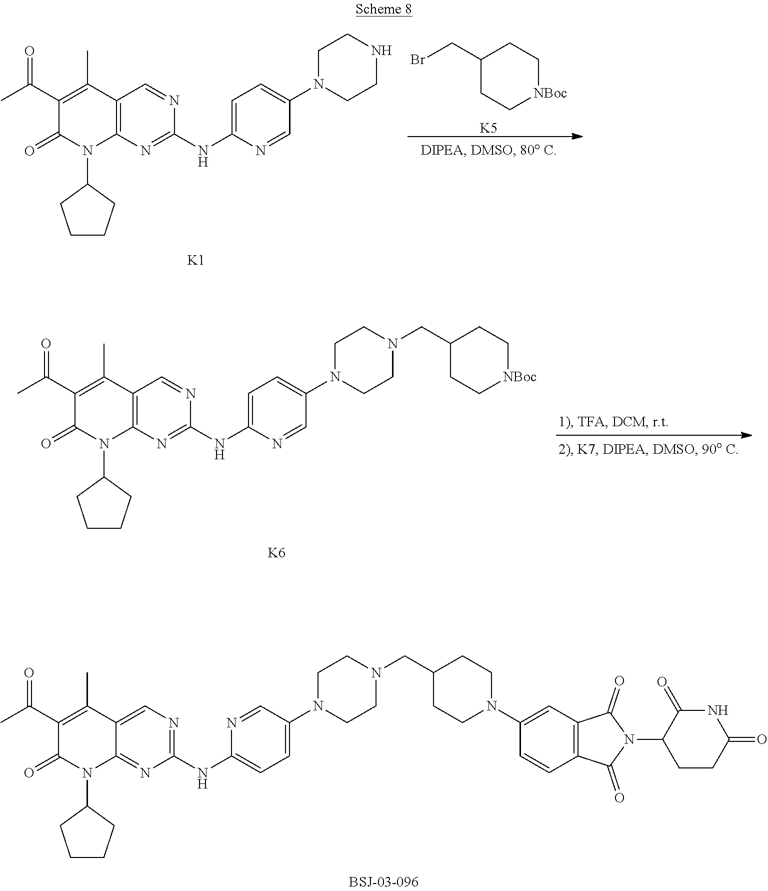

To a solution of compound K1 (100 mg, 0.22 mmol) in DMSO (5 mL) was added tert-butyl 4-(bromomethyl) piperidine-1-carboxylate K5 (153 mg, 0.55 mmol) and DIPEA (0.115 mL, 0.66 mmol). The mixture was heated to 80° C. and kept stirring for 48 h. The mixture was then cooled down to room temperature, extracted, dried, filtered and concentrated. The residue was purified by reverse phase HPLC (5-95% MeOH in H₂O) to give intermediate K6 (TFA salt) as a yellow solid (61 mg, 43%). LC-MS: m/z 645 [M+1].

To a solution of intermediate K6 (27.2 mg, 0.0422 mmol) in DCM (1 mL) was added TFA (1 mL) and the resulting solution was stirred at room temperature for 0.5h. The mixture was concentrated, and the residue was then dissolved in DMSO (1 mL) followed by addition of 2-(2,6-dioxopiperidin-3-yl)-5-fluoroisoindoline-1,3-dione (K7, 11.6 mg, 0.0422 mmol) and DIPEA (37 L, 0.211 mmol). The reaction was heat to 90° C. and kept stirring overnight. The mixture was then cooled to room temperature and purified by reverse phase HPLC (5-95% MeOH in H₂O) to give Compound (31) (TFA salt) as a yellow solid (34.4 mg, 87%). LC-MS: m/z 801 [M+1]. ¹H NMR (500 MHz, DMSO-d₆) δ 11.08 (s, 1H), 10.49 (s, 1H), 9.69 (s, 1H), 8.98 (s, 1H), 8.13 (d, J=3.1 Hz, 1H), 7.89 (d, J=9.1 Hz, 1H), 7.72-7.65 (m, 2H), 7.37 (d, J=2.3 Hz, 1H), 7.28 (dd, J=8.7, 2.3 Hz, 1H), 5.84 (p, J=8.9 Hz, 1H), 5.07 (dd, J=12.8, 5.4 Hz, 1H), 4.11 (d, J=13.1 Hz, 2H), 3.86 (d, J=11.0 Hz, 2H), 3.70-3.59 (m, 2H), 3.24-3.09 (m, 3H), 3.07-2.95 (m, 2H), 2.64-2.51 (m, 3H), 2.43 (s, 3H), 2.33 (s, 3H), 2.29-2.12 (m, 4H), 2.05-1.96 (m, 1H), 1.94-1.83 (m, 4H), 1.83-1.69 (m, 3H), 1.65-1.50 (m, 2H), 1.34-1.18 (m, 4H).

### Compound (32): 5-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)azetidin-1-yl)-2-(2,6-dioxopiperidin-3-yl)isoindoline-1,3-dione (BSJ-05-009)

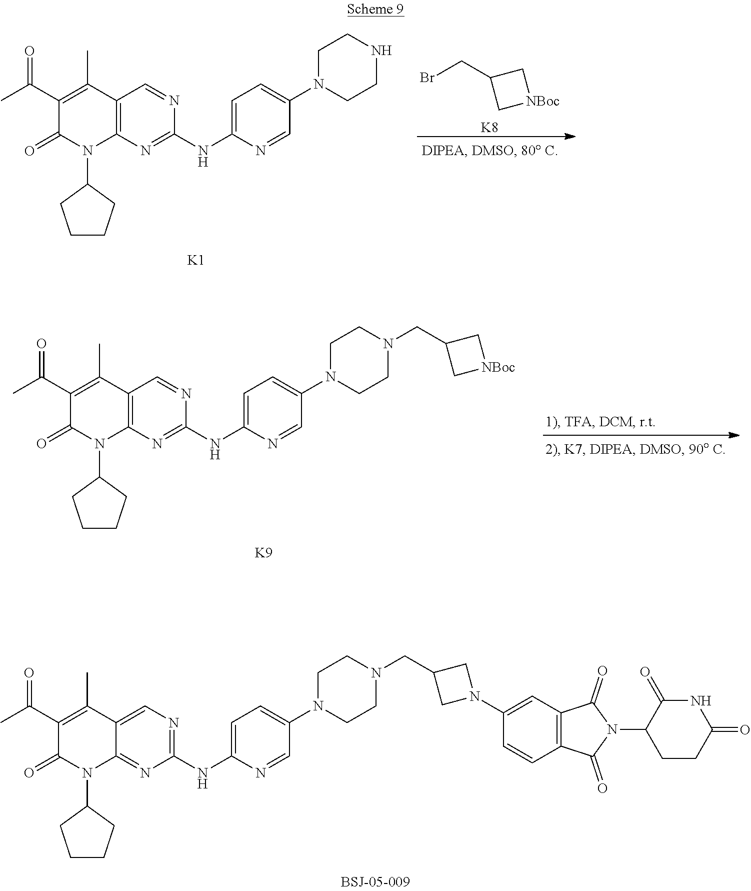

Compound (32) was synthesized with similar procedures as Compound (31) from compound K1, tert-butyl 3-(bromomethyl) azetidine-1-carboxylate (K8) and 2-(2,6-dioxopiperidin-3-yl)-5-fluoroisoindoline-1,3-dione (K7). BSJ-05-009 was obtained as a yellow solid. LC-MS: m/z 773 [M+1]. ¹H NMR (500 MHz, DMSO-d₆) δ 11.08 (s, 1H), 10.46 (s, 1H), 9.97 (s, 1H), 8.98 (d, J=2.0 Hz, 1H), 8.18-8.08 (m, 1H), 7.89 (d, J=9.1 Hz, 1H), 7.71-7.64 (m, 2H), 6.83 (d, J=2.1 Hz, 1H), 6.69 (dd, J=8.4, 2.1 Hz, 1H), 5.88-5.77 (m, 1H), 5.06 (dd, J=12.8, 5.5 Hz, 1H), 4.27 (t, J=8.3 Hz, 2H), 3.95-3.81 (m, 2H), 3.64-3.54 (m, 2H), 3.37-3.18 (m, 2H), 3.17-2.97 (m, 2H), 2.69 (d, J=0.8 Hz, 1H), 2.62-2.52 (m, 2H), 2.43 (s, 3H), 2.33 (s, 3H), 2.28-2.12 (m, 4H), 2.05-1.96 (m, 1H), 1.94-1.84 (m, 2H), 1.83-1.70 (m, 2H), 1.64-1.49 (m, 2H), 1.25 (q, J=7.3 Hz, 2H).

### Compound (33) (2S,4S)-1-((S)-1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-17-(tert-butyl)-2,15-dioxo-6,9,12-trioxa-3,16-diazaoctadecan-18-oyl)-4-hydroxy-N—((S)-1-(4-(4-methylthiazol-5-yl)phenyl)ethyl)pyrrolidine-2-carboxamide (BSJ-03-095)

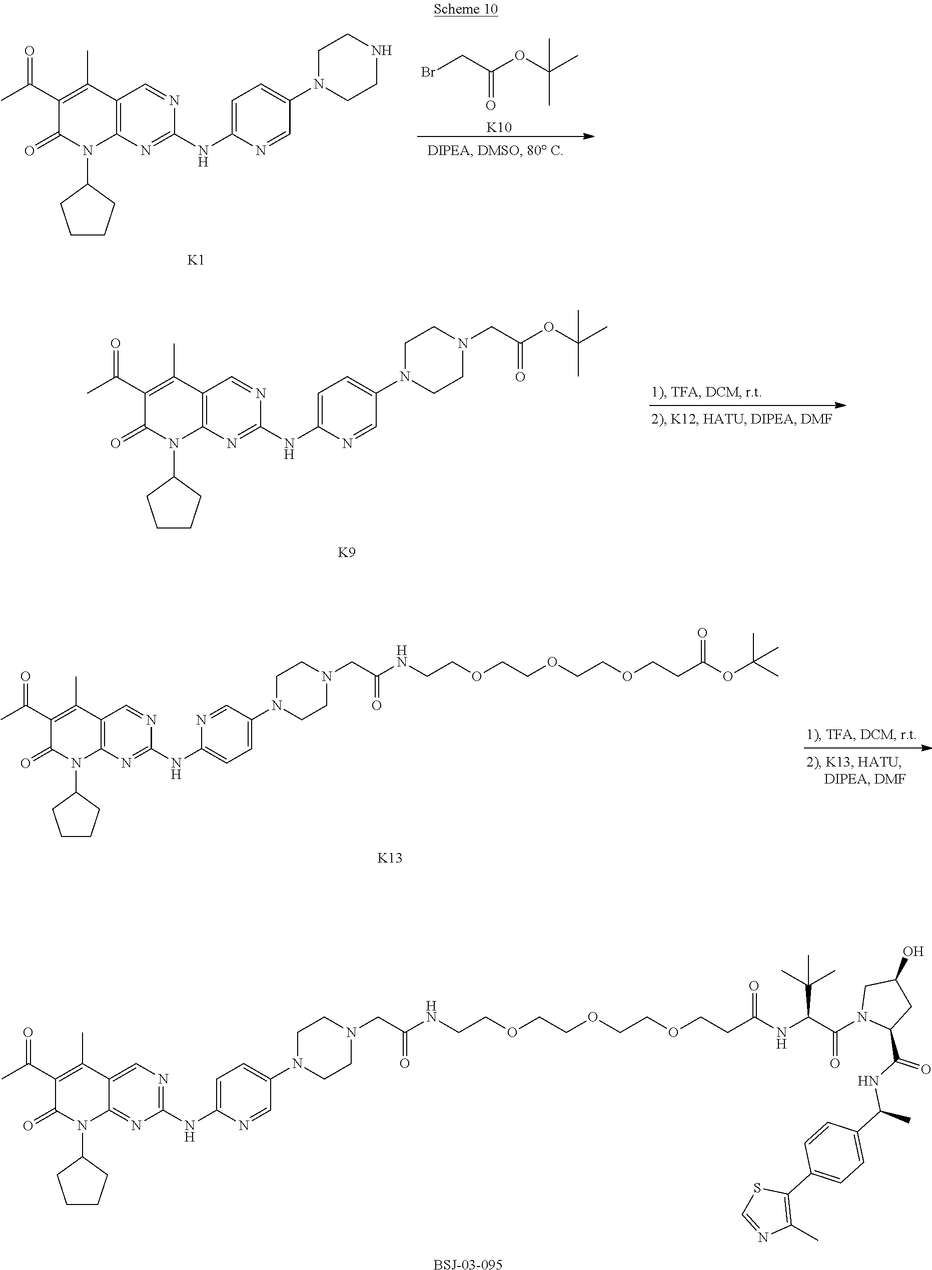

To a solution of compound K1 (50 mg, 0.11 mmol) in DMSO (5 mL) was added tert-butyl bromoacetate (K10, 0.033 mL, 0.22 mmol) and DIPEA (0.058 mL, 0.33 mmol). The mixture was heated to 80° C. and kept stirring for 48 h. The mixture was then cooled down to room temperature, diluted with EtOAc and washed once with water then twice with brine. The organic layer was dried over anhydrous sodium sulfate, filtered and concentrated under reduced pressure. The residue was purified by reverse phase HPLC (5-95% MeOH in H₂O) to give K11 as a yellow solid (48.1 mg, 78%). LC-MS: m/z 562 [M+1].

To a solution of compound K11 (47.4 mg, 0.0844 mmol) in DCM (1 mL) was added TFA (1 mL) and the resulting solution was stirred for 1h at room temperature. The mixture was concentrated to give the acid residue which was directly dissolved into 2.0 mL of DMF. To the DMF solution was then added tert-butyl 3-(2-(2-(2-aminoethoxy)ethoxy)ethoxy)propanoate (K12, 23.5 mg, 0.0844 mmol), HATU (48 mg, 0.126 mmol) and DIPEA (0.058 mL, 0.338 mmol). The resulting mixture was stirred at room temperature for 0.5h, then diluted with EtOAc and washed once with water then twice with brine. The organic layer was dried over anhydrous sodium sulfate, filtered and concentrated under reduced pressure to give the ester intermediate K13. LC-MS: m/z 767 [M+1]. This intermediated was re-dissolved into 1.0 mL of DCM, followed by addition of 1.0 mL of TFA, and stirred for 1h at room temperature. The solvent was evaporated under reduced pressure to give an acid intermediate. LC-MS: m/z 709 [M+1]. The acid was then dissolved into 2.0 mL of DMF, followed by addition of (2S,4S)-1-((S)-2-amino-3,3-dimethylbutanoyl)-4-hydroxy-N—((S)-1-(4-(4-methylthiazol-5-yl)phenyl)ethyl)pyrrolidine-2-carboxamide (K14, 36.3 mg, 0.0844 mmol), HATU (48 mg, 0.126 mmol) and DIPEA (0.058 mL, 0.338 mmol). The resulting mixture was stirred at room temperature for 0.5h, then purified by reverse phase HPLC (5-95% MeOH in H₂O) to give Compound (33) as a yellow solid (10 mg, 10.5% in 4 steps). LC-MS: m/z 1121 [M+1]. ¹H NMR (500 MHz, DMSO-d₆) δ 10.45 (s, 1H), 8.98 (d, J=5.0 Hz, 2H), 8.68 (t, J=5.6 Hz, 1H), 8.56 (t, J=6.1 Hz, 1H), 8.10 (d, J=3.0 Hz, 1H), 7.95-7.85 (m, 2H), 7.64 (dd, J=9.2, 3.1 Hz, 1H), 7.44-7.33 (m, 4H), 5.83 (p, J=8.9 Hz, 1H), 4.55 (d, J=9.4 Hz, 1H), 4.47-4.38 (m, 2H), 4.36-4.32 (m, 1H), 4.31-4.08 (br, 12H), 4.02 (s, 2H), 3.71-3.55 (m, 4H), 3.54-3.41 (m, 10H), 3.37-3.26 (m, 2H), 2.59-2.52 (m, 1H), 2.44 (s, 3H), 2.43 (s, 3H), 2.32 (s, 3H), 2.27-2.18 (m, 2H), 2.09-1.97 (m, 1H), 1.95-1.84 (m, 3H), 1.83-1.71 (m, 2H), 1.64-1.52 (m, 2H), 1.36 (d, J=7.0 Hz, 3H), 0.93 (s, 9H).

### Compound (34): (2S,4R)-1-((S)-2-(5-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pentanamido)-3,3-dimethylbutanoyl)-4-hydroxy-N—((S)-1-(4-(4-methylthiazol-5-yl)phenyl)ethyl)pyrrolidine-2-carboxamide (BSJ-05-017)

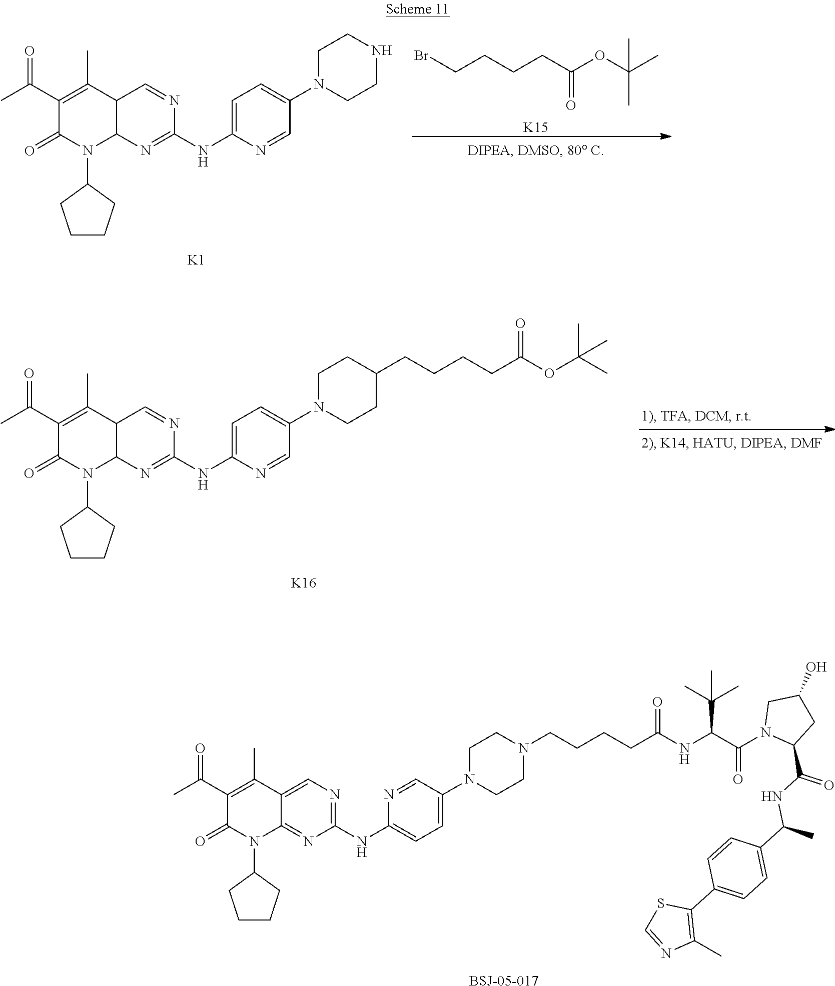

To a solution of compound K1 (50 mg, 0.11 mmol) in DMSO (5 mL) was added tert-butyl 5-bromopentanoate (K15, 51.9 mg, 0.22 mmol) and DIPEA (0.058 mL, 0.33 mmol). The mixture was heated to 80° C. and kept stirring for 48 h. The mixture was then cooled down to room temperature, diluted with EtOAc and washed once with water then twice with brine. The organic layer was dried over anhydrous sodium sulfate, filtered and concentrated under reduced pressure. The residue was purified by reverse phase HPLC (5-95% MeOH in H₂O) to give K16 as a yellow solid (27.9 mg, 42%). LC-MS: m/z 604 [M+1].

To a solution of compound K16 (25.5 mg, 0.0422 mmol) in DCM (1 mL) was added TFA (1 mL) and the resulting solution was stirred for 1h at room temperature. The mixture was concentrated to give the acid residue, which was directly dissolved into 1.0 mL of DMF, followed by addition of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (K14, 18.7 mg, 0.0422 mmol), HATU (24 mg, 0.063 mmol) and DIPEA (0.058 mL, 0.269 mmol). The resulting mixture was stirred at room temperature for 0.5h, then purified by reverse phase HPLC (5-95% MeOH in H₂O) to give Compound (34) as a yellow solid (26.3 mg, 64%). LC-MS: m/z 974 [M+1]. ¹H NMR (500 MHz, DMSO-d₆) δ 10.37 (s, 1H), 9.60 (s, 1H), 8.97 (dd, J=7.6, 5.7 Hz, 2H), 8.35 (d, J=7.8 Hz, 1H), 8.14-8.02 (m, 1H), 7.96-7.80 (m, 2H), 7.71-7.55 (m, 1H), 7.47-7.40 (m, 2H), 7.39-7.32 (m, 2H), 5.89-5.75 (m, 1H), 4.95-4.87 (m, 1H), 4.51 (dd, J=14.3, 9.2 Hz, 1H), 4.42 (t, J=8.1 Hz, 1H), 4.30 (dt, J=6.8, 3.0 Hz, 1H), 3.70 (br, 5H), 3.16 (d, J=9.8 Hz, 5H), 3.03 (t, J=12.4 Hz, 2H), 2.45 (s, 3H), 2.43 (s, 3H), 2.32 (s, 3H), 2.29-2.16 (m, 4H), 2.08-1.98 (m, 1H), 1.95-1.85 (m, 2H), 1.83-1.72 (m, 3H), 1.71-1.63 (m, 2H), 1.62-1.52 (m, 4H), 1.37 (d, J=7.0 Hz, 3H), 0.95 (s, 9H). ¹³C NMR (126 MHz, DMSO) δ 202.88, 172.05, 171.04, 170.03, 161.16, 158.76, 158.52, 155.27, 151.98, 148.20, 145.54, 145.05, 142.43, 142.28, 131.57, 130.18, 130.15, 129.30, 127.01, 126.86, 115.72, 107.50, 69.25, 59.06, 57.00, 56.77, 55.72, 53.44, 51.10, 48.16, 46.11, 38.27, 35.72, 34.55, 31.77, 28.05, 26.94, 26.89, 25.63, 23.35, 22.90, 22.84, 16.43, 14.14.

### Compound (35): (2S,4R)-1-((R)-2-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanamido)-3,3-dimethylbutanoyl)-4-hydroxy-N—((S)-1-(4-(4-methylthiazol-5-yl)phenyl)ethyl)pyrrolidine-2-carboxamide (BSJ-05-059)

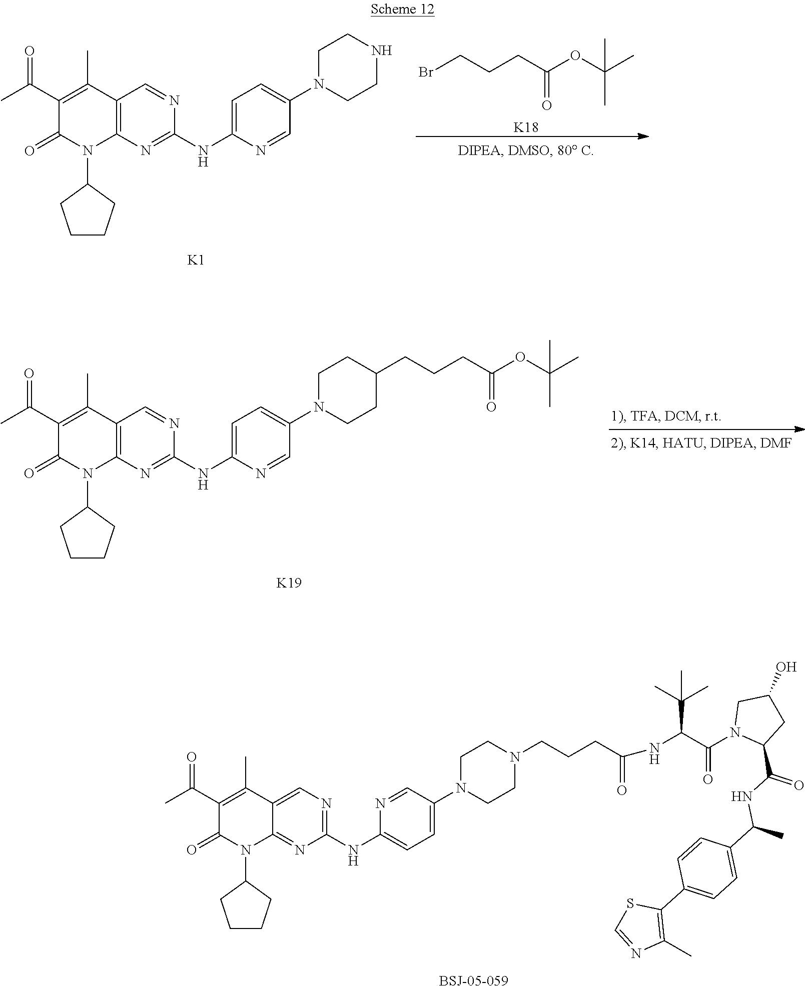

Compound (35) was synthesized from compound 1, tert-butyl 3-(bromomethyl)azetidine-1-carboxylate (K18) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (K14). Compound (35) was obtained as a yellow solid. LC-MS: m/z 960 [M+1]. ¹H NMR (500 MHz, DMSO-d₆) δ 10.46 (s, 1H), 9.60 (s, 1H), 9.01-8.95 (m, 2H), 8.8.31 (d, J=7.6 Hz, 1H), 8.16-8.10 (m, 1H), 7.99-7.89 (m, 2H), 7.69 (dd, J=8.9, 3.2 Hz, 1H), 7.41-7.32 (m, 2H), 7.31-7.23 (m, 2H), 5.89-5.75 (m, 1H), 4.95-4.87 (m, 1H), 4.57-4.48 (m, 1H), 4.43-4.34 (m, 1H), 4.30-4.19 (m, 1H), 3.69 (br, 5H), 3.25-2.98 (m, 6H), 2.45 (s, 3H), 2.42 (s, 3H), 2.32 (s, 3H), 2.28-2.15 (m, 4H), 2.10-1.98 (m, 1H), 1.95-1.85 (m, 2H), 1.81-1.69 (m, 2H), 1.62-1.52 (m, 4H), 1.34 (d, J=7.0 Hz, 3H), 0.97 (s, 9H).

### Compound (36): (2R,4S)-1-((S)-2-(5-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pentanamido)-3,3-dimethylbutanoyl)-4-hydroxy-N—((S)-1-(4-(4-methylthiazol-5-yl)phenyl)ethyl)pyrrolidine-2-carboxamide (BSJ-05-017NC)

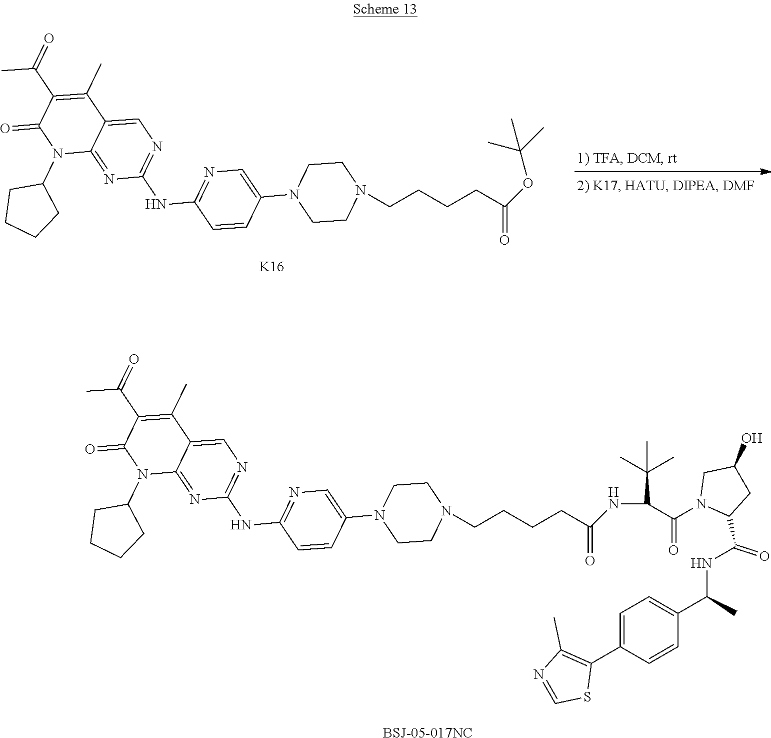

To a solution of compound K16 (25.5 mg, 0.0422 mmol) in DCM (1 mL) was added TFA (1 mL) and the resulting solution was stirred for 1h at room temperature. The mixture was concentrated to give the acid residue, which was directly dissolved into 1.0 mL of DMF, followed by addition of (2R,4S)-1-((S)-2-amino-3,3-dimethylbutanoyl)-4-hydroxy-N—((S)-1-(4-(4-methylthiazol-5-yl)phenyl)ethyl)pyrrolidine-2-carboxamide (K17, 18.7 mg, 0.0422 mmol), HATU (24 mg, 0.063 mmol) and DIPEA (0.058 mL, 0.269 mmol). The resulting mixture was stirred at room temperature for 0.5h, then purified by reverse phase HPLC (5-95% MeOH in H₂O) to give Compound (36) as a yellow solid (26.1 mg, 63%). LC-MS: m/z 974 [M+1]. ¹H NMR (500 MHz, DMSO-d₆) δ 10.49 (s, 1H), 9.59 (s, 1H), 8.99 (d, J=7.5 Hz, 2H), 8.21 (d, J=8.0 Hz, 1H), 8.10 (d, J=3.0 Hz, 1H), 7.99 (d, J=8.4 Hz, 1H), 7.88 (d, J=9.1 Hz, 1H), 7.66 (dd, J=9.2, 3.1 Hz, 1H), 7.45 (s, 4H), 5.89-5.78 (m, 1H), 4.93-4.83 (m, 1H), 4.50 (d, J=8.5 Hz, 1H), 4.41 (dd, J=8.2, 5.8 Hz, 1H), 4.38-4.29 (m, 1H), 3.85 (d, J=12.6 Hz, 2H), 3.77 (dd, J=10.4, 5.2 Hz, 1H), 3.64-3.48 (m, 3H), 3.22-2.95 (m, 6H), 2.46 (s, 3H), 2.43 (s, 3H), 2.32 (s, 3H), 2.30-2.15 (m, 4H), 2.09-1.98 (m, 1H), 1.96-1.83 (m, 3H), 1.83-1.72 (m, 2H), 1.67-1.44 (m, 6H), 1.32 (d, J=7.0 Hz, 3H), 0.97 (s, 9H).

### Synthesis of Biotinylated Palbociclib (BSJ-03-163)

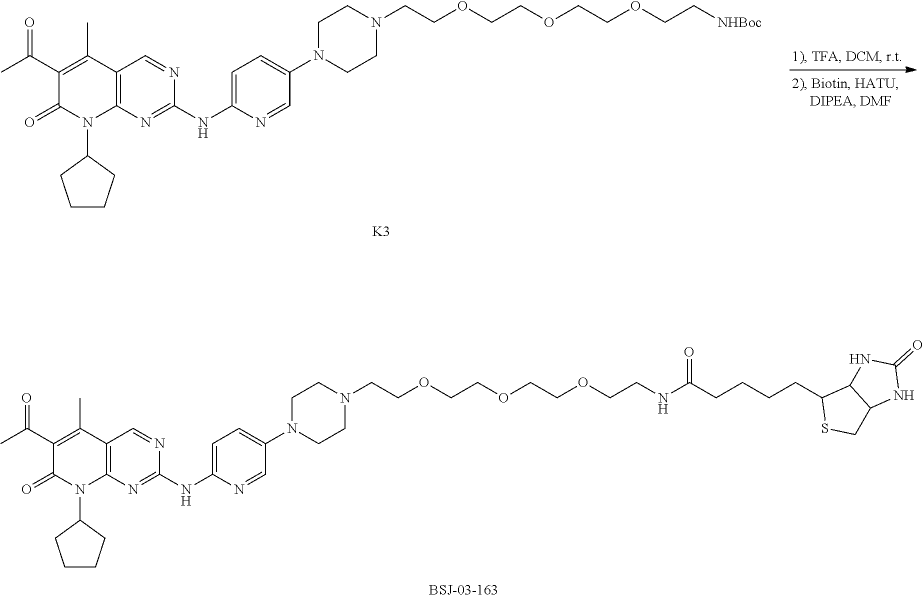 [SMILES: CC(=O)c1c(C)c2cnc(Nc3ccc(N4CCN(CCOCCOCCOCCNC(=O)OC(C)(C)C)CC4)cn3)nc2n(C2CCCC2)c1=O ; "K3" ; CC(=O)c1c(C)c2cnc(Nc3ccc(N4CCN(CCOCCOCCOCCNC(=O)CCCCC5SCC6NC(=O)NC65)CC4)cn3)nc2n(C2CCCC2)c1=O ; "BSJ-03-163"]

To a solution of tert-butyl (2-(2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethoxy)ethoxy)ethoxy)ethyl)carbamate (K3, 30.5 mg, 0.0422 mmol) in DCM (1 mL) was added TFA (1 mL) and the resulting solution was stirred for 0.5h at room temperature. The mixture was concentrated to give the primary amine intermediate, which was directly dissolved into 1.0 mL of DMF, followed by addition of Biotin (10.3 mg, 0.0422 mmol), HATU (24 mg, 0.063 mmol) and DIPEA (0.058 mL, 0.269 mmol). The resulting mixture was stirred at room temperature for 0.5h, then purified by reverse phase HPLC (5-95% MeOH in H₂O) to give BSJ-3-163 as a yellow solid (26.1 mg, 81%). LC-MS: m/z 849 [M+1].

### Example 2. Procedures and Methods

### Co-Immunoprecipitation (IP)

IP-mass spectrometry. Cell pellets were lysed in IP lysis buffer (Pierce, #87787) supplemented with 1× protease and phosphatase inhibitors (Pierce, #78444). After 10 min incubation on ice, lysates were centrifuged at maximum speed for 10 min at 4° C. and the supernatants were obtained for the measurement of protein concentration. 1 mg of lysates were immunoprecipitated by incubating 1 g CDK4 (# sc-23896, Santa Cruz Biotechnology) or CDK6 (# sc-177-G, Santa Cruz Biotechnology) antibody at 4° C. overnight. 20 μl of ChIP-grade magnetic beads (Thermo Fisher Scientific) was added into each IP tube and incubated for 2 hours. IP samples were washed 3 times with IP lysis buffer, resuspended in 2×LDS sample buffer (Invitrogen) and boiled for 5 min at 100° C. before loading onto SDS-PAGE gels. The gel was stained with SimplyBlue safestain (Thermo Fisher Scientific) and used for mass spectrometry. Mass spectrometry was conducted through the Q Exactive Plus mass spectrometer (Thermo Scientific) platform. MS raw files were converted into MGF by Proteome Discover (Thermo Scientific) and processed using Mascot 2.4 (Matrix Science, U.K.) by searching against the UniProt human database supplemented with common contaminant proteins. Mascot data were assembled by Scaffold and X!-Tandem software and search criteria for identification was 4 minimum peptides and 1% FDR at the peptide and protein level. Scaffold_4.8.3 was used to visualize and analyze the mass spectrometry data. A protein threshold above 99% and peptide threshold above 95% were used to isolate proteins of interest. Gene ontology analysis was performed using the gene ontology website (http://geneontology.org/).

IP-in vitro kinase assay. For IP-kinase assay, cells were lysed on ice for 10 minutes in kinase lysis buffer (20 mM Tris-HCl (pH 7.5), 150 mM NaCl, 1 mM Na₂EDTA, 1 mM EGTA, 1% Triton, 2.5 mM sodium pyrophosphate, 1 mM beta-glycerophosphate, 1 mM Na₃VO₄, 1 μg/ml leupeptin, from Cell Signaling Technology, #9803) supplemented with 1× protease and phosphatase inhibitors. Lysates were collected as described above. 300 g of cell lysates were incubated with 1 g CDK4 (# sc-23896, Santa Cruz Biotechnology) or CDK6 (# sc-177-G, Santa Cruz Biotechnology) antibody at 4° C. overnight. 20 μl of ChIP-grade magnetic beads (Thermo Fisher Scientific) was added into each IP tube and incubated for 2 hours. IP samples were washed 2 times with kinase lysis buffer and 2 times with kinase reaction buffer (40 mM Tris-HCl [pH 7.5], 20 mM MgCl₂, 0.1 mg/ml BSA, from SignalChem, #K03-09, with 50 M DTT adding freshly). 100 μl of kinase reaction buffer with 0.5 μg of recombinant human Rb1 protein and 100 μM of ATP was added into each tube. The kinase reaction system was incubated at 30° C. for 30 minutes on a thermomixer. 20 μl of reaction mixture (without beads) was mixed with 20 μl of ADP-Glo reagent and incubated for 1 hour at room temperature. Then 40 μl of kinase detection reagent was added and incubated for 40 minutes at room temperature. Samples were read on the Glomax luminometer (Promega) and kinase activities were calculated. The remaining reaction mixture (without beads) was denatured by LDS and DTT and western blotting was performed to detect phosphorylation of Rb protein. The remaining proteins on beads were eluted by 2×LDS buffer and western blotting was used to confirm the kinase pull-down.

### In Vitro Kinase Assay Using Recombinant Proteins.

In vitro kinase assay was performed in a final volume of 5 μl kinase buffer (SignalChem, #K03-09) supplemented with 50 M DTT, 100 M ATP and 5 ng/l Rb1 recombinant protein. CDK4/Cyclin D3 (SignalChem #C31-18G) and CDK6/Cyclin D3 (SignalChem #C35-10G) were used as kinases. CDK4/6 inhibitors and/or INK4 proteins were pre-incubated with the kinases for 10 min at room temperature before adding ATP and Rb1 substrate. After 1 h incubation at room temperature, 5 μl ADP-Glo reagent was added to the kinase reaction mixture and incubated for 1 h, followed by incubating with 10 μl kinase detection reagent for 40 min. The luminescence is detected on SpectraMax iD5 microplate reader.

### Cloning and Plasmids

LentiCRISPRv2 or lenti-sgRNA backbone were used for generating knockout cell lines. LentiCRISPRv2 puro, lentiCRISPRv2 hygro and lenti-sgRNA neo were gifts from Brett Stringer (Addgene plasmid #98290, #98291 and #104992). Single guide RNAs were designed through MIT CRISPR Designer (crispr.mit.edu) and the sequences are: FAT1-CRISPR: CACGGTGACGTTGTACTCGG; CDKN2B (p15)-CRISPR: ACGGAGTCAACCGTTTCGGG and CTCCACTAGTCCCCGCGCCG; CDKN2C (p18)-CRISPR: GAATGACAGCGAAACCAGTT and TTAACATCGAGGATAATGAA; PTEN-CRISPR: TCATCTGGATTATAGACCAG. Instructions for using the lentiCRISPRv2 plasmids are as described by the Zhang laboratory (https://media.addgene.org/cms/filer_public/53/09/53091cde-blee-47ee-97cf-9b3b05d290f2/lenticrisprv2-and-lentiguide-oligo-cloning-protocol.pdf). Oligos were annealed and ligated with BsmBI digested lentiviral vector. Then the ligation system was transformed into Stbl3 bacteria and plasmids were extracted for sequencing.

pLKO-PTEN-shRNA-1320 and pLKO-PTEN-shRNA-3001 were gifts from Todd Waldman (Addgene plasmid #25638 and #25639). We obtained them from Dr. Neal Rosen's lab. Other shRNA sequences are as follows: Renilla-sh: TGCTGTTGACAGTGAGCGCAGGAATTATAATGCTTATCTATAGTGAAGCCACAGA TGTATAGATAAGCATTATAATTCCTATGCCTACTGCCTCGGA; ARID1A-sh-1: TGCTGTTGACAGTGAGCGCAAGCGAGACACAGCTATTTAATAGTGAAGCCACAG ATGTATTAAATAGCTGTGTCTCGCTTTTGCCTACTGCCTCGGA; YAP-sh: TGCTGTTGACAGTGAGCGCTAGGTTGATCACTCATAATAATAGTGAAGCCACAG ATGTATTATTATGAGTGATCAACCTATTGCCTACTGCCTCGGA. Renilla, ARID1A and YAP1 shRNAs were put into mir-E, an optimized microRNA backbone, as previously described (50). Briefly, hairpin ultramers were amplified and put into lentiviral SGEP or SGEN vectors, which are gifts from the Charles Sawyers lab. Proper insertions were verified by Sanger sequencing. ARID1A siRNA was purchased from Invitrogen (Cat #4392420). pDONR223-CDK6 was cloned into MSCV—N-Flag-HA-IRES-PURO (a gift from William Hahn and David Root; Addgene #23688) and pLenti PGK Neo DEST (w531-1) (a gift from Eric Campeau & Paul Kaufman; Addgene plasmid #19067) using the Gateway LR Clonase II Enzyme Mix (Invitrogen, Waltham, MA, USA) (9). Single-site mutagenesis was performed using the QuikChange II XL Site-Directed Mutagenesis Kit (Agilent Technologies #200522). Proper mutations were verified by Sanger sequencing.

### Lentiviral and Retroviral Infection and Generation of Stable Cell Lines

HEK293T cells were transfected with 4.5 μg of lentiviral vector, 4.5 μg of psPAX2/pCL-Ampho and 1 μg of pVSVG with 40 μl X-tremeGENE HP (Roche) according to the manufacturer's protocol. Conditioned medium containing recombinant lentivirus was collected 48 hrs after transfection and filtered through non-pyrogenic filters with a pore size of 0.45 m (Merck Millipore, Billerica, MA, USA). Samples of these supernatants were applied immediately to target cells together with Polybrene (Sigma-Aldrich, St. Louis, MO, USA) at a final concentration of 8 g/ml, and supernatants were incubated with cells for 12 h. After infection, cells were placed in fresh growth medium and cultured as usual. Selection with 2 μg/ml puromycin (Thermo Fisher Scientific), 1 mg/ml G418 (InvivoGen, San Diego, CA, USA) or 200 μg/ml hygromycin (InvivoGen, San Diego, CA, USA) was initiated 48 h after infection.

### Cell Viability Assay

Cell viability was measured by Resazurin (R&D Systems, Minneapolis, MN, USA) as described previously (51). Briefly, 1,500 cells were seeded in a 96-well plate and allowed to recover overnight. Cells were treated with drugs at day 0. Resazurin was added to the cells 4 hours prior to the measurements on day 3, day 5 and day 7. Fluorescent intensity was measured using a microplate reader (SpectraMax M5, Molecular Devices, Sunnyvale, CA, USA). IC50 was calculated by GraphPad Prism 7.0 using a sigmoidal regression model.

### Western Blotting

Cell lysates were collected in RIPA buffer (Thermo Fisher Scientific) supplemented with protease and phosphatase inhibitors (Pierce). Protein concentration was quantified by using the BCA kit (Fisher Scientific). 60-100 g of protein lysates were loaded onto 4-12% SDS-PAGE gels (Invitrogen) for electrophoresis and transferred onto nitrocellular membranes. Blots were blocked with Intercept™ (TBS) Blocking Buffer (LI-COR Bioscience #927-60001) and incubated with primary antibody at 4° C. overnight. Secondary antibodies conjugated with fluorescence (LI-COR Bioscience #926-68071 and #926-32210) were incubated for 1 h at room temperature and blots were scanned by Odyssey Clx Imaging System from LI-COR Bioscience.

### Immunohistochemistry (IHC)

Immunohistochemistry was performed on formalin-fixed paraffin-embedded tumor specimens from patient-derived xenografts provided by Dr. Violeta Serra from VHIO in Barcelona, Spain. A standard multimer/diaminobenzidine (DAB) detection protocol was performed on Ventana BenchMark ULTRA Automated stainer as previously described (8), with appropriate negative and positive controls. 2 μg/ml FAT1 (Abcam #ab190242), 1 μg/ml YAP (Cell Signaling Technology #14074), 1 μg/ml CDK6 (Sigma-Aldrich, #HPA002637), 1 ug/ml p15 (R&D systems, #MAB6798) and 1 μg/ml p18 (Cell signaling technology, #2896) antibodies were used. Images were taken under Leica DMi8 microscope and evaluated by a pathologist at MSKCC. Quantification of the staining was based on the percentage of positive staining and staining intensity at the indicated location. The immunoreactive scores were recorded as previously described (52,53).

### Computational Structural Analysis

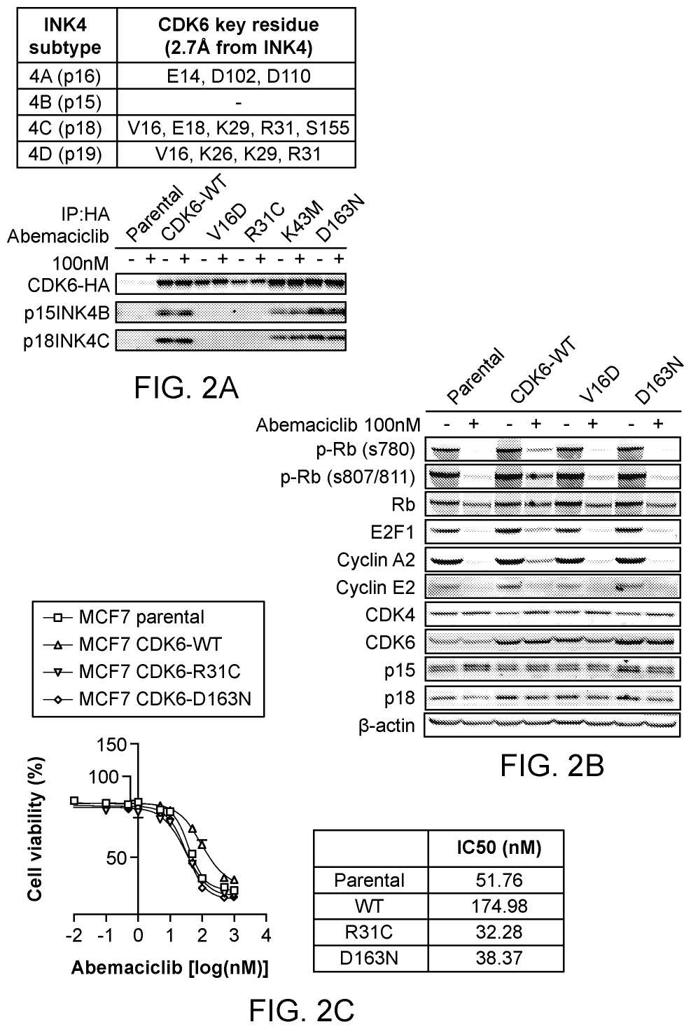

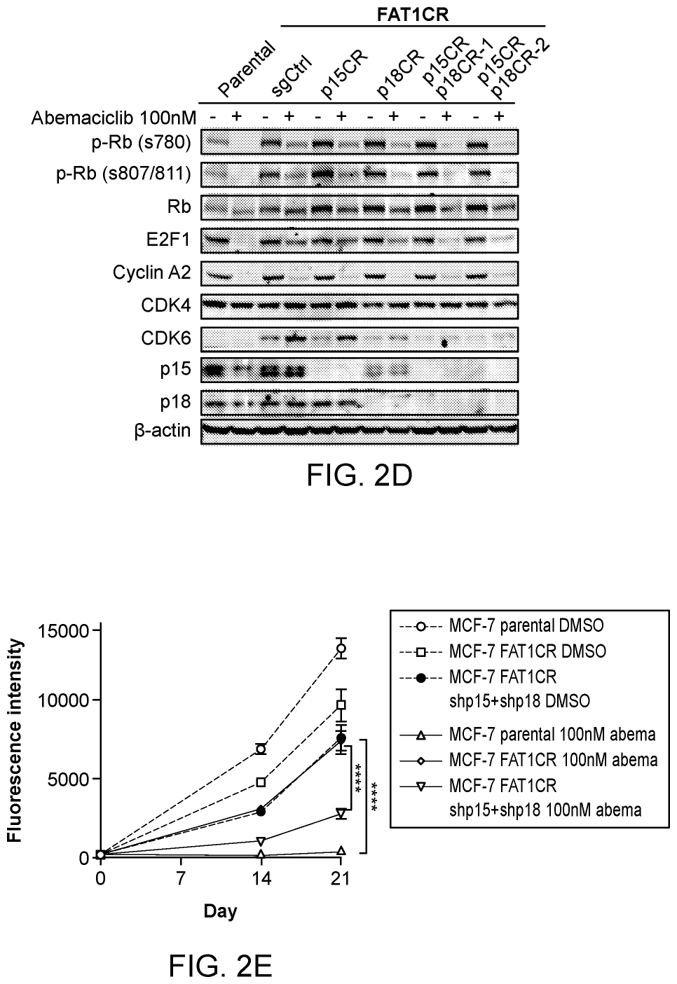

INK4-CDK6 interface analysis. Three crystallographic structures were superposed in the PDB database of CDK6-INK4 (PDBIDs: 1B1I7, 1B1I8, 1G3N (19,20)) using UCSF-Chimera v1.14 (54) and CDK6 residues that are in proximity of INK4 (<2.7 Å) were selected (listed in FIG. 2A) as candidates for mutagenesis experiments.

Quantification of the change in CDK6 binding pocket volume upon INK4 binding. Besides the existing INK4-bound CDK6 structures as listed in the table in FIG. 2A, we also selected PDBID: 2EUF (30) as the structure that cognately binds to palbociclib without INK4 bound. Structural superposition was performed using UCSF-Chimera v1.14. For each structure, we docked AMP-PNP and palbociclib into the CDK6 binding pocket using the flexible docking protocol in the software DOCK6.9 (55). We also used DOCK6.9 to generate spheres (i.e. probes along the protein surface) and selected those that were occupied by the predicted binding pose of the ligand to approximate the volume of the binding pocket. We elected to use the docked pose for AMP-PNP and the superposed pose for palbociclib due to incomplete pose generation in the distorted binding pocket. Input and raw docking results can be found in the following GitHub repository: https://github.com/choderalab/CDK_PROTAC.

Manual construction of the structural models of the ternary complex. Various existing structures (wild type, human proteins) from the PDB were used to construct the complex. The catalytic domain of CDK6 and CDK4 were from PDBIDs 1G3N and 3G33, respectively. CRBN was from PDBID 5FQD and VHL was from PDBID 5T35. CyclinD1 was from PDBID 6P8E. For each PROTAC degrader, first, the two warheads were docked to the binding pocket of the E3 ligase adapter and CDK4 (superposed for CDK6 due to the distorted binding pocket). Each palbociclib posed in the CDK binding pockets was then relaxed with a short (20 ns) molecular dynamics simulation (at 310.15 K, 1.0 atm, 4 fs timesteps with heavy hydrogen masses) (to further open the pocket to increase compatibility with the degrader linker) using OpenMM package v7.4.2 (56). Then the docked poses for the two warheads were superimposed to common rotatable bonds in an extended pose of the degrader linker using UCSF-Chimera v1.14. Once clashes in the protein targets were eliminated by manual rotation and reorientation of side chains, the two warheads and the linker were manually bonded.

Molecular dynamics simulations and post-analysis. The manually constructed model structures were solvated in TIP3P water (57) and neutralized with an amount of NaCl equivalent to the ionic strength of 20 mM MgCl₂ to match the experimental condition. The small molecule ligands were parameterized using the GAFF forcefield (58) and the AM1-BCC charging method (59) implemented in the software package Antechamber (60). Molecular dynamics simulations were run using the Amber14SB forcefield (61) through the OpenMM package v7.4.2 (56). Short equilibration (5 ns) was performed before the production run (ended up with ˜300 ns) using the Langevin integrator at 400.15 K and 1.0 atm with a timestep of 4 fs (using heavy hydrogens with a mass of 4 atomic mass units). The arbitrarily high temperature (127° C.) was used for the simulations to ensure that the complexes were not trapped in initial conformations and were able to reach reasonable equilibration. Trajectories from the simulations were post-analyzed (imaged on one of the protein components and converted to the pdb format) using MDTraj v1.9.4 (62) and visualized using the software package PyMOL v2.2.0. Hydrogen bonds in each trajectory were identified using the Baker-Hubbard criterion in MDTraj and the union of the three sets of the most frequently observed hydrogen bonds was identified. Coordinates for the equilibrated structures of the four ternary-complex models can be found in the following GitHub repository: https://github.com/choderalab/CDK_PROTAC.

### Microscale Thermophoresis (MST) Assay.

MST assay was done by Reaction Biology Corp. (Malvern, PA, USA). Briefly, protein CDK6 was labeled using the Protein Labeling Kit RED-NHS (NanoTemper Technologies). The labeling reaction was performed according to the manufacturer's instructions in the supplied labeling buffer applying a concentration of 15 μM protein (molar dye:protein ratio ˜3:1) at room temperature for 30 min. Unreacted dye was removed with the supplied dye removal column equilibrated with storage buffer (50 mM Hepes pH 7.5, 500 mM NaCl, 10% glycerol, 0.25 mM TCEP, 0.01% tween 20). The degree of labeling was determined using UV/VIS spectrophotometry at 650 and 280 nm. A degree of labeling of 0.6 was achieved. The labeled protein CDK6 was adjusted to 12 nM with assay buffer (20 mM K phosphate, pH 8.0, 50 mM NaCl, 0.05% Pluronic). 250 nM p18 was pre-incubated with CDK6 for 15 min prior to the addition of ligand. The ligand abemaciclib was dissolved in assay buffer and a series of 16 1:1 dilution was prepared using the same buffer, producing ligand concentrations ranging from 122 μM to 4 μM. Each ligand dilution was mixed with one volume of labeled protein, resulting in a final labeled CDK6 concentration of 6 nM and final ligand concentrations ranging from 61 μM to 2 nM. After 20 min incubation, the samples were loaded into standard Monolith NT.115 Capillaries (NanoTemper Technologies). MST was measured using a Monolith NT.115 instrument (NanoTemper Technologies) at an ambient temperature of 25° C. Instrument parameters were adjusted to 10% LED power and medium MST power. Data of three independently pipetted measurements were analyzed (MO.Affinity Analysis software version 2.1.3, NanoTemper Technologies) using the signal from an MST-on time of 5 s. The data was expressed as baseline Corrected Normalized Fluorescence ΔFnorm [‰]. To obtain ΔFnorm, the baseline Fnorm value is subtracted from all data points of the same curve. (The baseline Fnorm value is equivalent to the mean Fnorm value of the unbound target, usually in capillaries 14-16, and is given by the MO.Affinity Analysis software as the ‘unbound’ value when a fit is performed.)

### Flow Cytometry: Senescence Green and Cell Cycle

Senescence analysis. Cells were treated with DMSO, abemaciclib (100 nM), palbociclib (500 nM) and BSJ-05-017 (500 nM) for 8 days. Cells were harvested using trypsin/EDTA, then stained with the CellEvent Senescence Green Flow Cytometry Assay Kit (Invitrogen) according to the manufacturer's instructions. Briefly, cells were fixed with 2% paraformaldehyde for 15 min at room temperature and stained with the CellEvent Senescence Green Probe for 2 hours at 37° C. without CO₂. After incubation, cells were washed with PBS 3 times and resuspended in FACS buffer (PBS with 2% FBS) for analysis on BD Biosciences LSR Fortessa using a 488 nm laser and 530 nm/30 filter (BD Biosciences). Data analysis was performed with FCS Express 7 (De Novo Software).

Cell cycle analysis. Cells were treated with DMSO, abemaciclib (100 nM), palbociclib (500 nM) and BSJ-05-017 (500 nM) for 24 hours. Cells were detached from the cell culture dish with trypsin/EDTA, then washed with PBS and fixed in 70% ice-cold EtOH overnight. Prior to staining, EtOH was removed, and cells were washed twice with FACS buffer. Cells were then resuspended in staining buffer containing 1000 μl FACS buffer with 2 μg/ml propidium iodide (Invitrogen) and 100 μg/ml RNase A (Invitrogen). Cell cycle profiles were measured with BD Biosciences LSR Fortessa and analyzed with FCS Express 7.

### Proteomics

Molt4 cells were treated with 250 nM of compounds BSJ-05-017 or BSJ-03-096 (singlicate) or DMSO control (biological triplicate) for 5 hours. Cells were harvested by centrifugation and prepared for mass spectrometry as described previously (28). Data were collected as reported (28). LC-MS data were analyzed using Proteome Discoverer 2.4 (Thermo Fisher Scientific), as previously described (28). Reporter ion intensities were normalized and scaled using in-house scripts in the R framework (63). Statistical analysis was carried out using the limma package within the R framework (64).

### In Vivo Studies

Pharmacokinetic study. C57BL/6 male mice were dosed with BSJ-05-017 solution formulation (i.p., 5/95 DMSO/10% captisol, dose 25 mg/kg) and BSJ-03-096 solution formulation (p.o., 5/95 DMSO/10%, dose 10 mg/kg). Blood samples were collected at 0.08, 0.25, 0.5, 1, 2, 4, 6, 8 and 24 hr. The blood samples were collected from sets of three mice at each time point in microcentrifuge tubes containing K₂EDTA as an anticoagulant. Plasma samples were separated by centrifugation and stored below −70° C. until bioanalysis. All samples were processed for analysis by precipitation using acetonitrile and analyzed with a partially validated LC/MS/MS method (LLOQ −1.22 ng/mL for IV and PO, LLOQ −5.02 ng/mL for IP). Pharmacokinetic parameters were calculated using the noncompartmental analysis tool of WinNonlin Enterprise software (Version 6.3).

Pharmacodynamic study. NOD.Cg-Prkdc<scid> Il2rg<tm1Wjl>/SzJ (NSG) mice were obtained from the Jackson Laboratory (Stock #: 005557). Each mouse was injected with FAT1 loss cells subcutaneously 1 week after the implantation of estradiol pellets (25 mg). After the tumors reached 200 mm³, mice were treated for 3 consecutive days with BSJ-05-017 at 25 mg/kg. Tumors were collected at 6 h. Lysates were prepared by homogenization in SDS-lysis buffer (˜1 ml/mg tissue) (50 mM Tris-HCl pH 7.4, 2% SDS) and boiled for 10 min, followed by brief sonication as described previously (65). Lysates were cleared by centrifugation at 14,000 g for 10 min and the supernatant was collected for western blotting.

Efficacy study. NOD.Cg-Prkdc<scid> Il2rg<tm1Wjl>/SzJ (NSG) mice were obtained from the Jackson Laboratory (Stock #: 005557). Each mouse was injected with MCF7 parental, CDK6-ovexpressing or PTEN loss cells subcutaneously 1 week after the implantation of estradiol pellets (25 mg). After the tumors reached 150-200 mm³, mice were treated at 5 days on/2 days off schedule for 25-35 days with ribociclib at 25 mg/kg (p.o.), BSJ-05-017 at 50 mg/kg (i.v.) and BSJ-03-096 at 50 mg/kg (p.o.). Tumor volumes were recorded every 3-4 days. Mice were sacrificed if tumors reached 1000 mm³ or at the end of the experiment. Tumors were collected and processed as described above.

Human subjects. A total of 1366 metastatic tumors from 1115 patients with HR+/HER2− metastatic breast cancer who underwent prospective clinical genomic profiling between April 2014 and March 2020 were analyzed. This study was approved by the Memorial Sloan Kettering Cancer Center Institutional Review Board (IRB) and all patients provided written informed consent for tumor sequencing and review of patient medical records for detailed demographic, pathologic, and treatment information (NCT01775072). Detailed clinicopathologic data were obtained for each sample.

Prospective sequencing and analysis. For all 1366 tumors, matched tumor and normal DNA samples were extracted from either representative formalin-fixed paraffin embedded (FFPE) tumor biopsy samples or mononuclear cells from peripheral blood, respectively. All specimens underwent massively parallel next-generation sequencing in a CLIA-certified laboratory using MSK-IMPACT, an FDA-authorized hybridization capture-based next-generation sequencing assay, which analyzes all protein-coding exons and selected intronic and regulatory regions of 341 to 468 cancer-associated genes, all as previously described (66-68). Somatic mutations, DNA copy number alterations, and structural rearrangements were identified as previously described (67) and all mutations were manually reviewed.

### PRISM Cell Line Screening

Cell Lines. The current PRISM cell set consists of 931 cell lines representing more than 45 lineages including both adherent and suspension/hematopoietic cell lines. These cell lines largely overlap with and reflect the diversity of the Cancer Cell Line Encyclopedia (CCLE) cell lines (see https://portals.broadinstitute.org/ccle). Cell lines were grown in RPMI 10% FBS without phenol red for adherent lines and RPMI 20% FBS without phenol red for suspension lines. Parental cell lines were stably infected with a unique 24-nucleotide DNA barcode via lentiviral transduction and blasticidin selection. After selection, barcoded cell lines were expanded and QCed (mycoplasma contamination test, a SNP test for confirming cell line identity, and barcode ID confirmation). Passing barcoded lines were then pooled (20-25 cell lines per pool) based on doubling time and frozen in assay-ready vials.

PRISM Screening. Test compounds were added to 384-well plates and run at 8 pt. dose with 3-fold dilutions in triplicate. These assay ready plates were then seeded with the thawed cell line pools. Adherent cell pools were plated at 1250 cells per well, while suspension and mixed adherent/suspension pools were plated at 2000 cells per well. Treated cells were incubated for 5 days then lysed. Lysate plates were collapsed together prior to barcode amplification and detection.

Barcode Amplification and Detection. Each cell line's unique barcode is located at the end of the blasticidin resistance gene and gets expressed as mRNA. These mRNAs were then captured by using magnetic particles that recognize polyA sequences. mRNA was then reverse-transcribed into cDNA and then the sequence containing the unique PRISM barcode was amplified using PCR. Finally, Luminex beads that recognize the specific barcode sequences in the cell set were hybridized to the PCR products and then detected using a Luminex scanner which reports signal as a median fluorescent intensity (MFI).

Biomarker Identification. After data processing, we explored the univariate associations between the PRISM sensitivity profiles and the genomic features or genetic dependencies. In particular, we computed the Pearson correlations and associated p-values. Correlations and p-values for log-viability values at each dose, AUC scores and logIC50 values were tabulated. For each dataset, the q-values were computed from p-values using the Benjamini-Hochberg algorithm. Associations with q-values above 0.1 were filtered out and q-values below 1e−20 plotted at 1e−20 for plot readability. Univariate models were run on available feature sets including CCLE genomic characterization data such as gene expression, cell lineage, mutation, copy number, metabolomics, and proteomics, as well as loss-of-function genetic perturbation (both RNAi and CRISPR) data from the Dependency Map. In addition to these datasets, viability data from the PRISM drug repurposing project was used as a feature set for univariate analysis. For discrete data, such as mutation and lineage, a t-test was done to determine differential sensitivities. For continuous data, such as gene expression, correlations between sensitivity and the characteristic of interest were calculated to determine any association.

### Example 3. INK4 Proteins Interact with CDK6 in CDK4 6 Inhibitor-Resistant Cells

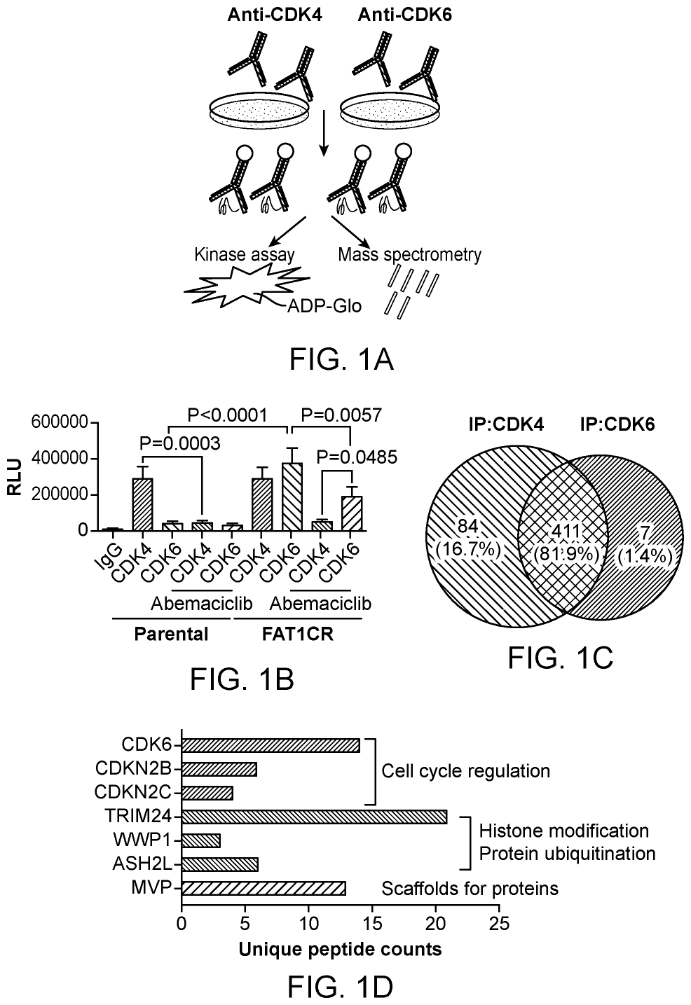

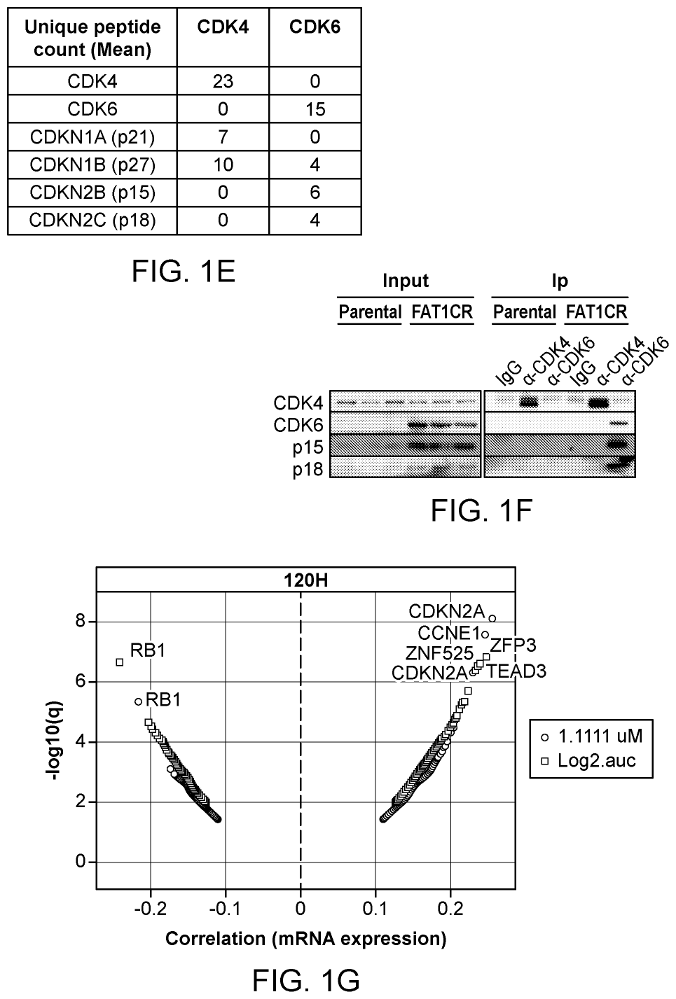

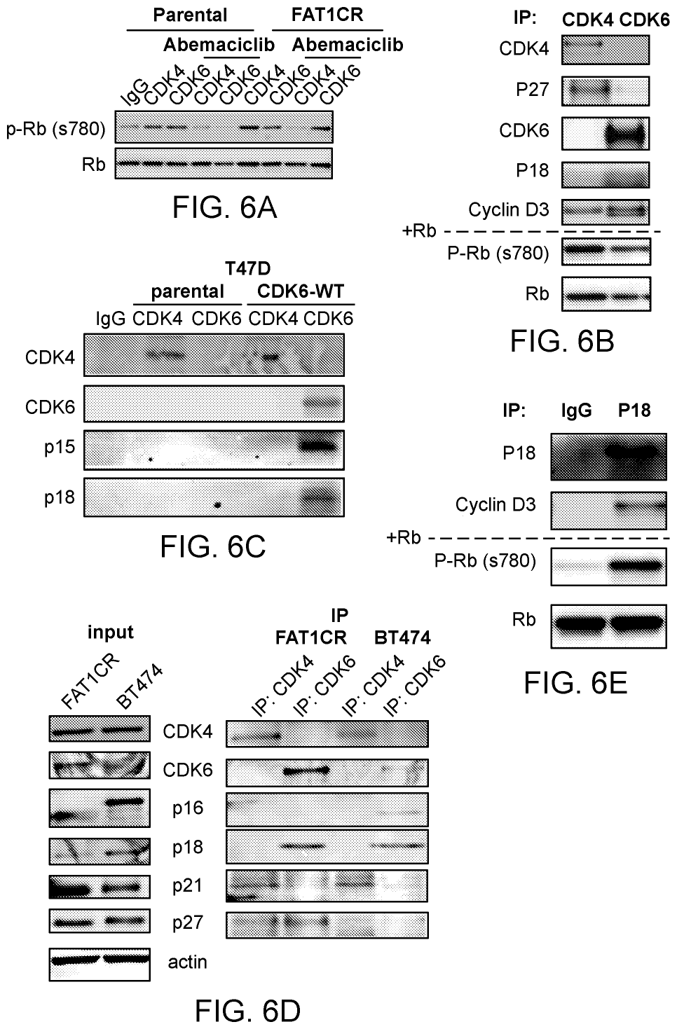

Upregulation of wild-type CDK6 expression as a recurrent mechanism by which tumors restore cell proliferation during CDK4/6i therapy (8-10). To determine if overexpression of CDK6 leads to reactivation of G1 checkpoint kinase activity, CDK4 and CDK6 were immunoprecipitated from isogenic drug sensitive (MCF7 parental cells with low CDK6) and resistant (MCF7 FAT1 loss cells with high CDK6) cells (8) and assayed their kinase activity using Rb substrate (FIGS. 1A and 1B, FIG. 6A). Drug-sensitive cells displayed higher basal levels of expression and activity of CDK4 compared to CDK6. By contrast, drug-resistant CDK6-high cells had similar levels of CDK4 and CDK6 kinase activity. Pre-treatment of cells with abemaciclib potently inhibited the kinase activity of CDK4 in both sensitive (84% reduced compared to untreated) and resistant (82%) cells, but could only partially reduce CDK6 activity (48%) in resistant cells, despite the near equal IC₅₀s derived from using recombinant CDK4 (2 nM) and CDK6 (5 nM) kinases as previously reported (11). As the composition of CDK4/6 complexes with specific members (e.g. D-cyclin, p16, p21, p27) can modify kinase activity and drug response (12-17), CDK4 and CDK6 interacting proteins were investigated by immunoprecipitation of CDK4 and CDK6 from drug-sensitive and resistant cells followed by mass spectrometry. Across three replicates, 7 proteins that were found in association with CDK6 were identified, but not CDK4, in CDK4/6i-resistant cells (FIG. 1C). Among proteins known to interact with CDK4/6 and regulate cell cycle, the INK4 proteins, p15^{INK4B} and p18^{INK4C} (n.b. parental cells lack endogenous p16^{INK4A}) appeared as the top two that associated with CDK6 but not CDK4 (FIGS. 1D and 1E). These findings were verified by immunoprecipitation and immunoblotting and again found that both p15^{INK4B} and p^{INK4C} associate with CDK6 in the resistant CDK6-high MCF7 FAT1CR cells (FIG. 1F and FIG. 6B). This strong association was verified in other INK4+/CDK6 expressing breast cancer cell lines that were resistant to CDK4/6i (FIG. 6C and FIG. 6D). Of note, it was observed that the INK4-associated CDK6 was an active kinase by demonstrating its phosphorylation of Rb (FIG. 6E). Accordingly, expression of the INK4 proteins was also upregulated in resistant cells as compared to sensitive cells (8). Further, an unbiased screen of over 1000 cell lines (PRISM) was conducted and it was found that INK4 overexpression (along with RB1 loss) to be among the top genomic alterations associated with CDK4/6i resistance (FIG. 1G). These data reveal that INK4 proteins strongly associate with CDK6 in CDK6-high cells that are resistant to CDK4/6i (18).

### Example 4. Interaction of INK4s and CDK6 Promotes Resistance to CDK4 6i

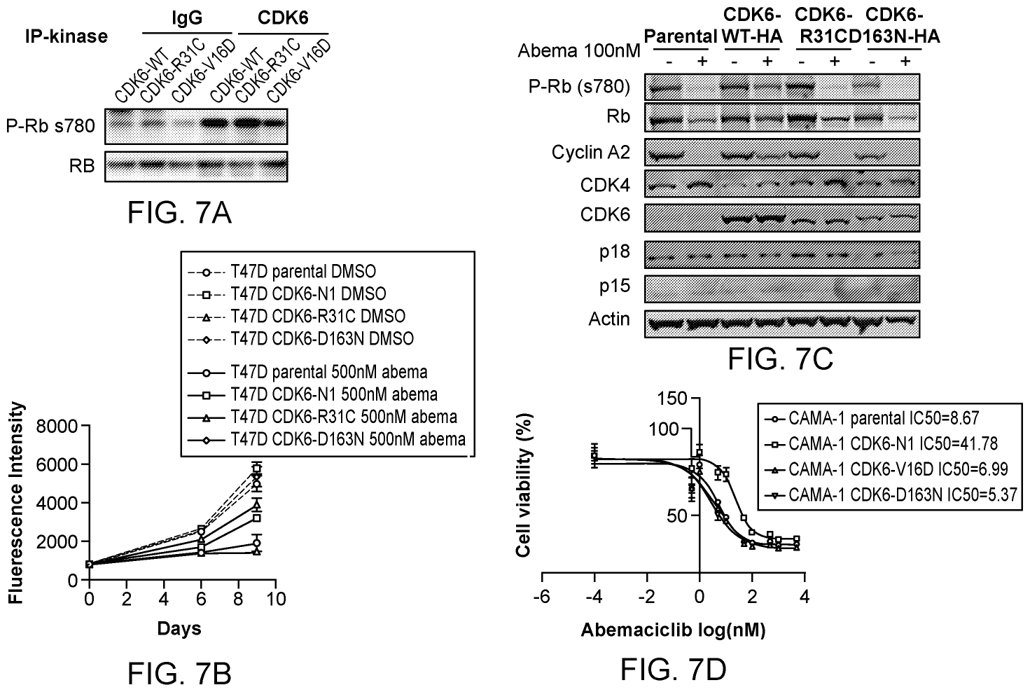

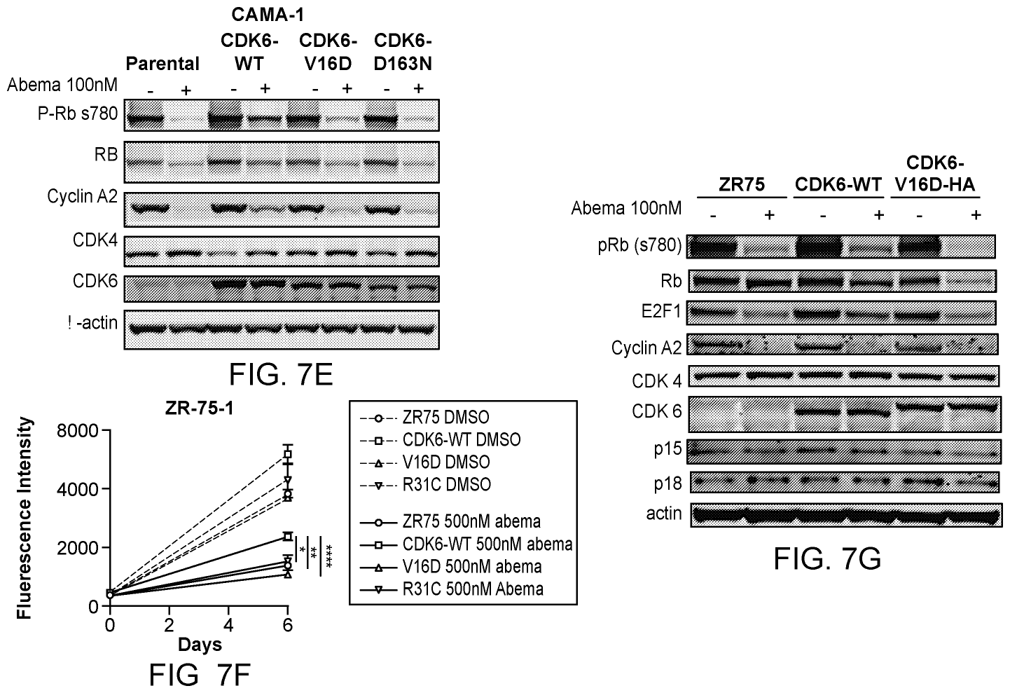

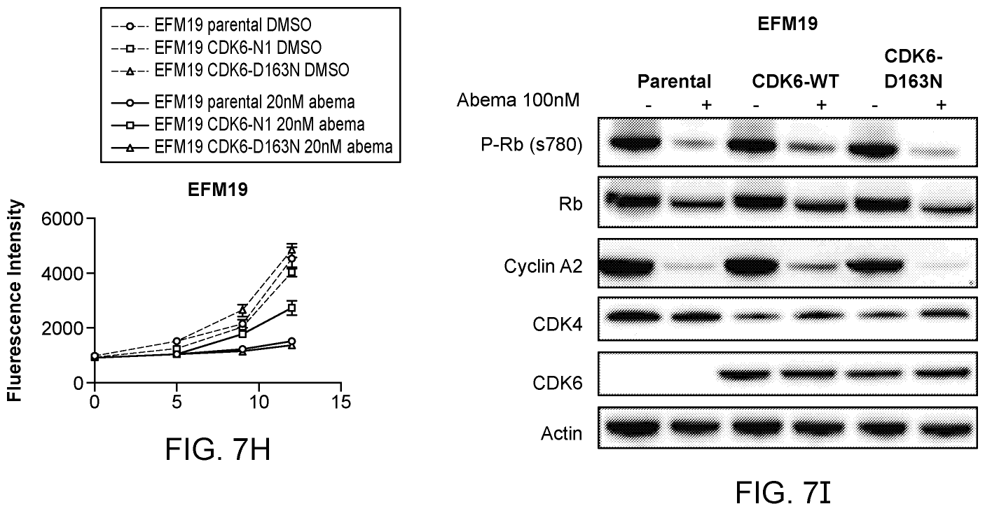

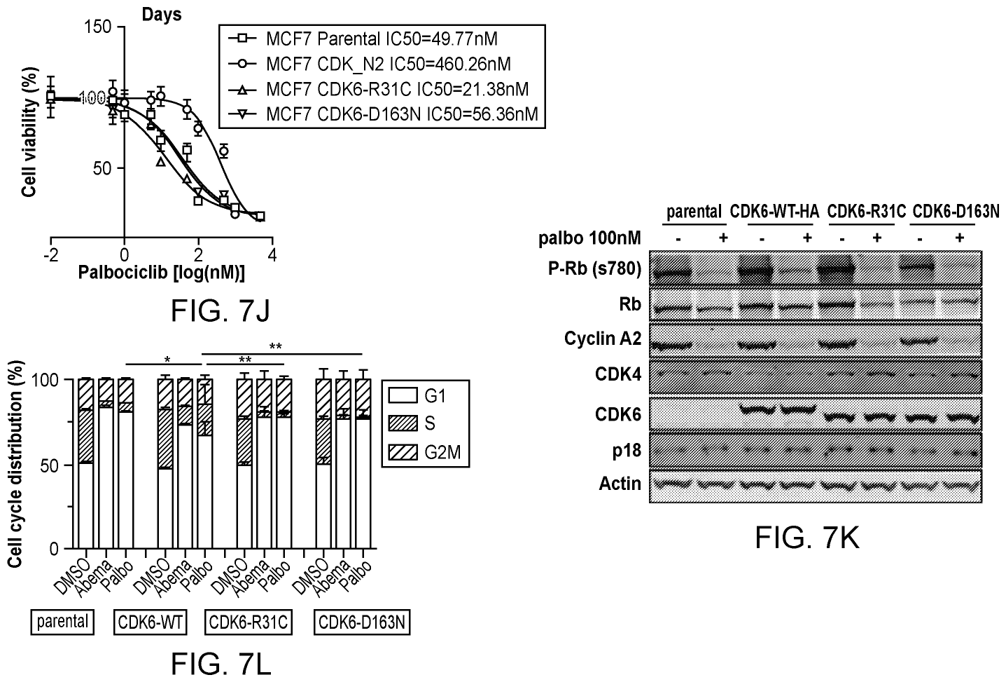

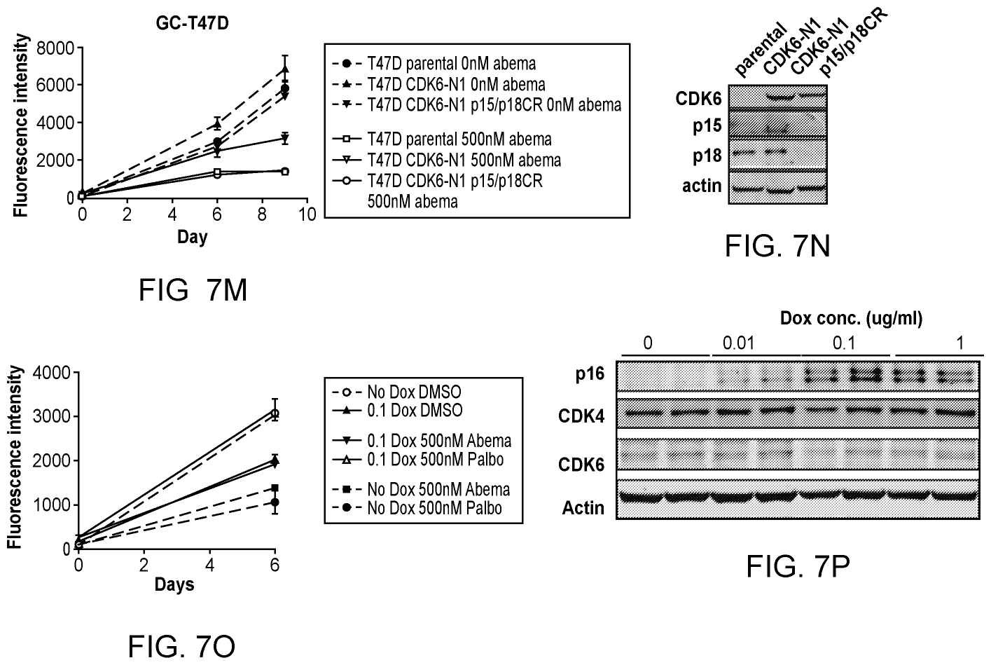

Based on previous crystallographic structures of CDK6-INK4 (19,20), candidate residues were selected in CDK6 that are in proximity of the INK4 binding site and performed site-directed mutagenesis of apparent CDK6-INK4 interface residues. By co-immunoprecipitation, it was confirmed that V16D and R31C alterations disrupted the interaction of CDK6 with p15^{INK4B} and p18^{INK4C} but with intact kinase activity (FIG. 2A and FIG. 7A). By contrast, classical kinase-dead mutations (K43M and D163N), far from the interface, did not disrupt the interaction. Consistent with a functional role for the INK4 interaction in drug resistance, mutations in the CDK6-INK4 interface decreased phosphorylation of Rb and downstream signaling in response to abemaciclib and palbociclib to the same extent as kinase-dead mutations (FIG. 2B). Cell viability assays also showed restored cellular sensitivity to abemaciclib in cells expressing mutant forms of CDK6 impaired at binding INK4 (R31C or V16D) (FIG. 2C). These finding were recapitulated in multiple ER+ breast cancer cell lines, including MCF7, T47D, ZR-75-1, CAMA-1 and EFM19 (FIG. 7B-7K). Moreover, overexpression of WT but not mutant forms of CDK6 (R31C or D163N) reduced the accumulation of cells in G1 after treatment with abemaciclib or palbociclib (FIG. 7L). To confirm the role of INK4 proteins in mediating drug resistance, CDKN2B (p15) and CDKN2C (p18) were genetically knocked out in CDK6-high cells and it was found that p18^{INK4C} loss could partially restore the responsiveness of p-Rb to abemaciclib treatment, while loss of both p15^{INK4B} and p18^{INK4C} could do so almost entirely (FIG. 2D). Concordantly, long term growth assays demonstrated that the loss of INK4 proteins rendered CDK6-high cells sensitive to CDK4/6i. (FIG. 2E; FIG. 7M and FIG. 7N). Conversely, overexpression of p16^{INK4A} in T47D cells lowered the potency of both abemaciclib and palbociclib (FIG. 7O and FIG. 7P). Taken together, these data implicate a drug insensitive INK4/CDK6 complex in driving persistent Rb phosphorylation in CDK4/6i resistant tumors.

### Example 5. INK4 CDK6 Complex is Insensitive to CDK4 6 Inhibitors

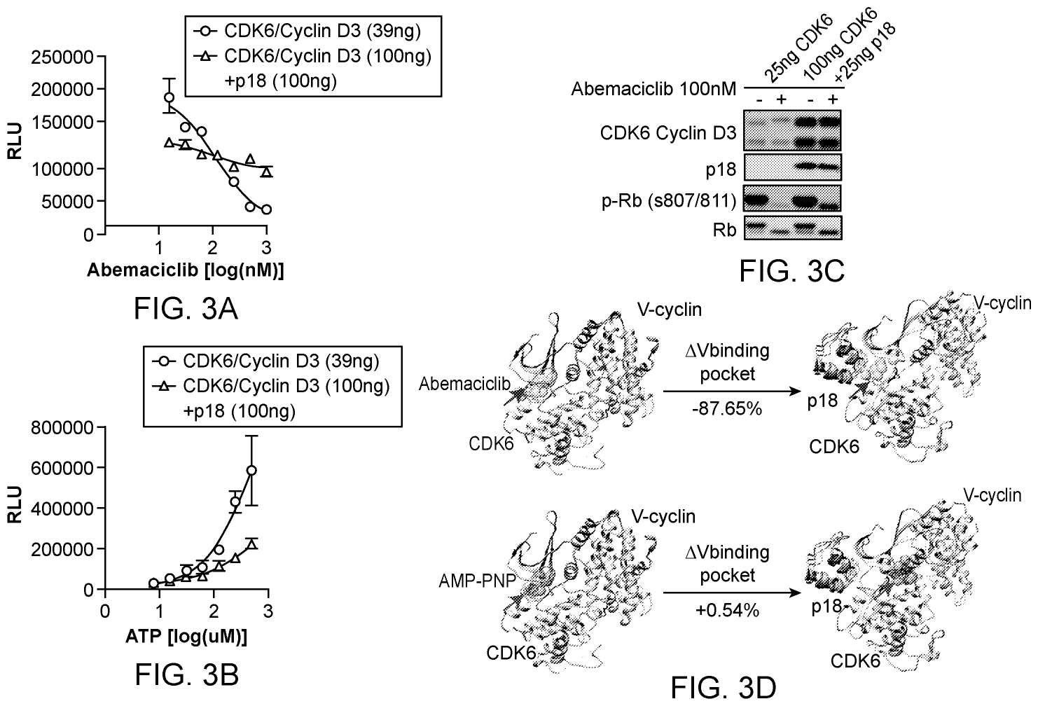

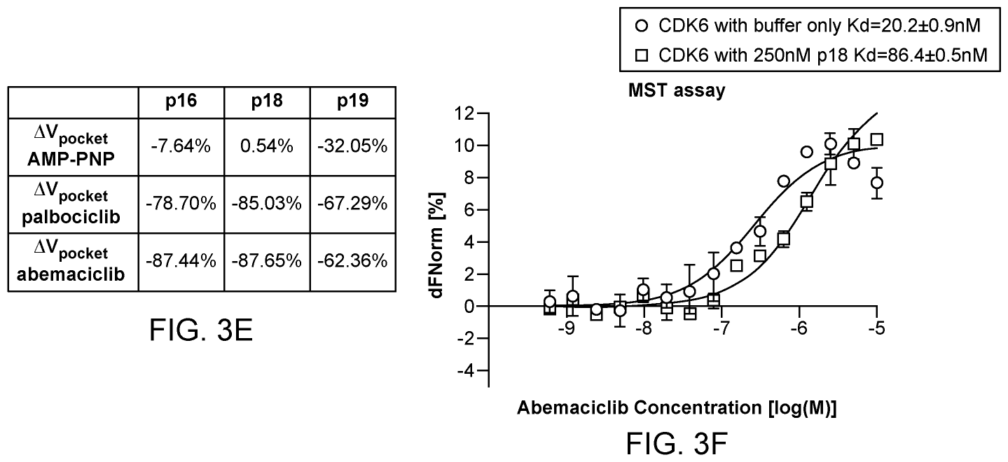

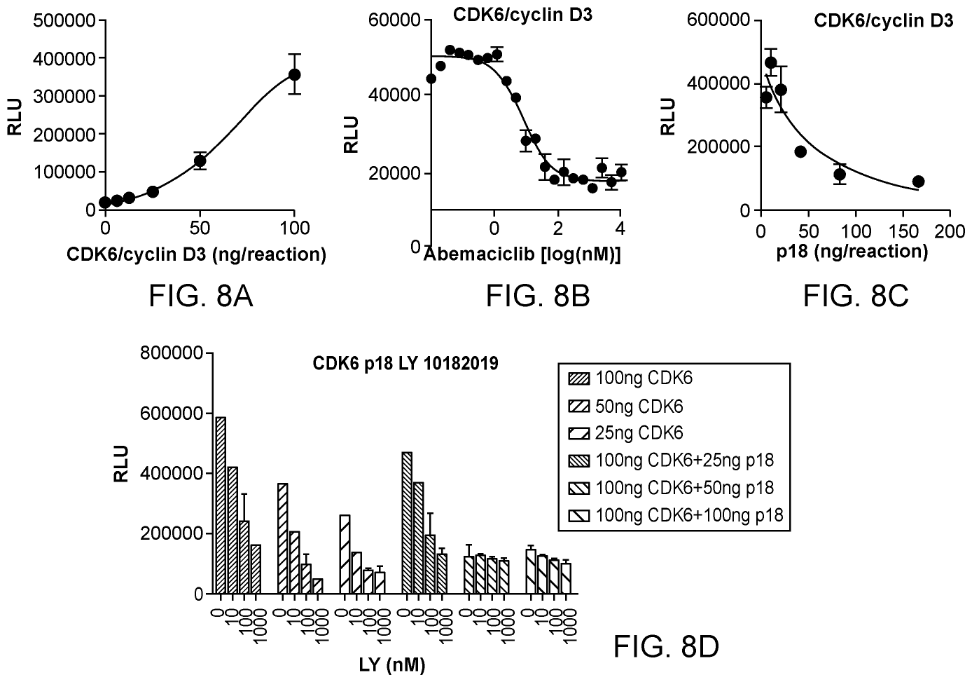

To further establish the role of the INK4 interaction in mediating the CDK4/6i-insensitivity of CDK6, recombinant CDK6/cyclin D3 and p18^{INK4C} were utilized in vitro kinase assay was performed (FIG. 3A-3B; FIG. 8A-8C). It was found that abemaciclib potently inhibited CDK6/cyclinD3 kinase activity with an IC₅₀ of 8 nM (FIG. 8B), approximating published reports (21) and addition of recombinant p18^{INK4C} protein inhibited CDK6 kinase activity (FIG. 8C). While addition of p18 did lower CDK6-cyclinD activity, it also prevented the near-complete suppression by abemaciclib observed in the absence of p18 (FIG. 3A-3B; FIG. 8D). Immunoblotting for Rb phosphorylation confirmed that preincubation with p18 impairs abemaciclib inhibition of CDK6 activity (FIG. 3C).

To elucidate structural mechanisms underlying the effect of INK4 proteins on CDK6 drug inhibition, existing CDK6 structures alone (PDBID: 2EUF (22)) or in complex with INK4s was inspected (listed in the table of FIG. 2A). Structural superimposition indicates that the N-lobe of CDK6 is twisted towards cyclin D upon binding of INK4 (FIG. 3D). It was found that INK4 (p16, p18 or p19) binding to CDK6 caused distortion of the N-lobe of CDK6 thereby more significantly decreasing the effective binding pocket volume for CDK4/6i than for AMP-PNP, a non-hydrolysable analogue of ATP (FIG. 3D). This effect is most prominent in p18-bound CDK6 where p18 binding causes a drastic reduction of the CDK4/6i binding volume (−87.65% for abemaciclib and −85.03% for palbociclib), but minimally impacts the binding volume of AMP-PNP (+0.54%) (FIG. 3D). The p16-bound CDK6 (−87.44%/−7.64%) and p19-bound CDK6 (−62.36%/−32.05%) led to similar reductions in CDK4/6i and AMP-PNP binding volumes (FIG. 3E). The reduction of binding pocket volume upon association of INK4 compellingly explains the impaired CDK4/6i inhibition and residual kinase activity in the presence of INK4 observed in our biochemical assays. To directly test whether p18^{INK4C} alters the binding affinity of CDK4/6i to CDK6, microscale thermophoresis (MST) assays were performed and it was found that the K_{d} of abemaciclib to CDK6 was increased by 4-fold in the presence of p18^{INK4C} (FIG. 3F). Taken together, these data reveal that the addition of p18^{INK4C} to the D-cyclin/CDK6 complex suppresses CDK4/6i binding, likely mediating drug resistance.

### Example 6. Multiple Genetic Alterations Lead to CDK6-Mediated Resistance Inpatients

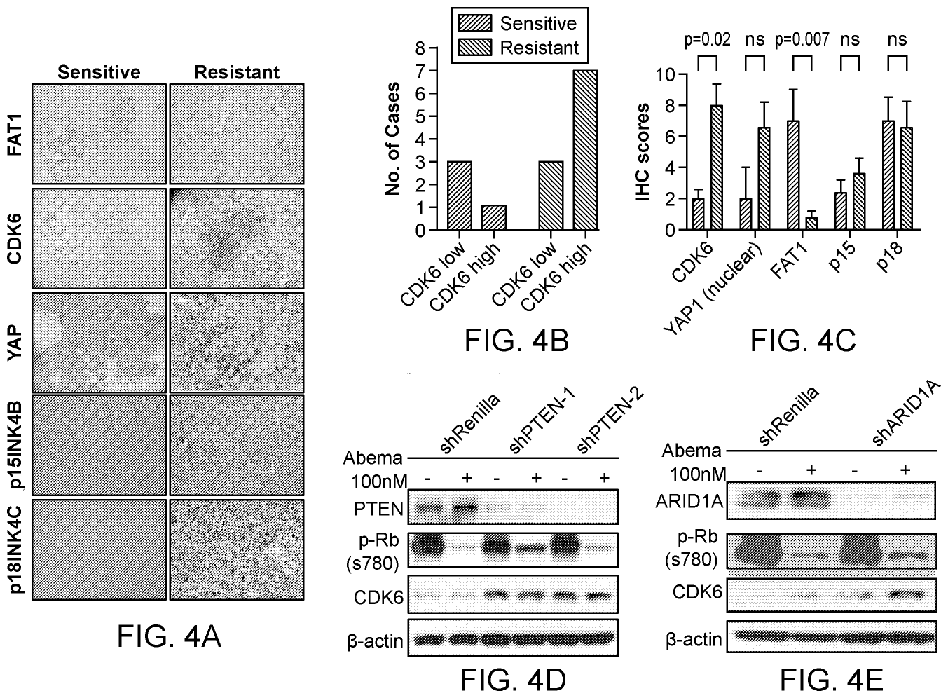

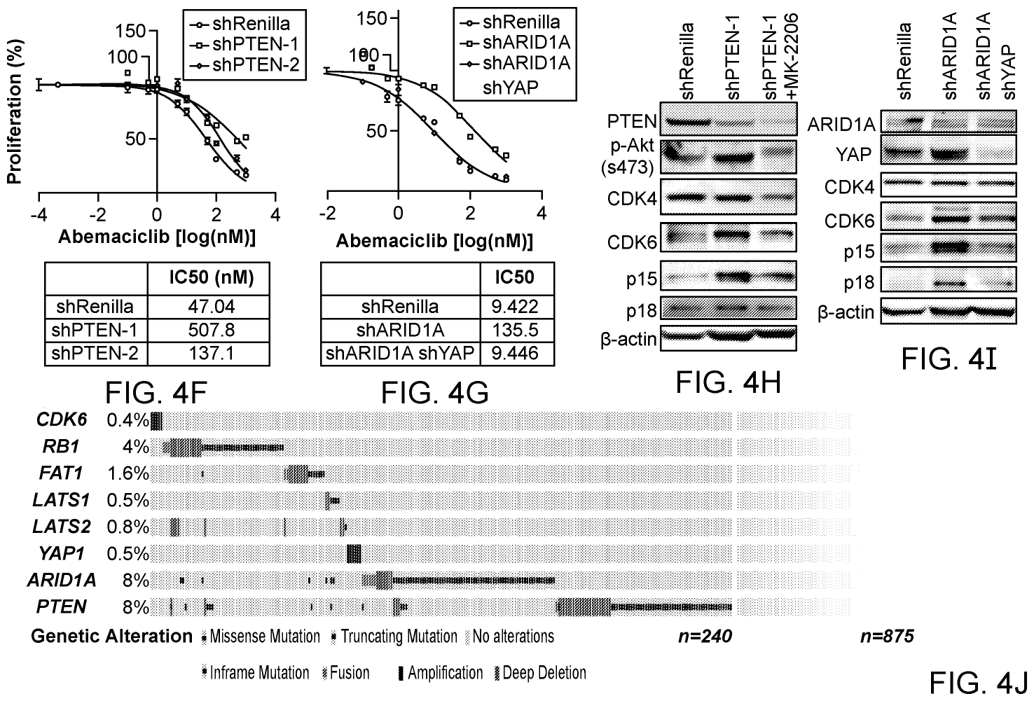

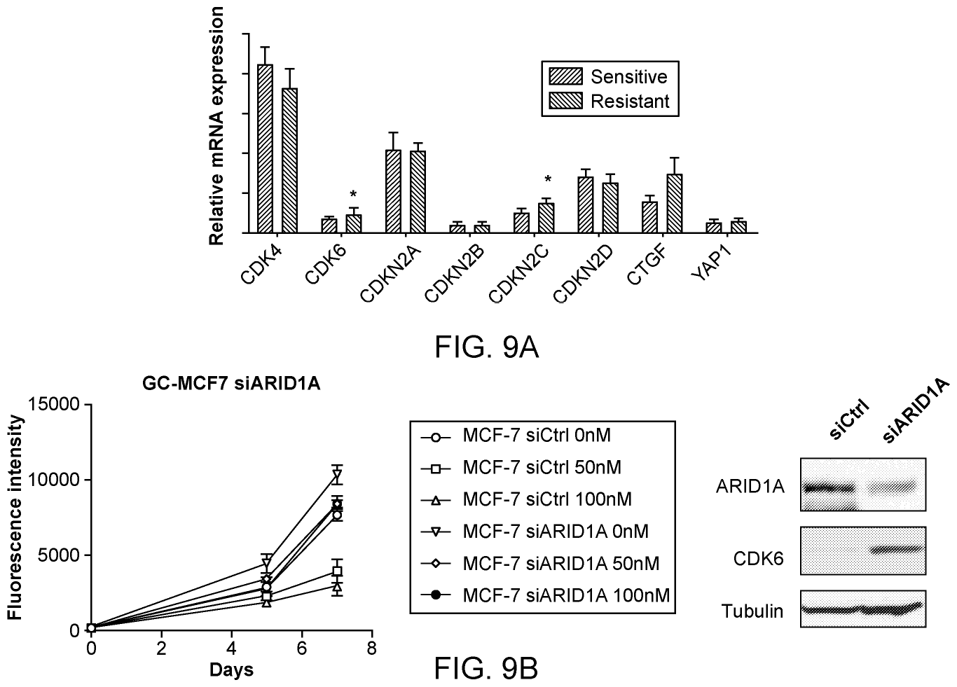

To define the prevalence of the CDK6-high, CDK4/6i-resistant state in clinically relevant samples, CDK6 and INK4 protein expression were analyzed by immunohistochemistry, using a panel of patient-derived ER+ breast cancer xenografts (FIG. 4A). It was found that among 14 distinct models, eight models displayed intense CDK6 staining. Of these, seven out of eight were found to be resistant to CDK4/6i (FIG. 4B). Based on prior work establishing Hippo pathway suppression as a mechanism of CDK6 upregulation (8), FAT1 and nuclear YAP protein levels in these samples were further analyzed and it was indeed found that a subset of high CDK6 tumors featured low FAT1, high nuclear YAP1 and high p15/p18 (FIG. 4C and FIG. 9A). However, high CDK6 was found in some FAT1 wild-type tumors as well, suggesting that additional genetic alterations might promote high CDK6 expression. Indeed, both PTEN and ARID1A have been implicated as potential regulators of this pathway, and therefore whether loss of these might also increase CDK6 abundance was tested (23,24). Knockdown of either PTEN or ARID1A in CDK4/6i sensitive cell lines led to upregulation of CDK6 expression and resistance to abemaciclib (FIG. 4D-G, FIG. 9B). Given the canonical role of PTEN in suppressing AKT activation, the effects of an AKT inhibitor MK-2206 were tested and it was found that its administration suppressed the expression of CDK6 in PTEN knockdown cells (FIG. 411). With respect to ARID1A and its known role in sequestering YAP (24), it was found that knockdown of YAP reduced CDK6 expression in the ARID1A knockdown cells (FIG. 4I). Moreover, it was found that an induction of INK4 protein expression concomitant with CDK6 upregulation in both PTEN and ARID1A knockdown cells (FIG. 411 and FIG. 4I), suggesting that coordinate upregulation of INK4 and CDK6 may be responsible for mediating drug resistance in this context. Given these results, the genomic landscape of 1366 metastatic tumors from 1115 patients with HR+/HER2− metastatic breast cancer (MSK-IMPACT) was analyzed and it was found that 190 out of 1115 (17%) patients showed at least one of the genetic alterations that might be associated with CDK6 upregulation and resistance to CDK4/6 inhibitors (FIG. 4J). These findings demonstrate that mutations that can promote a CDK6-INK4 complex represent a significant cohort of patients in which current ATP-competitive CDK4/6i may prove ineffective.

### Example 7. Degraders Targeting CDK6 Complexes Inhibit Resistant Cells

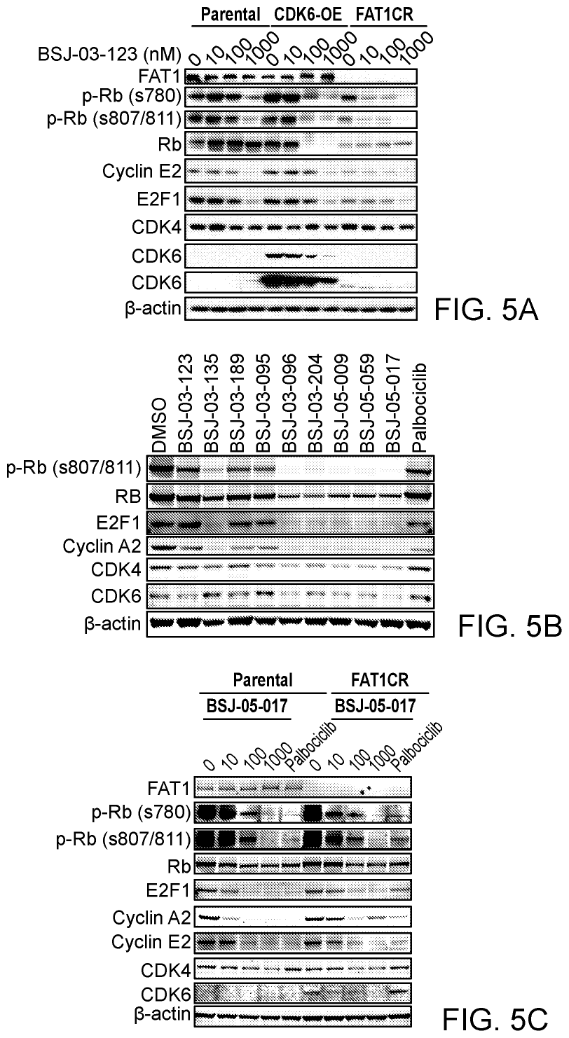

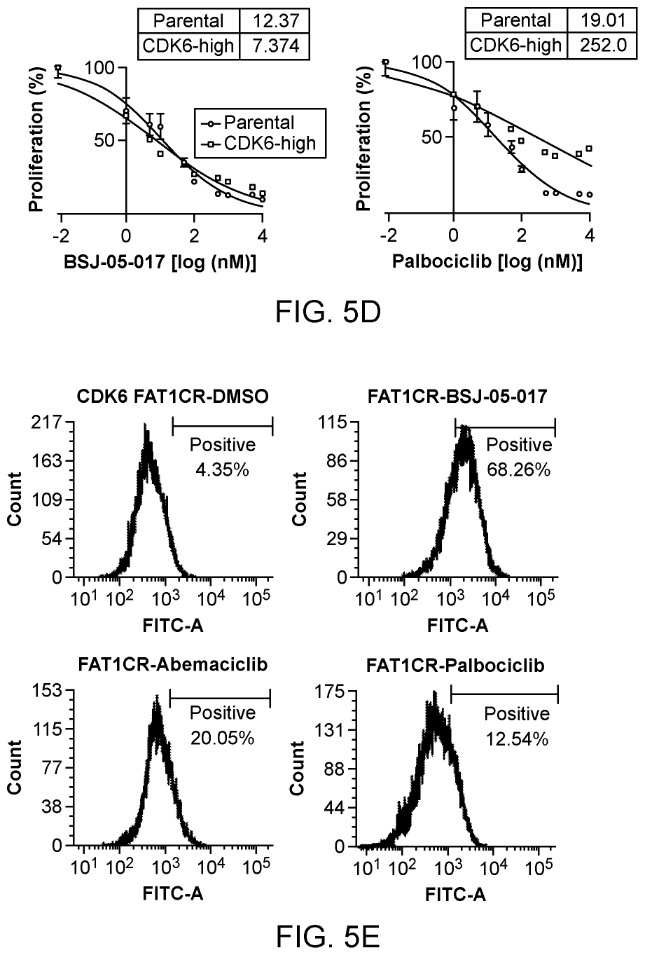

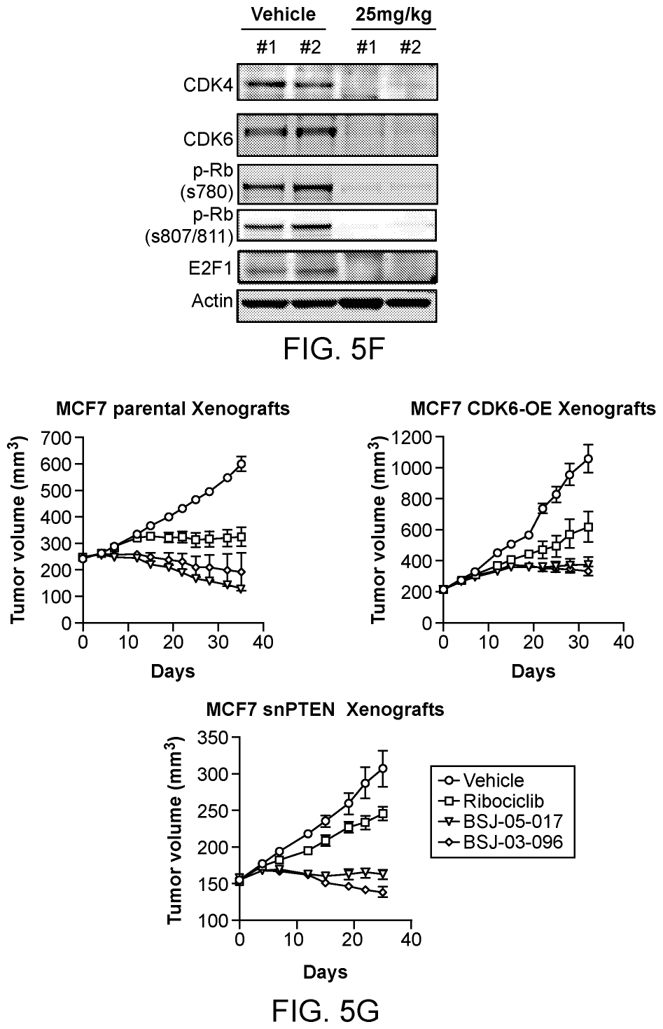

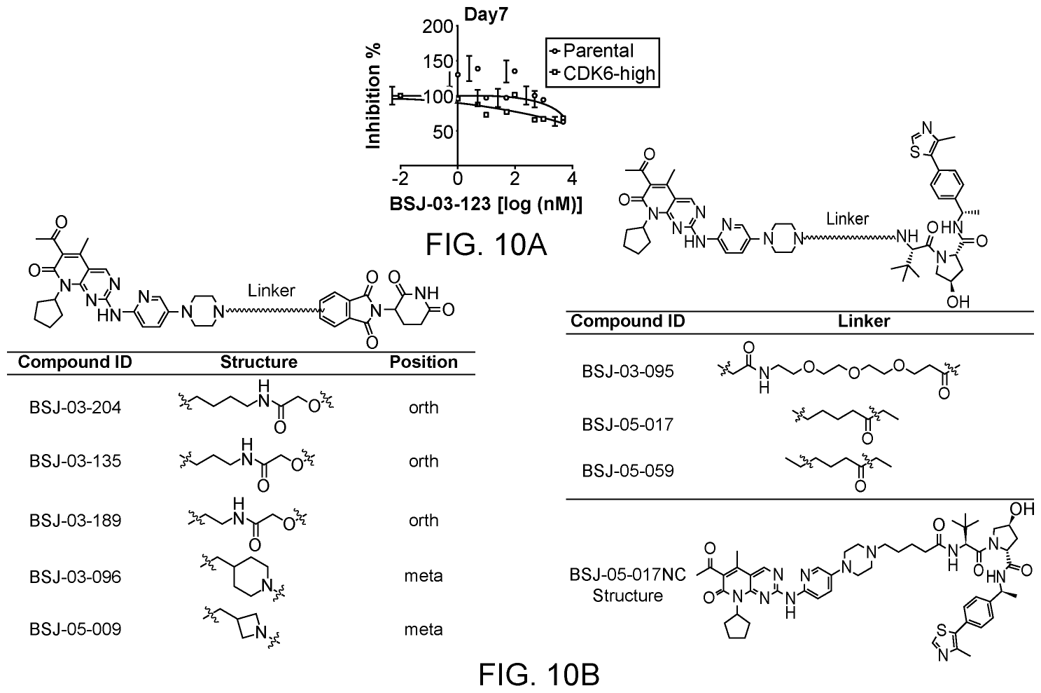

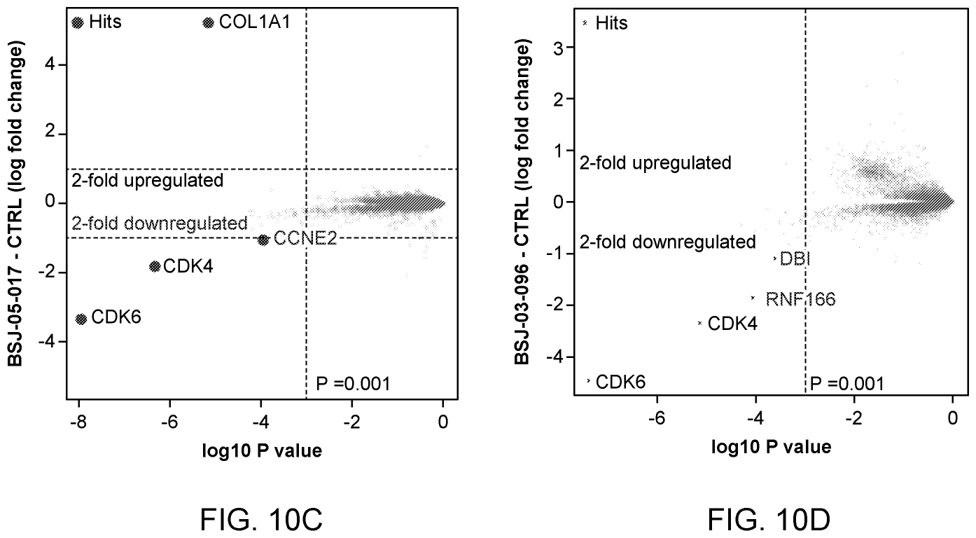

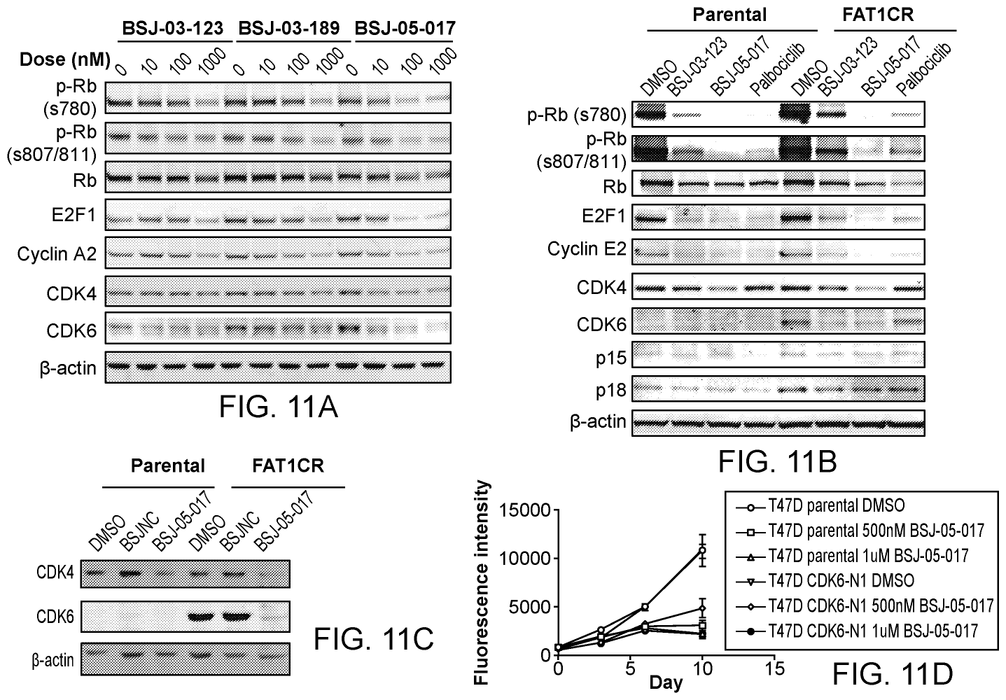

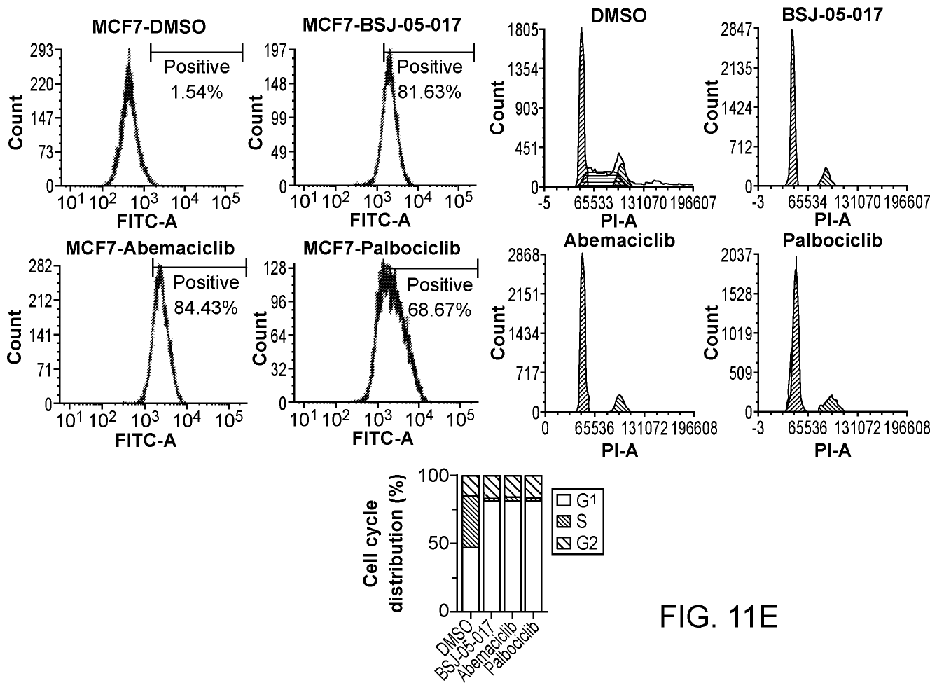

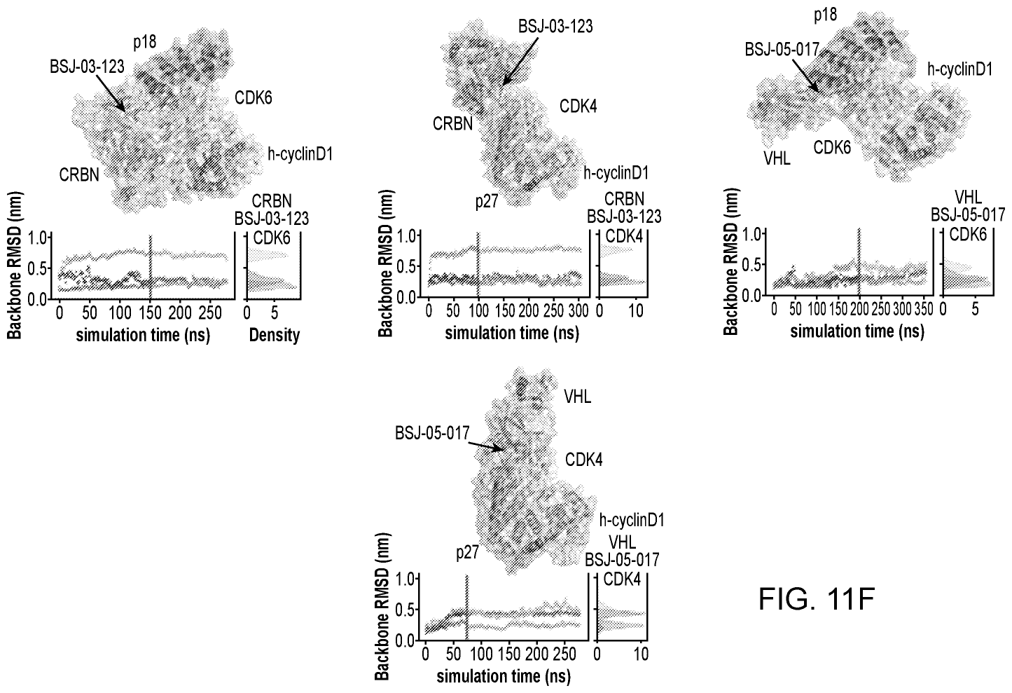

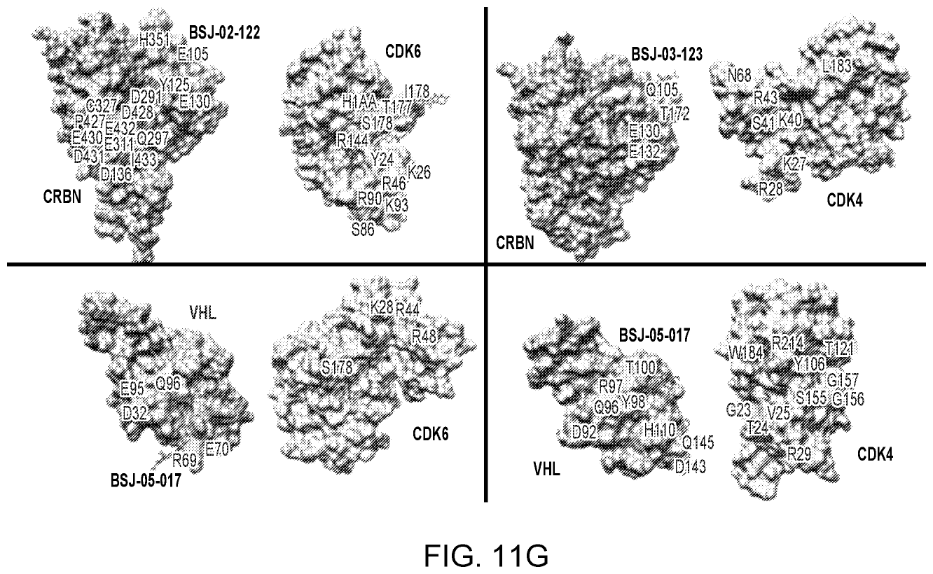

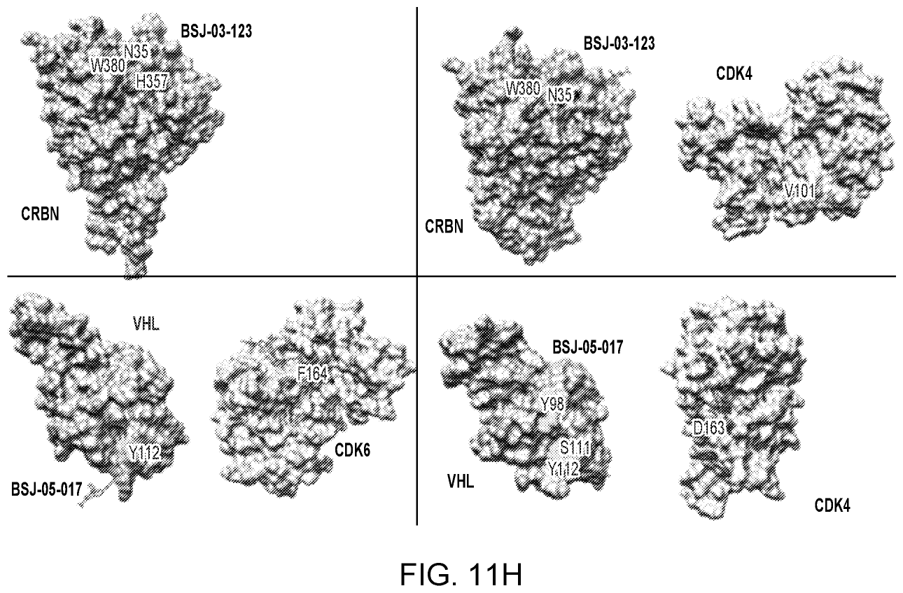

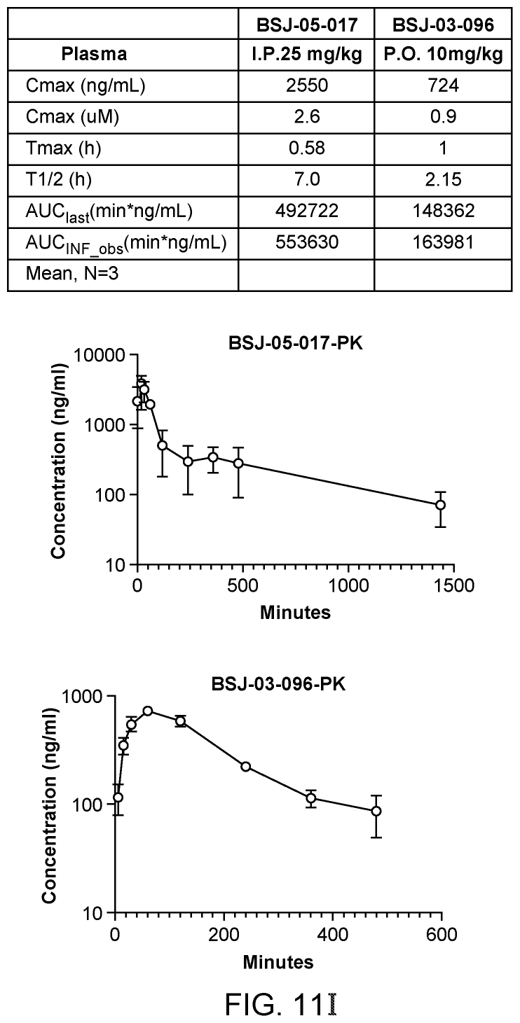

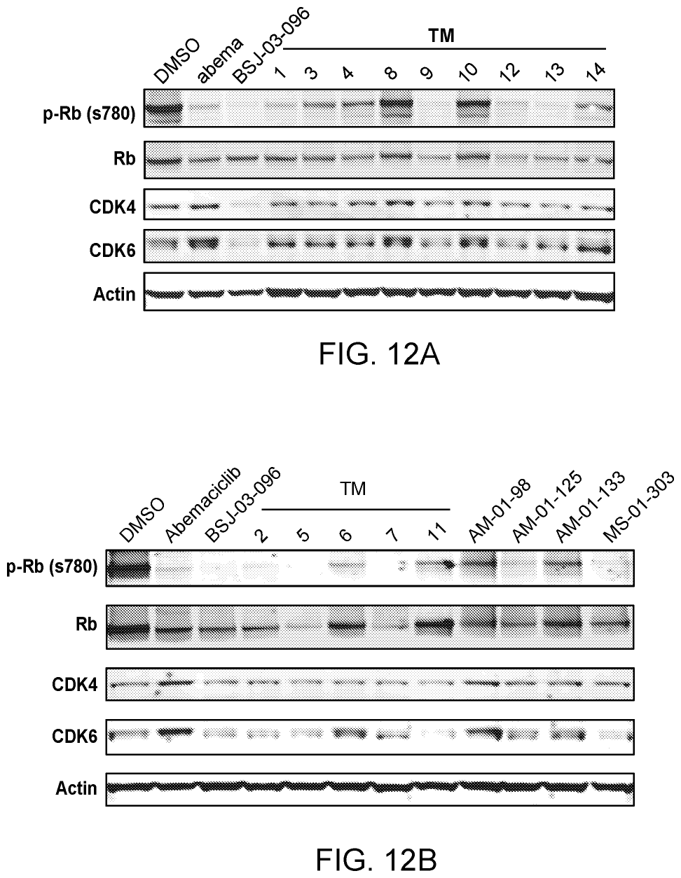

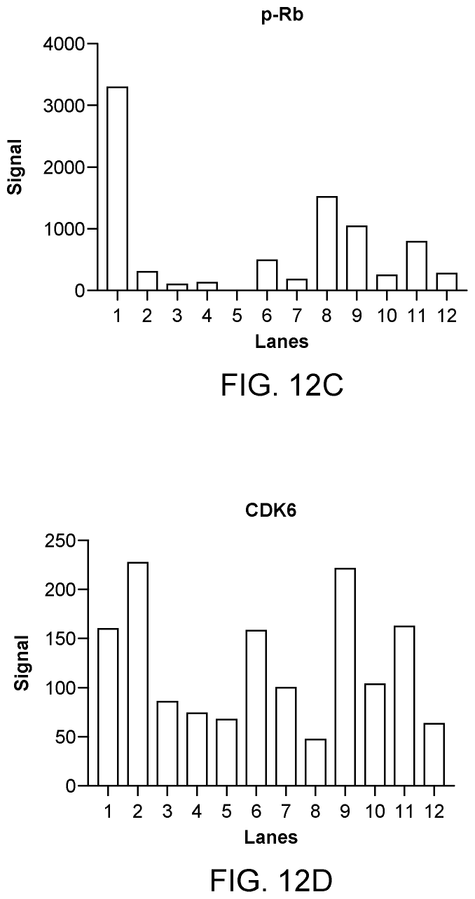

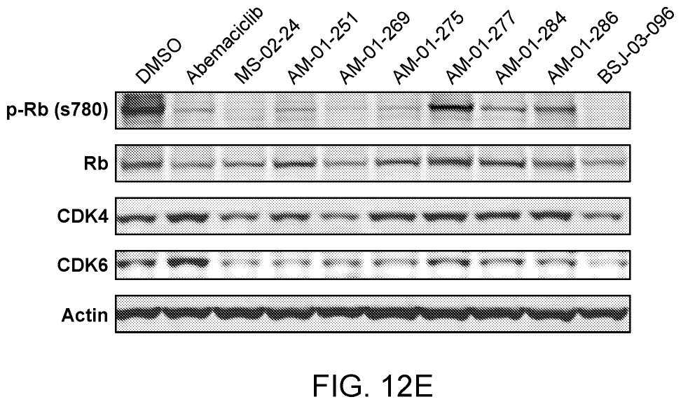

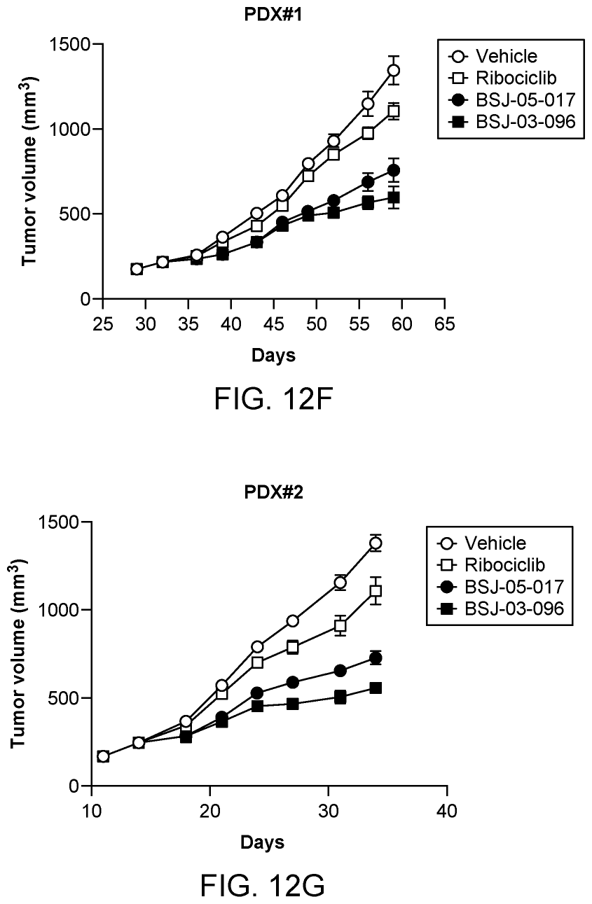

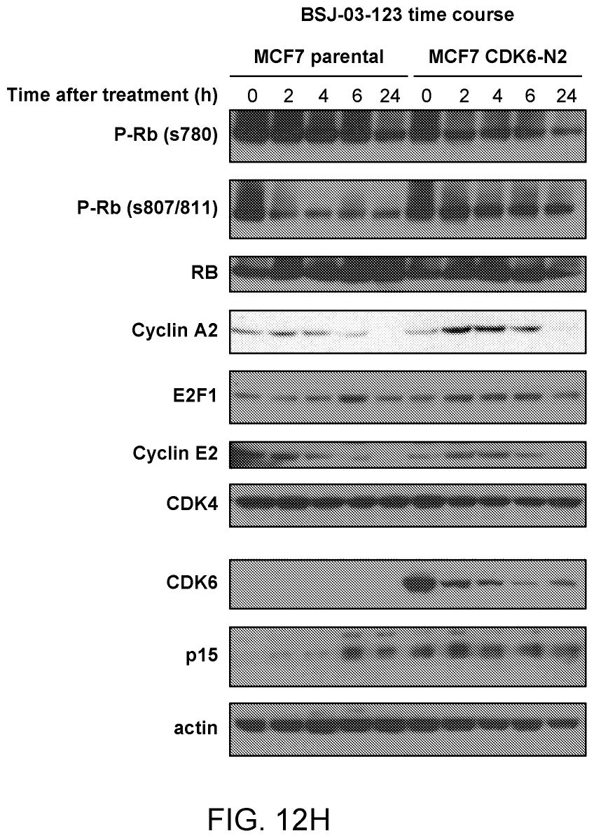

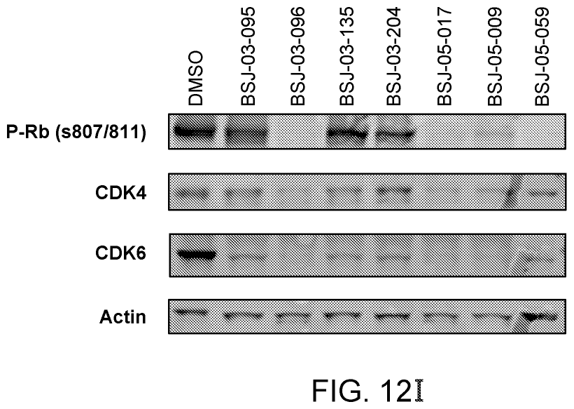

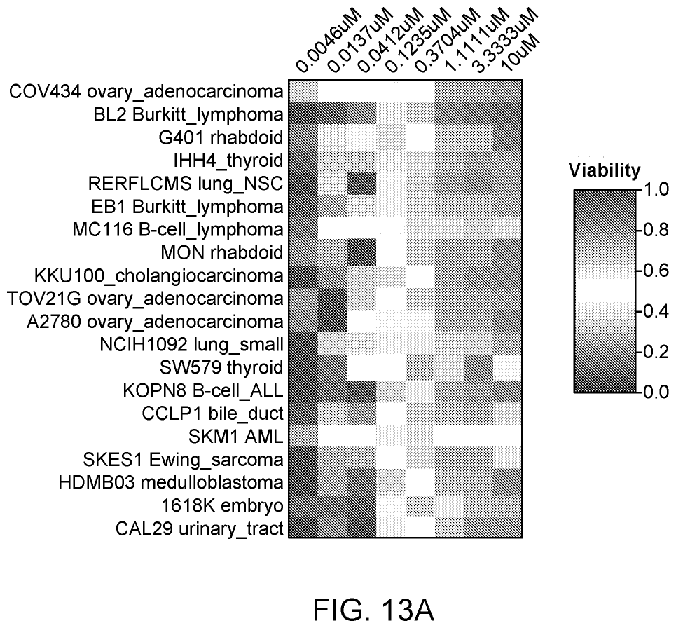

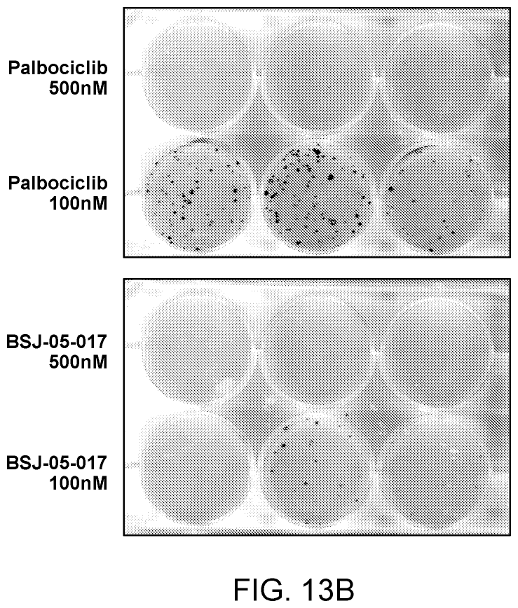

As the INK4/CDK6 complex confers resistance to current generation of CDK4/6i, the potential of other compounds was explored to target this pathway. Bifunctional degraders (proteolysis targeting chimera, PROTAC) have emerged as a promising approach to target “undruggable” proteins and overcome resistance to small molecule inhibitors (25,26). A selective CDK6 degrader BSJ-03-123 (27) was previously identified and was here examined its effect in CDK6-high, CDK4/6i-resistant cells. BSJ-03-123 led to dose-dependent degradation of CDK6 but had no effect on CDK4. As a result, BSJ-03-123 could inhibit the phosphorylation of Rb and expression of downstream cell cycle signaling components (e.g. cyclin A2 and E2F1) in CDK4/6i-resistant cells (FIG. 5A). However, when the same cells were grown in long-term culture with effective CDK6 inhibition or knockdown (9), significant growth suppression was not observed due to preserved CDK4 activity (FIG. 10A). A panel of CDK4/6 selective degraders were generated by linking palbociclib to e.g., Cereblon (CRBN)-binding (28) or von Hippel-Landau (VHL)-binding ligands (FIG. 10B). For example, BSJ-05-017 and BSJ-03-096 showed high potency in degrading CDK4 and CDK6, acting at doses as low as 10 nM (FIG. 5B; FIG. 10C and FIG. 10D). The two compounds demonstrated effective inhibition of the phosphorylation of Rb and the expression of E2F1/cyclin A2 in both CDK4/6i-sensitive and resistant cells (FIGS. 5B and 5C; FIG. 11A and FIG. 11B). The degradation of CDK4 and CDK6 was abolished due to loss of binding to VHL with a reversal of the two chiral centers in the VHL ligand (FIG. 11C) (29). To assess the therapeutic potential of BSJ-05-017 in CDK6-driven cells, cell proliferation assays were performed and it was found that BSJ-05-017 to be equipotent in suppressing proliferation (FIG. 5D and FIG. 11D) and inducing cell cycle arrest and senescence (FIG. 5E and FIG. 11E) of CDK4/6i-sensitive and -resistant breast cancer cells. To understand how these degraders could induce degradation and do so despite the presence of INK4, atomic-level molecular models of CDK4/6 complexed with degraders were manually constructed: E3 ligase adapter pairs (BSJ-03-123: CRBN, BSJ-05-017: VHL) in the presence of p18 or p27 and cyclin D based on existing crystallographic data and previously reported PROTAC degrader binding models (12,30). The E3 ligase adapters shifted ˜0.5 nm from their initial conformation to adopt new stable conformation in all four models (FIG. 11F). To dissect the binding patterns of each of the ternary complex models, hydrogen bonds between the CDKs and the E3 ligase adapters or the degraders were identified for each simulation trajectory (FIG. 11G and FIG. 1111). Comparing the CRBN: CDK4 vs CRBN: CDK6 conformations, it appears that CDK4 and CDK6 are engaging distinct regions of the CRBN surface with minimal overlap. The CDK6 binding region on the CRBN surface is closer to the binding pocket of BSJ-03-123 and one of the key residues identified here, H₃₅₃, was previously reported to be important for CRBN to recruit and interact with various substrates (31-33). By contrast, CDK4 engages a set of CRBN residues that are distal to the degrader binding pocket. This difference potentially explains the selective degradation of CDK6 over CDK4 induced by BSJ-03-123. To investigate how both BSJ-03-123 and BSJ-05-017 target CDK6, the modeled binding modes of the degrader warhead to CDK6 in complex with E3 ligase adapters were examined in detail. There is minimal interaction between BSJ-03-123 and the kinase binding pocket (no interaction with CDK6 and only one h-bond with the hinge region V101 in CDK4) (top prow of FIG. 11H). Instead, the stabilization of the ternary complex and the effective degradation of CDK6 appears to result from protein-protein interactions between CRBN and CDK6. In the case of the BSJ-05-017 (bottom row of FIG. 1111), the degrader appears to partially interact with the kinase binding pocket (F164 in CDK6 and D163 in CDK4, both residues in the DFG motif) despite the distorted binding pocket and DFG-out conformation of CDK6 in the presence of INK4. In this case, both the degrader-CDK interactions and VHL-CDK interactions contribute to the stabilization of the complex, explaining the robust CDK degradation by BSJ-05-017 observed experimentally. The capacity of these molecules to effectively degrade CDK6 is also consistent with prior reports that even PROTAC compounds with weak ligand binding affinity for the target protein can still achieve formation of a stable complex through additional interactions, leading to potent protein degradation (34).

To ascertain the potential for in vivo use of BSJ-05-017 and BSJ-03-096, its pharmacokinetic (PK) properties were assessed following a single dose in mice through i.p. or p.o. Both BSJ-05-017 and BSJ-03-096 displayed high drug exposure in plasma, achieving a Cmax of 2.6 M and 0.9 M, and good metabolic stability, as near equivalent to ribociciclib at 20 mg/kg in previous report (35). At 24h post dosing, the compounds concentration remained near 100 nM, still above the IC₅₀ for in vitro CDK4/6 degradation (FIG. 11I). Given these promising results, the in vivo effects of both compounds in CDK6-low and CDK6-high cell-derived xenografts were next evaluated. Compared to the vehicle control, BSJ-05-017 induced near complete degradation of CDK4 and CDK6, leading to significant suppression of Rb1 phosphorylation and E2F1 expression (FIG. 5F). In the long-term, MCF7 parental cell-derived xenografts with low CDK6 expression were sensitive to ribociclib, BSJ-05-017 and BSJ-03-096, while CDK6-overexpressing and shPTEN xenografts were durably inhibited by BSJ-03-096 (−68.9% and −54.9%) and BSJ-05-017 (−64.9% and ˜47.4%) while showing tumor outgrowth after initial response to ribociclib (FIG. 5G). These results reveal that in multiple models of CDK4/6i resistance, more potent and complete inhibition of the CDK4/6 kinases has substantial antitumor effects.

### REFERENCES

8. Li Z, Razavi P, Li Q, Toy W, Liu B, Ping C, et al. Loss of the FAT1 Tumor Suppressor Promotes Resistance to CDK4/6 Inhibitors via the Hippo Pathway. Cancer cell 2018; 34(6):893-905 e8 doi 10.1016/j.ccell.2018.11.006.
9. Yang C, Li Z, Bhatt T, Dickler M, Giri D, Scaltriti M, et al. Acquired CDK6 amplification promotes breast cancer resistance to CDK4/6 inhibitors and loss of E R signaling and dependence. Oncogene 2017; 36(16):2255-64 doi 10.1038/onc.2016.379.
10. Cornell L, Wander S A, Visal T, Wagle N, Shapiro G I. MicroRNA-Mediated Suppression of the TGF-β Pathway Confers Transmissible and Reversible CDK4/6 Inhibitor Resistance. Cell Reports 2019; 26(10):2667-80.e7 doi https://doi.org/10.1016/j.celrep.2019.02.023.
11. O'Leary B, Finn R S, Turner N C. Treating cancer with selective CDK4/6 inhibitors. Nat Rev Clin Oncol 2016; 13(7):417-30 doi 10.1038/nrclinonc.2016.26.
12. Guiley K Z, Stevenson J W, Lou K, Barkovich K J, Kumarasamy V, Wijeratne T U, et al. p27 allosterically activates cyclin-dependent kinase 4 and antagonizes palbociclib inhibition. Science 2019; 366(6471) doi 10.1126/science.aaw2106.
13. Baldin V, Lukas J, Marcote M J, Pagano M, Draetta G. Cyclin D1 is a nuclear protein required for cell cycle progression in G1. Genes & development 1993; 7(5):812-21 doi 10.1101/gad.7.5.812.
14. Blain S W, Montalvo E, Massague J. Differential interaction of the cyclin-dependent kinase (Cdk) inhibitor p27Kip1 with cyclin A-Cdk2 and cyclin D2-Cdk4. The Journal of biological chemistry 1997; 272(41):25863-72 doi 10.1074/jbc.272.41.25863.
15. LaBaer J, Garrett M D, Stevenson L F, Slingerland J M, Sandhu C, Chou H S, et al. New functional activities for the p21 family of CDK inhibitors. Genes & development 1997; 11(7):847-62 doi 10.1101/gad.11.7.847.
16. Parry D, Mahony D, Wills K, Lees E. Cyclin D-CDK Subunit Arrangement Is Dependent on the Availability of Competing INK4 and p21 Class Inhibitors. Molecular and cellular biology 1999; 19(3):1775 doi 10.1128/MCB.19.3.1775.
17. Grimmler M, Wang Y, Mund T, Cilensek Z, Keidel E-M, Waddell M B, et al. Cdk-Inhibitory Activity and Stability of p27Kip1 Are Directly Regulated by Oncogenic Tyrosine Kinases. Cell 2007; 128(2):269-80 https://doi.org/10.1016/j.cell.2006.11.047.
18. Swarbrick A, Lee C S, Sutherland R L, Musgrove E A. Cooperation of p27(Kipl) and p18(INK4c) in progestin-mediated cell cycle arrest in T-47D breast cancer cells. Molecular and cellular biology 2000; 20(7):2581-91 doi 10.1128/mcb.20.7.2581-2591.2000.
19. Jeffrey P D, Tong L, Pavletich N P. Structural basis of inhibition of CDK-cyclin complexes by INK4 inhibitors. Genes & development 2000; 14(24):3115-25 doi 10.1101/gad.851100.
20. Russo A A, Tong L, Lee J O, Jeffrey P D, Pavletich N P. Structural basis for inhibition of the cyclin-dependent kinase Cdk6 by the tumour suppressor p16INK4a. Nature 1998; 395(6699):237-43 doi 10.1038/26155.
21. Chen P, Lee N V, Hu W, Xu M, Ferre R A, Lam H, et al. Spectrum and Degree of CDK Drug Interactions Predicts Clinical Performance. Molecular cancer therapeutics 2016; 15(10):2273-81 doi 10.1158/1535-7163.MCT-16-0300.
22. Lu H, Schulze-Gahmen U. Toward Understanding the Structural Basis of Cyclin-Dependent Kinase 6 Specific Inhibition. Journal of Medicinal Chemistry 2006; 49(13):3826-31 doi 10.1021/jm0600388.
23. Tumaneng K, Schlegelmilch K, Russell R C, Yimlamai D, Basnet H, Mahadevan N, et al. YAP mediates crosstalk between the Hippo and PI(3)K-TOR pathways by suppressing PTEN via miR-29. Nat Cell Biol 2012; 14(12):1322-9 doi 10.1038/ncb2615.
24. Chang L, Azzolin L, Di Biagio D, Zanconato F, Battilana G, Lucon Xiccato R, et al. The SWI/SNF complex is a mechanoregulated inhibitor of YAP and TAZ. Nature 2018; 563(7730):265-9 doi 10.1038/s41586-018-0658-1.
25. Pettersson M, Crews C M. PROteolysis TArgeting Chimeras (PROTACs)—Past, present and future. Drug Discov Today Technol 2019; 31:15-27 doi 10.1016/j.ddtec.2019.01.002.
26. Konstantinidou M, Li J, Zhang B, Wang Z, Shaabani S, Ter Brake F, et al. PROTACs—a game-changing technology. Expert Opin Drug Discov 2019; 14(12):1255-68 doi 10.1080/17460441.2019.1659242.
27. Brand M, Jiang B, Bauer S, Donovan K A, Liang Y, Wang E S, et al. Homolog-Selective Degradation as a Strategy to Probe the Function of CDK6 in AML. Cell Chem Biol 2019; 26(2):300-6.e9 doi 10.1016/j.chembiol.2018.11.006.
28. Jiang B, Wang E S, Donovan K A, Liang Y, Fischer E S, Zhang T, et al. Development of Dual and Selective Degraders of Cyclin-Dependent Kinases 4 and 6. Angew Chem Int Ed Engl 2019; 58(19):6321-6 doi 10.1002/anie.201901336.
29. Khan S, Zhang X, Lv D, Zhang Q, He Y, Zhang P, et al. A selective BCL-X(L) PROTAC degrader achieves safe and potent antitumor activity. Nature medicine 2019; 25(12):1938-47 doi 10.1038/s41591-019-0668-z.
30. Lu H, Schulze-Gahmen U. Toward understanding the structural basis of cyclin-dependent kinase 6 specific inhibition. J Med Chem 2006; 49(13):3826-31 doi 10.1021/jm0600388.
31. Petzold G, Fischer E S, Thoma N H. Structural basis of lenalidomide-induced CKla degradation by the CRL4CRBN ubiquitin ligase. Nature 2016; 532(7597):127-30 doi 10.1038/nature16979.
32. Matyskiela M E, Lu G, Ito T, Pagarigan B, Lu C-C, Miller K, et al. A novel cereblon modulator recruits GSPT1 to the CRL4CRBN ubiquitin ligase. Nature 2016; 535(7611):252-7 doi 10.1038/nature18611.
33. Guo W—H, Qi X, Yu X, Liu Y, Chung C—I, Bai F, et al. Enhancing intracellular accumulation and target engagement of PROTACs with reversible covalent chemistry. Nature Communications 2020; 11(1):4268 doi 10.1038/s41467-020-17997-6.
34. Bondeson D P, Smith B E, Burslem G M, Buhimschi A D, Hines J, Jaime-Figueroa S, et al. Lessons in PROTAC Design from Selective Degradation with a Promiscuous Warhead. Cell Chem Biol 2018; 25(1):78-87 e5 doi 10.1016/j.chembiol.2017.09.010.
35. Martinez-Chavez A, van Hoppe S, Rosing H, Lebre M C, Tibben M, Beijnen J H, et al. P-glycoprotein Limits Ribociclib Brain Exposure and CYP3A4 Restricts Its Oral Bioavailability. Mol Pharm 2019; 16(9):3842-52 doi 10.1021/acs.molpharmaceut.9b00475.
36. Kollmann K, Heller G, Schneckenleithner C, Warsch W, Scheicher R, Ott R G, et al. A kinase-independent function of CDK6 links the cell cycle to tumor angiogenesis. Cancer cell 2013; 24(2):167-81 doi 10.1016/j.ccr.2013.07.012.
37. Adachi M, Roussel M F, Havenith K, Sherr C J. Features of macrophage differentiation induced by p191NK4d, a specific inhibitor of cyclin D-dependent kinases. Blood 1997; 90(1):126-37.
38. Hirai H, Roussel M F, Kato J Y, Ashmun R A, Sherr C J. Novel INK4 proteins, p19 and p18, are specific inhibitors of the cyclin D-dependent kinases CDK4 and CDK6. Molecular and cellular biology 1995; 15(5):2672-81 doi 10.1128/MCB.15.5.2672.
39. Reynisdottir I, Massague J. The subcellular locations of p15(Ink4b) and p27(Kipl) coordinate their inhibitory interactions with cdk4 and cdk2. Genes & development 1997; 11(4):492-503 doi 10.1101/gad.11.4.492.
40. Yao Z, Gao Y, Su W, Yaeger R, Tao J, Na N, et al. RAF inhibitor PLX8394 selectively disrupts BRAF dimers and RAS-independent BRAF-mutant-driven signaling. Nature medicine 2019; 25(2):284-91 doi 10.1038/s41591-018-0274-5.
41. Bondeson D P, Smith B E, Burslem G M, Buhimschi A D, Hines J, Jaime-Figueroa S, et al. Lessons in PROTAC Design from Selective Degradation with a Promiscuous Warhead. Cell Chem Biol 2018; 25(1):78-87.e5 doi 10.1016/j.chembiol.2017.09.010.
42. Brand M, Jiang B, Bauer S, Donovan K A, Liang Y, Wang E S, et al. Homolog-Selective Degradation as a Strategy to Probe the Function of CDK6 in AML. Cell Chem Biol 2019; 26(2):300-6.e9 doi https://doi.org/10.1016/j.chembiol.2018.11.006.
43. Costa C, Wang Y, Ly A, Hosono Y, Murchie E, Walmsley C S, et al. PTEN Loss Mediates Clinical Cross-Resistance to CDK4/6 and PI3Kalpha Inhibitors in Breast Cancer. Cancer discovery 2020; 10(1):72-85 doi 10.1158/2159-8290.CD-18-0830.
44. Gary J M, Simmons J K, Xu J, Zhang S, Peat T J, Watson N, et al. Hypomorphic mTOR Downregulates CDK6 and Delays Thymic Pre-T LBL Tumorigenesis. Molecular cancer therapeutics 2020; 19(10):2221-32 doi 10.1158/1535-7163.MCT-19-0671.
45. Liu J, Duan Z, Guo W, Zeng L, Wu Y, Chen Y, et al. Targeting the BRD4/FOXO3a/CDK6 axis sensitizes AKT inhibition in luminal breast cancer. Nat Commun 2018; 9(1):5200 doi 10.1038/s41467-018-07258-y.
46. Kato S, Schwaederle M, Daniels G A, Piccioni D, Kesari S, Bazhenova L, et al. Cyclin-dependent kinase pathway aberrations in diverse malignancies: clinical and molecular characteristics. Cell Cycle 2015; 14(8):1252-9 doi 10.1080/15384101.2015.1014149.
47. Taylor-Harding B, Aspuria P J, Agadjanian H, Cheon D J, Mizuno T, Greenberg D, et al. Cyclin E1 and RTK/RAS signaling drive CDK inhibitor resistance via activation of E2F and ETS. Oncotarget 2015; 6(2):696-714 doi 10.18632/oncotarget.2673.
48. Fry D W, Harvey P J, Keller P R, Elliott W L, Meade M, Trachet E, et al. Specific inhibition of cyclin-dependent kinase 4/6 by P D 0332991 and associated antitumor activity in human tumor xenografts. Molecular cancer therapeutics 2004; 3(11):1427-38.
49. Formisano L, Lu Y, Servetto A, Hanker A B, Jansen V M, Bauer J A, et al. Aberrant FGFR signaling mediates resistance to CDK4/6 inhibitors in ER+ breast cancer. Nat Commun 2019; 10(1):1373 doi 10.1038/s41467-019-09068-2.
50. Fellmann C, Hoffmann T, Sridhar V, Hopfgartner B, Muhar M, Roth M, et al. An optimized microRNA backbone for effective single-copy RNAi. Cell Rep 2013; 5(6):1704-13 doi 10.1016/j.celrep.2013.11.020.
51. Toy W, Weir H, Razavi P, Lawson M, Goeppert A U, Mazzola A M, et al. Activating ESR1 Mutations Differentially Affect the Efficacy of E R Antagonists. Cancer Discov 2017; 7(3):277-87 doi 10.1158/2159-8290.CD-15-1523.
52. Li Q, Wang H, Zogopoulos G, Shao Q, Dong K, Lv F, et al. Reg proteins promote acinar-to-ductal metaplasia and act as novel diagnostic and prognostic markers in pancreatic ductal adenocarcinoma. 2016 2016.
53. Kaemmerer D, Peter L, Lupp A, Schulz S, Sanger J, Baum R P, et al. Comparing of IRS and Her2 as immunohistochemical scoring schemes in gastroenteropancreatic neuroendocrine tumors. Int J Clin Exp Pathol 2012; 5(3):187-94.
54. Pettersen E F, Goddard T D, Huang C C, Couch G S, Greenblatt D M, Meng E C, et al. UCSF Chimera—a visualization system for exploratory research and analysis. J Comput Chem 2004; 25(13):1605-12 doi 10.1002/jcc.20084.
55. Allen W J, Balius T E, Mukherjee S, Brozell S R, Moustakas D T, Lang P T, et al. DOCK 6: Impact of new features and current docking performance. J Comput Chem 2015; 36(15):1132-56 doi 10.1002/jcc.23905.
56. Eastman P, Swails J, Chodera J D, McGibbon R T, Zhao Y, Beauchamp K A, et al. OpenMM 7: Rapid development of high performance algorithms for molecular dynamics. PLOS Computational Biology 2017; 13(7):e1005659 doi 10.1371/journal.pcbi.1005659.
57. Jorgensen W L, Chandrasekhar J, Madura J D, Impey R W, Klein M L. Comparison of simple potential functions for simulating liquid water. The Journal of Chemical Physics 1983; 79(2):926-35 doi 10.1063/1.445869.
58. Wang J, Wolf R M, Caldwell J W, Kollman P A, Case D A. Development and testing of a general amber force field. J Comput Chem 2004; 25(9):1157-74 doi 10.1002/jcc.20035.
59. Jakalian A, Jack D B, Bayly C I. Fast, efficient generation of high-quality atomic charges. AM1-BCC model: I I. Parameterization and validation. Journal of Computational Chemistry 2002; 23(16):1623-41 doi 10.1002/jcc.10128.
60. Wang J, Wang W, Kollman P A, Case D A. Automatic atom type and bond type perception in molecular mechanical calculations. Journal of molecular graphics & modelling 2006; 25(2):247-60 doi 10.1016/j.jmgm.2005.12.005.
61. Maier J A, Martinez C, Kasavajhala K, Wickstrom L, Hauser K E, Simmerling C. ff14S B: Improving the Accuracy of Protein Side Chain and Backbone Parameters from ff99S B. Journal of Chemical Theory and Computation 2015; 11(8):3696-713 doi 10.1021/acs.jctc.5b00255.
62. McGibbon R T, Beauchamp K A, Harrigan M P, Klein C, Swails J M, Hernindez C X, et al. MDTraj: A Modern Open Library for the Analysis of Molecular Dynamics Trajectories. Biophysical journal 2015; 109(8):1528-32 doi 10.1016/j.bpj.2015.08.015.
63. R Core Team. R: A language and environment for statistical computing: R Foundation for Statistical Computing; 2014.
64. Ritchie M E, Phipson B, Wu D, Hu Y, Law C W, Shi W, et al. limma powers differential expression analyses for RNA-sequencing and microarray studies. Nucleic acids research 2015; 43(7):e47 doi 10.1093/nar/gkv007.
65. Chandarlapaty S, Sawai A, Scaltriti M, Rodrik-Outmezguine V, Grbovic-Huezo O, Serra V, et al. AKT inhibition relieves feedback suppression of receptor tyrosine kinase expression and activity. Cancer cell 2011; 19(1):58-71 doi 10.1016/j.ccr.2010.10.031.
66. Zehir A, Benayed R, Shah R H, Syed A, Middha S, Kim H R, et al. Mutational landscape of metastatic cancer revealed from prospective clinical sequencing of 10,000 patients. Nature medicine 2017; 23(6):703-13 doi 10.1038/nm.4333.
67. Cheng D T, Mitchell T N, Zehir A, Shah R H, Benayed R, Syed A, et al. Memorial Sloan Kettering-Integrated Mutation Profiling of Actionable Cancer Targets (MSK-IMPACT): A Hybridization Capture-Based Next-Generation Sequencing Clinical Assay for Solid Tumor Molecular Oncology. The Journal of molecular diagnostics: JMD 2015; 17(3):251-64 doi 10.1016/j.jmoldx.2014.12.006.
68. Razavi P, Chang M T, Xu G, Bandlamudi C, Ross D S, Vasan N, et al. The Genomic Landscape of Endocrine-Resistant Advanced Breast Cancers. Cancer cell 2018; 34(3):427-38 e6 doi 10.1016/j.ccell.2018.08.008.
69. Berman H M, Westbrook J, Feng Z, Gilliland G, Bhat T N, Weissig H, et al. The Protein Data Bank. Nucleic Acids Research 2000; 28(1):235-42 doi 10.1093/nar/28.1.235.

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:
A. A compound according to Formula (I)

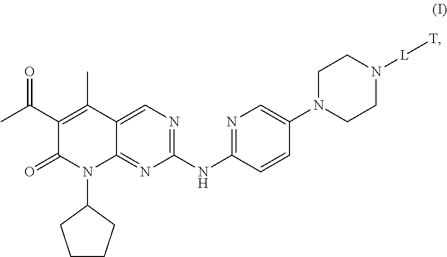

or a pharmaceutically acceptable salt and/or solvate thereof, wherein
   L is selected from the group consisting of

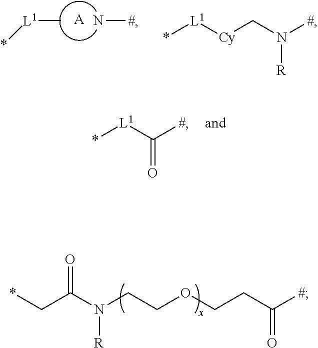

T is selected from the group consisting of

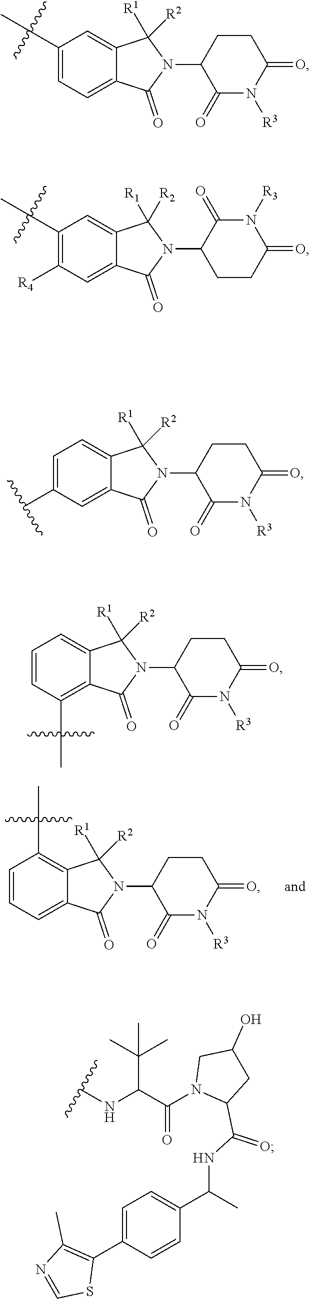

ring A is a 4- to 7-membered N-containing heterocycloalkylene optionally substituted with one or more groups selected from halogen and C₁-C₃ alkyl;
Cy is a C₄-C₆ cycloalkylene optionally substituted with one or more groups selected from halogen and C₁-C₃ alkyl;
R and R³ are each independently H or C₁-C₃ alkyl;
R¹ and R² are both H, or R¹ and R² are taken together to form an oxo (=O) group;
R⁴ is H, halo, C₁-C₄ alkyl, or C₃-C₆ cycloalkyl;
L¹ is a C₁-C₆ alkylene;
* is the linkage site to the nitrogen atom of the piperazine moiety;
# is the linkage site to the T group; and
x is 1, 2, or 3.
B. The compound of Paragraph A, having a structure according to Formula (II)

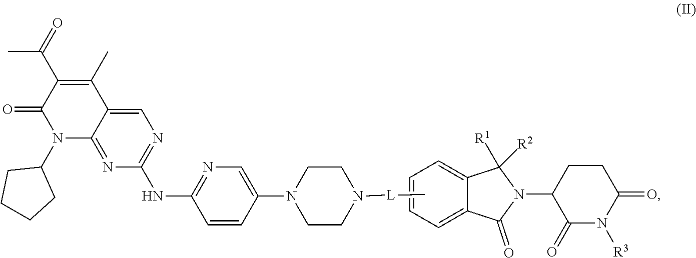

or a pharmaceutically acceptable salt and/or solvate thereof.
C. The compound Paragraph B, wherein L is

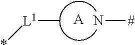

D. The compound Paragraph B, wherein L is

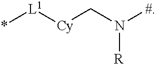

E. The compound Paragraph B, wherein L is

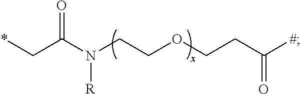

F. The compound of any one of Paragraphs A-C, having a structure according to Formula (IIa)

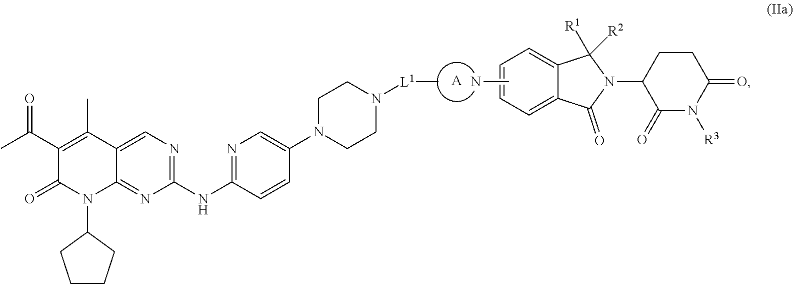

or a pharmaceutically acceptable salt and/or solvate thereof.
G. The compound of Paragraph F, wherein ring A is selected from the group consisting of

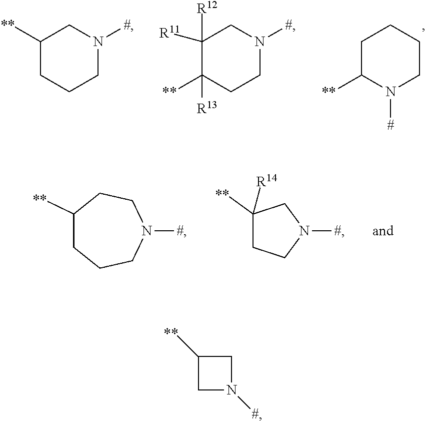

wherein
   R¹³ and R¹⁴ are each independently H, halogen, or C₁-C₃ alkyl;
   is the linkage site to the L¹ group; and
   # is the linkage site to the T group.
H. The compound of Paragraph G, wherein R¹³ is H, F or Me.
I. The compound of Paragraph G, wherein R¹⁴ is H or F.
J. The compound of Paragraph G, wherein R¹¹ is R¹² are each independently H or F.
K. The compound of any one of Paragraphs F-J, wherein L¹ is a methylene.
L. The compound of Paragraph B, wherein L is selected from the group consisting of

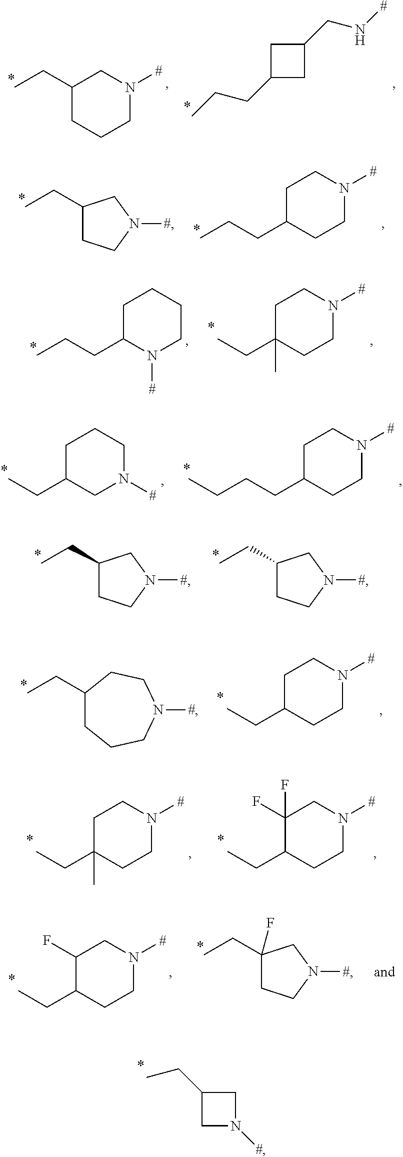

wherein
* is the linkage site to the nitrogen atom of the piperazine moiety; and
# is the linkage site to the T group.
M. The compound of any one of Paragraphs B-L, wherein
   R¹ and R² taken together form an oxo (=O) group; and
   R³ is H.
N. The compound of Paragraph A, having a structure according to Formula (III)

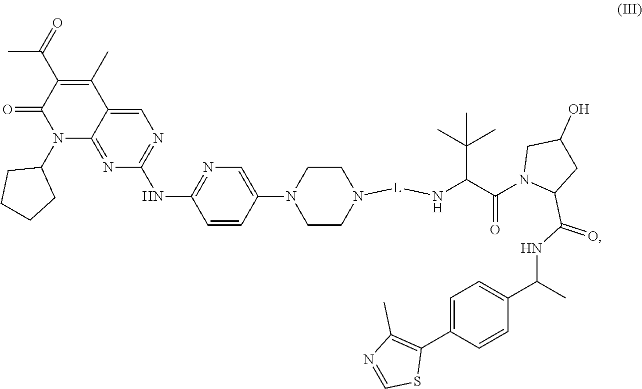

or a pharmaceutically acceptable salt and/or solvate thereof.
O. The compound of Paragraph N, wherein L is

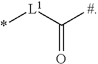

P. The compound of Paragraph N, wherein L is

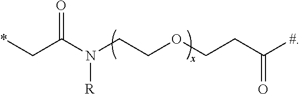

Q. The compound of any one of Paragraphs A, N and O, having a structure according to Formula (IIIa)

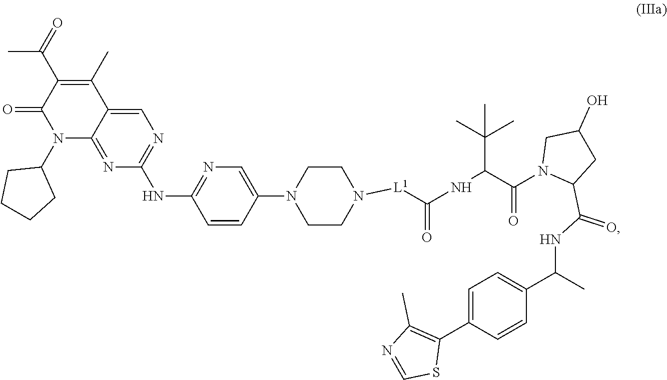

or a pharmaceutically acceptable salt and/or solvate thereof.
R. The compound of Paragraph N, wherein L is selected from the group consisting of
   wherein

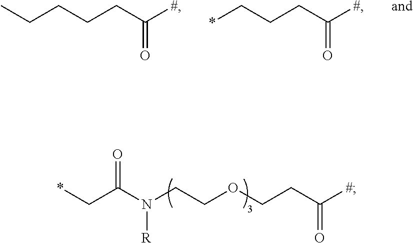

* is the linkage site to the nitrogen atom of the piperazine moiety; and
# is the linkage site to the T group.
S. The compound of Paragraph A, wherein the compound is

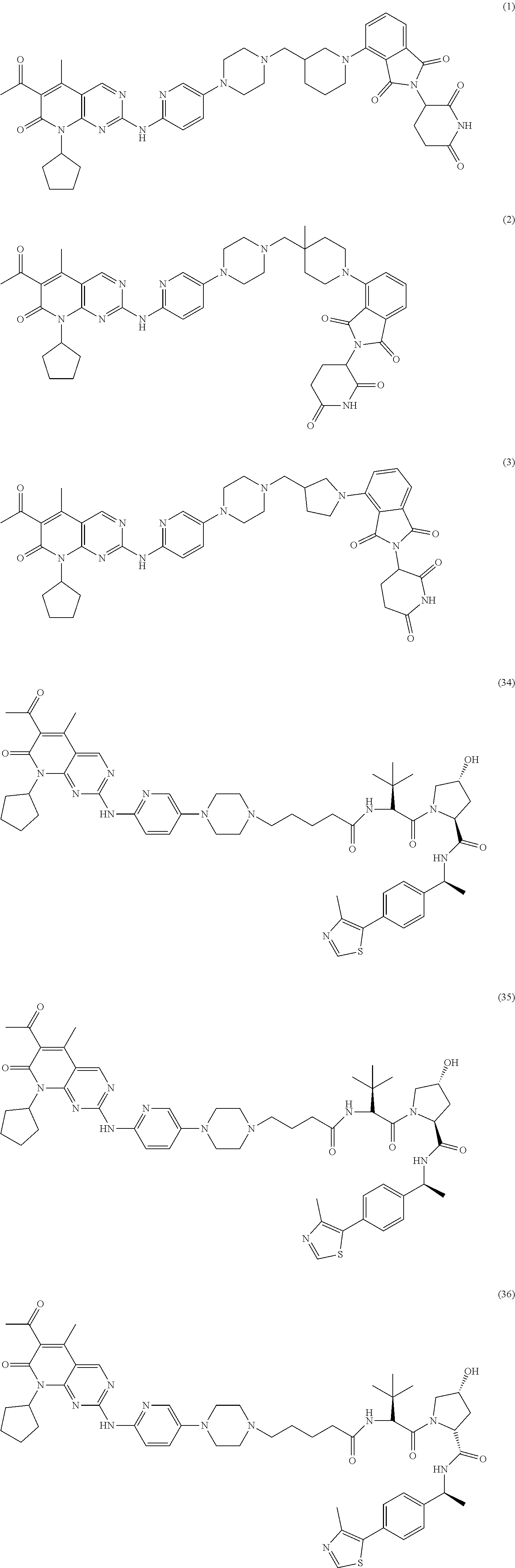

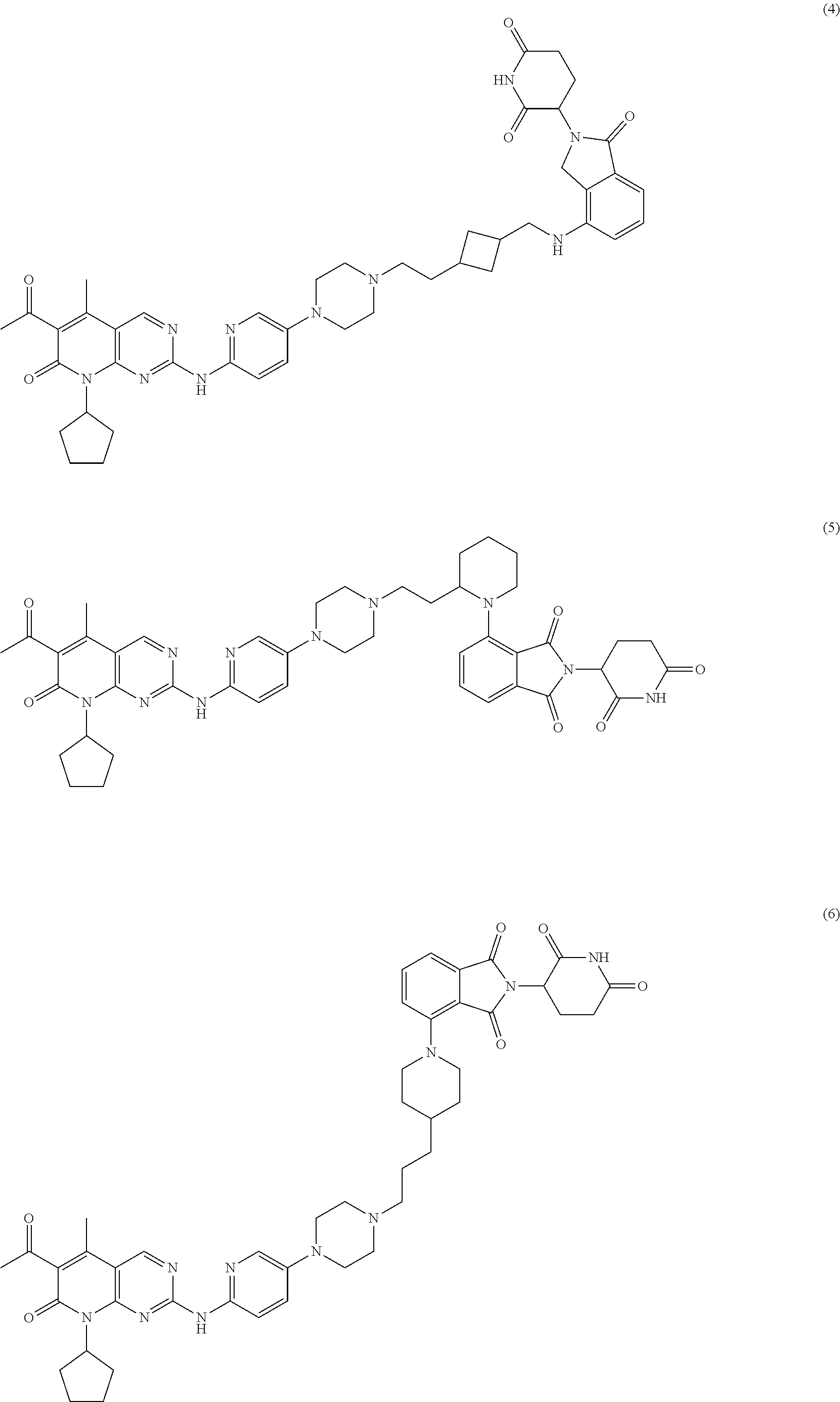 [SMILES: CC(=O)c1c(C)c2cnc(Nc3ccc(N4CCN(CCC5CC(CNc6cccc7c6CN(C6CCC(=O)NC6=O)C7=O)C5)CC4)cn3)nc2n(C2CCCC2)c1=O ; CC(=O)c1c(C)c2cnc(Nc3ccc(N4CCN(CCC5CCCCN5c5cccc6c5C(=O)N(C5CCC(=O)NC5=O)C6=O)CC4)cn3)nc2n(C2CCCC2)c1=O ; CC(=O)c1c(C)c2cnc(Nc3ccc(N4CCN(CCCC5CCN(c6cccc7c6C(=O)N(C6CCC(=O)NC6=O)C7=O)CC5)CC4)cn3)nc2n(C2CCCC2)c1=O]

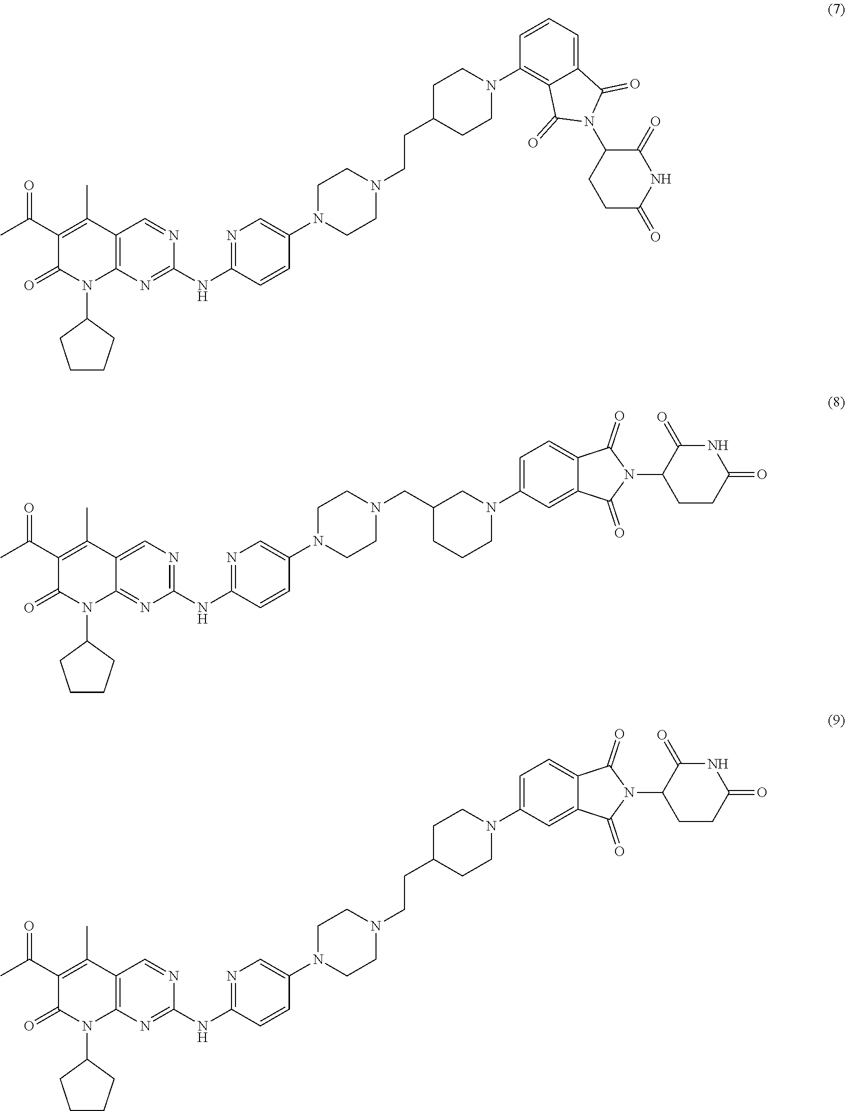 [SMILES: CC(=O)c1c(C)c2cnc(Nc3ccc(N4CCN(CCC5CCN(c6cccc7c6C(=O)N(C6CCC(=O)NC6=O)C7=O)CC5)CC4)cn3)nc2n(C2CCCC2)c1=O ; CC(=O)c1c(C)c2cnc(Nc3ccc(N4CCN(CC5CCCN(c6ccc7c(c6)C(=O)N(C6CCC(=O)NC6=O)C7=O)C5)CC4)cn3)nc2n(C2CCCC2)c1=O ; CC(=O)c1c(C)c2cnc(Nc3ccc(N4CCN(CCC5CCN(c6ccc7c(c6)C(=O)N(C6CCC(=O)NC6=O)C7=O)CC5)CC4)cn3)nc2n(C2CCCC2)c1=O]

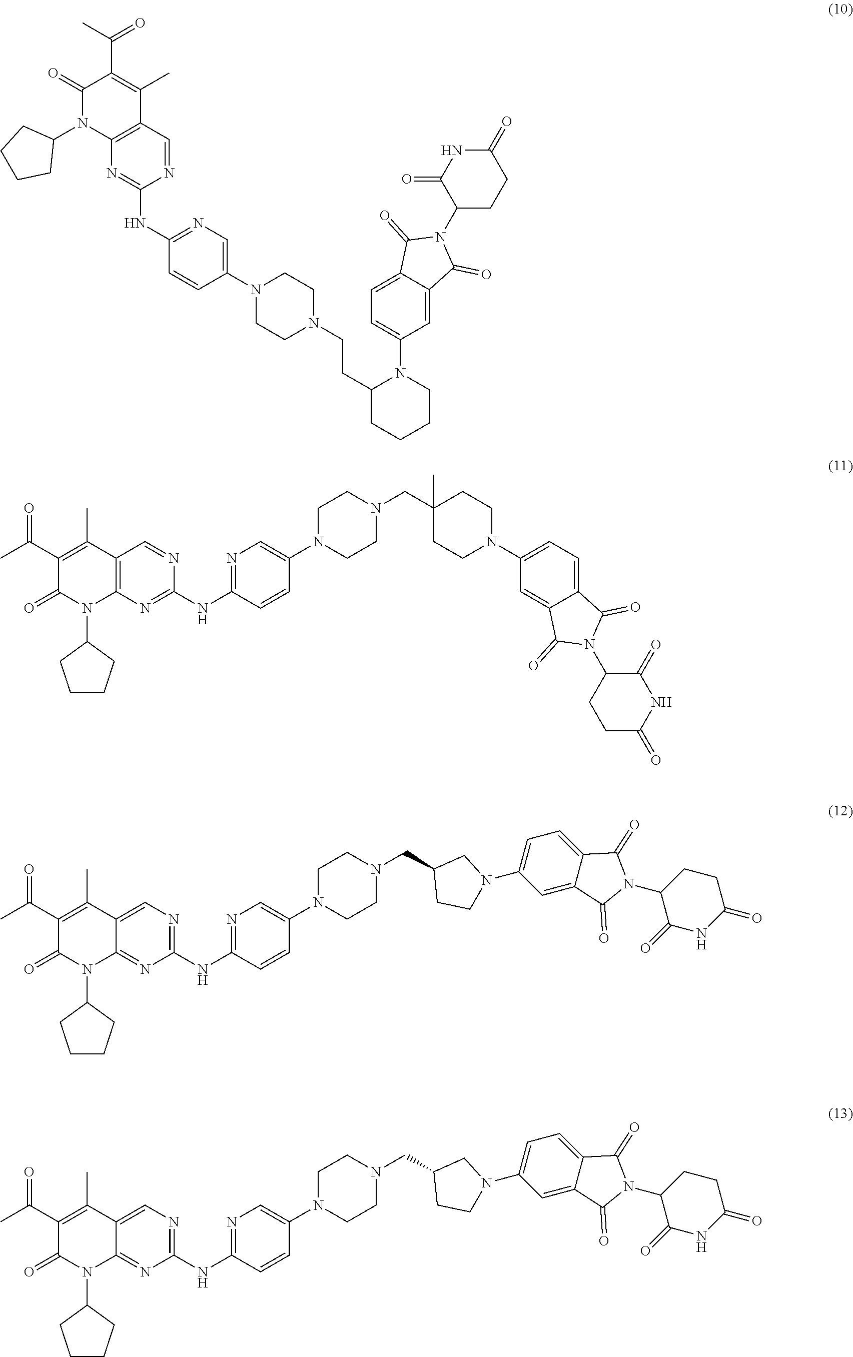 [SMILES: CC(=O)c1c(C)c2cnc(Nc3ccc(N4CCN(CCC5CCCCN5c5ccc6c(c5)C(=O)N(C5CCC(=O)NC5=O)C6=O)CC4)cn3)nc2n(C2CCCC2)c1=O ; CC(=O)c1c(C)c2cnc(Nc3ccc(N4CCN(CC5(C)CCN(c6ccc7c(c6)C(=O)N(C6CCC(=O)NC6=O)C7=O)CC5)CC4)cn3)nc2n(C2CCCC2)c1=O ; CC(=O)c1c(C)c2cnc(Nc3ccc(N4CCN(C[C@@H]5CCN(c6ccc7c(c6)C(=O)N(C6CCC(=O)NC6=O)C7=O)C5)CC4)cn3)nc2n(C2CCCC2)c1=O ; CC(=O)c1c(C)c2cnc(Nc3ccc(N4CCN(C[C@H]5CCN(c6ccc7c(c6)C(=O)N(C6CCC(=O)NC6=O)C7=O)C5)CC4)cn3)nc2n(C2CCCC2)c1=O]

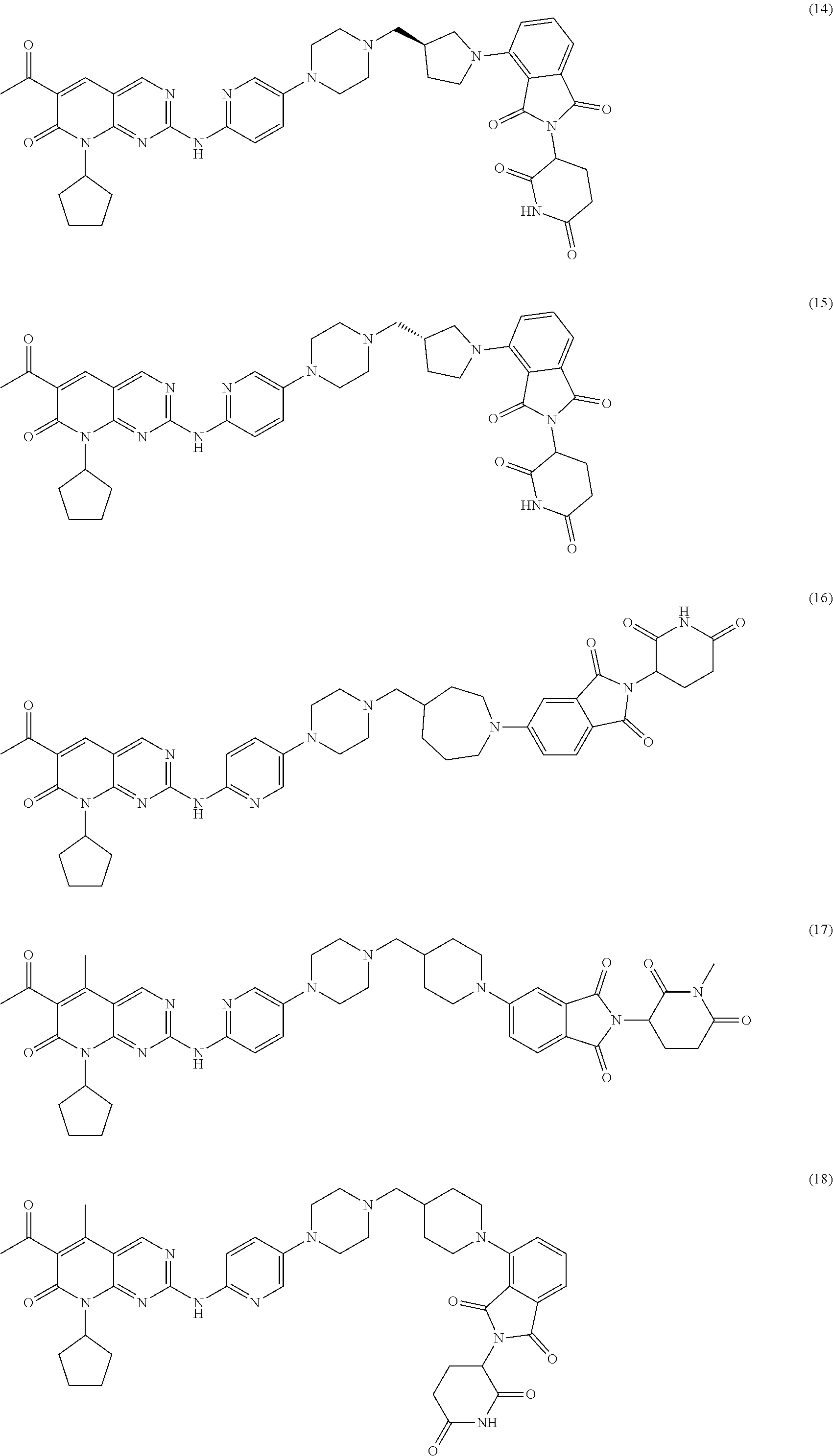

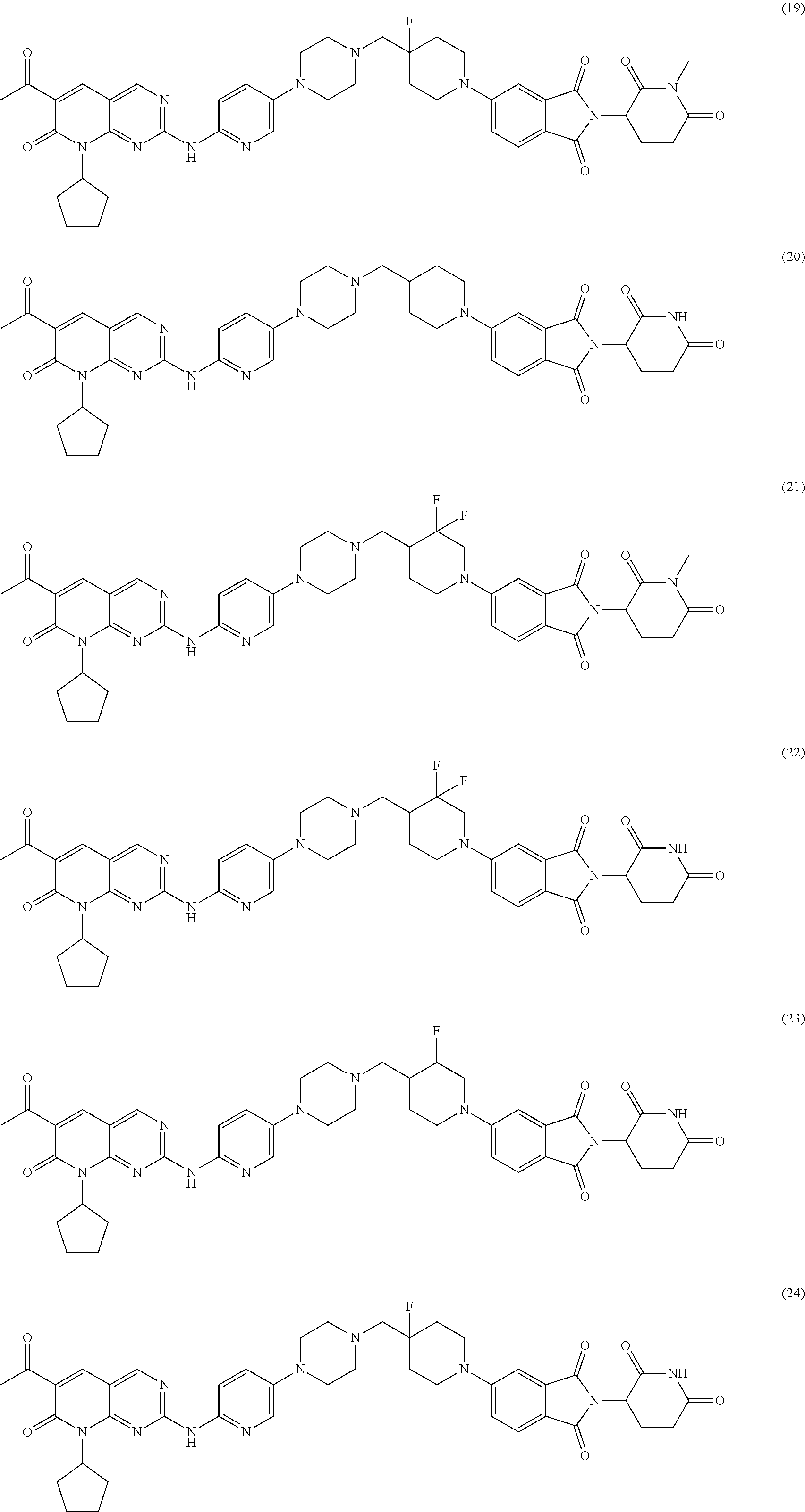

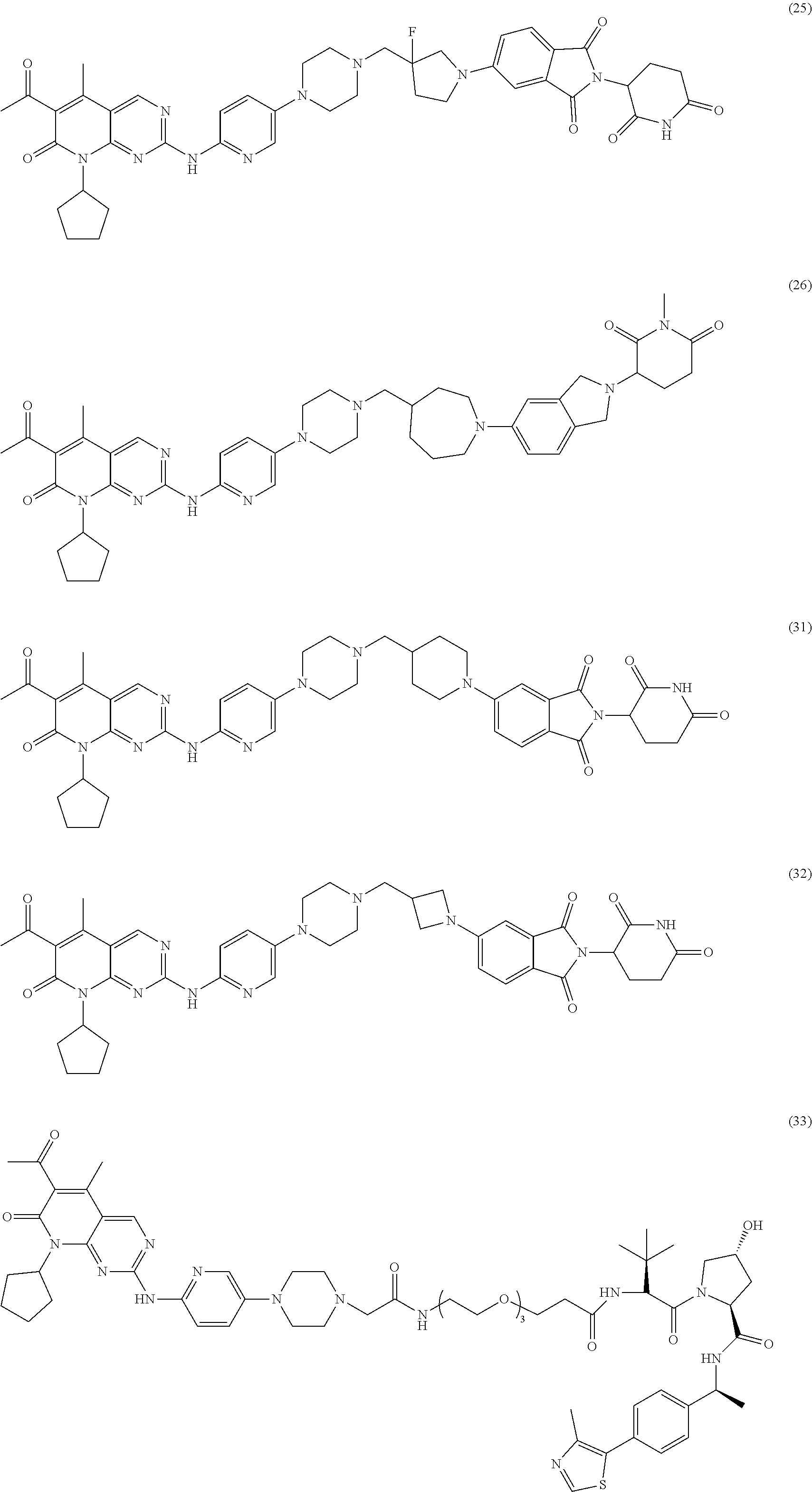

or a pharmaceutically acceptable salt and/or solvate thereof.
T. A pharmaceutical composition comprising a compound according to any one of Paragraphs A-S, or a pharmaceutically acceptable salt and/or solvate thereof.
U. The pharmaceutical composition of Paragraph T, further comprising a pharmaceutically acceptable carrier.
V. A method for inducing degradation of CDK4 and/or CDK6 in a subject in need thereof, the method comprising administering to the subject a therapeutically effective amount of a compound of any one of Paragraphs A-S, or a pharmaceutically acceptable salt and/or solvate thereof.
W. The method of Paragraph V, wherein the method induces degradation of both CDK4 and CDK6.
X. A method for treating a CDK4 and/or CDK6-mediated disorder, disease, or condition in a subject in need thereof, the method comprising administering to the subject a therapeutically effective amount of a compound of any one of Paragraphs A-S, or a pharmaceutically acceptable salt and/or solvate thereof.
Y. The method of Paragraph X, wherein the disorder, disease, or condition is cancer.
Z. The method of Paragraph Y, wherein the cancer comprises breast cancer, prostate cancer, adenocarcinoma, lymphoma, thyroid cancer, lung-NSC (non-small cell lung cancer), rhabdoid tumor, cholangiocarcinoma, small cell lung cancer, bile-duct cancer, acute myeloid leukemia, sarcoma, medulloblastoma, embryonal tumors, and urinary-tract cancer.
AA. The method of Paragraph Y or Z, wherein the cancer is breast cancer.
BB. A method for treating breast cancer in a subject in need thereof, the method comprising administering to the subject a therapeutically effective amount of a compound of any one of Paragraphs A-S, or a pharmaceutically acceptable salt and/or solvate thereof.
Other embodiments are set forth in the following claims, along with the full scope of equivalents to which such claims are entitled.

1. A compound according to Formula (I) 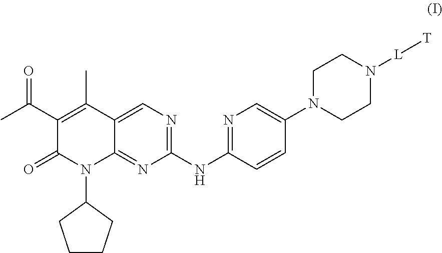
or a pharmaceutically acceptable salt and/or solvate thereof, wherein
L is selected from the group consisting of 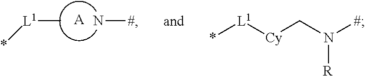
T is selected from the group consisting of 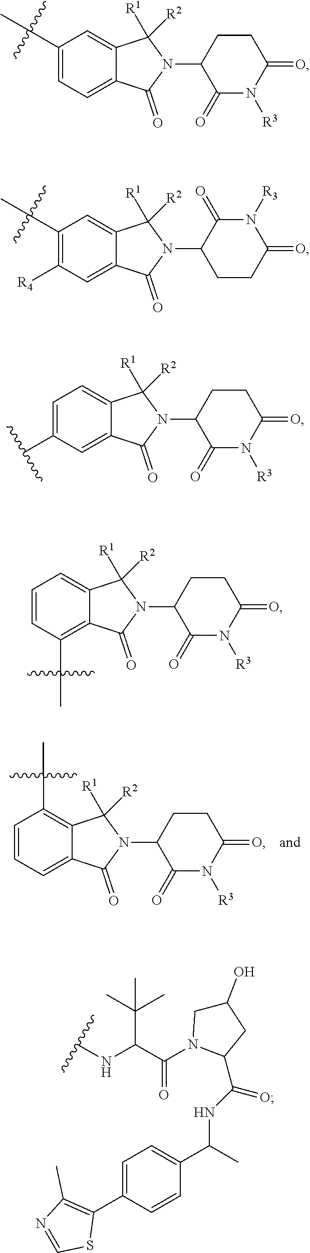
ring A is a 4- to 7-membered N-containing heterocycloalkylene optionally substituted with one or more groups selected from halogen and C₁-C₃ alkyl;
Cy is a C₄-C₆ cycloalkylene optionally substituted with one or more groups selected from halogen and C₁-C₃ alkyl;
R and R³ are each independently H or C₁-C₃ alkyl;
R¹ and R² are taken together to form an oxo (=O) group;
R⁴ is H, halo, C₁-C₄ alkyl, or C₃-C₆ cycloalkyl;
L¹ is a C₁-C₆ alkylene;
* is the linkage site to the nitrogen atom of the piperazine moiety; and
# is the linkage site to the T group.

2. The compound of claim 1, having a structure according to Formula (II) 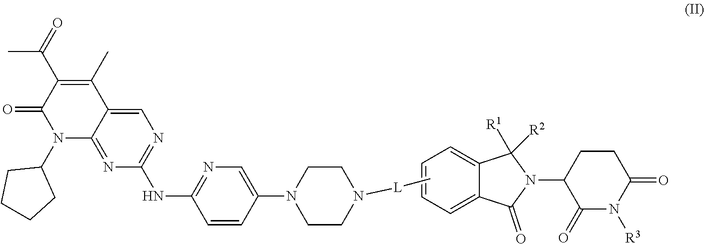
or a pharmaceutically acceptable salt and/or solvate thereof.

3. The compound claim 2, wherein L is

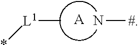

4. The compound claim 2, wherein L is

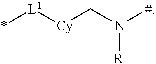

5. The compound of claim 2, having a structure according to Formula (IIa) 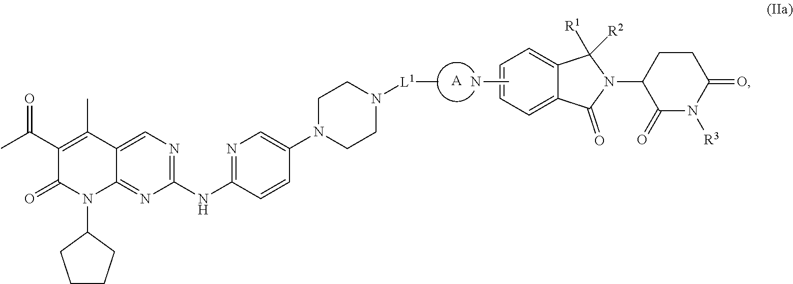
or a pharmaceutically acceptable salt and/or solvate thereof.

6. The compound of claim 5, wherein ring A is selected from the group consisting of 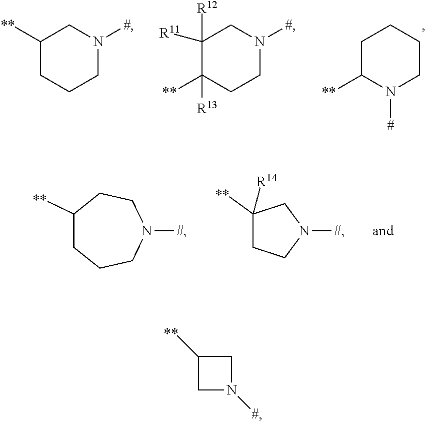
wherein
R¹¹ and R¹² are each independently H or halogen;
R¹³ and R¹⁴ are each independently H, halogen, or C₁-C₃ alkyl;
** is the linkage site to the L¹ group; and
# is the linkage site to the T group.

7. The compound of claim 6, wherein R¹³ is H, F, or Me.

8. The compound of claim 6, wherein R¹⁴ is H or F.

9. The compound of claim 6, wherein R¹¹ and R¹² are each independently H or F.

10. The compound of claim 5, wherein L¹ is a methylene.

11. The compound of claim 2, wherein L is selected from the group consisting of 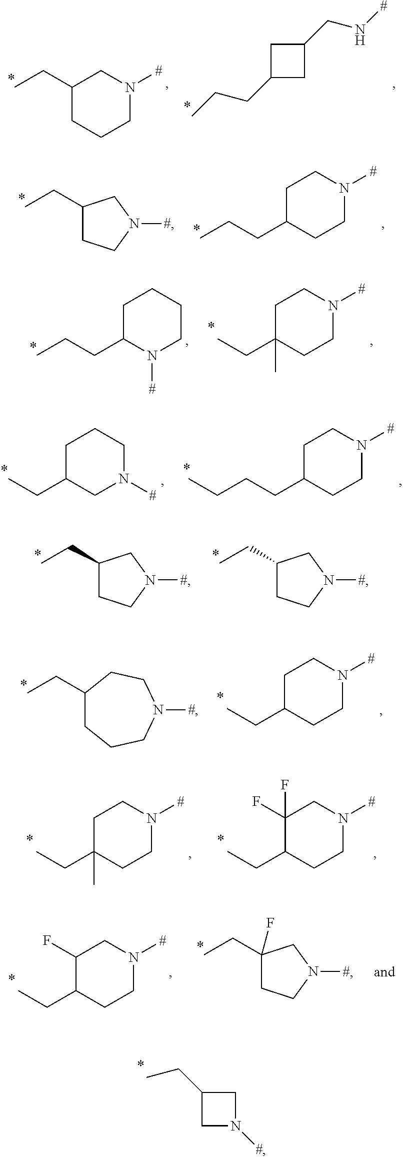
wherein
* is the linkage site to the nitrogen atom of the piperazine moiety; and
# is the linkage site to the T group.

12. The compound of claim 2, wherein
R³ is H.

13. The compound of claim 1, having a structure according to Formula (III) 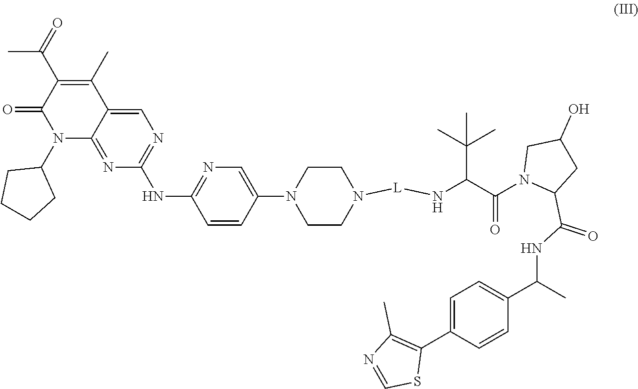
or a pharmaceutically acceptable salt and/or solvate thereof.

14. The compound of claim 1, wherein the compound is 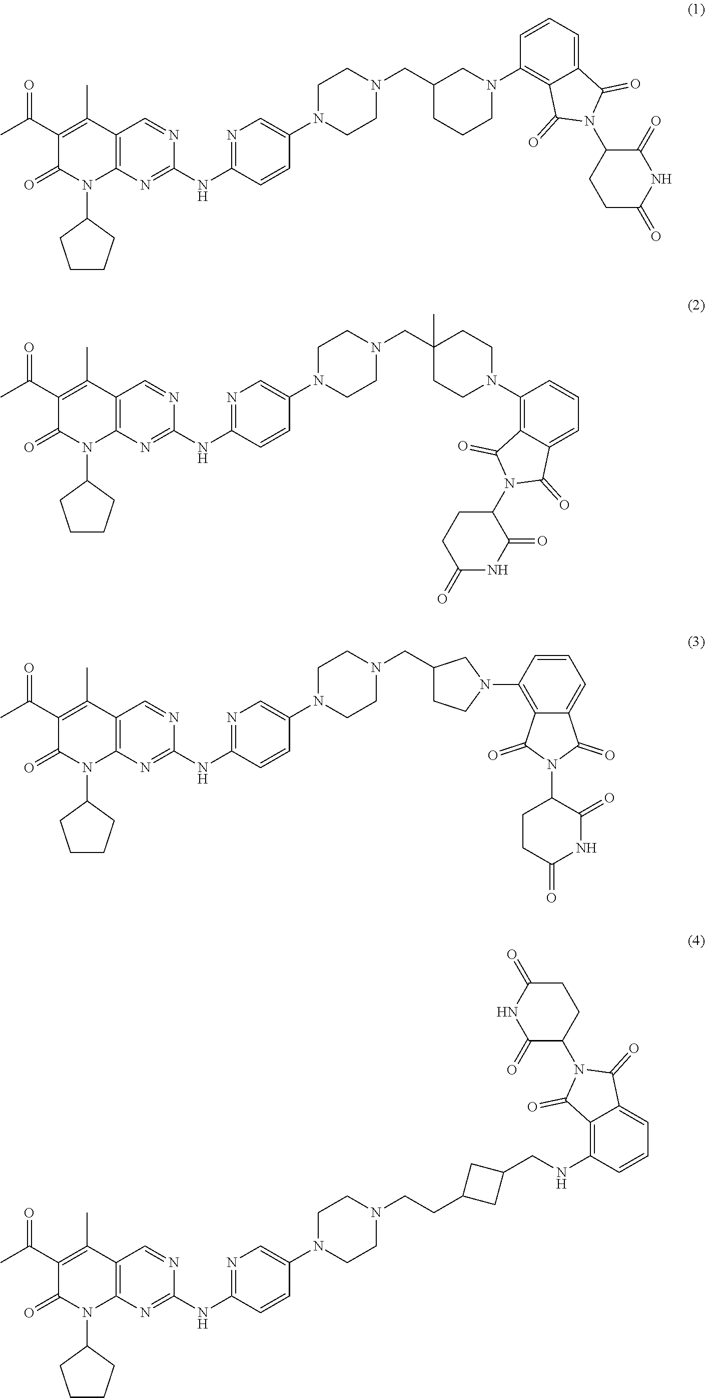 [SMILES: CC(=O)c1c(C)c2cnc(Nc3ccc(N4CCN(CC5CCCN(c6cccc7c6C(=O)N(C6CCC(=O)NC6=O)C7=O)C5)CC4)cn3)nc2n(C2CCCC2)c1=O ; CC(=O)c1c(C)c2cnc(Nc3ccc(N4CCN(CC5(C)CCN(c6cccc7c6C(=O)N(C6CCC(=O)NC6=O)C7=O)CC5)CC4)cn3)nc2n(C2CCCC2)c1=O ; CC(=O)c1c(C)c2cnc(Nc3ccc(N4CCN(CC5CCN(c6cccc7c6C(=O)N(C6CCC(=O)NC6=O)C7=O)C5)CC4)cn3)nc2n(C2CCCC2)c1=O ; CC(=O)c1c(C)c2cnc(Nc3ccc(N4CCN(CCC5CC(CNc6cccc7c6C(=O)N(C6CCC(=O)NC6=O)C7=O)C5)CC4)cn3)nc2n(C2CCCC2)c1=O] 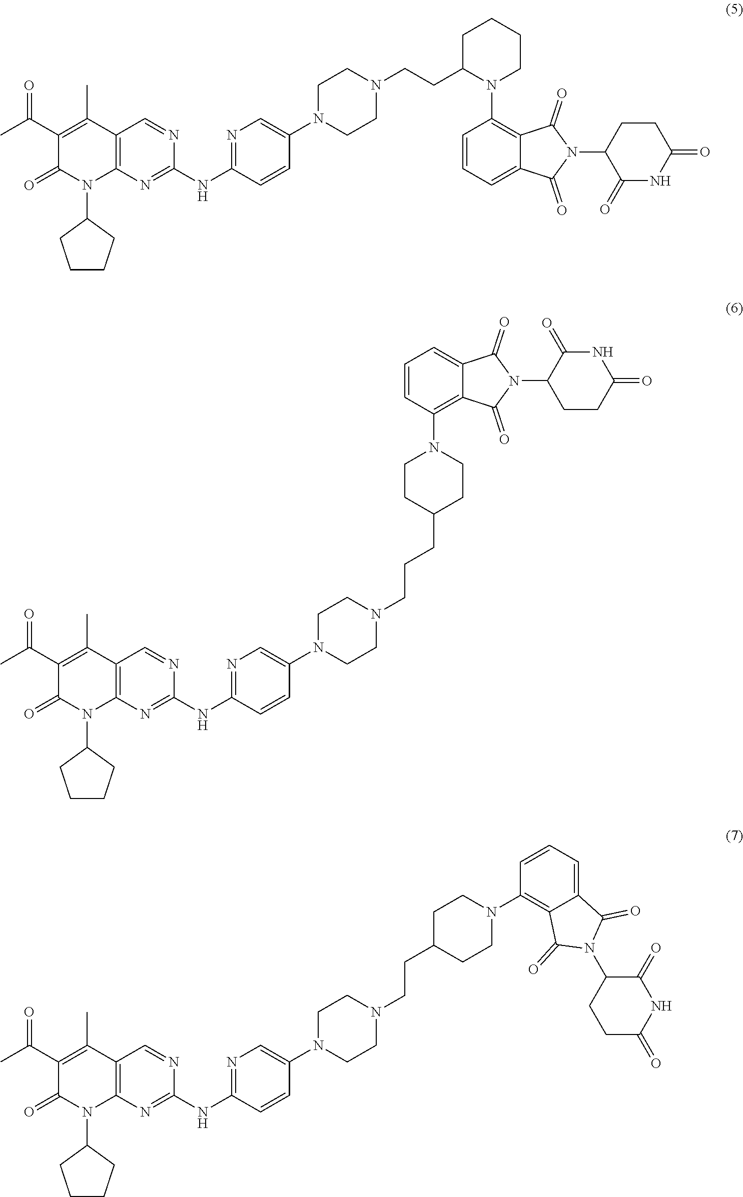 [SMILES: CC(=O)c1c(C)c2cnc(Nc3ccc(N4CCN(CCC5CCCCN5c5cccc6c5C(=O)N(C5CCC(=O)NC5=O)C6=O)CC4)cn3)nc2n(C2CCCC2)c1=O ; CC(=O)c1c(C)c2cnc(Nc3ccc(N4CCN(CCCC5CCN(c6cccc7c6C(=O)N(C6CCC(=O)NC6=O)C7=O)CC5)CC4)cn3)nc2n(C2CCCC2)c1=O ; CC(=O)c1c(C)c2cnc(Nc3ccc(N4CCN(CCC5CCN(c6cccc7c6C(=O)N(C6CCC(=O)NC6=O)C7=O)CC5)CC4)cn3)nc2n(C2CCCC2)c1=O] 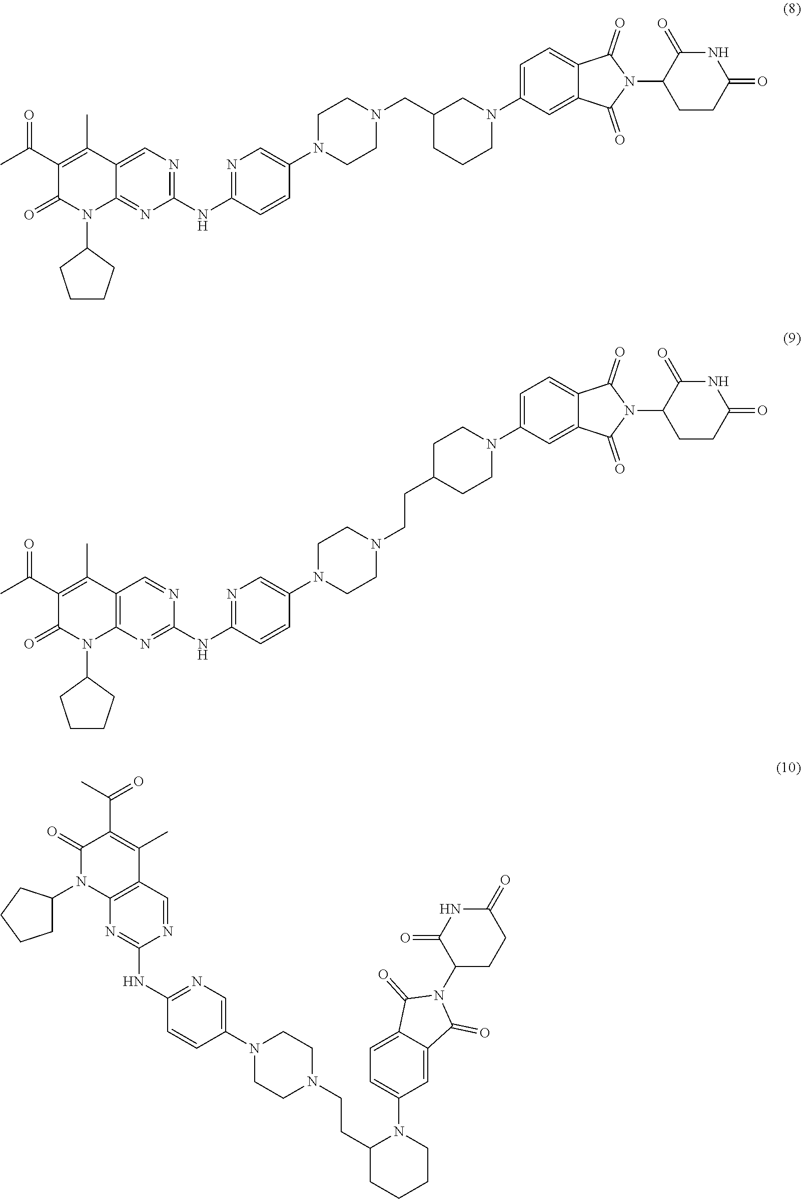 [SMILES: CC(=O)c1c(C)c2cnc(Nc3ccc(N4CCN(CC5CCCN(c6ccc7c(c6)C(=O)N(C6CCC(=O)NC6=O)C7=O)C5)CC4)cn3)nc2n(C2CCCC2)c1=O ; CC(=O)c1c(C)c2cnc(Nc3ccc(N4CCN(CCC5CCN(c6ccc7c(c6)C(=O)N(C6CCC(=O)NC6=O)C7=O)CC5)CC4)cn3)nc2n(C2CCCC2)c1=O ; CC(=O)c1c(C)c2cnc(Nc3ccc(N4CCN(CCC5CCCCN5c5ccc6c(c5)C(=O)N(C5CCC(=O)NC5=O)C6=O)CC4)cn3)nc2n(C2CCCC2)c1=O] 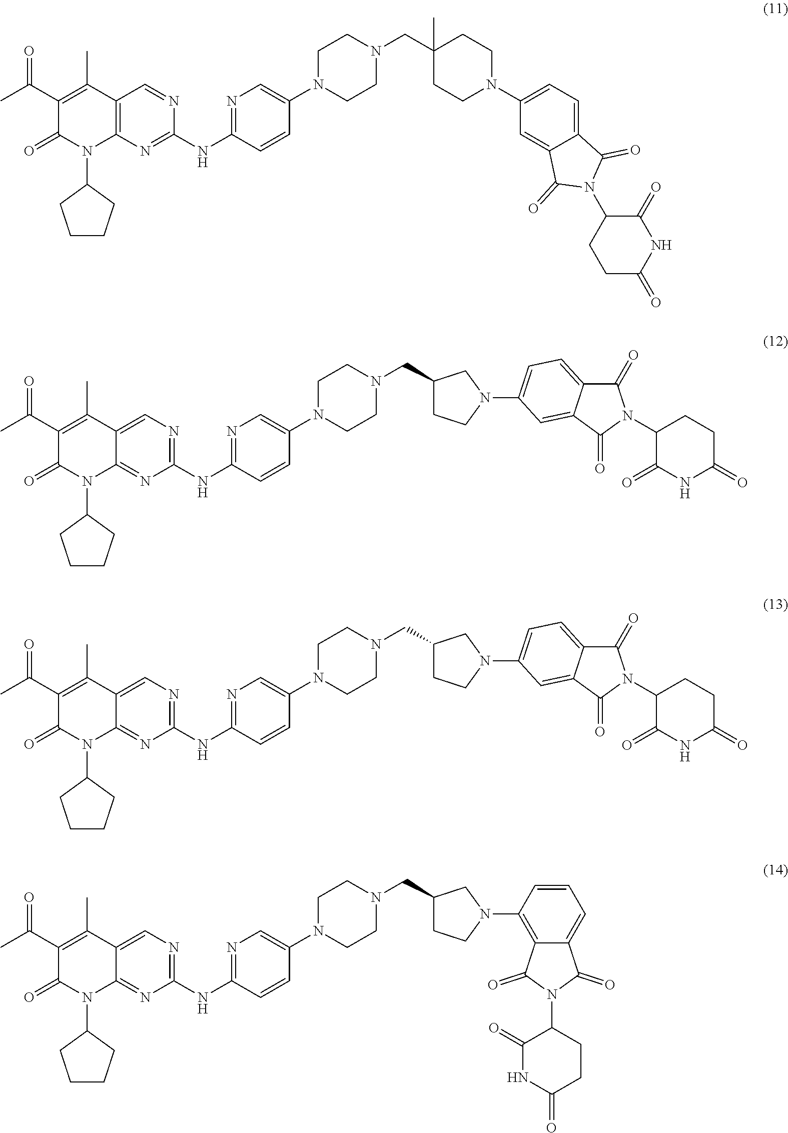 [SMILES: CC(=O)c1c(C)c2cnc(Nc3ccc(N4CCN(CC5(C)CCN(c6ccc7c(c6)C(=O)N(C6CCC(=O)NC6=O)C7=O)CC5)CC4)cn3)nc2n(C2CCCC2)c1=O ; CC(=O)c1c(C)c2cnc(Nc3ccc(N4CCN(C[C@@H]5CCN(c6ccc7c(c6)C(=O)N(C6CCC(=O)NC6=O)C7=O)C5)CC4)cn3)nc2n(C2CCCC2)c1=O ; CC(=O)c1c(C)c2cnc(Nc3ccc(N4CCN(C[C@H]5CCN(c6ccc7c(c6)C(=O)N(C6CCC(=O)NC6=O)C7=O)C5)CC4)cn3)nc2n(C2CCCC2)c1=O ; CC(=O)c1c(C)c2cnc(Nc3ccc(N4CCN(C[C@@H]5CCN(c6cccc7c6C(=O)N(C6CCC(=O)NC6=O)C7=O)C5)CC4)cn3)nc2n(C2CCCC2)c1=O] 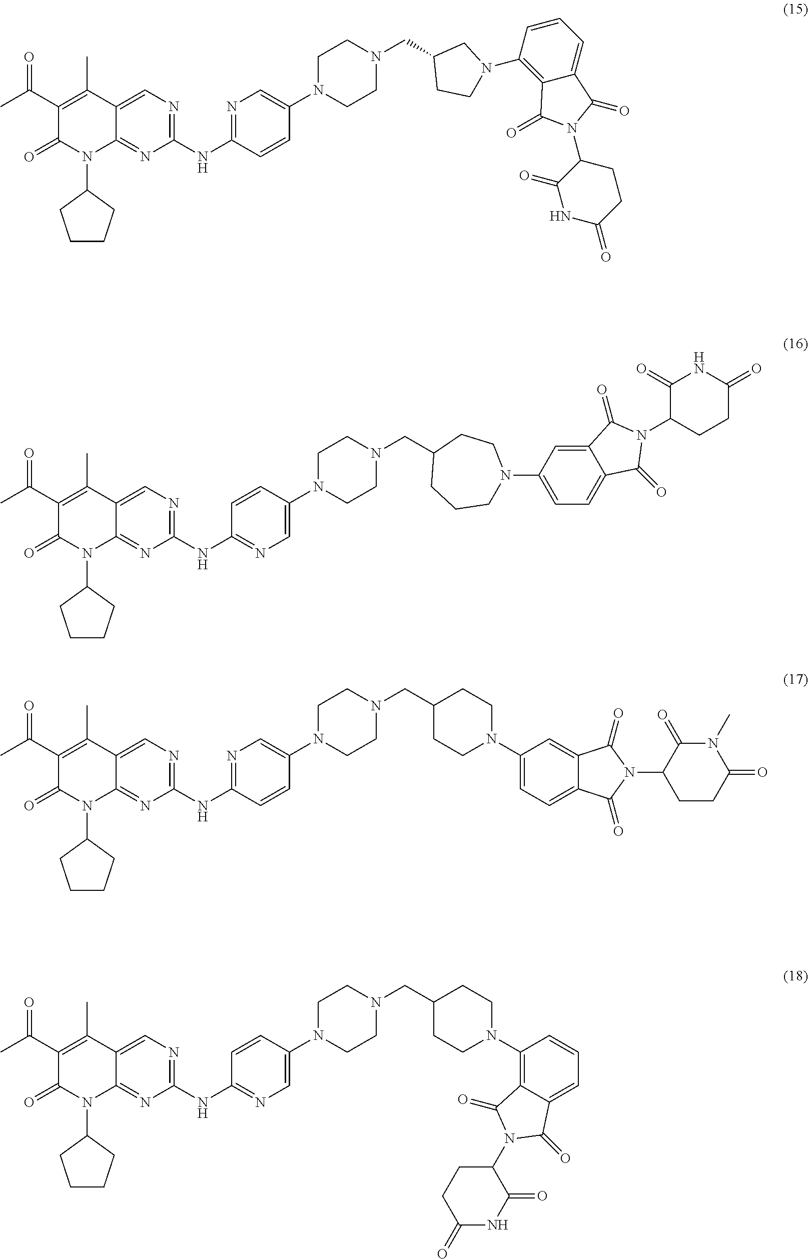 [SMILES: CC(=O)c1c(C)c2cnc(Nc3ccc(N4CCN(C[C@H]5CCN(c6cccc7c6C(=O)N(C6CCC(=O)NC6=O)C7=O)C5)CC4)cn3)nc2n(C2CCCC2)c1=O ; CC(=O)c1c(C)c2cnc(Nc3ccc(N4CCN(CC5CCCN(c6ccc7c(c6)C(=O)N(C6CCC(=O)NC6=O)C7=O)CC5)CC4)cn3)nc2n(C2CCCC2)c1=O ; CC(=O)c1c(C)c2cnc(Nc3ccc(N4CCN(CC5CCN(c6ccc7c(c6)C(=O)N(C6CCC(=O)N(C)C6=O)C7=O)CC5)CC4)cn3)nc2n(C2CCCC2)c1=O ; CC(=O)c1c(C)c2cnc(Nc3ccc(N4CCN(CC5CCN(c6cccc7c6C(=O)N(C6CCC(=O)NC6=O)C7=O)CC5)CC4)cn3)nc2n(C2CCCC2)c1=O] 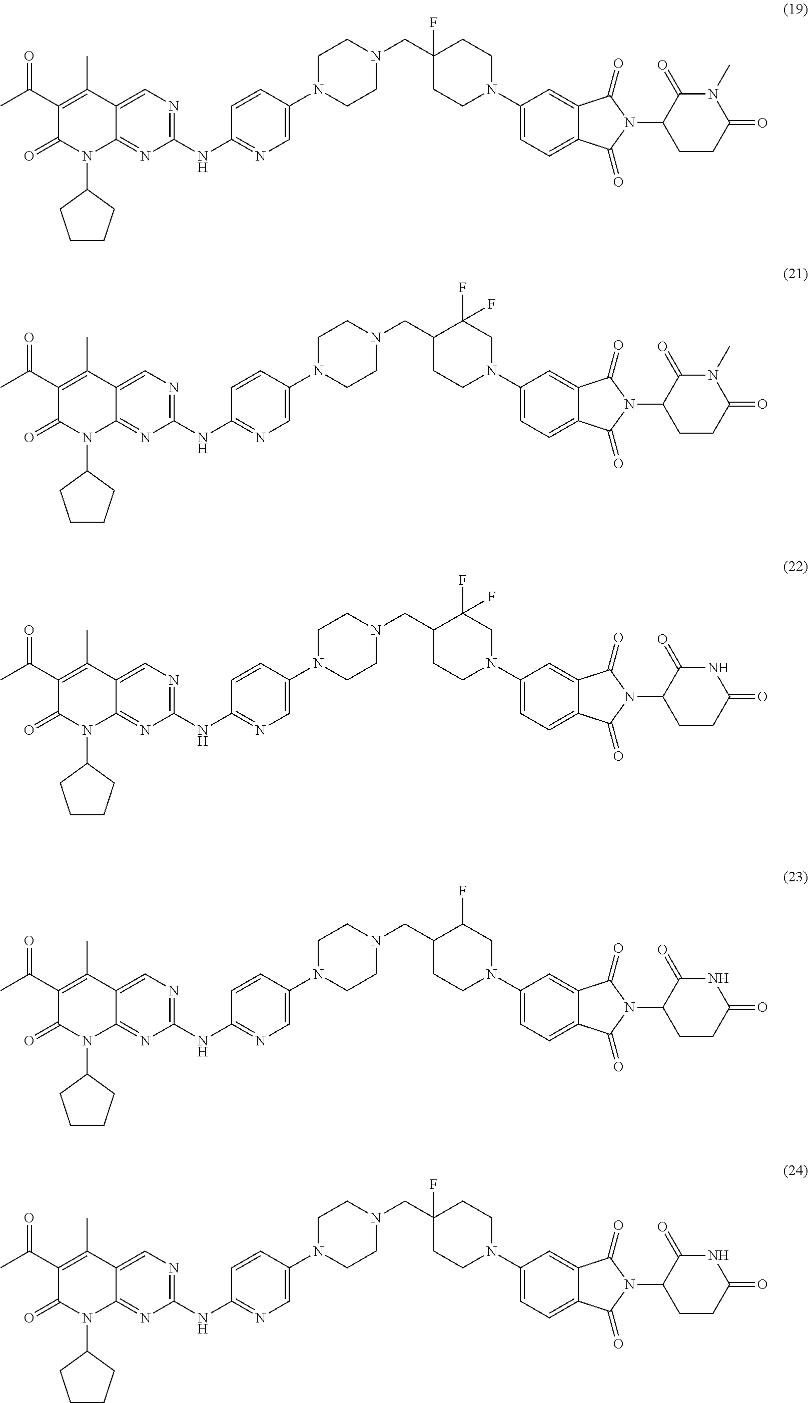 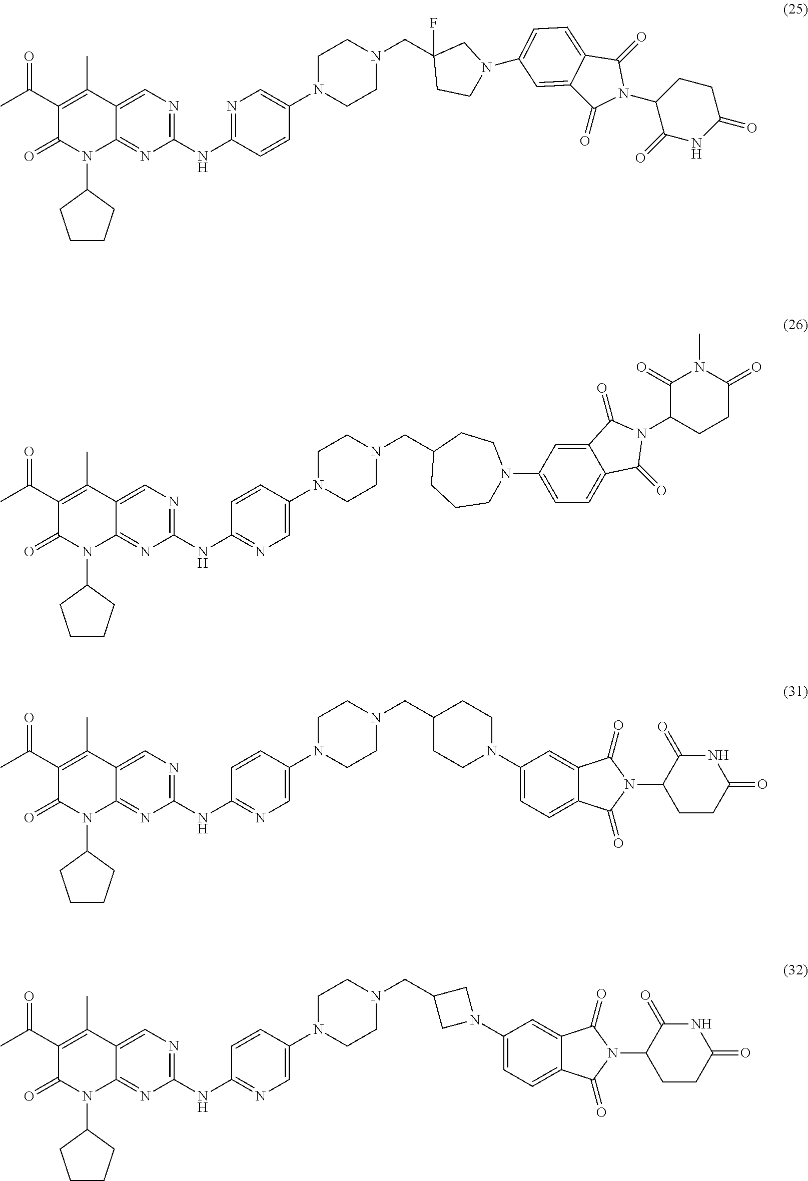 [SMILES: CC(=O)c1c(C)c2cnc(Nc3ccc(N4CCN(CC5(F)CCN(c6ccc7c(c6)C(=O)N(C6CCC(=O)NC6=O)C7=O)C5)CC4)cn3)nc2n(C2CCCC2)c1=O ; CC(=O)c1c(C)c2cnc(Nc3ccc(N4CCN(CC5CCCN(c6ccc7c(c6)C(=O)N(C6CCC(=O)N(C)C6=O)C7=O)CC5)CC4)cn3)nc2n(C2CCCC2)c1=O ; CC(=O)c1c(C)c2cnc(Nc3ccc(N4CCN(CC5CCN(c6ccc7c(c6)C(=O)N(C6CCC(=O)NC6=O)C7=O)CC5)CC4)cn3)nc2n(C2CCCC2)c1=O ; CC(=O)c1c(C)c2cnc(Nc3ccc(N4CCN(CC5CN(c6ccc7c(c6)C(=O)N(C6CCC(=O)NC6=O)C7=O)C5)CC4)cn3)nc2n(C2CCCC2)c1=O]
or a pharmaceutically acceptable salt and/or solvate thereof.

15. A pharmaceutical composition comprising
a compound according to claim 1 or a pharmaceutically acceptable salt and/or solvate thereof; and
a pharmaceutically acceptable carrier.

16. (canceled)

17. A method for inducing degradation of CDK4 and/or CDK6 in a subject in need thereof, the method comprising administering to the subject a therapeutically effective amount of a compound of claim 1 or a pharmaceutically acceptable salt and/or solvate thereof.

18. The method of claim 17, wherein the method induces degradation of both CDK4 and CDK6.

19. A method for treating a CDK4 and/or CDK6-mediated disorder, disease, or condition in a subject in need thereof, the method comprising administering to the subject a therapeutically effective amount of a compound of claim 1 or a pharmaceutically acceptable salt and/or solvate thereof.

20. The method of claim 19, wherein the disorder, disease, or condition is cancer.

21.-22. (canceled)

23. A method for treating breast cancer in a subject in need thereof, the method comprising administering to the subject a therapeutically effective amount of a compound of claim 1 or a pharmaceutically acceptable salt and/or solvate thereof.


SEQUENCE LISTING (9 sequences)

SEQ ID NO: 1 - RNA, 20 nt, synthetic construct
  source 1..20: mol_type = other RNA
cacggtgacgttgtactcgg

SEQ ID NO: 2 - RNA, 20 nt, synthetic construct
  source 1..20: mol_type = other RNA
acggagtcaaccgtttcggg

SEQ ID NO: 3 - RNA, 20 nt, synthetic construct
  source 1..20: mol_type = other RNA
ctccactagtccccgcgccg

SEQ ID NO: 4 - RNA, 20 nt, synthetic construct
  source 1..20: mol_type = other RNA
gaatgacagcgaaaccagtt

SEQ ID NO: 5 - RNA, 20 nt, synthetic construct
  source 1..20: mol_type = other RNA
ttaacatcgaggataatgaa

SEQ ID NO: 6 - RNA, 20 nt, synthetic construct
  source 1..20: mol_type = other RNA
tcatctggattatagaccag

SEQ ID NO: 7 - RNA, 97 nt, synthetic construct
  source 1..97: mol_type = other RNA
tgctgttgacagtgagcgcaggaattataatgcttatctatagtgaagccacagatgtatagataagcattataattcctatgcctactgcctcgga

SEQ ID NO: 8 - RNA, 97 nt, synthetic construct
  source 1..97: mol_type = other RNA
tgctgttgacagtgagcgcaagcgagacacagctatttaatagtgaagccacagatgtattaaatagctgtgtctcgcttttgcctactgcctcgga

SEQ ID NO: 9 - RNA, 97 nt, synthetic construct
  source 1..97: mol_type = other RNA
tgctgttgacagtgagcgctaggttgatcactcataataatagtgaagccacagatgtattattatgagtgatcaacctattgcctactgcctcgga